(19) United States
(12) Patent Application Publication
(10) Pub. No.: US 2024/0342284 A1
(43) Pub. Date: Oct. 17, 2024

(54) SYSTEMS TARGETING PSMA AND CA9

(71) Applicant: Arsenal Biosciences, Inc., South San Francisco, CA (US)

(72) Inventors: Angela Boroughs, San Mateo, CA (US); Levi Gray-Rupp, Philadelphia, PA (US); Samuel A. Williams, Burlingame, CA (US); Zhen Jian Zhou, San Francisco, CA (US); Matthew Drever, Concord, CA (US); Duy Nguyen, San Diego, CA (US); Amy-Jo Casbon, San Francisco, CA (US); Anzhi Yao, San Francisco, CA (US); Thomas Gardner, San Francisco, CA (US)

(21) Appl. No.: 18/593,711

(22) Filed: Mar. 1, 2024

Related U.S. Application Data
(60) Provisional application No. 63/618,233, filed on Jan. 5, 2024.
(60) Provisional application No. 63/613,712, filed on Dec. 21, 2023.
(60) Provisional application No. 63/601,617, filed on Nov. 21, 2023.
(60) Provisional application No. 63/578,854, filed on Aug. 25, 2023.
(60) Provisional application No. 63/495,869, filed on Apr. 13, 2023.
(60) Provisional application No. 63/489,837, filed on Mar. 13, 2023.
(60) Provisional application No. 63/488,386, filed on Mar. 3, 2023.

(51) Int. Cl. A61K 39/00; A61P 35/00; C07K 14/725; C07K 16/30; C07K 16/40; C12N 9/22; C12N 15/11; C12N 15/90

(52) U.S. Cl. CPC A61K 39/464495; A61K 39/4631; A61K 39/464454; A61P 35/00; C07K 14/7051; C07K 16/3069; C07K 16/40; C12N 9/22; C12N 15/11; C12N 15/907; C07K 2317/31; C12N 2310/20

(57) ABSTRACT

Provided herein are antibodies and chimeric priming receptors that bind PSMA and antibodies and chimeric antigen receptors that bind CA9. Also provided are systems of chimeric priming receptors that bind PSMA and chimeric antigen receptors that bind CA9, cells expressing such systems, and methods of use thereof.

### CROSS REFERENCE TO RELATED APPLICATIONS

This application claims the benefit of U.S. Provisional Application No. 63/488,386, filed Mar. 3, 2023; U.S. Provisional Application No. 63/489,837, filed Mar. 13, 2023; U.S. Provisional Application No. 63/495,869, filed Apr. 13, 2023; U.S. Provisional Application No. 63/578,854, filed Aug. 25, 2023; U.S. Provisional Application No. 63/601,617, filed Nov. 21, 2023; U.S. Provisional Application No. 63/613,712, filed Dec. 21, 2023; and U.S. Provisional Application No. 63/618,233, filed Jan. 5, 2024, each of which are hereby incorporated in their entirety by reference.

### SEQUENCE LISTING

The instant application contains a Sequence Listing which is hereby incorporated by reference in its entirety. Said XML copy, created on Feb. 23, 2024, is named ANB-221WO_SL.XML, and is 443,727 bytes in size.

### BACKGROUND

Cancer is a disease characterized by uncontrollable growth of cells. Many approaches to treating cancer have been tried, including drugs and radiation therapies. Recent cancer treatments have sought to use the body's own immune cells to attack cancer cells. One promising approach uses T cells that are taken from a patient and genetically engineered to produce chimeric antigen receptors, or CARs, receptor proteins that give the T cells a new ability to target a specific protein. The receptors are chimeric because they combine antigen-binding and T-cell activating functions into a single receptor.

Immunotherapy using CAR-T cells is promising because the modified T cells have the potential to recognize cancer cells in order to more effectively target and destroy them.

After the T cells are engineered with the CARs, the resulting CAR-T cells are introduced into patients to attack tumor cells. CAR-T cells can be either derived from T cells in a patient's own blood (autologous) or derived from the T cells of another healthy donor (allogeneic). Once CAR-T cells are infused into a patient, they come in contact with their targeted antigen on a cell. The CAR-T cells bind to the antigen and become activated. Upon antigen engagement, CAR T cells can proliferate exponentially, initiate antitumor cytokine production, and target tumor cell killing.

However, there remain some concerns and limitations to CAR T cell-based immunotherapy. Some CAR T cells may engage with normal cells expressing low levels of target antigens, leading to off target toxicity. For example, both primary and metastatic sites of ccRCC are highly vascularized, with the majority of tumor cells expressing elevated levels of carbonic anhydrase IX (CA9). However, CA9 is also expressed in healthy bile ducts and stomach tissue which has led to on-target, off-tumor toxicities in patients treated with constitutive CA9 CAR T cells. Thus, additional therapies that reduce off-target toxicity remain desirable.

### SUMMARY

In one aspect, provided herein are isolated antibodies or antigen binding fragments thereof that binds to Carbonic Anhydrase IX (CA9) (SEQ ID NO: 1), comprising a variable heavy (VH) chain sequence comprising three heavy chain CDR sequences, CDR-H1, CDR-H2, and CDR-H3, of the VH sequences set forth in SEQ ID NOs: 99 or 110, and, optionally, a variable light (VL) chain sequence comprising three light chain CDR sequences, CDR-L1, CDR-L2, and CDR-L3, of the VL sequence set forth in SEQ ID NO: 100, optionally wherein:
i. CDR-H1 comprises the sequence set forth in SEQ ID NO: 101, CDR-H2 comprises the sequence set forth in SEQ ID NO: 102, CDR-H3 comprises the sequence set forth in SEQ ID NO: 103, CDR-L1 comprises the sequence set forth in SEQ ID NO: 104, CDR-L2 comprises the sequence set forth in SEQ ID NO: 105, and CDR-L3 comprises the sequence set forth in SEQ ID NO: 106; or
ii. CDR-H1 comprises the sequence set forth in SEQ ID NO: 111, CDR-H2 comprises the sequence set forth in SEQ ID NO: 112, CDR-H3 comprises the sequence set forth in SEQ ID NO: 113.

In some embodiments, the VH chain sequence comprises the VH sequence set forth in SEQ ID NO: 99, and the VL chain sequence comprises the VL sequence set forth in SEQ ID NO: 100.

In some embodiments, the antibody comprises an scFv.

In some embodiments, the scFv comprises the sequence set forth in SEQ ID NO: 98.

In some embodiments, the VH chain sequence comprises the VH sequence set forth in SEQ ID NO: 110.

In one aspect, provided herein are isolated receptors comprising an extracellular antigen-binding domain that binds to Carbonic Anhydrase IX (CA9) (SEQ ID NO: 1), comprising a variable heavy (VH) chain sequence comprising three heavy chain CDR sequences, CDR-H1, CDR-H2, and CDR-H3, of the VH sequence set forth in SEQ ID NO: 99 or 110, and, optionally, a variable light (VL) chain sequence comprising three light chain CDR sequences, CDR-L1, CDR-L2, and CDR-L3, of the VL sequence set forth in SEQ ID NO: 100, optionally wherein:
i. CDR-H1 comprises the sequence set forth in SEQ ID NO: 101, CDR-H2 comprises the sequence set forth in SEQ ID NO: 102, CDR-H3 comprises the sequence set forth in SEQ ID NO: 103, CDR-L1 comprises the sequence set forth in SEQ ID NO: 104, CDR-L2 comprises the sequence set forth in SEQ ID NO: 105, and CDR-L3 comprises the sequence set forth in SEQ ID NO: 106; or
ii. CDR-H1 comprises the sequence set forth in SEQ ID NO: 111, CDR-H2 comprises the sequence set forth in SEQ ID NO: 112, CDR-H3 comprises the sequence set forth in SEQ ID NO: 113.

In some embodiments, the VH chain sequence comprises the VH sequence set forth in SEQ ID NO: 99, and the VL chain sequence comprises the VL sequence set forth in SEQ ID NO: 100.

In some embodiments, the CAR comprises an scFv.

In some embodiments, the scFv comprises the sequence set forth in SEQ ID NO: 98.

In some embodiments, the VH chain sequence comprises the VH sequence set forth in SEQ ID NO: 110.

In some embodiments, the receptor is a chimeric antigen receptor (CAR) comprising, from N-terminus to C-terminus,
i. the extracellular antigen-binding domain;
ii. a transmembrane domain;
iii. an optional intracellular co-stimulatory domain; and
iv. an intracellular activation domain.

In some embodiments, the CAR further comprises a hinge domain.

In some embodiments, the hinge domain comprises a CD8α, truncated CD8α, or CD28 hinge domain.

In some embodiments, the transmembrane domain comprises a CD8α transmembrane domain or a CD28 transmembrane domain.

In some embodiments, the intracellular co-stimulatory domain comprises a 4-1BB domain.

In some embodiments, the intracellular activation domain comprises a CD3ζ domain.

In some embodiments, the CAR comprises a sequence as set forth in SEQ ID NOs: 108, 115, 250, or 251.

In one aspect, provided herein are isolated antibodies or antigen binding fragments thereof that binds to Prostate-Specific Membrane Antigen (PSMA) (SEQ ID NO: 2), comprising a variable heavy (VH) chain sequence comprising three heavy chain CDR sequences, CDR-H1, CDR-H2, and CDR-H3, of the VH sequences set forth in SEQ ID NOs: 118 or 130, and a variable light (VL) chain sequence comprising three light chain CDR sequences, CDR-L1, CDR-L2, and CDR-L3, of the VL sequences set forth in SEQ ID NOs: 119 or 131, optionally wherein:
i. CDR-H1 comprises the sequence set forth in SEQ ID NO: 120, CDR-H2 comprises the sequence set forth in SEQ ID NO: 121, CDR-H3 comprises the sequence set forth in SEQ ID NO: 122, CDR-L1 comprises the sequence set forth in SEQ ID NO: 123, CDR-L2 comprises the sequence set forth in SEQ ID NO: 124, and CDR-L3 comprises the sequence set forth in SEQ ID NO: 125; or
ii. CDR-H1 comprises the sequence set forth in SEQ ID NO: 132, CDR-H2 comprises the sequence set forth in SEQ ID NO: 133, CDR-H3 comprises the sequence set forth in SEQ ID NO: 134, CDR-L1 comprises the sequence set forth in SEQ ID NO: 135, CDR-L2 comprises the sequence set forth in SEQ ID NO: 136, and CDR-L3 comprises the sequence set forth in SEQ ID NO: 137.

In some embodiments, the VH chain sequence comprises the VH sequence set forth in SEQ ID NO: 118, and the VL chain sequence comprises the VL sequence set forth in SEQ ID NO: 119.

In some embodiments, the VH chain sequence comprises the VH sequence set forth in SEQ ID NO: 130, and the VL chain sequence comprises the VL sequence set forth in SEQ ID NO: 131.

In some embodiments, the antibody comprises an scFv.

In some embodiments, the scFv comprises the sequence set forth in SEQ ID NO: 117 or 129.

In one aspect, provided herein are isolated receptors comprising an extracellular antigen-binding domain that binds to Prostate-Specific Membrane Antigen (PSMA) (SEQ ID NO: 2), comprising a variable heavy (VH) chain sequence comprising three heavy chain CDR sequences, CDR-H1, CDR-H2, and CDR-H3, of the VH sequences set forth in SEQ ID NOs: 118 or 130, and a variable light (VL) chain sequence comprising three light chain CDR sequences, CDR-L1, CDR-L2, and CDR-L3, of the VL sequences set forth in SEQ ID NOs: 119 or 131, optionally wherein:
i. CDR-H1 comprises the sequence set forth in SEQ ID NO: 120, CDR-H2 comprises the sequence set forth in SEQ ID NO: 121, CDR-H3 comprises the sequence set forth in SEQ ID NO: 122, CDR-L1 comprises the sequence set forth in SEQ ID NO: 123, CDR-L2 comprises the sequence set forth in SEQ ID NO: 124, and CDR-L3 comprises the sequence set forth in SEQ ID NO: 125; or
ii. CDR-H1 comprises the sequence set forth in SEQ ID NO: 132, CDR-H2 comprises the sequence set forth in SEQ ID NO: 133, CDR-H3 comprises the sequence set forth in SEQ ID NO: 134, CDR-L1 comprises the sequence set forth in SEQ ID NO: 135, CDR-L2 comprises the sequence set forth in SEQ ID NO: 136, and CDR-L3 comprises the sequence set forth in SEQ ID NO: 137.

In some embodiments, the VH chain sequence comprises the VH sequence set forth in SEQ ID NO: 118, and the VL chain sequence comprises the VL sequence set forth in SEQ ID NO: 119.

In some embodiments, the VH chain sequence comprises the VH sequence set forth in SEQ ID NO: 130, and the VL chain sequence comprises the VL sequence set forth in SEQ ID NO: 131.

In some embodiments, the priming receptor comprises an scFv.

In some embodiments, the scFv comprises the sequence set forth in SEQ ID NO: 117 or 129.

In some embodiments, the receptor is a priming receptor comprising, from N-terminus to C-terminus,
i. the extracellular antigen-binding domain;
ii. a transmembrane domain comprising one or more ligand-inducible proteolytic cleavage sites; and
iii. an intracellular domain comprising a human or humanized transcriptional effector, wherein binding of PSMA by the first extracellular antigen-binding domain results in cleavage at the one or more ligand-inducible proteolytic cleavage sites.

In some embodiments, the priming receptor further comprises a hinge domain positioned between the extracellular antigen-binding domain and the transmembrane domain.

In some embodiments, the hinge domain comprises a CD8α or truncated CD8α hinge domain.

In some embodiments, the hinge domain comprises the sequence as set forth in SEQ ID NO: 85.

In some embodiments, the transmembrane domain comprises a Notch1 transmembrane domain.

In some embodiments, the transmembrane domain comprises the sequence as set forth in SEQ ID NO: 86.

In some embodiments, the intracellular domain comprises an HNF1α/p65 domain or a Gal4/VP64 domain.

In some embodiments, the intracellular domain comprises the sequence as set forth in SEQ ID NO: 88, 89, or 90.

In some embodiments, rein the priming receptor further comprises a stop-transfer-sequence or juxtamembrane domain between the transmembrane domain and the intracellular domain.

In some embodiments, the stop-transfer-sequence or juxtamembrane domain comprises the sequence as set forth in SEQ ID NO: 87.

In some embodiments, the priming receptor comprises a sequence as set forth in SEQ ID NO: 127, 138, 252, or 253.

In one aspect, provided herein are systems comprising:
i. a first chimeric polypeptide comprises a priming receptor comprising a first extracellular antigen-binding domain that specifically binds Prostate-Specific Membrane Antigen (PSMA) (SEQ ID NO: 2);
ii. a second chimeric polypeptide comprising a chimeric antigen receptor (CAR) comprising a second extracellular antigen-binding domain that specifically binds to Carbonic Anhydrase IX (CA9) (SEQ ID NO: 1);
iii. an optional third chimeric polypeptide comprising a synthetic pathway activator (SPA); and
iv. at least one or more nucleic acids comprising a nucleic acid sequence at least 15 nucleotides in length complementary to:
   1. a nucleic acid encoding human Fas Cell Surface Death Receptor (FAS) comprising the sequence set forth in SEQ ID NO: 3; and
   2. a nucleic acid encoding human Transforming Growth factor (TGF)-β Receptor 2 (TGFBR2) comprising the sequence set forth in SEQ ID NO: 4.

In some embodiments, the first extracellular antigen-binding domain comprises a first variable heavy (VH) chain sequence comprising three heavy chain CDR sequences, CDR-H1, CDR-H2, and CDR-H3, of the VH sequences set forth in SEQ ID NOs: 118 or 130, and a first variable light (VL) chain sequence comprising three light chain CDR sequences, CDR-L1, CDR-L2, and CDR-L3, of the VL sequences set forth in SEQ ID NOs: 119 or 131, optionally wherein:
i. CDR-H1 comprises the sequence set forth in SEQ ID NO: 120, CDR-H2 comprises the sequence set forth in SEQ ID NO: 121, CDR-H3 comprises the sequence set forth in SEQ ID NO: 122, CDR-L1 comprises the sequence set forth in SEQ ID NO: 123, CDR-L2 comprises the sequence set forth in SEQ ID NO: 124, and CDR-L3 comprises the sequence set forth in SEQ ID NO: 125; or
ii. CDR-H1 comprises the sequence set forth in SEQ ID NO: 132, CDR-H2 comprises the sequence set forth in SEQ ID NO: 133, CDR-H3 comprises the sequence set forth in SEQ ID NO: 134, CDR-L1 comprises the sequence set forth in SEQ ID NO: 135, CDR-L2 comprises the sequence set forth in SEQ ID NO: 136, and CDR-L3 comprises the sequence set forth in SEQ ID NO: 137.

In some embodiments, the first VH chain sequence comprises the sequence set forth in SEQ ID NO: 118 or 130.

In some embodiments, the first VL chain sequence comprises the sequence set forth in SEQ ID NO: 119 or 131.

In some embodiments, the first extracellular antigen-binding domain comprises the sequence set forth in SEQ ID NO: 117 or 129.

In some embodiments, the second extracellular antigen-binding domain comprises a second variable heavy (VH) chain sequence comprising three heavy chain CDR sequences, CDR-H1, CDR-H2, and CDR-H3, of the VH sequences set forth in SEQ ID NOs: 99 or 110, and, optionally, a second variable light (VL) chain sequence comprising three light chain CDR sequences, CDR-L1, CDR-L2, and CDR-L3, of the VL sequence set forth in SEQ ID NO: 100, optionally wherein:
i. CDR-H1 comprises the sequence set forth in SEQ ID NO: 101, CDR-H2 comprises the sequence set forth in SEQ ID NO: 102, CDR-H3 comprises the sequence set forth in SEQ ID NO: 103, CDR-L1 comprises the sequence set forth in SEQ ID NO: 104, CDR-L2 comprises the sequence set forth in SEQ ID NO: 105, and CDR-L3 comprises the sequence set forth in SEQ ID NO: 106; or
ii. CDR-H1 comprises the sequence set forth in SEQ ID NO: 111, CDR-H2 comprises the sequence set forth in SEQ ID NO: 112, CDR-H3 comprises the sequence set forth in SEQ ID NO: 113.

In some embodiments, the second VH comprises the sequence as set forth in SEQ ID NO: 99 or 110.

In some embodiments, the second VL comprises the sequence set forth in SEQ ID NO: 100.

In some embodiments, the second extracellular domain comprises the sequence set forth in SEQ ID NO: 98 or 110.

In one aspect, provided herein are systems comprising:
i. a first chimeric polypeptide comprises a priming receptor comprising a first extracellular antigen-binding domain that specifically binds Prostate-Specific Membrane Antigen (PSMA) (SEQ ID NO: 2), wherein the first extracellular antigen-binding domain comprises a variable heavy (VH) chain sequence comprising three heavy chain CDR sequences, CDR-H1, CDR-H2, and CDR-H3, of the VH sequences set forth in SEQ ID NOs: 118 or 130, and a variable light (VL) chain sequence comprising three light chain CDR sequences, CDR-L1, CDR-L2, and CDR-L3, of the VL sequences set forth in SEQ ID NOs: 119 or 131, optionally wherein:
ii. CDR-H1 comprises the sequence set forth in SEQ ID NO: 120, CDR-H2 comprises the sequence set forth in SEQ ID NO: 121, CDR-H3 comprises the sequence set forth in SEQ ID NO: 122, CDR-L1 comprises the sequence set forth in SEQ ID NO: 123, CDR-L2 comprises the sequence set forth in SEQ ID NO: 124, and CDR-L3 comprises the sequence set forth in SEQ ID NO: 125; or
iii. CDR-H1 comprises the sequence set forth in SEQ ID NO: 132, CDR-H2 comprises the sequence set forth in SEQ ID NO: 133, CDR-H3 comprises the sequence set forth in SEQ ID NO: 134, CDR-L1 comprises the sequence set forth in SEQ ID NO: 135, CDR-L2 comprises the sequence set forth in SEQ ID NO: 136, and CDR-L3 comprises the sequence set forth in SEQ ID NO: 137.
iv. a second chimeric polypeptide comprises a chimeric antigen receptor (CAR);
v. an optional third chimeric polypeptide comprising a synthetic pathway activator (SPA); and
vi. at least one or more nucleic acids comprising a nucleic acid sequence at least 15 nucleotides in length complementary to:
   1. a nucleic acid encoding human Fas Cell Surface Death Receptor (FAS) comprising the sequence set forth in SEQ ID NO: 3; and
   2. a nucleic acid encoding human Transforming Growth factor (TGF)-β Receptor 2 (TGFBR2) comprising the sequence set forth in SEQ ID NO: 4.

In some embodiments, the VH chain sequence comprises the sequence set forth in SEQ ID NO: 118 or 130.

In some embodiments, the VL chain sequence comprises the sequence set forth in SEQ ID NO: 119 or 131.

In some embodiments, the first extracellular antigen-binding domain comprises the sequence set forth in SEQ ID NO: 117 or 129.

In some embodiments, the CAR comprises a second extracellular antigen-binding domain that specifically binds to Carbonic Anhydrase IX (CA9) (SEQ ID NO: 1).

In some embodiments, the second extracellular antigen-binding domain comprises a variable heavy (VH) chain sequence comprising three heavy chain CDR sequences, CDR-H1, CDR-H2, and CDR-H3, of the VH sequences set forth in SEQ ID NOs: 99 or 110, and optionally, a variable light (VL) chain sequence comprising three light chain CDR sequences, CDR-L1, CDR-L2, and CDR-L3, of the VL sequence set forth in SEQ ID NO: 100, optionally wherein:
i. CDR-H1 comprises the sequence set forth in SEQ ID NO: 101, CDR-H2 comprises the sequence set forth in SEQ ID NO: 102, CDR-H3 comprises the sequence set forth in SEQ ID NO: 103, CDR-L1 comprises the sequence set forth in SEQ ID NO: 104, CDR-L2 comprises the sequence set forth in SEQ ID NO: 105, and CDR-L3 comprises the sequence set forth in SEQ ID NO: 106; or
ii. CDR-H1 comprises the sequence set forth in SEQ ID NO: 111, CDR-H2 comprises the sequence set forth in SEQ ID NO: 112, CDR-H3 comprises the sequence set forth in SEQ ID NO: 113.

In some embodiments, the VH comprises the sequence as set forth in SEQ ID NO: 99 or 110.

In some embodiments, the VL comprises the sequence set forth in SEQ ID NO: 100.

In some embodiments, the second extracellular domain comprises the sequence set forth in SEQ ID NO: 98 or 110.

In one aspect, provided herein are systems comprising:
i. a first chimeric polypeptide comprises a priming receptor, and
ii. a second chimeric polypeptide comprises a chimeric antigen receptor (CAR) comprising a second extracellular antigen-binding domain that specifically binds to Carbonic Anhydrase IX (CA9) (SEQ ID NO: 1), wherein the extracellular antigen-binding domain comprises a single domain antibody comprising a variable heavy (VH) chain sequence comprising three heavy chain CDR sequences, CDR-H1, CDR-H2, and CDR-H3 of the VH sequences set forth in SEQ ID NOs: 99 or 110, and optionally a variable light (VL) chain sequence comprising three light chain CDR sequences, CDR-L1, CDR-L2, and CDR-L3 of the VL sequence set forth in SEQ ID NOs: 110, optionally wherein:
iii. CDR-H1 comprises the sequence set forth in SEQ ID NO: 101, CDR-H2 comprises the sequence set forth in SEQ ID NO: 102, CDR-H3 comprises the sequence set forth in SEQ ID NO: 103, CDR-L1 comprises the sequence set forth in SEQ ID NO: 104, CDR-L2 comprises the sequence set forth in SEQ ID NO: 105, and CDR-L3 comprises the sequence set forth in SEQ ID NO: 106; or
iv. CDR-H1 comprises the sequence set forth in SEQ ID NO: 111, CDR-H2 comprises the sequence set forth in SEQ ID NO: 112, CDR-H3 comprises the sequence set forth in SEQ ID NO: 113;
v. an optional third chimeric polypeptide comprising a synthetic pathway activator (SPA); and
vi. at least one or more nucleic acids comprising a nucleic acid sequence at least 15 nucleotides in length complementary to:
   1. a nucleic acid encoding human Fas Cell Surface Death Receptor (FAS) comprising the sequence set forth in SEQ ID NO: 3; and
   2. a nucleic acid encoding human Transforming Growth factor (TGF)-β Receptor 2 (TGFBR2) comprising the sequence set forth in SEQ ID NO: 4.

In some embodiments, the VH chain sequence comprises the sequence set forth in SEQ ID NO: 99 or 110.

In some embodiments, the VL comprises the sequence set forth in SEQ ID NO: 100.

In some embodiments, the second extracellular domain comprises the sequence set forth in SEQ ID NO: 98 or 110.

In some embodiments, the priming receptor comprises a first extracellular antigen-binding domain that specifically binds to Prostate-Specific Membrane Antigen (PSMA).

In some embodiments, the first extracellular antigen-binding domain comprises a variable heavy (VH) chain sequence comprising three heavy chain CDR sequences, CDR-H1, CDR-H2, and CDR-H3, of the VH sequences set forth in SEQ ID NOs: 118 or 130, and a variable light (VL) chain sequence comprising three light chain CDR sequences, CDR-L1, CDR-L2, and CDR-L3, of the VL sequences set forth in SEQ ID NOs: 119 or 131, optionally wherein:
i. CDR-H1 comprises the sequence set forth in SEQ ID NO: 120, CDR-H2 comprises the sequence set forth in SEQ ID NO: 121, CDR-H3 comprises the sequence set forth in SEQ ID NO: 122, CDR-L1 comprises the sequence set forth in SEQ ID NO: 123, CDR-L2 comprises the sequence set forth in SEQ ID NO: 124, and CDR-L3 comprises the sequence set forth in SEQ ID NO: 125; or
ii. CDR-H1 comprises the sequence set forth in SEQ ID NO: 132, CDR-H2 comprises the sequence set forth in SEQ ID NO: 133, CDR-H3 comprises the sequence set forth in SEQ ID NO: 134, CDR-L1 comprises the sequence set forth in SEQ ID NO: 135, CDR-L2 comprises the sequence set forth in SEQ ID NO: 136, and CDR-L3 comprises the sequence set forth in SEQ ID NO: 137.

In some embodiments, the VH comprises the sequence as set forth in SEQ ID NO: 118 or 130.

In some embodiments, the VL comprises the sequence set forth in SEQ ID NO: 119 or 131.

In some embodiments, the first extracellular domain comprises the sequence set forth in SEQ ID NO: 117 or 129.

In some embodiments, the priming receptor comprises, from N-terminus to C-terminus,
i. the first extracellular antigen-binding domain;
ii. a first transmembrane domain comprising one or more ligand-inducible proteolytic cleavage sites; and
iii. an intracellular domain comprising a human or humanized transcriptional effector, wherein binding of PSMA by the first extracellular antigen-binding domain results in cleavage at the one or more ligand-inducible proteolytic cleavage sites.

In some embodiments, the priming receptor further comprises a first hinge domain positioned between the first extracellular antigen-binding domain and the first transmembrane domain.

In some embodiments, the first hinge domain comprises a CD8α or truncated CD8α hinge domain.

In some embodiments, the first hinge comprises the sequence as set forth in SEQ ID NO: 85.

In some embodiments, the first transmembrane domain comprises a Notch1 transmembrane domain.

In some embodiments, the transmembrane domain comprises the sequence as set forth in SEQ ID NO: 86.

In some embodiments, the intracellular domain comprises an HNF1α/p65 domain or a Gal4/VP64 domain.

In some embodiments, the intracellular domain comprises the sequence as set forth in SEQ ID NO: 88, 89, or 90.

In some embodiments, the priming receptor further comprises a stop-transfer-sequence or juxtamembrane domain between the first transmembrane domain and the intracellular domain.

In some embodiments, the stop-transfer-sequence or juxtamembrane domain comprises the sequence as set forth in SEQ ID NO: 87.

In some embodiments, the priming receptor comprises a sequence as set forth in SEQ ID NO: 127, 138, 252, or 253.

In some embodiments, the CAR comprises, from N-terminus to C-terminus,
i. a second extracellular antigen-binding domain;
ii. a second transmembrane domain;
iii. an intracellular co-stimulatory domain; and
iv. an intracellular activation domain.

In some embodiments, the CAR comprises a second hinge domain.

In some embodiments, the second hinge domain comprises a CD8α or truncated CD8α hinge domain.

In some embodiments, the second transmembrane domain comprises a CD8α transmembrane domain.

In some embodiments, the intracellular co-stimulatory domain comprises a 4-1BB domain.

In some embodiments, the intracellular activation domain comprises a CD3ζ domain.

In some embodiments, the CAR comprises a sequence as set forth in SEQ ID NOs: 108, 115, 250, or 251.

In some embodiments, the priming receptor and the CAR are capable of binding to a same target cell if the target cell expresses PSMA and CA9.

In some embodiments, the at least one or more nucleic acid sequences are at least 16, 17, 18, 19, 20, 21, or 22 nucleotides in length.

In some embodiments, the at least one or more nucleic acid sequences are a short hairpin RNA (shRNA), a small interfering RNA (siRNA), a double stranded RNA (dsRNA), or an antisense oligonucleotide.

In some embodiments, the at least one or more nucleic acid sequences are shRNA.

In some embodiments, the at least one or more nucleic acids comprises a sequence selected from the group consisting of the sequences set forth in SEQ ID NOs: 6-82.

In some embodiments, the nucleic acid sequence complementary to human FAS comprises a sequence selected from the group consisting of the sequences set forth in SEQ ID NOs: 6-20.

In some embodiments, the nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid sequence complementary to human TGFBR2 comprises a sequence selected from the group consisting of the sequences set forth in SEQ ID NOs: 34-82.

In some embodiments, the nucleic acid reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the system comprises at least two nucleic acid sequences complementary to human TGFBR2 selected from the group consisting of the sequences set forth in SEQ ID NOs: 34-82.

In some embodiments, the system comprises at least a first nucleic acid sequence complementary to a nucleic acid encoding human FAS comprising the sequence set forth in SEQ ID NO: 3, a second nucleic acid sequence complementary to a nucleic acid encoding human TGF-β Receptor 2 (TGFBR2) comprising the sequence set forth in SEQ ID NO: 4.

In some embodiments, the nucleic acid sequence is complementary to nucleotides 1126 to 1364 of a nucleic acid encoding human FAS comprising the sequence set forth in SEQ ID NO: 3.

In some embodiments, the first nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid and the second nucleic acid reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the system further comprises a third nucleic acid sequence complementary to mRNA encoding human TGF-β Receptor 2 (TGFBR2) comprising the sequence set forth in SEQ ID NO: 4.

In some embodiments, the third nucleic acid reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, further comprising at least one nucleic acid sequence complementary to a nucleic acid encoding human Protein Tyrosine Phosphatase Non-Receptor Type 2 (PTPN2) comprising the sequence set forth in SEQ ID NO: 5.

In some embodiments, the nucleic acid sequence is complementary to nucleotides 518 to 559 of a nucleic acid encoding human Protein Tyrosine Phosphatase Non-Receptor Type 2 (PTPN2) comprising the sequence set forth in SEQ ID NO: 5.

In some embodiments, the nucleic acid sequence complementary to human PTPN2 comprises a sequence selected from the group consisting of the sequences set forth in SEQ ID NOs: 21-33.

In some embodiments, the nucleic acid reduces expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the SPA is an activator of STAT phosphorylation, optionally STAT1, STAT3, and/or STAT5 phosphorylation.

In some embodiments, the SPA comprises an extracellular domain linked to an intracellular signaling domain.

In some embodiments, the intracellular signaling domain comprises an intracellular signaling region derived from a cytokine receptor.

In some embodiments, the intracellular signaling domain comprises a polypeptide sequence derived from an interleukin receptor.

In some embodiments, the cytokine receptor comprises interleukin-6 signal transducer (IL6ST).

In some embodiments, the SPA comprises the sequence as set forth in SEQ ID NO: 141 or 254.

In some embodiments, the extracellular domain conveys constitutive activity to the intracellular signaling domain.

In some embodiments, the target cell is a human cell.

In some embodiments, the target cell is a cancer cell.

In some embodiments, the cancer cell is a solid cancer cell or a liquid cancer cell.

In some embodiments, the cancer cell is a kidney cell, a colon cell, or a lung cell.

In one aspect, provided herein are nucleic acids comprising a nucleotide sequence encoding the antibodies disclosed herein.

In one aspect, provided herein are nucleic acids comprising a nucleotide sequence encoding the antibodies disclosed herein.

In one aspect, provided herein are nucleic acids comprising a nucleotide sequence encoding the chimeric antigen receptors disclosed herein.

In one aspect, provided herein are nucleic acids comprising a nucleotide sequence encoding the priming receptors disclosed herein.

In one aspect, provided herein are one or more nucleic acids comprising at least one nucleic acid fragment comprising a nucleotide sequence encoding the systems disclosed herein.

In one aspect, provided herein are one or more nucleic acids, wherein the one or more nucleic acids encode:
i. a first chimeric polypeptide comprising a priming receptor comprising a first extracellular antigen-binding domain that specifically binds Prostate-Specific Membrane Antigen (PSMA);
ii. a second chimeric polypeptide comprising a chimeric antigen receptor (CAR) comprising a second extracellular antigen-binding domain that specifically binds to Carbonic Anhydrase IX (CA9);
iii. an optional third chimeric polypeptide comprising a synthetic pathway activator (SPA) and
iv. at least one nucleic acid sequence at least 15 nucleotides in length complementary to:
   1. a nucleic acid encoding human Fas Cell Surface Death Receptor (FAS) comprising the sequence set forth in SEQ ID NO: 3; and
   2. a nucleic acid encoding human Transforming Growth factor (TGF)-β Receptor 2 (TGFBR2) comprising the sequence set forth in SEQ ID NO: 4.

In some embodiments, the nucleic acid sequence is complementary to nucleotides 1126 to 1364 of a nucleic acid encoding human FAS comprising the sequence set forth in SEQ ID NO: 3.

In some embodiments, the first extracellular antigen-binding domain comprises a heavy chain comprising a first variable heavy (VH) chain sequence comprising three heavy chain CDR sequences, CDR-H1, CDR-H2, and CDR-H3, of the VH sequences set forth in SEQ ID NOs: 118 or 130, and a light chain comprising a first variable light (VL) chain sequence comprising three light chain CDR sequences, CDR-L1, CDR-L2, and CDR-L3, of the VL sequences set forth in SEQ ID NOs: 119 or 131, optionally wherein:
i. CDR-H1 comprises the sequence set forth in SEQ ID NO: 120, CDR-H2 comprises the sequence set forth in SEQ ID NO: 121, CDR-H3 comprises the sequence set forth in SEQ ID NO: 122, CDR-L1 comprises the sequence set forth in SEQ ID NO: 123, CDR-L2 comprises the sequence set forth in SEQ ID NO: 124, and CDR-L3 comprises the sequence set forth in SEQ ID NO: 125; or
ii. CDR-H1 comprises the sequence set forth in SEQ ID NO: 132, CDR-H2 comprises the sequence set forth in SEQ ID NO: 133, CDR-H3 comprises the sequence set forth in SEQ ID NO: 134, CDR-L1 comprises the sequence set forth in SEQ ID NO: 135, CDR-L2 comprises the sequence set forth in SEQ ID NO: 136, and CDR-L3 comprises the sequence set forth in SEQ ID NO: 137.

In some embodiments, the first VH chain sequence comprises the VH sequence set forth in SEQ ID NO: 118 or 130, and the first VL chain sequence comprises the VL sequence set forth in SEQ ID NO: 119 or 131.

In some embodiments, the first extracellular antigen-binding domain comprises the sequence set forth in SEQ ID NO: 117 or 129.

In some embodiments, the second extracellular antigen-binding domain comprises a heavy chain comprising a second variable heavy (VH) chain sequence comprising three heavy chain CDR sequences, CDR-H1, CDR-H2, and CDR-H3, of the VH sequences set forth in SEQ ID NOs: 99 or 110, and, optionally, a light chain comprising a second variable light (VL) chain sequence comprising three light chain CDR sequences, CDR-L1, CDR-L2, and CDR-L3, of the VL sequence set forth in SEQ ID NO: 100, optionally wherein
i. CDR-H1 comprises the sequence set forth in SEQ ID NO: 101, CDR-H2 comprises the sequence set forth in SEQ ID NO: 102, CDR-H3 comprises the sequence set forth in SEQ ID NO: 103, CDR-L1 comprises the sequence set forth in SEQ ID NO: 104, CDR-L2 comprises the sequence set forth in SEQ ID NO: 105, and CDR-L3 comprises the sequence set forth in SEQ ID NO: 106; or
ii. CDR-H1 comprises the sequence set forth in SEQ ID NO: 111, CDR-H2 comprises the sequence set forth in SEQ ID NO: 112, CDR-H3 comprises the sequence set forth in SEQ ID NO: 113.

In some embodiments, the second VH comprises the sequence as set forth in SEQ ID NO: 99 or 110.

In some embodiments, the second VL comprises the sequence set forth in SEQ ID NO: 100.

In some embodiments, the second extracellular domain comprises the sequence set forth in SEQ ID NO: 98 or 110.

In some embodiments, the at least one nucleic acid sequences are at least 16, 17, 18, 19, 20, 21, or 22 nucleotides in length.

In some embodiments, the at least one nucleic acid sequences are a short hairpin RNA (shRNA), a small interfering RNA (siRNA), a double stranded RNA (dsRNA), or an antisense oligonucleotide.

In some embodiments, the at least one nucleic acid sequences are shRNA.

In some embodiments, the at least one or more nucleic acids comprises a sequence selected from the group consisting of the sequences set forth in SEQ ID NOs: 6-82.

In some embodiments, the at least one or more nucleic acids comprises a sequence selected from the group consisting of the sequences set forth in SEQ ID NOs: 6-20.

In some embodiments, the at least one or more nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the at least one or more nucleic acid comprises a sequence selected from the group consisting of the sequences set forth in SEQ ID NOs: 34-82.

In some embodiments, the at least one or more nucleic acid reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the at least one or more nucleic acids further comprise at least one nucleic acid sequence complementary to a nucleic acid encoding human Protein Tyrosine Phosphatase Non-Receptor Type 2 (PTPN2) comprising the sequence set forth in SEQ ID NO: 5.

In some embodiments, the nucleic acid sequence is complementary to nucleotides 518 to 559 of a nucleic acid encoding human Protein Tyrosine Phosphatase Non-Receptor Type 2 (PTPN2) comprising the sequence set forth in SEQ ID NO: 5.

In some embodiments, the nucleic acid sequence complementary to human PTPN2 comprises a sequence selected from the group consisting of the sequences set forth in SEQ ID NOs: 21-33.

In some embodiments, the nucleic acid reduces expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid

In some embodiments, the at least one or more nucleic acid sequence is encoded in at least one intron region of the nucleic acid.

In some embodiments, the nucleic acid is selected from the group consisting of the sequences set forth in SEQ ID NOs: 143-147.

In one aspect, provided herein are one or more nucleic acids comprising at least one nucleic acid fragment comprising a nucleotide sequence encoding a priming receptor comprising a first extracellular antigen-binding domain that specifically binds to PSMA, a nucleotide sequence encoding a chimeric antigen receptor comprising an second extracellular antigen-binding domain that specifically binds to CA9, a synthetic pathway activator (SPA); and at least one nucleic acid sequence at least 15 nucleotides in length, wherein the at least one nucleic acid sequence comprises one or more of: (1) a first nucleic acid sequence complementary to a nucleic acid encoding human Fas Cell Surface Death Receptor (FAS) comprising the sequence set forth in SEQ ID NO: 3, and (2) a second nucleic acid sequence complementary to a nucleic acid encoding human Transforming Growth factor (TGF)-0 Receptor 2 (TGFBR2) comprising the sequence set forth in SEQ ID NO: 4.

In some embodiments, the nucleic acid sequence is complementary to nucleotides 1126 to 1364 of a nucleic acid encoding human FAS comprising the sequence set forth in SEQ ID NO: 3.

In one aspect, provided herein are one or more nucleic acids comprising at least one nucleic acid fragment comprising the nucleic acid(s) disclosed herein.

In some embodiments, the nucleic acid comprises two or more nucleic acid fragments.

In some embodiments, the nucleic acid further comprises an inducible promoter operably linked to the nucleotide sequence encoding the CAR.

In some embodiments, the nucleic acid further comprises a constitutive promoter operably linked to the nucleotide sequence encoding the priming receptor.

In some embodiments, the nucleic acid further comprises an inducible promoter operably linked to the nucleotide sequence encoding the chimeric antigen receptor and a constitutive promoter operably linked to the nucleotide sequence encoding the priming receptor.

In some embodiments, the constitutive promoter is EF1α.

In some embodiments, the EF1α promoter comprises a sequence as set forth in SEQ ID NO: 179.

In some embodiments, the inducible promoter comprises one or more Hepatocyte Nuclear Factor 1α (HNF1α) enhancer element(s).

In some embodiments, the inducible promoter the inducible promoter further comprises a YB-TATA promoter sequence.

In some embodiments, the inducible promoter comprises a sequence as set forth in SEQ ID NO: 256.

In some embodiments, the nucleic acid comprises, in a 5′ to 3′ direction,
i. the constitutive promoter;
ii. the nucleotide sequence encoding priming receptor;
iii. the inducible promoter;
iv. the nucleotide sequence encoding chimeric antigen receptor; and
v. the optional nucleotide sequence encoding the SPA.

In some embodiments, the nucleic acid comprises, in a 5′ to 3′ direction,
i. the inducible promoter;
ii. the nucleotide sequence encoding chimeric antigen receptor;
iii. the constitutive promoter; and
iv. the nucleotide sequence encoding priming receptor; and
v. the optional nucleotide sequence encoding the SPA.

In some embodiments, the nucleic acid comprises, in a 5′ to 3′ direction,
i. the first constitutive promoter;
ii. the nucleotide sequence encoding the priming receptor;
iii. the second constitutive promoter;
iv. the nucleotide sequence encoding the at least one nucleic acid complementary to human FAS or human TGFBR2;
v. the inducible promoter;
vi. the optional nucleotide sequence encoding the chimeric antigen receptor; and
vii. the nucleotide sequence encoding the SPA.

In some embodiments, the nucleic acid comprises, in a 5′ to 3′ direction,
i. the first constitutive promoter;
ii. the nucleotide sequence encoding the priming receptor;
iii. the second constitutive promoter;
iv. the nucleotide sequence encoding the first nucleic acid complementary to human FAS or the nucleotide sequence encoding the second nucleic acid complementary to human TGFBR2;
v. the nucleotide sequence encoding the first nucleic acid complementary to human FAS or the nucleotide sequence encoding the second nucleic acid complementary to human TGFBR2;
vi. the inducible promoter;
vii. the nucleotide sequence encoding the chimeric antigen receptor; and
viii. the optional nucleotide sequence encoding the SPA.

In some embodiments, the nucleic acid comprises, in a 5′ to 3′ direction,
i. the inducible promoter;
ii. the nucleotide sequence encoding the chimeric antigen receptor;
iii. the second constitutive promoter;
iv. the nucleotide sequence encoding the first nucleic acid complementary to human FAS or the nucleotide sequence encoding the second nucleic acid complementary to human TGFBR2;
v. the nucleotide sequence encoding the first nucleic acid complementary to human FAS or the nucleotide sequence encoding the second nucleic acid complementary to human TGFBR2;
vi. the first constitutive promoter; and
vii. the nucleotide sequence encoding the priming receptor; and
viii. the optional nucleotide sequence encoding the SPA.

In some embodiments, the nucleic acid is selected from the group consisting of the sequences set forth in SEQ ID NOs: 143-147.

In some embodiments, the nucleic acid further comprises a 5′ homology directed repair arm and a 3′ homology directed repair arm complementary to an insertion site in a host cell chromosome.

In some embodiments, the nucleic acid further comprises a nucleotide sequence encoding a self-excising 2A peptide (P2A).

In some embodiments, the P2A is at the 3′ end of the nucleotide sequence encoding chimeric antigen receptor.

In some embodiments, the P2A is at the 3′ end of the nucleotide sequence encoding priming receptor.

In some embodiments, the nucleic acid further comprises a woodchuck hepatitis virus post-translational regulatory element (WPRE).

In some embodiments, the WPRE is at the 3′ end of the nucleotide sequence encoding chimeric antigen receptor and at the 5′ end of the nucleotide sequence encoding priming receptor or wherein the WPRE is at the 3′ end of the nucleotide sequence encoding priming receptor and at the 5′ end of the nucleotide sequence encoding chimeric antigen receptor.

In some embodiments, the nucleic acid further comprises an SV40 or a human growth hormone (GH1) polyA element.

In some embodiments, the nucleic acid is incorporated into an expression cassette or an expression vector.

In some embodiments, the expression vector is a non-viral vector.

In one aspect, provided herein are vectors comprising the nucleic acids disclosed herein.

In some embodiments, the 5′ and 3′ ends of the nucleic acid comprise nucleotide sequences that are homologous to genomic sequences flanking an insertion site in a genome of a primary cell.

In some embodiments, the insertion site is located at a T Cell Receptor Alpha Constant (TRAC) locus or a genomic safe harbor (GSH) locus.

In one aspect, provided herein are cells comprising:
i. the systems disclosed herein;
ii. at least one nucleic acids disclosed herein; and/or
iii. the vectors disclosed herein.

In some embodiments, the cell is an immune cell.

In one aspect, provided herein are immune cells comprising:
i. the systems disclosed herein;
ii. at least one nucleic acids disclosed herein; and/or
iii. the vectors disclosed herein.

In some embodiments, the immune cell is a primary human immune cell.

In some embodiments, the immune cell is an allogeneic immune cell.

In some embodiments, the immune cell is an autologous immune cell.

In some embodiments, the primary immune cell is a natural killer (NK) cell, a T cell, a CD8+ T cell, a CD4+ T cell, a primary T cell, or a T cell progenitor.

In some embodiments, the primary immune cell is a primary T cell.

In some embodiments, the primary immune cell is a primary human T cell.

In some embodiments, the primary immune cell is virus-free. A primary immune cell comprising at least one nucleic acid comprising a priming receptor comprising a first extracellular antigen-binding domain that specifically binds to PSMA, a chimeric antigen receptor comprising a second extracellular antigen-binding domain that specifically binds to CA9, and optionally a synthetic pathway activator inserted into a target region of the genome of the primary immune cell, and wherein the primary immune cell does not comprise a viral vector for introducing the nucleic acid into the primary immune cell.

In one aspect, provided herein are primary immune cells comprising at least one nucleic acid comprising a priming receptor comprising a first extracellular antigen-binding domain that specifically binds to PSMA, a chimeric antigen receptor comprising a second extracellular antigen-binding domain that specifically binds to CA9, and optionally a synthetic pathway activator inserted into a target region of the genome of the primary immune cell, and wherein the primary immune cell does not comprise a viral vector for introducing the nucleic acid into the primary immune cell.

In one aspect, provided herein are viable, virus-free, primary cells comprising a ribonucleoprotein complex (RNP)-nucleic acid complex, wherein the RNP comprises a nuclease domain and a guide RNA, wherein nucleic acid comprises a priming receptor comprising a first extracellular antigen-binding domain that specifically binds to PSMA and a chimeric antigen receptor comprising a second extracellular antigen-binding domain that specifically binds to CA9, and optionally a synthetic pathway activator and wherein the 5′ and 3′ ends of the nucleic acid comprise nucleotide sequences that are homologous to genomic sequences flanking an insertion site in the genome of the primary cell.

In some embodiments, the cell further comprises at least one nucleic acid sequence at least 15 nucleotides in length, wherein the at least one nucleic acid sequence comprises one or more of a first nucleic acid sequence complementary to a nucleic acid encoding human Fas Cell Surface Death Receptor (FAS) comprising the sequence set forth in SEQ ID NO: 3, and a second nucleic acid sequence complementary a nucleic acid encoding human Transforming Growth factor (TGF)-β Receptor 2 (TGFBR2) comprising the sequence set forth in SEQ ID NO: 4.

In some embodiments, the nucleic acid comprising a sequence with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 143-147.

In one aspect, provided herein are populations of cells comprising a plurality of cells or immune cells disclosed herein.

In one aspect, provided herein are pharmaceutical compositions comprising the cells or immune cell disclosed herein or the population of cells or immune cells disclosed herein, and a pharmaceutically acceptable excipient.

In one aspect, provided herein are pharmaceutical compositions comprising the nucleic acid disclosed herein or the vector disclosed herein, and a pharmaceutically acceptable excipient.

In one aspect, provided herein are methods of editing a cell, comprising: inserting the nucleic acid disclosed herein into an insertion site in the genome of the cell.

In some embodiments, the nucleic acid is introduced to the cell non-virally.

In one aspect, provided herein are methods of editing a cell, comprising:
i. providing a nuclease domain and a guide RNA, wherein the nucleic acid comprises the nucleic acid disclosed herein, and wherein the 5′ and 3′ ends of the nucleic acid comprise nucleotide sequences that are homologous to genomic sequences flanking an insertion site in the genome of the cell;
ii. introducing the nuclease domain and nucleic acid into the cell, wherein the guide RNA specifically hybridizes to a target region of the genome of the cell, and wherein the nuclease domain cleaves the target region to create the insertion site in the genome of the cell; and
iii. editing the cell via insertion of the nucleic acid into the insertion site in the genome of the cell.

In some embodiments, the nuclease domain and nucleic acid are introduced to the cell non-virally.

In one aspect, provided herein are methods of editing an immune cell, comprising:
i. providing a ribonucleoprotein complex (RNP)-nucleic acid complex, wherein the RNP comprises a nuclease domain and a guide RNA, wherein the nucleic acid comprises the nucleic acids disclosed herein, and wherein the 5′ and 3′ ends of the nucleic acid comprise nucleotide sequences that are homologous to genomic sequences flanking an insertion site in the genome of the immune cell;
ii. non-virally introducing the RNP-nucleic acid complex into the immune cell, wherein the guide RNA specifically hybridizes to a target region of the genome of the primary immune cell, and wherein the nuclease domain cleaves the target region to create the insertion site in the genome of the immune cell; and
iii. editing the immune cell via insertion of the nucleic acids disclosed herein into the insertion site in the genome of the immune cell.

In some embodiments, non-virally introducing comprises electroporation.

In some embodiments, the nuclease domain comprises a CRISPR-associated endonuclease (Cas), optionally a Cas9 nuclease.

In some embodiments, the target region of the genome of the cell is a T Cell Receptor Alpha Constant (TRAC) locus or a genomic safe harbor (GSH) locus.

In some embodiments, the nucleic acid is a double-stranded nucleic acid or a single-stranded nucleic acid.

In some embodiments, the nucleic acid is a linear nucleic acid or a circular nucleic acid, optionally wherein the circular nucleic acid is a plasmid.

In some embodiments, the immune cell is a primary human immune cell.

In some embodiments, the immune cell is an autologous immune cell.

In some embodiments, the immune cell is an allogeneic immune cell.

In some embodiments, the immune cell is a natural killer (NK) cell, a T cell, a CD8+ T cell, a CD4+ T cell, a primary T cell, or a T cell progenitor.

In some embodiments, the immune cell is a primary T cell.

In some embodiments, the immune cell is a primary human T cell.

In some embodiments, the immune cell is virus-free.

In some embodiments, further comprising obtaining the immune cell from a patient and introducing the nucleic acid in vitro.

In one aspect, provided herein are methods of treating a disease in a subject comprising administering the immune cells disclosed herein or the pharmaceutical compositions disclosed herein to the subject.

In some embodiments, the disease is cancer.

In some embodiments, the cancer is a solid cancer or a liquid cancer.

In some embodiments, the cancer is kidney cancer, clear cell renal cell carcinoma (ccRcc), colorectal cancer, or lung cancer.

In some embodiments, the administration of the immune cell enhances an immune response in the subject.

In some embodiments, the enhanced immune response is an adaptive immune response.

In some embodiments, the enhanced immune response is an innate immune response.

In some embodiments, the enhanced immune response is an increased expression of at least one cytokine or chemokine.

In some embodiments, the cytokine is interferon-gamma (IFNγ).

In some embodiments, the method further comprises administering an immunotherapy to the subject concurrently with the immune cell or subsequently to the immune cell.

In one aspect, provided herein are methods of inhibiting a target cell in a subject comprising administering the immune cells disclosed herein to the subject, wherein the immune cell inhibits the target cell.

In some embodiments, the target cell expresses PSMA and CA9.

In some embodiments, the target cell is a cancer cell.

In one aspect, provided herein are methods of inducing expression of a chimeric antigen receptor with a priming receptor in a cell or immune cell comprising:
i. obtaining a cell or immune cell comprising:
ii. the systems disclosed herein;
iii. the nucleic acids disclosed herein; and/or
iv. the vectors disclosed herein; and
v. contacting the immune cell with a target cell expressing PSMA and CA9, wherein binding of the priming receptor to PSMA on the target cell induces activation of the priming receptor and expression of the chimeric antigen receptor.

In one aspect, provided herein are methods of modulating the activity of a cell or immune cell comprising:
i. obtaining a cell or immune cell comprising:
ii. the systems disclosed herein;
iii. the nucleic acids disclosed herein; and/or
iv. the vectors disclosed herein; and
v. contacting the cell or immune cell with a target cell expressing PSMA and CA9, wherein binding of the priming receptor to PSMA on the target cell induces activation of the priming receptor and expression of the chimeric antigen receptor and wherein binding of the chimeric antigen receptor to CA9 on the target cell modulates the activity of the immune cell.

In some embodiments, the modulation of the immune cell activity comprises enhancing an immune response.

In some embodiments, the enhanced immune response is an adaptive immune response.

In some embodiments, the enhanced immune response is an innate immune response.

In some embodiments, the immune cell activity is an increased expression of at least one cytokine or chemokine.

In some embodiments, the cytokine is interferon-gamma (IFNγ).

In one aspect, provided herein are methods of treating a disease in a subject comprising:
i. determining or having determined the presence of PSMA-positive (PSMA+) cells from a cancer sample obtained from the subject;
ii. determining or having determined the presence of CA9-positive (CA+) cells from a cancer sample obtained from the subject; and
iii. administering a cell or immune cell disclosed herein or a pharmaceutical composition disclosed herein to the subject.

In some embodiments, the disease is cancer.

In some embodiments, the cancer is a solid cancer or a liquid cancer.

In some embodiments, the cancer is kidney cancer, clear cell renal cell carcinoma (ccRcc), colorectal cancer, or lung cancer.

In some embodiments, the administration of the immune cell enhances an immune response in the subject.

In some embodiments, the enhanced immune response is an adaptive immune response.

In some embodiments, the enhanced immune response is an innate immune response.

In some embodiments, the enhanced immune response is an increased expression of at least one cytokine or chemokine.

In some embodiments, the cytokine is interferon-gamma (IFNγ).

In some embodiments, the method further comprises administering an immunotherapy to the subject concurrently with the immune cell or subsequently to the immune cell.

### BRIEF DESCRIPTION OF THE SEVERAL VIEWS OF THE DRAWINGS

These and other features, aspects, and advantages of the present disclosure will become better understood with regard to the following description, and accompanying drawings, where:

FIG. 1A provides diagram of the various ICT transgene cassettes expressing Logic Gate 1-5 Integrated Circuits (ICs), shRNA, and SPAs. FIG. 1B shows an exemplary insertion cassette encoding a logic gate (priming receptor (primeR) and CAR) and an shRNA module.

FIG. 2A shows binding of the CA9 1 antigen biding fragment to HEK293T-CA9 cells (expressing human CA9) but not to HEK293T parental cells. FIG. 2B shows that the CA9 1 antigen biding fragment did not bind to HEK293T-mCA9 cells (expressing mouse CA9). FIG. 2C shows that CA9 1 antigen biding fragment had no interaction with HEK293T-ASGR1+ cells across a range of concentration up to 5ug/mL. FIG. 2D shows that CA9 1 and CA9 2 both bound to wt CA9-expressing cells (293T-LP81-CA9) as well as cells expressing three different isoforms of CA9: 91-96del CA9, R131Q CA9, and Q326R CA9.

FIG. 3A shows binding of the PSMA 1 antigen biding fragment to HEK293T-PSMA (expressing human PSMA) cells but not HEK293T parental cells. FIG. 3B shows that the PSMA 1 antigen biding fragment did not bind to HEK293T-mPSMA cells (expressing mouse PSMA9). FIG. 3C shows that both PSMA 1 and PSMA 2 scFvs bound to cells expressing the PSMA isoform SNP Y75H and wt PSMA-expressing cells (293T-LP81-PSMA).

FIG. 4A shows that all ICT cells constitutively expressed the priming receptor (PrimeR) construct. FIG. 4B shows that the ICT cells induced CA9 CAR expression when co-cultured with PSMA expressing cell lines.

FIG. 5 shows that inclusion of the shRNA module in ICT cells resulted in lower MFI for both FAS and TGFBR2 in ICT cells expressing the priming receptor-CAR logic gate (PrimeR+) normalized to non-edited cells (PrimeR−).

FIG. 6 shows that ICT cells expressing the SPA exhibit approximately two logs higher pSTAT3 expression when compared to the PrimeR− cells lacking a SPA (EGFRt).

FIG. 7A shows cytotoxicity against parental K562 cells expressing neither CA9 or PSMA, FIG. 7B shows cytotoxicity against K562 cells expressing only CA9, FIG. 7C cytotoxicity against K562 cells expressing only PSMA. FIG. 7D shows cytotoxicity against K562 cells expressing both PSMA and CA9.

FIG. 8 shows IFN-γ production from ICTs expressing Logic Gates 1-5 only in supernatants taken from co-cultures where the target cells expressed either PSMA only (left bar) and target cells with PSMA and CA9 (right bar).

FIG. 9A shows that ICTs expressing Logic Gates 1-5 demonstrated in vitro cytotoxicity against the A498-PSMAmed cell line expressing endogenous CA9 antigen FIG. 9B shows IFNγ, TNFα, GM-CSF, and IL-2 secretion by ICT cells after co-culture with A498-PSMA cells.

FIG. 10 shows that co-culture with HUVEC-PSMA induced expression of the CAR protein on ICT cells and specific killing of CA9+ cells.

FIG. 11A shows that ICT cytotoxicity was not affected by soluble CA9. FIG. 11B shows that ICT cytotoxicity was not affected by soluble PSMA.

FIG. 12A shows the tumor volume post tumor implant in mice treated with ICTs expressing Logic Gates 1-5, RNP or PBS generated from donor 1. FIG. 12B shows the total T cells and expansion of the ICTs on day 12 post inoculation followed by contraction by day 21. FIG. 12C shows total T cells expressing the priming receptor on days 12 and 21. FIG. 12D show the tumor volume post tumor implant in mice treated with ICTs expressing Logic Gates 1-5, RNP or PBS generated from donor 2. FIG. 12E shows the total T cells and expansion of the ICTs on day 12 post inoculation followed by contraction by day 21. FIG. 12F shows total T cells expressing the priming receptor on days 12 and 21.

FIG. 13A shows tumor growth inhibition (TGI) in the single positive CA9-only flank. FIG. 13B shows tumor growth inhibition (TGI) in the dual positive PSMA-CA9 flank.

FIG. 14 shows that TGFBR knockdown protects ICT cells against TGFβ-mediated inhibition.

FIG. 15 shows an exemplary synthetic pathway activator and that synthetic pathway activators increase potency and T stem memory phenotype.

FIG. 16 shows that the ICT was more potent than a conventional CAR-T benchmark.

FIG. 17A shows that ICT cells can be primed during transmigration and can kill CA9+ tumor cells. FIG. 17B shows that T cells can be primed during transmigration and can kill CA9+ tumor cells while unprimed ICT cells demonstrated no cytotoxicity

FIG. 18 shows the co-expression of PSMA and CA9 mRNA in ccRCC with limited overlapping expression in normal tissues

FIG. 19 shows representative images of endothelial cells in ccRCC tumor samples stained with anti-PSMA at different staining intensities with 40× magnification and maximum PSMA intensity according to different ccRCC disease stages.

FIG. 20 shows representative images of endothelial cells in ccRCC tumor samples stained with anti-CA9 at different staining intensities with 40× magnification and percent positivity of CA9 in ccRCC tumor samples according to different ccRCC disease stages.

FIG. 21A provides representative images of ccRCC tumor samples co-stained for PSMA and CA9 with 20× magnification. FIG. 21B shows co-expression of PSMA and CA9 in ccRCC via IHC (n=416). FIG. 21C provides co-positivity measurement of PSMA and CA9 in metastatic specimens.

FIG. 22 provides images of CA9 and PSMA IHC staining in ccRCC samples versus normal adjacent kidney samples.

FIG. 23 shows the percentage of colorectal cancer patient samples, based on the CO1922 tissue microarray (TMA), that exhibited expression of both PSMA and CA9.

FIG. 24 shows the percentage of lung cancer patients, based on the LC819a TMA, that exhibited expression of both PSMA and CA9. The number of patient specimens exhibiting positivity for both PSMA and CA9 is listed as n, with the percentage (%) underneath.

FIG. 25 provides an estimated number of patients that may benefit from treatment with a PSMA primeR/CA9 CAR Logic Gate T cell described herein. The numbers are calculated based on estimated new deaths from the indicated cancer types in 2023 from the American Cancer Society.

FIG. 26 provides a phase 1 study design.

### DETAILED DESCRIPTION

### Definitions

Terms used in the claims and specification are defined as set forth below unless otherwise specified.

As used herein, the term “gene” refers to the basic unit of heredity, consisting of a segment of DNA arranged along a chromosome, which codes for a specific protein or segment of protein. A gene typically includes a promoter, a 5′ untranslated region, one or more coding sequences (exons), optionally introns, and a 3′ untranslated region. The gene may further comprise a terminator, enhancers and/or silencers.

As used herein, the term “locus” refers to a specific, fixed physical location on a chromosome where a gene or genetic marker is located.

The term “safe harbor locus” refers to a locus at which genes or genetic elements can be incorporated without disruption to expression or regulation of adjacent genes. These safe harbor loci are also referred to as safe harbor sites (SHS). As used herein, a safe harbor locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site. Examples of integration sites contemplated are provided in Table D.

As used herein, the term “insert” refers to a nucleotide sequence that is integrated (inserted) at a target locus or safe harbor site. The insert can be used to refer to the genes or genetic elements that are incorporated at the target locus or safe harbor site using, for example, homology-directed repair (HDR) CRISPR/Cas9 genome-editing or other methods for inserting nucleotide sequences into a genomic region known to those of ordinary skill in the art.

The term “inserting” refers to a manipulation of a nucleotide sequence to introduce a non-native sequence. This is done, for example, via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chiroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. The Cas9 nuclease domain can be optimized for efficient activity or enhanced stability in the host cell.

As used herein, the term “Cas9” refers to an RNA-mediated nuclease (e.g., of bacterial or archeal orgin, or derived therefrom). Exemplary RNA-mediated nuclases include the foregoing Cas9 proteins and homologs thereof, and include but are not limited to, CPF1 (See, e.g., Zetsche et al., Cell, Volume 163, Issue 3, p759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA).

As used herein, the phrase “immune cell” is inclusive of all cell types that can give rise to immune cells, including hematopoietic cells such hematopoietic stem cells, pluripotent stem cells, and induced pluripotent stem cells (iPSCs). In some embodiments, the immune cell is a B cell, macrophage, a natural killer (NK) cell, an induced pluripotent stem cell (iPSC), a human pluripotent stem cell (HSPC), a T cell or a T cell progenitor or dendritic cell. In some embodiments, the cell is an innate immune cell.

As used herein, the term “primary” in the context of a primary cell or primary stem cell refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized, e.g., directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used herein, the terms “T lymphocyte” and “T cell” are used interchangeably and refer to cells that have completed maturation in the thymus, and identify certain foreign antigens in the body. The terms also refer to the major leukocyte types that have various roles in the immune system, including activation and deactivation of other immune cells. The T cell can be any T cell such as a cultured T cell, e.g., a primary T cell, or a T cell derived from a cultured T cell line, e.g., a Jurkat, SupT1, etc., or a T cell obtained from a mammal. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. The T cell can be a CD3+ cell. T cells can be CD4⁺, CD8⁺, or CD4⁺ and CD8⁺. The T cell can be any type of T cell, CD4+/CD8+ double positive T cells, CD4+ helper T cells (e.g. Th1 and Th2 cells), CD8+ T cells (e.g. cytotoxic T cells), peripheral Including but not limited to blood mononuclear cells (PBMC), peripheral blood leukocytes (PBL), tumor infiltrating lymphocytes (TIL), memory T cells, naive T cells, regulatory T cells, 76 T cells, etc. It can be any T cell at any stage of development. Additional types of helper T cells include Th3 (Treg) cells, Th17 cells, Th9 cells, or Tfh cells. Additional types of memory T cells include cells such as central memory T cells (Tcm cells), effector memory T cells (Tem cells and TEMRA cells). A T cell can also refer to a genetically modified T cell, such as a T cell that has been modified to express a T cell receptor (TCR) or a chimeric antigen receptor (CAR). T cells can also be differentiated from stem cells or progenitor cells.

“CD4+ T cells” refers to a subset of T cells that express CD4 on their surface and are associated with a cellular immune response. CD4+ T cells are characterized by a post-stimulation secretion profile that can include secretion of cytokines such as IFN-γ, TNF-α, IL-2, IL-4 and IL-10. “CD4” is a 55 kD glycoprotein originally defined as a differentiation antigen on T lymphocytes, but was also found on other cells including monocytes/macrophages. The CD4 antigen is a member of the immunoglobulin superfamily and has been implicated as an associative recognition element in MHC (major histocompatibility complex) class II restricted immune responses. On T lymphocytes, the CD4 antigen defines a helper/inducer subset.

“CD8+ T cells” refers to a subset of T cells that express CD8 on their surface, are MHC class I restricted, and function as cytotoxic T cells. The “CD8” molecule is a differentiation antigen present on thymocytes, as well as on cytotoxic and suppressor T lymphocytes. The CD8 antigen is a member of the immunoglobulin superfamily and is an associative recognition element in major histocompatibility complex class I restriction interactions.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes.

As used herein, the term “construct” refers to a complex of molecules, including macromolecules or polynucleotides.

As used herein, the term “integration” refers to the process of stably inserting one or more nucleotides of a construct into the cell genome, i.e., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

As used herein, the term “exogenous” refers to a molecule or activity that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

The term “heterologous” refers to a nucleic acid or polypeptide sequence or domain which is not native to a flanking sequence, e.g., wherein the heterologous sequence is not found in nature coupled to the nucleic acid or polypeptide sequences occurring at one or both ends.

The term “homologous” refers to a nucleic acid or polypeptide sequence or domain which is native to a flanking sequence, e.g., wherein the homologous sequence is found in nature coupled to the nucleic acid or polypeptide sequences occurring at one or both ends.

As used herein, a “polynucleotide donor construct” refers to a nucleotide sequence (e.g. DNA sequence) that is genetically inserted into a polynucleotide and is exogenous to that polynucleotide. The polynucleotide donor construct is transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can be a miRNA, shRNA, natural polypeptide (i.e., a naturally occurring polypeptide) or fragment thereof or a variant polypeptide (e.g. a natural polypeptide having less than 100% sequence identity with the natural polypeptide) or fragments thereof.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence in a cell.

As used herein, the term “transgene” refers to a polynucleotide that has been transferred naturally, or by any of a number of genetic engineering techniques from one organism to another. It is optionally translated into a polypeptide. As used, transgene can refer to a polynucleotide that encodes a polypeptide.

The terms “protein,” “polypeptide,” and “peptide” are used herein interchangeably.

As used herein, the term “operably linked” or “operatively linked” refers to the binding of a nucleic acid sequence to a single nucleic acid fragment such that one function is affected by the other. For example, if a promoter is capable of affecting the expression of a coding sequence or functional RNA (i.e., the coding sequence or functional RNA is under transcriptional control by the promoter), the promoter is operably linked thereto. Coding sequences can be operably linked to control sequences in both sense and antisense orientation.

As used herein, the term “developmental cell states” refers to, for example, states when the cell is inactive, actively expressing, differentiating, senescent, etc. developmental cell state may also refer to a cell in a precursor state (e.g., a T cell precursor).

As used, the term “encoding” refers to a sequence of nucleic acids which codes for a protein or polypeptide of interest. The nucleic acid sequence may be either a molecule of DNA or RNA. In preferred embodiments, the molecule is a DNA molecule. In other preferred embodiments, the molecule is a RNA molecule. When present as a RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art.

As used herein, the term “subject” refers to a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an engineered cell provided herein or population thereof. In some aspects, the disease or condition is a cancer.

As used herein, the term “promoter” refers to a nucleotide sequence (e.g. DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue specific Or developmentally regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, Hepatocyte Nuclear Factor 1α (HNF1α), etc.). The promoter can be a constitutive promoter (e.g., CMV promoter, UBC promoter, EF1α promoter). In some embodiments, the promoter can be a spatially restricted and/or temporally restricted promoter (e.g., a tissue specific promoter, a cell type specific promoter, etc.). See for example US Publication 20180127786, the disclosure of which is herein incorporated by reference in its entirety.

Gene editing, as contemplated herein, may involve a gene (or nucleotide sequence) knock-in or knock-out. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a polynucleotide donor construct encoding a protein may be inserted into the genome of a cell carrying a mutant gene. In some embodiments, a knock-in strategy involves substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant (e.g., non-coding) sequence.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a “DNA template,” “DNA template insert,” “single-stranded DNA template,” a “single-stranded DNA template insert,” a “double-stranded DNA template,” or a “double-stranded DNA template insert” refers to a DNA oligonucleotide that can be used by a cell as a template for HDR. Generally, the single-stranded DNA template or a double-stranded DNA template has at least one region of homology to a target site. In some cases, the single-stranded DNA template or double-stranded DNA template has two homologous regions flanking a region that contains a heterologous sequence to be inserted at a target cut site. In some embodiments, the DNA template or DNA template insert comprises a cassette or expression cassette comprising one or more modules that encode for transgenes and/or RNAi molecules disclosed herein.

The terms “expression vector,” “vector,” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette or cassette. Vectors and plasmids include, but are not limited to, integrating vectors, prokaryotic plasmids, eukaryotic plasmids, plant synthetic chromosomes, episomes, cosmids, and artificial chromosomes.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein the term “expression cassette” or “cassette” is a polynucleotide construct, generated recombinantly or chemically synthesized, comprising regulatory sequences operably linked to a selected polynucleotide to facilitate expression of the selected polynucleotide in a host cell. Such cassettes may include one or more modules comprising the selected polynucleotide, such as the transgenes (e.g., a CAR, priming receptor, and/or synthetic pathway activator described herein) and/or RNAi molecules (e.g., an shRNA) disclosed herein. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector. In some embodiments, the cassette is part of a DNA template insert.

As used herein, the phrase “subject in need thereof” refers to a subject that exhibits and/or is diagnosed with one or more symptoms or signs of a disease or disorder as described herein.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “composition” refers to a mixture that contains, e.g., an engineered cell or protein contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The term “in situ” refers to processes that occur in a living cell growing separate from a living organism, e.g., growing in tissue culture.

The term “in vivo” refers to processes that occur in a living organism.

As used herein, the term “ex vivo” generally includes experiments or measurements made in or on living tissue, preferably in an artificial environment outside the organism, preferably with minimal differences from natural conditions.

The term “mammal” as used herein includes both humans and non-humans and include but is not limited to humans, non-human primates, canines, felines, murines, bovines, equines, and porcines.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

The term “ameliorating” refers to any therapeutically beneficial result in the treatment of a disease state, e.g., a cancer disease state, lessening in the severity or progression, remission, or cure thereof.

As used herein, the term “effective amount” refers to the amount of a compound (e.g., a compositions described herein, cells described herein) sufficient to effect beneficial or desired results. An effective amount can be administered in one or more administrations, applications or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). For example, an antibody that “selectively binds” or “specifically binds” an antigen is an antigen-binding moiety that binds the antigen with high affinity and does not significantly bind other unrelated antigens. Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule.

“Affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including, but not limited to, surface plasmon resonance (SPR) technology (e.g., BIACORE®) or biolayer interferometry (e.g., FORTEBIO®).

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The term “CDR” denotes a complementarity determining region as defined by at least one manner of identification to one of skill in the art.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); Martin (Enhanced Chothia or AbM) Abhinandan and Martin, Mol Immunol. 2008 August; 45(14):3832-9; MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegger and Plückthun, J. Mol. Biol., 2001, 309:657-70 (“AHo” numbering scheme); each of which is incorporated by reference in its entirety.

Table A provides the positions of CDR-L1, CDR-L2, CDR-L3, CDR-H1, CDR-H2, and CDR-H3 as identified by the Kabat, Chothia, AbM, Contact, and IMGT schemes. For CDR-H1, residue numbering is provided using both the Kabat and Chothia numbering schemes.

CDRs may be assigned, for example, using antibody numbering software, such as Abnum, available at bioinf.org.uk/abs/abnum/and described in Abhinandan and Martin, Immunology, 2008, 45:3832-3839, incorporated by reference in its entirety, and AbYsis, available at abysis.org/abysis/sequence_input/key_annotation/key_annotation.cgi.

TABLE A
Residues in CDRs according to Kabat, Chothia,
AbM, Contact, and IMGT numbering schemes.

| CDR | Kabat | Chothia | AbM | Contact | IMGT |
|---|---|---|---|---|---|
| L1 | L24-L34 | L24-L34 | L24-L34 | L30-L36 | L27-L32 |
| L2 | L50-L56 | L50-L56 | L50-L56 | L46-L55 | L50-L51 |
| L3 | L89-L97 | L89-L97 | L89-L97 | L89-L96 | L89-L97 |
| H1 (Kabat | H31-H35B | H26-H32 or | H26-H35B | H30-H35B | H26-H35B |
| Numbering) | | H34* | | | |
| H1 | H31-H35 | H26-H32 | H26-H35 | H30-H35 | H26-H33 |
| (Chothia/Martin | | | | | |
| Numbering) | | | | | |
| H2 | H50-H65 | H52-H56 | H50-H58 | H47-H58 | H51-H56 |
| H3 | H95-H102 | H95-H102 | H95-H102 | H93-H101 | H93-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

As used herein, the term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)₂” fragments contain two Fab′ fragments joined, near the hinge region, by disulfide bonds. F(ab′)₂ fragments may be generated, for example, by recombinant or synthetic methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

The “Single-chain Fv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22). The terms “single domain antibody” and “sdAb” are used interchangeably herein to refer to an antibody comprising at least one monomeric domain, such as a VHH domain, or a VNAR domain (from a shark antibody) without a light chain, and an Fc region.

The term “VHH” or “VHH domain” or “VHH antigen-binding domain” as used herein refers to the antigen-binding portion of a single-domain antibody (sdAb), such as a camelid antibody. In some embodiments, a VHH comprises three CDRs and four framework regions, designated FR1, CDR1, FR2, CDR2, FR3, CDR3, and FR4.

It must be noted that, as used in the specification and the appended claims, the singular forms “a,” “an” and “the” include plural referents unless the context clearly dictates otherwise.

### Antibodies and Antigen Binding Fragments

PSMA antibodies, antigen binding fragments, CDRs, VH, and VL Domains

In some aspects, provided herein are isolated antibodies or antigen binding fragments thereof that bind to Prostate-Specific Membrane Antigen (PSMA) (SEQ ID NO: 2). PSMA is also known as FOLH1 or Folate Hydrolase 1 (HGNC: 3788, NCBI Entrez Gene: 2346, Ensembl: ENSG00000086205, UniProtKB/Swiss-Prot: Q04609). The amino acid sequence of PSMA is provided in SEQ ID NO: 3.

In some aspects, provided herein are antibodies or antigen binding fragments thereof that bind to PSMA. In some aspects, provided herein are means for binding to PSMA. In some embodiments, the means for binding to PSMA comprises an antibody or antigen-binding fragment provided herein. In some embodiments, a PSMA antibody or antigen-binding fragment or equivalent thereof comprises means for binding a PSMA protein, optionally binding a human PSMA protein in the region(s) of human PSMA bound by the PSMA binders (e.g., as described in the Examples below). In some embodiments, the means binds a PSMA protein. In some embodiments, the means binds a human PSMA protein. In some embodiments, the means is a PSMA antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof) means for binding a PSMA protein. In some embodiments, the means for binding PSMA includes the anti-PSMA antibodies and antigen-binding fragments or equivalents thereof described herein.

In some embodiments, a PSMA antibody or antigen-binding fragment or equivalent thereof provided herein binds to human PSMA. In some embodiments, a PSMA antibody or antigen-binding fragment or equivalent thereof provided herein does not bind to mouse PSMA. In some embodiments, a PSMA antibody or antigen-binding fragment or equivalent thereof provided herein binds to isoforms of human PSMA, including, but not limited to, a PSMA protein comprising a Y75H single nucleotide polymorphism (SNP) as compared to SEQ ID NO: 2.

In some aspects, the PSMA antibody or antigen-binding fragment or equivalent thereof comprises a variable heavy (VH) chain sequence comprising three heavy chain CDR sequences, CDR-H1, CDR-H2, and CDR-H3, of the VH sequences set forth in SEQ ID NOs: 118 or 130, and a variable light (VL) chain sequence comprising three light chain CDR sequences, CDR-L1, CDR-L2, and CDR-L3, of the VL sequences set forth in SEQ ID NOs: 119 or 131. In some embodiments, CDR-H1 comprises the sequence set forth in SEQ ID NO: 120, CDR-H2 comprises the sequence set forth in SEQ ID NO: 121, CDR-H3 comprises the sequence set forth in SEQ ID NO: 122, CDR-L1 comprises the sequence set forth in SEQ ID NO: 123, CDR-L2 comprises the sequence set forth in SEQ ID NO: 124, and CDR-L3 comprises the sequence set forth in SEQ ID NO: 125. In some embodiments, CDR-H1 comprises the sequence set forth in SEQ ID NO: 132, CDR-H2 comprises the sequence set forth in SEQ ID NO: 133, CDR-H3 comprises the sequence set forth in SEQ ID NO: 134, CDR-L1 comprises the sequence set forth in SEQ ID NO: 135, CDR-L2 comprises the sequence set forth in SEQ ID NO: 136, and CDR-L3 comprises the sequence set forth in SEQ ID NO: 137. In some embodiments, CDR-H1, CDR-H2, CDR-H3, CDR-L1, CDR-L2, and CDR-L3 comprise a sequence as set forth in Table B.

TABLE B
PSMA binder CDR sequences according to
different definitions

| SEQ | | | |
|---|---|---|---|
| ID | | Defi- | |
| NO | Name | nition | Sequence |
| 120 | PSMA 1 CDR-H1 | Chothia | GYTFTSY--- |
| 199 | PSMA 1 CDR-H1 | AbM | GYTFTSYGIT |
| 200 | PSMA 1 CDR-H1 | Kabat | -----SYGIT |
| 201 | PSMA 1 CDR-H1 | Contact | ----TSYGIT |
| 202 | PSMA 1 CDR-H1 | IMGT | GYTFTSYG-- |
| 121 | PSMA 1 CDR-H2 | Chothia | -----SEYNGN--------- |
| 203 | PSMA 1 CDR-H2 | AbM | ---WISEYNGNTN------- |
| 204 | PSMA 1 CDR-H2 | Kabat | ---WISEYNGNTNYAQKFQG |
| 205 | PSMA 1 CDR-H2 | Contact | WMGWISEYNGNTN------- |
| 206 | PSMA 1 CDR-H2 | IMGT | ----ISEYNGNT-------- |
| 122 | PSMA 1 CDR-H3 | Chothia | --GGILDYYFFYYMDV |
| 122 | PSMA 1 CDR-H3 | AbM | --GGILPYYFFYYMDV |
| 122 | PSMA 1 CDR-H3 | Kabat | --GGILPYYFFYYMDV |
| 207 | PSMA 1 CDR-H3 | Contact | ARGGILPYYFFYYMD- |
| 208 | PSMA 1 CDR-H3 | IMGT | ARGGILPYYFFYYMDV |
| 123 | PSMA 1 CDR-L1 | Chothia | RASQSVSSSYLA-- |
| 123 | PSMA 1 CDR-L1 | AbM | RASQSVSSSYLA-- |
| 123 | PSMA 1 CDR-L1 | Kabat | RASQSVSSSYLA-- |
| 209 | PSMA 1 CDR-L1 | Contact | ------SSSYLAWY |
| 210 | PSMA 1 CDR-L1 | IMGT | ---QSVSSSY---- |
| 124 | PSMA 1 CDR-L2 | Chothia | ----GASSRAT |
| 124 | PSMA 1 CDR-L2 | AbM | ----GASSRAT |
| 124 | PSMA 1 CDR-L2 | Kabat | ----GASSRAT |
| 211 | PSMA 1 CDR-L2 | Contact | LLIYGASSRA- |
| | PSMA 1 CDR-L2 | IMGT | ----GA----- |
| 125 | PSMA 1 CDR-L3 | Chothia | QQYGSSPYT |
| 125 | PSMA 1 CDR-L3 | AbM | QQYGSSPYT |
| 125 | PSMA 1 CDR-L3 | Kabat | QQYGSSPYT |
| 212 | PSMA 1 CDR-L3 | Contact | QQYGSSPY- |
| 125 | PSMA 1 CDR-L3 | IMGT | QQYGSSPYT |
| 132 | PSMA 2 CDR-H1 | Chothia | GYTFSSY--- |
| 263 | PSMA 2 CDR-H1 | AbM | GYTFSSYGVS |
| 213 | PSMA 2 CDR-H1 | Kabat | -----SYGVS |
| 214 | PSMA 2 CDR-H1 | Contact | ----SSYGVS |
| 215 | PSMA 2 CDR-H1 | IMGT | GYTFSSYG-- |
| 133 | PSMA 2 CDR-H2 | Chothia | -----SKYNGN--------- |
| 216 | PSMA 2 CDR-H2 | AbM | ---WISKYNGNTN------- |
| 217 | PSMA 2 CDR-H2 | Kabat | ---WISKYNGNTNYAQKFQG |
| 218 | PSMA 2 CDR-H2 | Contact | WMGWISKYNGNTN------- |
| 219 | PSMA 2 CDR-H2 | IMGT | ----ISKYNGNT-------- |
| 134 | PSMA 2 CDR-H3 | Chothia | --GGIHGDSYYFYYLDV |
| 134 | PSMA 2 CDR-H3 | AbM | --GGIHGDSYYFYYLDV |
| 134 | PSMA 2 CDR-H3 | Kabat | --GGIHGDSYYFYYLDV |
| 220 | PSMA 2 CDR-H3 | Contact | ARGGIHGDSYYFYYLD- |
| 221 | PSMA 2 CDR-H3 | IMGT | ARGGIHGDSYYFYYLDV |
| 135 | PSMA 2 CDR-L1 | Chothia | GASQSVSSSYLA-- |
| 135 | PSMA 2 CDR-L1 | AbM | GASQSVSSSYLA-- |
| 135 | PSMA 2 CDR-L1 | Kabat | GASQSVSSSYLA-- |
| 222 | PSMA 2 CDR-L1 | Contact | ------SSSYLAWY |
| 223 | PSMA 2 CDR-L1 | IMGT | ---QSVSSSY---- |
| 136 | PSMA 2 CDR-L2 | Chothia | ----DASSRAT |
| 136 | PSMA 2 CDR-L2 | AbM | ----DASSRAT |
| 136 | PSMA 2 CDR-L2 | Kabat | ----DASSRAT |
| 224 | PSMA 2 CDR-L2 | Contact | LLIYDASSRA- |
| | PSMA 2 CDR-L2 | IMGT | ----DA----- |
| 137 | PSMA 2 CDR-L3 | Chothia | QQYGSSPYT |
| 137 | PSMA 2 CDR-L3 | AbM | QQYGSSPYT |
| 137 | PSMA 2 CDR-L3 | Kabat | QQYGSSPYT |
| 225 | PSMA 2 CDR-L3 | Contact | QQYGSSPY- |
| 137 | PSMA 2 CDR-L3 | IMGT | QQYGSSPYT |

In some embodiments, the VH chain sequence comprises the sequence set forth in SEQ ID NO: 118. In some embodiments, the VH chain sequence comprises the sequence set forth in SEQ ID NO: 130. In some embodiments, the VL comprises the sequence set forth in SEQ ID NO: 119. In some embodiments, the VL comprises the sequence set forth in SEQ ID NO: 131. In some embodiments, the PSMA antibody or antigen-binding fragment or equivalent thereof comprises an extracellular domain comprises the sequence set forth in SEQ ID NO: 117. In some embodiments, the PSMA antibody or antigen-binding fragment or equivalent thereof comprises an extracellular domain comprises the sequence set forth in SEQ ID NO: 129.

In some embodiments, the PSMA antibody or antigen-binding fragment or equivalent thereof CDR-H3 has at least about 50%, 75%, 80%, 85%, 90%, or 95% identity with a CDR-H3 of SEQ ID NO: 122 or 134, the CDR-H2 has at least about 50%, 75%, 80%, 85%, 90%, or 95% identity with a CDR-H2 of SEQ ID NO: 121 or 133, the CDR-H1 has at least about 50%, 75%, 80%, 85%, 90%, or 95% identity with a CDR-H1 of SEQ ID NO: 120 or 132, the CDR-L3 has at least about 50%, 75%, 80%, 85%, 90%, or 95% identity with a CDR-L3 of SEQ ID NO: 125 or 137, the CDR-L2 has at least about 50%, 75%, 80%, 85%, 90%, or 95% identity with a CDR-L2 of SEQ ID NO: 124 or 136, and the CDR-L1 has at least about 50%, 75%, 80%, 85%, 90%, or 95% identity with a CDR-L1 of SEQ ID NO: 123 or 135. In some embodiments, the CDR-H3 is a CDR-H3 of SEQ ID NO: 122 or 134, with up to 1, 2, 3, 4, 5, 6, 7, or 8 amino acid substitutions; the CDR-H2 is a CDR-H2 of SEQ ID NO: 121 or 133, with up to 1, 2, 3, 4, 5, 6, 7, or 8 amino acid substitutions; the CDR-H1 is a CDR-H1 of SEQ ID NO: 120 or 132, with up to 1, 2, 3, 4, or 5 amino acid substitutions; the CDR-L3 is a CDR-L3 of SEQ ID NO: 125 or 137, with up to 1, 2, 3, 4, or 5 amino acid substitutions; the CDR-L2 is a CDR-L2 of SEQ ID NO: 124 or 136, with up to 1, 2, 3, or 4 amino acid substitutions; and the CDR-L1 is a CDR-L1 of SEQ ID NO: 123 or 135 with up to 1, 2, 3, 4, 5, or 6 amino acid substitutions.

In some embodiments, a PSMA antibody or antigen-binding fragment or equivalent thereof provided herein comprises one to three CDRs of a VH domain as set forth in SEQ ID NO: 118 or 130. In some embodiments, a PSMA antibody or antigen-binding fragment or equivalent thereof provided herein comprises two to three CDRs of a VH domain as set forth in SEQ ID Nos: 118 or 130. In some embodiments, a PSMA antibody or antigen-binding fragment or equivalent thereof provided herein comprises three CDRs of a VH domain as set forth in SEQ ID NO: 118 or 130. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a PSMA antibody or antigen-binding fragment or equivalent thereof provided herein comprises a VH sequence having at least about 50%, 60%, 70%, 80%, 90%, 95%, or 99% identity to an VH sequence set forth in SEQ ID NO: 118 or 130. In some embodiments, an antigen-binding domain provided herein comprises a VH sequence provided in SEQ ID NO: 118 or 130, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antigen-binding domains described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a PSMA antibody or antigen-binding fragment or equivalent thereof provided herein comprises one to three CDRs of a VL domain as set forth in SEQ ID NO: 119 or 131. In some embodiments, an antigen-binding domain provided herein comprises two to three CDRs of a VL domain as set forth in SEQ ID NO: 119 or 131. In some embodiments, an antigen-binding domain provided herein comprises three CDRs of a VL domain as set forth in SEQ ID NO: 119 or 131. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a PSMA antibody or antigen-binding fragment or equivalent thereof provided herein comprises a VL sequence having at least about 50%, 60%, 70%, 80%, 90%, 95%, or 99% identity to an VL sequence set forth in SEQ ID NO: 119 or 131. In some embodiments, a PSMA antibody or antigen-binding fragment or equivalent thereof provided herein comprises a VL sequence provided in SEQ ID NO: 119 or 131, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a PSMA antibody or antigen-binding fragment or equivalent thereof provided herein comprises a sequence having at least about 50%, 60%, 70%, 80%, 90%, 95%, or 99% identity to the sequence set forth in SEQ ID NO: 117 or 129. In some embodiments, a PSMA antibody or antigen-binding fragment or equivalent thereof antigen-binding domain provided herein comprises an scFv sequence provided in SEQ ID NO: 117 or 129, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, an PSMA antibody or antigen-binding fragment or equivalent thereof comprises means for binding a PSMA protein, optionally binding a human PSMA protein in the region(s) of human PSMA bound by the PSMA 1 or PSMA 2 binders (e.g., as described in the Examples below). In some embodiments, the means binds human PSMA protein. In some embodiments, the means is a PSMA antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof) means for binding human PSMA protein. In some embodiments, the means for binding PSMA includes the anti-PSMA antibodies and antigen-binding fragments or equivalents thereof described herein.

### CA9 Antibodies, Antigen Binding Fragments, CDRs, VH, and VL Domains

In some aspects, provided herein are isolated antibodies or antigen binding fragments thereof that bind to Carbonic Anhydrase IX (CA9) (SEQ ID NO: 1). The amino acid sequence of CA9 (HGNC: 1383, NCBI Entrez Gene: 768, Ensembl: ENSG00000107159, UniProtKB/Swiss-Prot: Q16790) is provided in SEQ ID NO: 1.

In some aspects, provided herein are antibodies or antigen binding fragments thereof that bind to CA9. In some aspects, provided herein are means for binding to CA9. In some embodiments, the means for binding to CA9 comprises an antibody or antigen-binding fragment provided herein. In some embodiments, a CA9 antibody or antigen-binding fragment or equivalent thereof comprises means for binding a CA9 protein, optionally binding a human CA9 protein in the region(s) of human CA9 bound by the CA9 binders (e.g., as described in the Examples below). In some embodiments, the means binds a CA9 protein. In some embodiments, the means binds a human CA9 protein. In some embodiments, the means is a CA9 antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof) means for binding a CA9 protein. In some embodiments, the means for binding CA9 includes the anti-CA9 antibodies and antigen-binding fragments or equivalents thereof described herein.

In some embodiments, a CA9 antibody or antigen-binding fragment or equivalent thereof provided herein binds to human CA9. In some embodiments, a CA9 antibody or antigen-binding fragment or equivalent thereof provided herein does not bind to mouse CA9. In some embodiments, a CA9 antibody or antigen-binding fragment or equivalent thereof provided herein binds to isoforms of human CA9, including, but not limited to, a CA9 protein comprising a R131 W single nucleotide polymorphism (SNP), a Q326R SNP or a 91-96 deletion (91-96del) as compared to SEQ ID NO: 1.

In some aspects, the CA9 antibody or antigen-binding fragment or equivalent thereof comprises a variable heavy (VH) chain sequence comprising three heavy chain CDR sequences, CDR-H1, CDR-H2, and CDR-H3, of the VH sequences set forth in SEQ ID NOs: 99 or 110. In some embodiments, the CA9 antibody or antigen-binding fragment or equivalent thereof comprises a variable light (VL) chain sequence comprising three light chain CDR sequences, CDR-L1, CDR-L2, and CDR-L3, of the VL sequence set forth in SEQ ID NO: 100. In some embodiments, CDR-H1, CDR-H2, CDR-H3, CDR-L1, CDR-L2, CDR-L3 comprise s sequence set forth in Table C. In some embodiments, CDR-H1 comprises the sequence set forth in SEQ ID NO: 101, CDR-H2 comprises the sequence set forth in SEQ ID NO: 102, CDR-H3 comprises the sequence set forth in SEQ ID NO: 103, CDR-L1 comprises the sequence set forth in SEQ ID NO: 104, CDR-L2 comprises the sequence set forth in SEQ ID NO: 105, and CDR-L3 comprises the sequence set forth in SEQ ID NO: 106. In some embodiments, CDR-H1 comprises the sequence set forth in SEQ ID NO: 111, CDR-H2 comprises the sequence set forth in SEQ ID NO: 112, CDR-H3 comprises the sequence set forth in SEQ ID NO: 113.

TABLE C
CA9 binder CDR sequences

| SEQ | | | |
|---|---|---|---|
| ID | | Defini- | |
| NO | Name | tion | Sequence |
| 101 | CA9 1 CDR-H1 | Chothia | GYTFTSY--- |
| 226 | CA9 1 CDR-H1 | AbM | GYTFTSYYMH |
| 227 | CA9 1 CDR-H1 | Kabat | -----SYYMH |
| 228 | CA9 1 CDR-H1 | Contact | ----TSYYMH |
| 229 | CA9 1 CDR-H1 | IMGT | GYTFTSYY-- |
| 102 | CA9 1 CDR-H2 | Chothia | -----NPSRGS--------- |
| 230 | CA9 1 CDR-H2 | AbM | ---IINPSRGSAS------- |
| 231 | CA9 1 CDR-H2 | Kabat | ---IINPSRGSASYAQKFQG |
| 232 | CA9 1 CDR-H2 | Contact | WMGIINPSRGSAS------- |
| 233 | CA9 1 CDR-H2 | IMGT | ----INPSRGSA-------- |
| 103 | CA9 1 CDR-H3 | Chothia | --DRNYYYYMDV |
| 103 | CA9 1 CDR-H3 | AbM | --DRNYYYYMDV |
| 103 | CA9 1 CDR-H3 | Kabat | --DRNYYYYMDV |
| 234 | CA9 1 CDR-H3 | Contact | ARDRNYYYYMD- |
| 235 | CA9 1 CDR-H3 | IMGT | ARDRNYYYYMDV |
| 104 | CA9 1 CDR-L1 | Chothia | RASQNISSNLA-- |
| 104 | CA9 1 CDR-L1 | AbM | RASQNISSNLA-- |
| 104 | CA9 1 CDR-L1 | Kabat | RASQNISSNLA-- |
| 236 | CA9 1 CDR-L1 | Contact | ------SSNLAWY |
| 237 | CA9 1 CDR-L1 | IMGT | ---QNISSN---- |
| 105 | CA9 1 CDR-L2 | Chothia | ----GASTRAT |
| 105 | CA9 1 CDR-L2 | AbM | ----GASTRAT |
| 105 | CA9 1 CDR-L2 | Kabat | ----GASTRAT |
| 238 | CA9 1 CDR-L2 | Contact | LLIYGASTRA- |
| | CA9 1 CDR-L2 | IMGT | ----GA----- |
| 106 | CA9 1 CDR-L3 | Chothia | QQYITWYT |
| 106 | CA9 1 CDR-L3 | AbM | QQYITWYT |
| 106 | CA9 1 CDR-L3 | Kabat | QQYITWYT |
| 239 | CA9 1 CDR-L3 | Contact | QQYITWY- |
| 106 | CA9 1 CDR-L3 | IMGT | QQYITWYT |
| 111 | CA9 2 CDR-H1 | Chothia | GFSFSDYS--- |
| 240 | CA9 2 CDR-H1 | AbM | GFSFSDYSGMS |
| 241 | CA9 2 CDR-H1 | Kabat | -----DYSGMS |
| 242 | CA9 2 CDR-H1 | Contact | ----SDYSGMS |
| 243 | CA9 2 CDR-H1 | IMGT | GFSFSDYSG-- |
| 112 | CA9 2 CDR-H2 | Chothia | -----SPGGGD--------- |
| 244 | CA9 2 CDR-H2 | AbM | ---AISPGGGDTY------- |
| 245 | CA9 2 CDR-H2 | Kabat | ---AISPGGGDTYYADSVKG |
| 246 | CA9 2 CDR-H2 | Contact | LVSAISPGGGDTY------- |
| 247 | CA9 2 CDR-H2 | IMGT | ----ISPGGGDT-------- |
| 113 | CA9 2 CDR-H3 | Chothia | --RWWYYSNHSGDYDYFDY |
| 113 | CA9 2 CDR-H3 | AbM | --RWWYYSNHSGDYDYFDY |
| 113 | CA9 2 CDR-H3 | Kabat | --RWWYYSNHSGDYDYFDY |
| 248 | CA9 2 CDR-H3 | Contact | ARRWWYYSNHSGDYDYFD- |
| 249 | CA9 2 CDR-H3 | IMGT | ARRWWYYSNHSGDYDYFDY |

In some embodiments, the VH chain sequence comprises the sequence set forth in SEQ ID NO: 99. In some embodiments, the VH chain sequence comprises the sequence set forth in SEQ ID NO: 110. In some embodiments, the VL comprises the sequence set forth in SEQ ID NO: 100. In some embodiments, the CA9 antibody or antigen-binding fragment or equivalent thereof comprises an extracellular domain comprises the sequence set forth in SEQ ID NO: 98. In some embodiments, the CA9 antibody or antigen-binding fragment or equivalent thereof comprises an extracellular domain comprises the sequence set forth in SEQ ID NO: 110.

In some embodiments, the CA9 antibody or antigen-binding fragment or equivalent thereof CDR-H3 has at least about 50%, 75%, 80%, 85%, 90%, or 95% identity with a CDR-H3 of SEQ ID NO: 103 or 113, the CDR-H2 has at least about 50%, 75%, 80%, 85%, 90%, or 95% identity with a CDR-H2 of SEQ ID NO: 102 or 112, the CDR-H1 has at least about 50%, 75%, 80%, 85%, 90%, or 95% identity with a CDR-H1 of SEQ ID NO: 101 or 111, the CDR-L3 has at least about 50%, 75%, 80%, 85%, 90%, or 95% identity with a CDR-L3 of SEQ ID NO: 106, the CDR-L2 has at least about 50%, 75%, 80%, 85%, 90%, or 95% identity with a CDR-L2 of SEQ ID NO: 105, and the CDR-L1 has at least about 50%, 75%, 80%, 85%, 90%, or 95% identity with a CDR-L1 of SEQ ID NO: 104. In some embodiments, the CDR-H3 is a CDR-H3 of SEQ ID NO: 103 or 113, with up to 1, 2, 3, 4, 5, 6, 7, or 8 amino acid substitutions; the CDR-H2 is a CDR-H2 of SEQ ID NO: 102 or 112, with up to 1, 2, 3, 4, 5, 6, 7, or 8 amino acid substitutions; the CDR-H1 is a CDR-H1 of SEQ ID NO: 101 or 111, with up to 1, 2, 3, 4, or 5 amino acid substitutions; the CDR-L3 is a CDR-L3 of SEQ ID NO: 106, with up to 1, 2, 3, 4, or 5 amino acid substitutions; the CDR-L2 is a CDR-L2 of SEQ ID NO: 105, with up to 1, 2, 3, or 4 amino acid substitutions; and the CDR-L1 is a CDR-L1 of SEQ ID NO: 104 with up to 1, 2, 3, 4, 5, or 6 amino acid substitutions.

In some embodiments, a CA9 antibody or antigen-binding fragment or equivalent thereof provided herein comprises one to three CDRs of a VH domain as set forth in SEQ ID NO: 99 or 110. In some embodiments, a CA9 antibody or antigen-binding fragment or equivalent thereof provided herein comprises two to three CDRs of a VH domain as set forth in SEQ ID Nos: 99 or 110. In some embodiments, a CA9 antibody or antigen-binding fragment or equivalent thereof provided herein comprises three CDRs of a VH domain as set forth in SEQ ID NO: 99 or 110. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a CA9 antibody or antigen-binding fragment or equivalent thereof provided herein comprises a VH sequence having at least about 50%, 60%, 70%, 80%, 90%, 95%, or 99% identity to an VH sequence set forth in SEQ ID NO: 99 or 110. In some embodiments, an antigen-binding domain provided herein comprises a VH sequence provided in SEQ ID NO: 99 or 110, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antigen-binding domains described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a CA9 antibody or antigen-binding fragment or equivalent thereof provided herein comprises one to three CDRs of a VL domain as set forth in SEQ ID NO: 100. In some embodiments, an antigen-binding domain provided herein comprises two to three CDRs of a VL domain as set forth in SEQ ID NO: 100. In some embodiments, an antigen-binding domain provided herein comprises three CDRs of a VL domain as set forth in SEQ ID NO: 100. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a CA9 antibody or antigen-binding fragment or equivalent thereof provided herein comprises a VL sequence having at least about 50%, 60%, 70%, 80%, 90%, 95%, or 99% identity to an VL sequence set forth in SEQ ID NO: 100. In some embodiments, a CA9 antibody or antigen-binding fragment or equivalent thereof provided herein comprises a VL sequence provided in SEQ ID NO: 100, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a CA9 antibody or antigen-binding fragment or equivalent thereof provided herein comprises a sequence having at least about 50%, 60%, 70%, 80%, 90%, 95%, or 99% identity to the sequence set forth in SEQ ID NO: 98 or 110. In some embodiments, a CA9 antibody or antigen-binding fragment or equivalent thereof antigen-binding domain provided herein comprises an scFv sequence provided in SEQ ID NO: 98 or 110, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a CA9 antibody or antigen-binding fragment or equivalent thereof comprises means for binding a CA9 protein, optionally binding human CA9 protein in the region(s) of human CA9 protein bound by the CA9 1 or CA9 2 binders (e.g., as described in the Examples below). In some embodiments, the means binds human CA9. In some embodiments, the means is a CA9 antibody or antigen-binding fragment or equivalent thereof (e.g., a full length antibody or a F(ab′)2 fragment, a Fab fragment, a single chain variable fragment (scFv), a VHH, and a single domain antibody (sdAb), or a functional fragment thereof) means for binding CA9. In some embodiments, the means for binding CA9 includes the anti-CA9 antibodies and antigen-binding fragments or equivalents thereof described herein.

### Logic Gate Systems

As used herein, a “logic gate,” “circuit,” “circuit receptor,” “system” or “system receptor” refers to a two part protein expression system comprising a priming receptor and a chimeric antigen receptor. The system can be encoded on at least one nucleic acid inserted into a cell, where the priming receptor is expressed in the cell. The intracellular domain of the priming receptor is cleaved from the transmembrane domain upon binding of the priming receptor to its target antigen. The intracellular domain is then capable of translocating into a cell nucleus where it induces expression of the chimeric antigen receptor.

In one aspect, provided herein are systems comprising a priming receptor that binds to PSMA and a chimeric antigen receptor that binds to CA9, wherein the transcription factor of the intracellular domain of the priming receptor is capable of inducing expression of the CAR. Such systems are alternatively termed “logic gates” or “circuits.” In some aspects, the system is encoded by nucleic acid transgenes inserted into an immune cell. The system can be encoded on a single nucleic acid insert or fragment that comprises both transgenes, or can be encoded on two nucleic acids that encode the system transgenes individually. The priming receptor and CAR of the system can be placed in any order on the single nucleic acid. For example, the priming receptor can be at the 5′ end and the CAR can be at the 3′ end, or the CAR can be at the 5′ end and the priming receptor can be at the 3′ end.

A constitutive promoter can be operably linked to the nucleotide sequence encoding the priming receptor. An inducible promoter can also be operably linked to the nucleotide sequence encoding the CAR. In some embodiments, when the system is encoded on a single nucleic acid insert or fragment that comprises both transgenes, the nucleic acid can comprise, in a 5′ to 3′ direction, the constitutive promoter; the nucleotide sequence encoding priming receptor; the inducible promoter; and the nucleotide sequence encoding chimeric antigen receptor. Alternatively, the nucleic acid can comprise, in a 5′ to 3′ direction, the inducible promoter; the nucleotide sequence encoding chimeric antigen receptor; the constitutive promoter; the nucleotide sequence encoding priming receptor.

In some embodiments, the constitutive promoter is an EF1α promoter. In some embodiments, the constitutive promoter comprises the sequence of SEQ ID NO: 179.

In some embodiments, the inducible promoter comprises one or more Hepatocyte Nuclear Factor 1α (HNF1α) enhancer element(s). For example, the inducible promoter can comprise 1, 2, 3, 4, 5, 6, 7, or more HNF1α enhancer element(s). In some embodiments, the inducible promoter further comprises a YB-TATA promoter sequence. In some embodiments, the inducible promoter comprises the sequence as set forth in SEQ ID NO: 256.

In some embodiments, the logic gate is encoded on a nucleic acid. In some embodiments, the nucleic acid is selected from the group consisting of the sequences set forth in SEQ ID NOs: 143-147. In some embodiments, the nucleic acid is a sequence as set forth in SEQ ID NO: 143. In some embodiments, the nucleic acid is a sequence as set forth in SEQ ID NO: 144. In some embodiments, the nucleic acid is a sequence as set forth in SEQ ID NO: 145. In some embodiments, the nucleic acid is a sequence as set forth in SEQ ID NO: 146. In some embodiments, the nucleic acid is a sequence as set forth in SEQ ID NO: 147.

In some embodiments, the nucleic acid comprises a sequence as set forth in SEQ ID NO: 143. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 143.

In some embodiments, the nucleic acid comprises a sequence as set forth in SEQ ID NO: 144. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 144.

In some embodiments, the nucleic acid comprises a sequence as set forth in SEQ ID NO: 145. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 145.

In some embodiments, the nucleic acid comprises a sequence as set forth in SEQ ID NO: 146. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 146.

In some embodiments, the nucleic acid comprises a sequence as set forth in SEQ ID NO: 147. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 147.

### Synthetic Receptors

In one aspect, synthetic receptors disclosed herein comprises a domain that specifically binds Prostate-Specific Membrane Antigen (PSMA). In one aspect, synthetic receptors disclosed herein comprises a domain that specifically binds (CA9). In some embodiments, the domain is an extracellular domain. In some embodiments, the domain includes the ligand-binding portion of a receptor. In some embodiments, the domain includes an antigen-binding moiety that binds to one or more target antigens. In some embodiments, the antigen-binding moiety includes one or more antigen-binding determinants of an antibody or a functional antigen-binding fragment or equivalent thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), a VHH, and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

In some embodiments, the synthetic receptor is a chimeric antigen receptor or a priming receptor.

### PSMA Receptors

In some embodiments, the antigen-binding domain specifically binds to Prostate-Specific Membrane Antigen. In some embodiments, the antigen-binding domain includes an antigen-binding moiety that binds to Prostate-Specific Membrane Antigen.

In some embodiments, provided herein are isolated synthetic receptors comprising an antigen-binding domain that binds to Prostate-Specific Membrane Antigen (PSMA) (SEQ ID NO: 2).

In some embodiments, the isolated synthetic receptor comprises the PSMA antibody or antigen binding fragments disclosed herein. In some embodiments, the isolated synthetic receptor comprises the sequence set forth in SEQ ID NO: 127. In some embodiments, the isolated synthetic receptor comprises the sequence set forth in SEQ ID NO: 138. In some embodiments, the isolated synthetic receptor comprises the sequence set forth in SEQ ID NO: 252. In some embodiments, the isolated synthetic receptor comprises the sequence set forth in SEQ ID NO: 253.

In some embodiments, the isolated receptor's extracellular antigen-binding domains comprises the variable heavy (VH) chain sequence comprising three heavy chain CDR sequences, CDR-H1, CDR-H2, and CDR-H3, and the variable light (VL) chain sequence comprising three light chain CDR sequences, CDR-L1, CDR-L2, and CDR-L3 of the PSMA antibody or antigen binding fragments disclosed herein.

### CA9 Receptors

In some embodiments, the antigen-binding domain specifically binds to Carbonic Anhydrase IX (CA9) (SEQ ID NO: 1). In some embodiments, the domain includes an antigen-binding moiety that binds to Carbonic Anhydrase IX (CA9) (SEQ ID NO: 1).

In some embodiments, provided herein are isolated synthetic receptors comprising an antigen-binding domain that binds to Carbonic Anhydrase IX (CA9) (SEQ ID NO: 1).

In some embodiments, the isolated synthetic receptor comprises any CA9 antibody or antigen binding fragments disclosed herein. In some embodiments, the isolated synthetic receptor comprises the sequence as set forth in SEQ ID NO: 98. In some embodiments, the isolated synthetic receptor comprises the sequence as set forth in SEQ ID NO: 108. In some embodiments, the isolated synthetic receptor comprises the sequence as set forth in SEQ ID NO: 250. In some embodiments, the isolated synthetic receptor comprises the sequence as set forth in SEQ ID NO: 110. In some embodiments, the isolated synthetic receptor comprises the sequence as set forth in SEQ ID NO: 115. In some embodiments, the isolated synthetic receptor comprises the sequence as set forth in SEQ ID NO: 251.

In some embodiments, the isolated synthetic receptor's extracellular antigen-binding domains comprises the variable heavy (VH) chain sequence comprising three heavy chain CDR sequences, CDR-H1, CDR-H2, and CDR-H3, of any CA9 antibody or antigen binding fragments disclosed herein. In some embodiments, the isolated receptor's extracellular antigen-binding domains comprises the variable light (VL) chain sequence comprising three light chain CDR sequences, CDR-L1, CDR-L2, and CDR-L3 of any CA9 antibody or antigen binding fragments disclosed herein.

### Priming Receptors

Provided herein are priming receptors comprising an extracellular antigen-binding domain that specifically binds Prostate-Specific Membrane Antigen (PSMA). In some embodiments, the priming receptor comprises an extracellular antigen-binding domain that specifically binds Prostate-Specific Membrane Antigen (PSMA). PSMA is also known as FOLH1 or Folate Hydrolase 1 (HGNC: 3788, NCBI Entrez Gene: 2346, Ensembl: ENSG00000086205, UniProtKB/Swiss-Prot: Q04609). The amino acid sequence of PSMA is provided in SEQ ID NO: 2.

In certain aspects of the present disclosure, the priming receptor is a synthetic receptor based on the Notch protein. Binding of a natural Notch receptor to a cognate ligand, such as those from the Delta family of proteins, causes intramembrane proteolysis that cleaves an intracellular fragment of the Notch protein. This intracellular fragment is a transcriptional regulator that only functions when cleaved from Notch. Cleavage may occur by sequential proteolysis by ADAM metalloprotease and the gamma-secretase complex. This intracellular fragment enters the nucleus of a cell and activates cell-cell signaling genes. In contrast to a natural Notch protein, a synthetic notch priming receptor replaces the natural Notch intracellular fragment with one that causes a gene encoding a protein of choice, such as a CAR, to be transcribed upon release of the intracellular fragment from the priming receptor.

Notch receptors have a modular domain organization. The ectodomains of Notch receptors consist of a series of N-terminal epidermal growth factor (EGF)-like repeats that are responsible for ligand binding. In synthetic Notch receptors or priming receptors, the Notch ligand-binding domain is replaced with a ligand binding domain that binds a selected target ligand or antigen. The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

Multiple forms of priming receptors can be used in the methods, cells, and nucleic acids as described herein. One type of priming receptor contemplated for use in the methods and cells herein comprise a heterologous extracellular ligand binding domain, a linking polypeptide having substantial sequence identity with a Notch receptor including the NRR, a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor (lacking the NRR), a TMD, and an ICD. “Minimal Linker Notch” receptors comprise a heterologous extracellular ligand binding domain, a linking polypeptide lacking substantial sequence identity with a Notch receptor (e.g., a synthetic (GGS)n polypeptide sequence), a TMD, and an ICD. “Hinge Notch” receptors comprise a heterologous extracellular ligand binding domain, a hinge sequence comprising an oligomerization domain (i.e., a domain that promotes dimerization, trimerization, or higher order multimerization with a synthetic receptor and/or an existing host receptor), a TMD, and an ICD. All of these receptor classes are synthetic, recombinant, and do not occur in nature. In some embodiments, the non-naturally occurring receptors disclosed herein bind a target cell-surface displayed ligand, which triggers proteolytic cleavage of the receptors and release of a transcriptional regulator that modulates a custom transcriptional program in the cell. In some embodiments, the priming receptor does not include a LIN-12-Notch repeat (LNR) and/or a heterodimerization domain (HD) of a Notch receptor.

### Priming Receptor Extracellular Domain

The priming receptor disclosed herein comprises an extracellular domain that specifically binds Prostate-Specific Membrane Antigen (PSMA). In some embodiments, provided herein are priming receptors comprising an extracellular antigen-binding domain that binds to Prostate-Specific Membrane Antigen (PSMA) (SEQ ID NO: 2). In some embodiments, the priming receptor extracellular antigen-binding domains comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3 of any PSMA antibody or antigen binding fragments disclosed herein.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment or equivalent thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), a VHH, and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

In some embodiments, the extracellular antigen-binding domain specifically binds to Prostate-Specific Membrane Antigen. In some embodiments, the extracellular domain includes an antigen-binding moiety that binds to Prostate-Specific Membrane Antigen.

In some embodiments, the priming receptor comprises the PSMA antibody or antigen binding fragments disclosed herein. In some embodiments, the isolated receptor comprises the sequence set forth in SEQ ID NO: 127. In some embodiments, the priming receptor comprises the sequence set forth in SEQ ID NO: 138. In some embodiments, the isolated receptor comprises the sequence set forth in SEQ ID NO: 252. In some embodiments, the isolated receptor comprises the sequence set forth in SEQ ID NO: 253.

The PSMA priming receptor sequence provided in SEQ ID NO: 127 includes the leader sequence, while the PSMA priming receptor sequence provided in SEQ ID NO: 252 excludes the leader sequence. The PSMA priming receptor sequence provided in SEQ ID NO: 138 includes the leader sequence, while the PSMA priming receptor sequence provided in SEQ ID NO: 253 excludes the leader sequence.

In various embodiments, a priming receptor comprises means for binding a PSMA protein, optionally binding a human PSMA protein in the region(s) of human PSMA bound by the PSMA binders (e.g., as described in the Examples below). In some embodiments, the means binds a PSMA protein. In some embodiments, the means binds a human PSMA protein. In some embodiments, the means is a PSMA antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof). In some embodiments, the means for binding PSMA includes the anti-PSMA antibodies and antigen-binding fragments or equivalents thereof described herein.

The extracellular domain of the priming receptor can further comprise a signal sequence, such as a CD8α signal sequence. In some embodiments, the priming receptor comprises a CD8α signal sequence as set forth in SEQ ID NO: 91.

### Transmembrane Domain

In some embodiments, the priming receptor comprises a hinge domain. In some embodiments, the hinge domain is a CD8 or a CD8α hinge. In some embodiments, the priming receptor hinge domain comprises the sequence as set forth in SEQ ID NO: 85.

As described above, the priming receptor comprises a transmembrane domain (TMD) comprising one or more ligand-inducible proteolytic cleavage sites.

In some embodiments, the TMD comprises a Notch1 transmembrane domain.

Generally, the TMD suitable for the chimeric receptors disclosed herein can be any transmembrane domain of a Type 1 transmembrane receptor including at least one gamma-secretase cleavage site. Detailed description of the structure and function of the gamma-secretase complex as well as its substrate proteins, including amyloid precursor protein (APP) and Notch, can, for example, be found in a recent review by Zhang et al, Frontiers Cell Neurosci (2014). Non limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one gamma secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, YASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma. In some embodiments, the TMD of the chimeric polypeptides or Notch receptors of the disclosure is a TMD known for Notch receptors. In some embodiments, the TMD of the chimeric polypeptides or Notch receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus gallus.

In some embodiments, the priming receptor comprises a Notch cleavage site, such as S2 or S3. Additional proteolytic cleavage sites suitable for the compositions and methods disclosed herein include, but are not limited to, ADAM10, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Yal-Gly-Arg. Another example of a protease cleavage site that can be included in a proteolytically cleavable linker is a tobacco etch vims (TEV) protease cleavage site, e.g., Glu-Asn-Leu-Tyr-Thr-Gln-Ser (SEQ ID NO: 264), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 265), where cleavage occurs after the lysine residue. Another example of a protease cleavage site that can be included in a proteolytically cleavable linker is a thrombin cleavage site, e.g., Leu-Val-Pro-Arg (SEQ ID NO: 266). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr vims proteas, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrixmetalloprotemase (MT-MMP), stromelysin 3 (or MMP-11), thermo lysin, fibroblast collagenase and stromelysin-1, matrix metalloproteinase 13 (collagenase-3), tissue-type plasminogen activator(tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

In some embodiments, the amino acid substitution(s) within the TMD includes one or more substitutions within a “GV” motif of the TMD. In some embodiments, at least one of such substitution(s) comprises a substitution to alanine. Additional sequences and substitutions are described in WO2021061872, hereby incorporated by reference in its entirety.

In some embodiments, the TMD domain comprises the sequence as set forth in SEQ ID NO: 86.

### Intracellular Domain

In some embodiments, the priming receptor comprises one or more intracellular domains from or derived from a transcriptional regulator and/or a DNA-binding domain. In some embodiments, the intracellular domain comprises means for modulating transcription of one or more genes. In some embodiments, the means for modulating transcription of one or more genes comprises a transcriptional regulator, e.g., a transcriptional regulator provided herein or an equivalent thereof. In some embodiments, the priming receptor comprises one or more intracellular domains from or derived from a transcriptional regulator and/or a DNA-binding domain. In some embodiments, the intracellular domain comprises an HNF1α/p65 domain or a Gal4/VP64 domain.

Transcriptional regulators either activate or repress transcription from cognate promoters. Transcriptional activators typically bind nearby to transcriptional promoters and recruit RNA polymerase to directly initiate transcription. Transcriptional repressors bind to transcriptional promoters and sterically hinder transcriptional initiation by RNA polymerase. Other transcriptional regulators serve as either an activator or a repressor depending on where it binds and cellular conditions. Accordingly, as used herein, a “transcriptional activation domain” or “TAD” refers to the domain of a transcription factor that interacts with transcriptional control elements and/or transcriptional regulatory proteins (i.e., transcription factors, RNA polymerases, etc.) to increase and/or activate transcription of one or more genes. Non-limiting examples of transcriptional activation domains include: a herpes simplex virus VP16 activation domain, VP64 (which is a tetrameric derivative of VP16), HIV TAT, a NFkB p65 activation domain, p53 activation domains 1 and 2, a CREB (cAMP response element binding protein) activation domain, an E2A activation domain, NFAT (nuclear factor of activated T-cells) activation domain, yeast Gal4, yeast GCN4, yeast HAP1, MLL, RTG3, GLN3, OAF1, PIP2, PDR1, PDR3, PHO4, LEU3 glucocorticoid receptor transcription activation domain, B-cell POU homeodomain protein Oct2, plant Ap2, or any others known to one or ordinary skill in the art. In some embodiments, the transcriptional regulator is selected from Gal4-VP16, Gal4-VP64, tetR-VP64, ZFHD1-YP64, Gal4-KRAB, and HAP1-VP16. In some embodiments, the transcriptional regulator is Gal4-VP64. In some embodiments, the transcriptional regulator is p65. A transcriptional activation domain can comprise a wild-type or naturally occurring sequence, or it can be a modified, mutant, or derivative version of the original transcriptional activation domain that has the desired ability to increase and/or activate transcription of one or more genes. In some embodiments, the transcriptional regulator can further include a nuclear localization signal.

In some embodiments, the priming receptor comprises one or more intracellular “DNA-binding domains” (or “DB domains”). Such “DNA-binding domains” refer to sequence-specific DNA binding domains that bind a particular DNA sequence element. Accordingly, as used herein, a “sequence-specific DNA-binding domain” refers to a protein domain portion that has the ability to selectively bind DNA having a specific, predetermined sequence. A sequence-specific DNA binding domain can comprise a wild-type or naturally occurring sequence, or it can be a modified, mutant, or derivative version of the original domain that has the desired ability to bind to a desired sequence. In some embodiments, the sequence-specific DNA binding domain is engineered to bind a desired sequence. Non-limiting examples of proteins having sequence-specific DNA binding domains that can be used in synthetic proteins described herein include HNF1α, Gal4, GCN4, reverse tetracycline receptor, THY1, SYN1, NSE/RU5′, AGRP, CALB2, CAMK2A, CCK, CHAT, DLX6A, EMX1, zinc finger proteins or domains thereof, CRISPR/Cas proteins, such as Cas9, Cas3, Cas4, Cas5, Cas5e (or CasD), Cash, Cas6e, Cas6f, Cas7, Cas8a1, Cas8a2, Cas8b, Cas8c, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu196, and TALES. In some embodiments the DNA binding domain (DBD) is HNF1α.

In those embodiments where a CRISPR/Cas-like protein is used, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the functions of the systems described herein. For example, a CRISPR enzyme that is used as a DNA binding protein or domain thereof can be mutated with respect to a corresponding wild-type enzyme such that the mutated CRISPR or domain thereof lacks the ability to cleave a nucleic acid sequence containing a DNA binding domain target site. For example, a D10A mutation can be combined with one or more of H840A, N854A, or N863A mutations to produce a Cas9 enzyme substantially lacking all DNA cleavage activity.

In some embodiments, the intracellular domain comprises the sequence as set forth in SEQ ID NOs: 88, 89, or 90. In some embodiments, the intracellular domain comprises an HNF1α DNA binding domain (DBD) sequence as set forth in SEQ ID NO: 88. In some embodiments, the intracellular domain comprises a p65 transcriptional activation domain (TAD) sequence as set forth in SEQ ID NO: 89. In some embodiments, the intracellular domain comprises an HNF1α/p65 DBD-TAD domain sequence as set forth in SEQ ID NO: 90.

### Juxtamembrane Domain

The ECD and the TMD, or the TMD and the ICD, can be linked to each other with a linking polypeptide, such as a juxtamembrane domain. “SynNotch” or synthetic notch receptors comprise a heterologous extracellular ligand-binding domain, a linking polypeptide having substantial sequence identity with a Notch receptor JMD (including the NRR), a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor JMD but lacking the NRR (the LIN-12-Notch repeat (LNR) modules, and the heterodimerization domain), a TMD, and an ICD. “Minimal Linker Notch” receptors comprise a heterologous extracellular ligand-binding domain, a linking polypeptide lacking substantial sequence identity with a Notch receptor (for example, without limitation, having a synthetic (GGS)n polypeptide sequence), a TMD, and an ICD. “Hinge Notch” receptors comprise a heterologous extracellular ligand-binding domain, a hinge sequence comprising an oligomerization domain (i.e., a domain that promotes dimerization, trimerization, or higher order multimerization with a synthetic receptor and/or an existing host receptor), a TMD, and an ICD.

In some embodiments, the priming receptor comprises a juxtamembrane domain (JMD) peptide in between the extracellular domain and the transmembrane domain. In some embodiments, the priming receptor comprises a juxtamembrane domain (JMD) peptide in between the transmembrane domain and the intracellular domain. In some embodiments, the JMD peptide comprises an LWF motif. The use of LWF motifs in receptor constructs is described in U.S. Pat. No. 10,858,443, hereby incorporated by reference in its entirety. In some embodiments, the JMD peptide has substantial sequence identity to the JMD of Notch1, Notch2, Notch3, and/or Notch4. In some embodiments, the JMD peptide has substantial sequence identity to the Notch1, Notch2, Notch3, and/or Notch4 JMD, but does not include a LIN-12-Notch repeat (LNR) and/or a heterodimerization domain (HD) of a Notch receptor. In some embodiments, the JMD peptide does not have substantial sequence identity to the Notch1, Notch2, Notch3, and/or Notch4 JMD. In some embodiments, the JMD peptide includes an oligimerization domain which promotes formation of dimers, trimers, or higher order assemblages of the receptor. Such JMD peptides are described in WO2021061872, hereby incorporated by reference in its entirety.

In the Mini Notch receptor, the linking polypeptide is derived from a Notch JMD sequence after deletion of the NRR and HD domain. The Notch JMD sequence may be the sequence from Notch1, Notch2, Notch3, or Notch4, and can be derived from a non-human homolog, such as those from Drosophila, Gallus, Danio, and the like. Four to 50 amino acid residues of the remaining Notch sequence can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide, such as the signal transduction level when ligand induced or in the absence of ligand.

In the Minimal Linker Notch receptor, the linking polypeptide does not have substantial sequence identity to a Notch JMD sequence, including the Notch JMD sequence from Notch1, Notch2, Notch3, or Notch4, or a non-human homolog thereof. Four to 50 amino acid residues can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide of the disclosure. The Minimal Linker sequence can be designed to include or omit a protease cleavage site, and can include or omit a glycosylation site or sites for other types of post-translational modification. In some embodiments, the Minimal Linker does not comprise a protease cleavage site or a glysosylation site.

In some embodiments, the priming receptor further comprises a hinge. Hinge linkers that can be used in the priming receptor can include an oligomerization domain (e.g., a hinge domain) containing one or more polypeptide motifs that promote oligomer formation of the chimeric polypeptides via intermolecular disulfide bonding. In these instances, within the chimeric receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs (SEQ ID NO: 267).

Hinge polypeptide sequences can also be derived from a CD8α hinge domain, a CD28 hinge domain, a CD152 hinge domain, a PD-1 hinge domain, a CTLA4 hinge domain, an OX40 hinge domain, and functional variants thereof. In some embodiments, the hinge domain includes a hinge polypeptide sequence derived from a CD8α hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The Fn Notch linking polypeptide is derived from the Robo1 JMD, which contains a fibronectin repeat (Fn) domain, with a short polypeptide sequence between the Fn repeats and the TMD. The Fn Notch linking polypeptide does not contain a Notch negative regulatory region (NRR), or the Notch HD domain. The Fn linking polypeptide can contain 1, 2, 3, 4, or 5 Fn repeats. In some embodiments, the chimeric receptor comprises a Fn linking polypeptide having about 1 to about 5 Fn repeats, about 1 to about 3 Fn repeats, or about 2 to about 3 Fn repeats. The short polypeptide sequence between the Fn repeats and the TMD can be from about 2 to about 30 amino acid residues. In some embodiments, the short polypeptide sequence can be between about 5 and about 20 amino acids, of any sequence. In some embodiments, the short polypeptide sequence can be between about 5 and about 20 naturally-occurring amino acids, of any sequence. In some embodiments, the short polypeptide sequence can be between about 5 and about 20 amino acids, of any sequence but having no more than one proline. In some embodiments, the short polypeptide sequence can be between about 5 and about 20 amino acids, and about 50% or more of the amino acids are glycine. In some embodiments, the short polypeptide sequence can be between about 5 and about 20 amino acids, where the amino acids are selected from glycine, serine, threonine, and alanine. In some embodiments, the length and amino acid composition of the Fn linking polypeptide sequence can be varied to alter the orientation and/or proximity of the ECD and the TMD relative to one another to achieve a desired activity of the chimeric polypeptide of the disclosure.

### Stop-Transfer Sequence

In some embodiments, the priming receptor further comprises a stop-transfer sequence (STS) in between the transmembrane domain and the intracellular domains. The STS comprises a charged, lipophobic sequence. Without being bound by any theory, the STS serves as a membrane anchor, and is believed to prevent passage of the intracellular domain into the plasma membrane. The use of STS domains in priming receptors is described in WO2021061872, hereby incorporated by reference in its entirety. Non-limiting exemplary STS sequences include APLP1, APLP2, APP, TGBR3, CSF1R, CXCL16, CX3CL1, DAG1, DCC, DNER, DSG2, CDH1, GHR, HLA-A, IFNAR2, IGF1R, IL1R1, ERN2, KCNE1, KCNE2, CHL1, LRP1, LRP2, LRP18, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKTFD1, NECTIN1, KL, IL6R, EFNB1, CD44, CLSTN1, LRP8, PCDHGC3, NRG1, LRP1B, JAG2, EFNB2, DLL1, CLSTN2, EPCAM, ErbB4, KCNE3, CDH2, NRG2, PTPRK, BTC, EPHA4, IL1R2, KCNE4, SCN2B, Nradd, PTPRM, Notch1, Notch2, Notch3, and Notch4 STS sequences. In some embodiments, the STS is heterologous to the transmembrane domain. In some embodiments, the STS is homologous to the transmembrane domain. STS sequences are described in WO2021061872, hereby incorporated by reference in its entirety.

In some embodiments, the STS domain comprises the sequence as set forth in SEQ ID NO: 87.

### Chimeric Antigen Receptors

In another aspect, provided herein are chimeric antigen receptors comprising an extracellular antigen-binding domain that specifically binds to Carbonic Anhydrase IX (CA9). The CAR can be a human CAR, comprising fully human sequences, e.g., natural human sequences. The amino acid sequence of CA9 (HGNC: 1383, NCBI Entrez Gene: 768, Ensembl: ENSG00000107159, UniProtKB/Swiss-Prot: Q16790) is provided in SEQ ID NO: 1.

In some embodiments, the chimeric antigen receptor includes an extracellular portion comprising an antigen binding domain. The antigen recognition domain of a receptor such as a CAR can be linked to one or more intracellular signaling components, such as signaling components that mimic activation through an antigen receptor complex, such as a TCR complex, in the case of a CAR, and/or signal via another cell surface receptor. Thus, in some embodiments, the extracellular binding component (e.g., ligand-binding or antigen-binding domain) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some aspects, the chimeric antigen receptor includes an extracellular portion comprising an antigen binding domain described herein and an intracellular signaling domain. In some embodiments, an antibody or fragment includes an scFv, a VH, or a single-domain VH antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD8α or CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

### Chimeric Antigen Receptor Extracellular Domain

The chimeric antigen receptors disclosed herein comprises an extracellular domain that specifically binds Carbonic Anhydrase IX (CA9). In some embodiments, provided herein are chimeric antigen receptors comprising an extracellular antigen-binding domain that binds to Carbonic Anhydrase IX (CA9) (SEQ ID NO: 1). In some embodiments, the chimeric antigen receptors extracellular antigen-binding domains comprises a variable heavy (VH) chain sequence comprising three heavy chain CDR sequences, CDR-H1, CDR-H2, and CDR-H3. In some embodiments, the chimeric antigen receptors extracellular antigen-binding domains comprises a variable light (VL) chain sequence comprising three light chain CDR sequences, CDR-L1, CDR-L2, and CDR-L3 of any CA9 antibody or antigen binding fragments disclosed herein.

In some embodiments, the chimeric antigen receptor comprises the sequence as set forth in SEQ ID NO: 98. In some embodiments, the chimeric antigen receptor comprises the sequence as set forth in SEQ ID NO: 108 or 250. In some embodiments, the chimeric antigen receptor comprises the sequence as set forth in SEQ ID NO: 110. In some embodiments, the chimeric antigen receptor comprises the sequence as set forth in SEQ ID NO: 115 or 251. The CA9 CAR sequence provided in SEQ ID NO: 108 includes the leader sequence, while the CA9 CAR sequence provided in SEQ ID NO: 250 excludes the leader sequence. The CA9 CAR sequence provided in SEQ ID NO: 115 includes the leader sequence, while the CA9 CAR sequence provided in SEQ ID NO: 251 excludes the leader sequence.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment or equivalent thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), a VHH, and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

In some embodiments, the extracellular antigen-binding domain specifically binds to Carbonic Anhydrase IX (CA9). In some embodiments, the extracellular domain includes an antigen-binding moiety that binds to Carbonic Anhydrase IX (CA9).

In various embodiments, a CAR comprises means for binding a CA9 protein, optionally binding a human CA9 protein in the region(s) of human CA9 bound by the CA9 binders (e.g., as described in the Examples below). In some embodiments, the means binds a CA9 protein. In some embodiments, the means binds a human CA9 protein. In some embodiments, the means is a CA9 antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof) means for binding a CA9 protein. In some embodiments, the means for binding CA9 includes the anti-CA9 antibodies and antigen-binding fragments or equivalents thereof described herein.

The extracellular domain of the CAR can further comprise a signal sequence, such as a CD8α signal sequence. In some embodiments, the CAR comprises a CD8α signal sequence as set forth in SEQ ID NO: 91.

### CAR Transmembrane Domain

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, the transmembrane domain (TMD) of the receptor, e.g., the CAR, is a transmembrane domain of human CD28 or variant thereof, e.g., a 27-amino acid transmembrane domain of a human CD28 (UniProt Accession No.: P10747).

In some embodiments, the transmembrane domain (TMD) of the receptor, e.g., the CAR, is a transmembrane domain of human CD8α or variant thereof, e.g., a 24-amino acid transmembrane domain of a human CD8α (UniProt Accession No.: P01732).

In some embodiments, the CAR comprises a CD8α or CD28 TMD. In some embodiments, the CD8α TMD comprises the sequence set forth in SEQ ID NO: 93. In some embodiments, the CD28 TMD comprises the sequence set forth in SEQ ID NO: 95.

### CAR Hinge

In some embodiments, the CAR further includes a spacer, which may be or include at least a portion of an immunoglobulin constant region or variant or modified version thereof, such as a hinge region, e.g., a CD8α hinge, a CD28 hinge, an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include CD8α hinge, CD28 hinge, IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the CAR hinge comprises a CD8α, truncated CD8α, or CD28 hinge domain. In some embodiments, the hinge domain comprises a CD8α hinge comprising the sequence as set forth in SEQ ID NO: 92. In some embodiments, the hinge domain comprises a CD28 hinge comprising the sequence as set forth in SEQ ID NO: 94.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

### CAR Intracellular Domain

In some embodiments, upon ligation of the CAR, the cytoplasmic domain or intracellular signaling domain of the receptor activates at least one of the normal effector functions or responses of the immune cell, e.g., T cell engineered to express the receptor. In some embodiments, the CAR comprises means for activating at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the means for at least one of the normal effector functions or responses of the immune cell comprises an CAR intracellular activation domain, e.g., an intracellular activation domain provided herein or an equivalent thereof. In some embodiments, the means for at least one of the normal effector functions or responses of the immune cell comprises an CAR intracellular activation domain and a CAR co-stimulatory domain, e.g., a co-stimulatory domain provided herein or an equivalent thereof.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d.

The receptor, e.g., the CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the extracellular domain is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor-gamma, CD8, CD4, CD25, or CD16. For example, in some aspects, the CAR includes a chimeric molecule between CD3-zeta or Fc receptor-gamma and CD8, CD4, CD25 or CD16.

In some embodiments, the intracellular signaling domain comprises a human CD3 zeta stimulatory signaling domain or functional variant thereof, such as a 112 AA cytoplasmic domain of isoform 3 of human CD3 zeta. (UniProt Accession No.: P20963.2) or a CD3 zeta signaling domain as described in U.S. Pat. No. 7,446,190 or U.S. Pat. No. 8,911,993.

In some embodiments, cytoplasmic signaling molecule(s) in the CAR contain(s) a cytoplasmic signaling domain, portion thereof, or sequence derived from CD3 zeta. In some embodiments, the intracellular activation domain comprises a CD3ζ domain. In some embodiments, the intracellular activation domain comprises a CD3ζ domain set forth in SEQ ID NO: 97.

In some embodiments, the intracellular domain comprises an intracellular costimulatory signaling domain of 41BB or functional variant or portion thereof, such as a 42-amino acid cytoplasmic domain of a human 4-1BB (UniProt Accession No. Q07011.1) or functional variant or portion thereof.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB. In some embodiments, the chimeric antigen receptor contains an intracellular domain of a T cell costimulatory molecule. In some aspects, the T cell costimulatory molecule is 4-1BB.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In certain embodiments, the intracellular signaling domain comprises a CD8a transmembrane and signaling domain linked to a CD3 (e.g., CD3-zeta) intracellular domain. In some embodiments, the intracellular signaling domain comprises a 4-1BB (CD137, TNFRSF9) co-stimulatory domains, linked to a CD3 zeta intracellular domain. In some embodiments, the CAR comprises a 4-1BB co-stimulatory domain. In some embodiments, the 4-1BB co-stimulatory domain comprises the sequence as set forth in SEQ ID NO: 96.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

For example, in some embodiments, the CAR includes an antibody or fragment thereof, including single chain antibodies (sdAbs, e.g. containing only the VH region, also called a VHH), VH domains, and scFvs, described herein, a spacer such as a CD8a hinge, a CD8a transmembrane domain, a 4-1BB intracellular signaling domain, and a CD3 zeta signaling domain. In some embodiments, the CAR includes an antibody or fragment, including sdAbs and scFvs described herein, a spacer such as a CD8a hinge, a CD8a transmembrane domain, a 4-1BB intracellular signaling domain, and a CD3 zeta signaling domain.

Transgenes expressing the priming receptor and CAR system may be introduced into cells, such as a T cell, using, for example, a site-specific technique. With site specific integration of the transgenes (e.g. priming receptor and CAR), the transgenes may be targeted to a safe harbor locus or TRAC. Examples of site-specific techniques for integration into the safe harbor loci include, without limitation, homology-dependent engineering using nucleases and homology independent targeted insertion using Cas9.

The engineered cells have applications to immune-oncology. The priming receptor and CAR, for example, can be selected to target different specific tumor antigens. Examples of cancers that can be effectively targeted using such cells are blood cancers or solid cancers. In some embodiments, immune cell therapy can be used to treat solid tumors.

### Synthetic Pathway Activators

In various aspects, systems disclosed herein employ one or more “synthetic pathway activators” (SPAs). CAR-expressing immune cells can be limited by the necessity for in vivo expansion following infusion. To achieve robust expansion, T cells require three signals: antigen-stimulation, co-stimulation, and cytokine-induced stimulation. Activation of CARs is sufficient to induce the first two signals, but cannot recapitulate cytokine signaling. Furthermore, the tumor microenvironment is often immunosuppressive and devoid of pro-inflammatory cytokines. SPAs can thus be used to stimulate robust in vivo expansion and enhance desirable properties (i.e., increased survival, persistence, and potency) of T cells expressing priming receptors and/or CARs as described herein.

### SPA Structure

In various embodiments, SPAs mimic activation of interleukin signaling. Interleukin receptors are cytokine receptors that signal through Signal Transducer and Activator of Transcription (STAT) transcription factors (e.g., STAT3 and STAT5). Interleukin receptors typically function by dimerization in response to ligand binding. Once dimerized, receptors can bind janus-associated kinases (JAKs) to induce JAK cross-phosphorylation and downstream “JAK/STAT” signaling. Accordingly, induced receptor agonism or ligand-independent dimerization of receptors can be utilized to induce constitutive receptor activity and thus, constitutive cytokine signaling.

In various embodiments, SPAs comprise interleukin receptors or functional fragments thereof. In some embodiments, SPAs comprise or are derived from interleukin receptor intracellular signaling domains or functional fragments thereof. In some embodiments, SPAs comprise or are derived from interleukin-6 signal transducer (IL6ST) polypeptides or functional fragments thereof. Interleukin-6 signal transducer (IL6ST) is also known as glycoprotein 130 (gp130)

In various embodiments, one or more structural alterations can be made to confer constitutive activity to a SPA or functional fragment thereof. In some embodiments, structures or mutations can be added to induce SPA multimerization. In some embodiments, one or more amino acids can be mutated to a cysteine to allow formation of one or more disulfide bond(s), e.g., between two receptor monomers. In some embodiments, one or more amino acids can be inserted into a wild-type receptor polypeptide to promote dimerization, e.g., through formation of one or more disulfide bond(s).

In some embodiments, an exogenous polypeptide is operatively linked to a cytokine receptor or functional fragment thereof to cause their multimerization. In some embodiments, a leucine zipper polypeptide is operatively linked to a cytokine receptor or functional fragment thereof. In some embodiments, the leucine zipper polypeptide is a c-Jun leucine zipper. In some embodiments, an exogenous scaffold is operatively linked to a cytokine receptor or functional fragment thereof (e.g., IL6ST or gp130).

In some embodiments, SPAs can comprise a ligand agonist (e.g., a cytokine, e.g., an interleukin) that allows constitutive activation of the SPA. In some embodiments, the cytokine receptor and a soluble agonist are expressed simultaneously. In some embodiments, the cytokine receptor and a membrane-bound agonist are expressed simultaneously.

In various embodiments, SPAs are anchored to the cellular membrane. In some embodiments SPAs comprise an extracellular domain, a transmembrane domain, and an intracellular signaling domain. In some embodiments, SPAs comprise a transmembrane domain of an interleukin receptor.

### Exemplary SPAs

In some embodiments, the SPA comprises a leucine zipper-gp130 (referred to herein as “L-gp130”) or an L-gp130 intracellular signaling domain. L-gp130 comprises a homodimer, with each monomer comprising (a) an extracellular domain comprising an inserted cysteine residue that forms a disulfide linkage with another monomer and a c-Jun leucine zipper; and (b) an IL6ST transmembrane domain and intracellular signaling domain. The cysteine residue and the leucine zipper on each polypeptide can induce the formation of stable homodimers that mimic constitutive IL-6R activation. Additional details on the construction of L-gp130 are described in Stuhlmann-Laeisz et al. Mol Biol Cell. 2006 July; 17(7):2986-95 and in WO2020200325, which are hereby incorporated by reference in their entirety. The sequence of L-gp130 is provided in SEQ ID NO: 259. In some embodiments, the SPA comprises L-gp130 comprising a sequence as set forth in SEQ ID NO: 259.

In some embodiments, the SPA comprises an extracellular domain comprising a CD34 epitope or CD34 extracellular domain or fragment thereof. In some embodiments, the SPA comprises an extracellular domain comprising a CD34 epitope, an unpaired cysteine residue for multimerization, an IL6ST (gp130) transmembrane domain, and an IL6ST (gp130) intracellular domain. In some embodiments, the SPA comprises a sequence as set forth in SEQ ID NO: 141 or 254. The IL6ST (gp130) transmembrane domain sequence is provided in SEQ ID NO: 258. In some embodiments, the SPA comprises an IL6ST (gp130) transmembrane domain sequence as set forth in SEQ ID NO: 258. The IL6ST (gp130) intracellular signaling domain sequence is provided in SEQ ID NO: 257. In some embodiments, the SPA comprises an IL6ST (gp130) intracellular signaling domain sequence as set forth in SEQ ID NO: 257. In some embodiments, the SPA comprises an extracellular domain comprising the sequence as set forth in SEQ ID NO: 260. A nucleotide sequence encoding an exemplary SPA is provided in SEQ ID NO: 261. A nucleotide sequence encoding the exemplary SPA protein with an N-terminus leader sequence is provided in SEQ ID NO: 262. In some embodiments, the one or more nucleic acid(s) encoding the system discloses herein comprise SPA comprising a nucleic acid as set forth in SEQ ID NOs: 261 or 262.

### Nucleic Acids and Vectors

In another aspect, provided herein are one or more nucleic acids, wherein the one or more nucleic acids encode: a first chimeric polypeptide comprises a priming receptor comprising a first extracellular antigen-binding domain that specifically binds Prostate-Specific Membrane Antigen (PSMA); a second chimeric polypeptide comprising a chimeric antigen receptor (CAR) comprising a second extracellular antigen-binding domain that specifically binds to Carbonic Anhydrase IX (CA9); an optional third chimeric polypeptide comprising a synthetic pathway activator (SPA); and at least one or more nucleic acids comprising a nucleic acid sequence at least 15 nucleotides in length complementary to: a nucleic acid encoding human Fas Cell Surface Death Receptor (FAS) comprising the sequence set forth in SEQ ID NO: 3; and a nucleic acid encoding human Transforming Growth factor (TGF)-β Receptor 2 (TGFBR2) comprising the sequence set forth in SEQ ID NO: 4. In some embodiments, the nucleic acid sequence at least 15 nucleotides in length is complementary to nucleotides 1126 to 1364 of a nucleic acid encoding human FAS comprising the sequence set forth in SEQ ID NO: 3. In some embodiments, the nucleic acid comprises a nucleic acid sequence at least 15 nucleotides in length complementary to a nucleic acid encoding human Protein Tyrosine Phosphatase Non-Receptor Type 2 (PTPN2) comprising the sequence set forth in SEQ ID NO: 5. In some embodiments, the nucleic acid sequence is complementary to nucleotides 518-559 of a nucleic acid encoding human PTPN2 comprising the sequence set forth in SEQ ID NO: 5.

### RNA Interference Molecules

Transforming Growth Factor Beta Receptor 1 (TGF-βR1 or TGFBR1; HGNC: 11772, NCBI Entrez Gene: 7046, UniProtKB/Swiss-Prot: P36897) is a transmembrane serine/threonine protein kinase and forms a heteromeric complex with TGF-beta receptor type II (TGFRB2) when bound to TGF-beta, transducing the TGF-beta signal from the cell surface to the cytoplasm.

Transforming Growth Factor Beta Receptor 2 (TGF-βR2 or TGFBR2; HGNC: 11773, NCBI Entrez Gene: 7048, UniProtKB/Swiss-Prot: P37173) is a transmembrane serine/threonine protein kinase and forms a heterodimeric complex with TGF-beta receptor type-1 (TGFBR1) when bound to TGF-beta, resulting in transduction of the TGF-beta signal from the cell surface to the cytoplasm.

Fas Cell Surface Death Receptor (or Fas Receptor, FAS, CD95, or TNFRSF6; HGNC: 11920, NCBI Entrez Gene: 355; UniProtKB/Swiss-Prot: P25445) is an apoptosis-inducing TNF receptor superfamily member.

Protein Tyrosine Phosphatase Non-Receptor Type 2 (PTPN2; HGNC: 9650, NCBI Entrez Gene: 5771; UniProtKB/Swiss-Prot: P17706) is a phosphatase that regulates interferon and many other signaling pathways.

As used herein, “target gene” refers to a nucleic acid sequence in a cell, wherein the expression of the sequence may be specifically and effectively modulated using the nucleic acid molecules and methods described herein. In certain embodiments, the target gene may be implicated in the growth (proliferation), maintenance (survival), and/or immune behavior of an individual's immune cells. In some embodiments, the target gene is FAS. In some embodiments, the target gene is PTPN2. In some embodiments, the target gene is Transforming Growth Factor Beta Receptor 2 (TGFBR2). In some embodiments, two or more nucleic acid molecules target the TFGBR2 gene. In some embodiments, the target gene is Transforming Growth Factor Beta Receptor 1 (TGFBR1). In some embodiments, more than one target gene is modulated using a nucleic acid molecule and methods described herein. In some embodiments, at least two target gene are modulated using the nucleic acid molecules and methods described herein. In some embodiments, the nucleic acid molecule(s) is an shRNA. In some embodiments, the target genes are at least TGFBR1 and TGFBR2. In some embodiments, the target genes are at least FAS and TGFBR2. In some embodiments, the target genes are at least FAS, TGFBR1, and TGFBR2. In some embodiments, the target genes are at least FAS, TGFBR2, and PTPN2. In some embodiments, the target genes are at least FAS, PTPN2, TGFBR1, and TGFBR2.

In one aspect, provided herein are nucleic acid comprising a nucleic acid sequence at least 15 nucleotides in length complementary to a nucleic acid encoding human Transforming Growth Factor Beta Receptor 2 (TGFBR2) (SEQ ID NO: 4). In some embodiments, the nucleic acid comprises a nucleic acid sequence at least 15 nucleotides in length complementary to nucleotides 2215-2236, 4430-4451, or 3761-3782 of a nucleic encoding human Transforming Growth Factor Beta Receptor 2 (TGFBR2) comprising the sequence set forth in SEQ ID NO: 4. In some embodiments, the nucleic acid comprises a sequence selected from the group consisting of the sequences set forth in SEQ ID NOs: 34-82. In some embodiments, the nucleic acid comprises at least two sequence selected from the group consisting of the sequences set forth in SEQ ID NOs: 34-82.

In one aspect, provided herein are nucleic acid comprising a nucleic acid sequence at least 15 nucleotides in length complementary to a nucleic acid encoding human Transforming Growth Factor Beta Receptor 2 (TGFBR2) (SEQ ID NO: 4), wherein the nucleic acid sequence at least 15 nucleotides in length is at least 80%, 85%, 90%, 91%, 92%, 93%, 94%, 95%, 96%, 97%, 98%, 99%, or 100% complementary to a nucleic acid encoding human Transforming Growth Factor Beta Receptor 2 (TGFBR2) (SEQ ID NO: 4). In some embodiments, the nucleic acid comprises at least 80%, 85%, 90%, 91%, 92%, 93%, 94%, 95%, 96%, 97%, 98%, 99%, or 100% identity to a sequence selected from the group consisting of the sequences set forth in SEQ ID NOs: 34-82. In some embodiments, the nucleic acid comprises at least two sequence with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 34-82.

In one aspect, provided herein are nucleic acid comprising a nucleic acid sequence at least 15 nucleotides in length complementary to a nucleic acid encoding human Transforming Growth Factor Beta Receptor 1 (TGFBR1) (SEQ ID NO: 148). In some embodiments, the nucleic acid comprises a sequence selected from the group consisting of the sequences set forth in SEQ ID NOs: 149-178.

In one aspect, provided herein are nucleic acid comprising a nucleic acid sequence at least 15 nucleotides in length complementary to a nucleic acid encoding human Transforming Growth Factor Beta Receptor 1 (TGFBR1) (SEQ ID NO: 148), wherein the nucleic acid sequence at least 15 nucleotides in length is at least 80%, 85%, 90%, 91%, 92%, 93%, 94%, 95%, 96%, 97%, 98%, 99%, or 100% complementary to a nucleic acid encoding human Transforming Growth Factor Beta Receptor 1 (TGFBR1) (SEQ ID NO: 148). In some embodiments, the nucleic acid comprises at least 80%, 85%, 90%, 91%, 92%, 93%, 94%, 95%, 96%, 97%, 98%, 99%, or 100% identity to a sequence selected from the group consisting of the sequences set forth in SEQ ID NOs: 149-178. In some embodiments, the nucleic acid comprises at least two sequence with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 149-178.

In some embodiments, the nucleic acid comprises a nucleic acid sequence at least 15 nucleotides in length complementary to a nucleic acid encoding human Fas Cell Surface Death Receptor (FAS) comprising the sequence set forth in SEQ ID NO: 3. In some embodiments, the nucleic acid comprises a sequence selected from the group consisting of the sequences set forth in SEQ ID NOs: 6-20. In some embodiments, the nucleic acid sequence is complementary to nucleotides 1126 to 1364 of a nucleic acid encoding human FAS comprising the sequence set forth in SEQ ID NO: 3

In one aspect, provided herein are nucleic acid comprising a nucleic acid sequence at least 15 nucleotides in length complementary to a nucleic acid encoding human FAS comprising the sequence set forth in SEQ ID NO: 3, wherein the nucleic acid sequence at least 15 nucleotides in length is at least 80%, 85%, 90%, 91%, 92%, 93%, 94%, 95%, 96%, 97%, 98%, 99%, or 100% complementary to a nucleic acid encoding human FAS comprising the sequence set forth in SEQ ID NO: 3. In some embodiments, the nucleic acid comprises at least 80%, 85%, 90%, 91%, 92%, 93%, 94%, 95%, 96%, 97%, 98%, 99%, or 100% identity to a sequence selected from the group consisting of the sequences set forth in SEQ ID NOs: 6-20. In some embodiments, the nucleic acid comprises at least two sequence with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 6-20.

In some embodiments, the nucleic acid comprises a nucleic acid sequence at least 15 nucleotides in length complementary to a nucleic acid encoding human Protein Tyrosine Phosphatase Non-Receptor Type 2 (PTPN2) comprising the sequence set forth in SEQ ID NO: 5. In some embodiments, the nucleic acid comprises a sequence selected from the group consisting of the sequences set forth in SEQ ID NOs: 21-33. In some embodiments, the nucleic acid sequence is complementary to nucleotides 518-559 of a nucleic acid encoding human PTPN2 comprising the sequence set forth in SEQ ID NO: 5.

In one aspect, provided herein are nucleic acid comprising a nucleic acid sequence at least 15 nucleotides in length complementary to a nucleic acid encoding human Protein Tyrosine Phosphatase Non-Receptor Type 2 (PTPN2) comprising the sequence set forth in SEQ ID NO: 5, wherein the nucleic acid sequence at least 15 nucleotides in length is at least 80%, 85%, 90%, 91%, 92%, 93%, 94%, 95%, 96%, 97%, 98%, 99%, or 100% complementary to a nucleic acid encoding human Protein Tyrosine Phosphatase Non-Receptor Type 2 (PTPN2) comprising the sequence set forth in SEQ ID NO: 5. In some embodiments, the nucleic acid comprises at least 80%, 85%, 90%, 91%, 92%, 93%, 94%, 95%, 96%, 97%, 98%, 99%, or 100% identity to a sequence selected from the group consisting of the sequences set forth in SEQ ID NOs: 21-33. In some embodiments, the nucleic acid comprises at least two sequence with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 21-33. In some embodiments, the nucleic acid sequence is complementary to nucleotides 518-559 of a nucleic acid encoding human PTPN2 comprising the sequence set forth in SEQ ID NO: 5.

In some embodiments, the nucleic acid comprises a sequence as set forth in SEQ ID NO: 13. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 13. In some embodiments, the nucleic acid comprises a sequence as set forth in SEQ ID NO: 49. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 49. In some embodiments, the nucleic acid comprises a sequence as set forth in SEQ ID NO: 79. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 79. In some embodiments, the nucleic acid comprises a sequence as set forth in SEQ ID NO: 83. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 83. In some embodiments, the nucleic acid comprises a sequence as set forth in SEQ ID NO: 84. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 84.

In some embodiments, the nucleic acid comprises a sequence as set forth in SEQ ID NO: 182. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 182.

In some embodiments, the nucleic acid comprises a sequence as set forth in SEQ ID NO: 181, 183, 193, 194, or 195. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 181, 183, 193, 194, or 195.

In some embodiments, the nucleic acid is capable of reducing expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid is capable of reducing expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid is capable of reducing expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid sequence is at least 16, 17, 18, 19, 20, 21, or 22 nucleotides in length.

In some embodiments, the nucleic acid is an RNA interference (RNAi) molecule. Exemplary RNAi molecules include short hairpin RNA (shRNA), a small interfering RNA (siRNA), a double stranded RNA (dsRNA), or an antisense oligonucleotide. In some embodiments, the nucleic acid is a short hairpin RNA (shRNA), a small interfering RNA (siRNA), a double stranded RNA (dsRNA), or an antisense oligonucleotide. In some embodiments, the nucleic acid is an shRNA.

Single-stranded hairpin ribonucleic acids (shRNAs) are short duplexes where the sense and antisense strands are linked by a hairpin loop. They consist of a stem-loop structure that can be transcribed in cells from an RNA polymerase II or RNA polymerase III promoter on a plasmid construct. Once expressed, shRNAs are processed into RNAi species. Expression of shRNA from a plasmid is known to be relatively stable, thereby providing strong advantages over, for example, the use of synthetic siRNAs. shRNA expression units may be incorporated into a variety of plasmids, liposomes, viral vectors, and other vehicles for delivery and integration into a target cell. Expression of shRNA from a plasmid can be stably integrated for constitutive expression. shRNAs are synthesized in the nucleus of cells, further processed and transported to the cytoplasm, and then incorporated into the RNA-induced silencing complex (RISC) for activity. The shRNAs are converted into active siRNA molecules (which are capable of binding to, sequestering, and/or preventing the translation of mRNA transcripts encoded by target genes).

The Argonaute family of proteins is the major component of RISC. Within the Argonaute family of proteins, only Ago2 contains endonuclease activity that is capable of cleaving and releasing the passenger strand from the stem portion of the shRNA molecule. The remaining three members of Argonaute family, Ago1, Ago3 and Ago4, which do not have identifiable endonuclease activity, are also assembled into RISC and are believed to function through a cleavage-independent manner. Thus, RISC can be characterized as having cleavage-dependent and cleavage-independent pathways.

RNAi (e.g., antisense RNA, siRNA, microRNA, shRNA, etc.) are described in International Publication Nos. WO2018232356A1, WO2019084552A1, WO2019226998A1, WO2020014235A1, WO2020123871A1, and WO2020186219A1, each of which is herein incorporated by reference for all purposes.

Antisense oligonucleotide structure and chemical modifications are described in International PCT Publication No. WO20/132521, which is hereby incorporated by reference.

dsRNA and shRNA molecules and methods of use and production are described in U.S. Pat. Nos. 8,829,264; 9,556,431; and 8,252,526, each of which are hereby incorporated by reference

siRNA molecules and methods of use and production are described in U.S. Pat. No. 7,361,752 and US Patent Application No. US20050048647, both of which are hereby incorporated by reference.

Additional methods and compositions for RNA interference such as shRNA, siRNA, dsRNA, and antisense oligonucleotides are generally known in the art, and are further described in U.S. Pat. Nos. 7,361,752; 8,829,264; 9,556,431; 8,252,526, International PCT Publication No. WO00/44895; International PCT Publication No. WO01/36646; International PCT Publication No. WO99/32619; International PCT Publication No. WO00/01846; International PCT Publication No. WO01/29058; and International PCT Publication No. WO00/44914; International PCT Publication No. WO04/030634; each of which are hereby incorporated by reference.

The nucleic acid sequences (or constructs) that may be used to encode the RNAi molecules, such as an shRNA described herein, may comprise a promoter, which is operably linked (or connected), directly or indirectly, to a sequence encoding the RNAi molecules. Such promoters may be selected based on the host cell and the effect sought. Non-limiting examples of suitable promoters include constitutive and inducible promoters, such as EF1α or inducible Hepatocyte Nuclear Factor 1α (HNF1α)-YB TATA or RNA polymerase II (pol II)-based promoters. In some embodiments, the constitutive promoter is EF1α. In some embodiments, the EF1α promoter comprises as sequence as set forth in SEQ ID NO: 179. Non-limiting examples of suitable promoters further include the tetracycline inducible or repressible promoter, RNA polymerase I or III-based promoters, the pol II dependent viral promoters, such as the CMV-IE promoter, and the pol III U6 and H1 promoters, as well as Hepatocyte Nuclear Factor 1α (HNF1α)-YB TATA promotor provided in SEQ ID NO: 256. The bacteriophage T7 promoter may also be used (in which case it will be appreciated that the T7 polymerase must also be present). The nucleic acid sequences need not be restricted to the use of any single promoter, especially since the nucleic acid sequences may comprise two or more shRNAs (i.e., a combination of effectors), including but not limited to incorporated shRNA molecules. Each incorporated promoter may control one, or any combination of, the shRNA molecule components.

In certain embodiments, the promoter may be preferentially active in the targeted cells, e.g., it may be desirable to preferentially express at least one nucleic acid in immune cells using an immune cell-specific promoter. Introduction of such constructs into host cells may be effected under conditions whereby the two or more nucleic acids that are contained within the nucleic acid precursor transcript initially reside within a single primary transcript, such that the separate RNA molecules (for example, shRNA each comprising its own stem-loop structure) are subsequently excised from such precursor transcript by an endogenous ribonuclease. The resulting mature nucleic acids (e.g., shRNAs) may then induce degradation, and/or translation repression, of target gene mRNA transcripts produced in the cell. Alternatively, each of the precursor stem-loop structures may be produced as part of a separate transcript, in which case each nucleic acid sequence will preferably include its own promoter and transcription terminator sequences. Additionally, the multiple nucleic acid precursor transcripts may reside within a single primary transcript.

The stem-loop structures of the shRNA nucleic acids described herein may be about 40 to 100 nucleotides long or, preferably, about 50 to 75 nucleotides long. The stem region may be about 15-45 nucleotides in length (or more), or about 20-30 nucleotides in length. In some embodiments, the stem region is 22 nucleotides in length. In some embodiments, the stem region is 15, 16, 17, 18, 19, 20, 21, 22, 23, 24, 25, 26, 27 28 29, 30, 31, 32, 33, 34, 35, 36, 37, 38, 39, 40, 41, 42, 43, 44, or 45 nucleotides in length.

The stem may comprise a perfectly complementary duplex (but for any 3′ tail), however, bulges or interior loops may be present on either arm of the stem. The number of such bulges and asymmetric interior loops are preferably few in number (e.g., 1, 2 or 3) and are about 3 nucleotides or less in size. The terminal loop portion may comprise about 4 or more nucleotides, but preferably not more than about 25. The loop portion will preferably be 6-15 nucleotides in size.

As described herein, the stem regions of the shRNAs comprise passenger strands and guide strands, whereby the guide strands contain sequences complementary to the target mRNA transcript encoded by the target gene(s). Preferably, the G-C content and matching of guide strand and passenger strand is carefully designed for thermodynamically-favorable strand unwind activity with or without endonuclease cleavage. Furthermore, the specificity of the guide strand is preferably confirmed via a BLAST search (www.ncbi.nim.nih.gov/BLAST).

The disclosure herein provides that the expression level of multiple target genes may be modulated using the methods and nucleic acids described herein. For example, the disclosure herein provides that a first set of nucleic acids may be designed to include a sequence (a guide strand) that is designed to reduce the expression level of a first target gene, whereas a second set of nucleic acids may be designed to include a sequence (a guide strand) that is designed to reduce the expression level of a second target gene. The different sets of nucleic acids may be expressed and reside within the same, or separate, preliminary transcripts. In certain embodiments, such multiplex approach, i.e., the use of the nucleic acids described herein to modulate the expression level of two or more target genes, may have an enhanced therapeutic effect on a patient. For example, if a patient is provided with cells expressing the nucleic acid molecules described herein to treat, prevent, or ameliorate the effects of cancer, it may be desirable to provide the patient with two or more types of nucleic acid molecules, which are designed to reduce the expression level of multiple genes that are implicated in activation or repression of immune cells.

The nucleic acid molecule(s) described herein may be capable of reducing target gene expression in a cell by at least more than about 50% as compared to a control cell that does not comprise the nucleic acid molecule(s). For example, the nucleic acid molecule(s) (e.g., shRNA) can be capable of reducing expression of a target gene selected from the group consisting of FAS and TGBFR2 in the immune cell by at least 10%, 15%, 20%, 25%, 30%, 35%, 40%, 45%, 50%, 55%, 60%, 65%, 75%, 80%, 85%, 90%, 95%, 97%, 98%, 99%, or more as compared to a control cell that does not comprise the nucleic acid molecule(s). The nucleic acid molecule(s) can be capable of reducing expression of a target gene selected from the group consisting of FAS and TGBFR2 in the immune cell by at least between about 50-100%, 50-99%, 50-95%, 50-90%, 50-85%, 50-80%, 50-75%, 50-70%, 50-65%, 50-60%, 50-55%, or as compared to a control cell that does not comprise the nucleic acid molecule(s). In some embodiments, the nucleic acid molecule(s) is capable of reducing expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid molecule(s). In some embodiments, the nucleic acid molecule(s) is capable of reducing expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid molecule(s). In some embodiments, the nucleic acid molecule(s) is capable of reducing expression of TGBFR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid molecule(s).

The nucleic acid molecule(s) may be chemically synthesized, or in vitro transcribed, and may further include one or more modifications to phosphate-sugar backbone or nucleosides residues.

Other methods known in the art for introducing nucleic acids to cells may be used, such as lipid-mediated carrier transport, chemical mediated transport, such as calcium phosphate, and the like. Thus, the nucleic acid molecule(s) construct may be introduced along with components that perform one or more of the following activities: enhance RNA uptake by the cell, promote annealing of the duplex strands for shRNA, stabilize the annealed shRNA strands, or otherwise increase inhibition of the target gene.

### Additional Elements

In some embodiments, the one or more nucleic acid(s) further comprises a 5′ homology directed repair arm and/or a 3′ homology directed repair arm complementary to an insertion site in a host cell chromosome. In some embodiments, the one or more nucleic acid(s) comprises the 5′ homology directed repair arm and the 3′ homology directed repair arm. In some embodiments, the one or more nucleic acid(s) is incorporated into an expression cassette or an expression vector. In some embodiments, the expression cassette or the expression vector further comprises a constitutive promoter upstream of the one or more nucleic acid(s).

In some embodiments, the priming receptor, CAR, first nucleic acid, and the second nucleic acid are incorporated into a single expression cassette or a single expression vector. In some embodiments, the priming receptor, CAR, first nucleic acid, and the second nucleic acid are incorporated into two or more expression cassettes or expression vectors. In some embodiments, the expression vector(s) is a non-viral vector.

In some embodiments, the expression cassette or expression vector comprises a sequence as set forth in SEQ ID NO: 184. In some embodiments, the expression cassette comprises a sequence at least 80%, 85%, 90%, 91%, 92%, 93%, 94%, 95%, 96%, 97%, 98%, 99%, or 100% identical to a sequence as set forth in SEQ ID NO: 184. The expression cassette or expression vector comprising a sequence at least 80%, 85%, 90%, 91%, 92%, 93%, 94%, 95%, 96%, 97%, 98%, 99%, or 100% identical to the sequence as set forth in SEQ ID NO: 184 can further comprise one or more transgenes encoding any priming receptor or CAR as disclosed herein. In some embodiments, the expression cassette or expression vector comprises the sequences as set forth in SEQ ID NO: 185 and 186. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 185 and 186. The expression cassette or expression vector comprising the sequence as set forth in SEQ ID NO: 185 and 186 can further comprise one or more transgenes encoding any priming receptor or CAR as disclosed herein.

In some embodiments, the expression cassette or expression vector comprises a sequence as set forth in SEQ ID NO: 187. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 187. The expression cassette or expression vector comprising a sequence at least 80%, 85%, 90%, 91%, 92%, 93%, 94%, 95%, 96%, 97%, 98%, 99%, or 100% identical to the sequence as set forth in SEQ ID NO: 187 can further comprise one or more transgenes encoding any priming receptor or CAR as disclosed herein. In some embodiments, the expression cassette or expression vector comprises the sequences as set forth in SEQ ID NO: 188 and 189. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 188 and 189. The expression cassette or expression vector comprising the sequence as set forth in SEQ ID NO: 188 and 189 can further comprise one or more transgenes encoding any priming receptor or CAR as disclosed herein.

In some embodiments, the expression cassette or expression vector comprises a sequence as set forth in SEQ ID NO: 190. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 190. The expression cassette or expression vector comprising a sequence at least 80%, 85%, 90%, 91%, 92%, 93%, 94%, 95%, 96%, 97%, 98%, 99%, or 100% identical to the sequence as set forth in SEQ ID NO: 190 can further comprise one or more transgenes encoding any priming receptor or CAR as disclosed herein. In some embodiments, the expression cassette or expression vector comprises the sequences as set forth in SEQ ID NO: 191 and 192. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 191 and 192. The expression cassette or expression vector comprising the sequence as set forth in SEQ ID NO: 191 and 192 can further comprise one or more transgenes encoding any priming receptor or CAR as disclosed herein.

In some embodiments, the expression cassette or expression vector comprises a sequence as set forth in SEQ ID NO: 196. In some embodiments, the expression cassette comprises a sequence at least 80%, 85%, 90%, 91%, 92%, 93%, 94%, 95%, 96%, 97%, 98%, 99%, or 100% identical to a sequence as set forth in SEQ ID NO: 196. The expression cassette or expression vector comprising a sequence at least 80%, 85%, 90%, 91%, 92%, 93%, 94%, 95%, 96%, 97%, 98%, 99%, or 100% identical to the sequence as set forth in SEQ ID NO: 196 can further comprise one or more transgenes encoding any priming receptor or CAR as disclosed herein. In some embodiments, the expression cassette or expression vector comprises the sequences as set forth in SEQ ID NO: 197 and 198. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 197 and 198. The expression cassette or expression vector comprising the sequence as set forth in SEQ ID NO: 197 and 198 can further comprise one or more transgenes encoding any priming receptor or CAR as disclosed herein.

For example, the transgene included in any one of the cassettes as provided in any one of the sequences set forth in SEQ ID NOs: 184, 187, 190, or 196 can comprise an extracellular domain that specifically binds Prostate-Specific Membrane Antigen (PSMA) and/or a chimeric antigen receptor (CAR) that specifically binds Carbonic Anhydrase IX (CA9). For example, a transgene comprising a priming receptor can comprise an extracellular antigen-binding domain comprising a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3 of any PSMA antibody or antigen binding fragments disclosed herein. Similarly, a transgene comprising a chimeric antigen receptor (CAR) can comprise an extracellular antigen-binding domain comprising a variable heavy (VH) chain sequence comprising three heavy chain CDR sequences, CDR-H1, CDR-H2, and CDR-H3, and optionally a a variable light (VL) chain sequence comprising three light chain CDR sequences, CDR-L1, CDR-L2, and CDR-L3 of any CA9 antibody or antigen binding fragments disclosed herein.

In some embodiments, the expression cassette or expression vector comprises a sequence as set forth in SEQ ID NO: 143. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 143.

In some embodiments, the expression cassette or expression vector comprises a sequence as set forth in SEQ ID NO: 144. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 144.

In some embodiments, the expression cassette or expression vector comprises a sequence as set forth in SEQ ID NO: 145. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 145.

In some embodiments, the expression cassette or expression vector comprises a sequence as set forth in SEQ ID NO: 146. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 146.

In some embodiments, the expression cassette or expression vector comprises a sequence as set forth in SEQ ID NO: 147. In some embodiments, the expression cassette or expression vector comprises a sequence at least 80%, 85%, 90%, 91%, 92%, 93%, 94%, 95%, 96%, 97%, 98%, 99%, or 100% identical to a sequence as set forth in SEQ ID NO: 147.

The one or more interfering nucleic acid sequences (e.g., one or more shRNA) can be encoded in the intron regions of the nucleic acid insert, DNA template, module, cassette, single expression cassette, or a single expression vector that also encodes the priming receptor and/or the CAR. For example, if the DNA template includes promoters, such as EF1α, or inducible promoters such as the HNF1α-YB TATA promoter, described herein, to drive expression of the CAR or priming receptor, the one or more nucleic acid sequences (e.g., shRNA sequences) can be encoded in the promoter intronic region. In some embodiments, the one or more nucleic acid sequences is encoded in at least one intron region of the nucleic acid insert, module, cassette, or DNA template. In some embodiments, the one or more nucleic acid sequences is encoded in at least one EF1α intron region of the nucleic acid insert, module, cassette, or DNA template.

In some embodiments, the present disclosure contemplates nucleic acid(s), modules, cassettes, or DNA template inserts that comprise one or more transgenes encoding the priming receptors and/or CARs as described herein. In some embodiments, the DNA template insert or cassette encodes a priming receptor transgene. In some embodiments, the DNA template insert or cassette encodes a chimeric antigen receptor transgene. In some embodiments, the DNA template insert or cassette encodes a first nucleic acid complementary to at least 15 nucleotides of a human FAS mRNA sequence, and a second nucleic acid complementary to at least 15 nucleotides of a human TGBFR2 mRNA sequence. In some embodiments, the DNA template insert or cassette comprises a priming receptor transgene and a chimeric antigen receptor transgene. In some embodiments, the DNA template insert comprises a priming receptor transgene, a chimeric antigen receptor transgene, a first nucleic acid complementary to at least 15 nucleotides of a human FAS mRNA sequence, and a second nucleic acid complementary to at least 15 nucleotides of a human TGBFR2 mRNA sequence. In some embodiments, the DNA template insert comprises a priming receptor transgene, a chimeric antigen receptor transgene, a first nucleic acid complementary to at least 15 nucleotides of a human FAS mRNA sequence, and a second nucleic acid complementary to at least 15 nucleotides of a human TGBFR2 mRNA sequence.

In some embodiments, the one or more nucleic acid(s) are encoded on a single DNA template insert or cassette. In some embodiments, the one or more nucleic acid(s) are encoded on multiple DNA template inserts or cassettes. For example, the one or more nucleic acid(s) can be encoded on two, three, or four DNA template inserts.

The DNA template insert can also comprise a self-cleaving peptide. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chang et al. “Cleavage efficient 2A peptides for high level monoclonal antibody expression in CHO cells,” MAbs 7(2): 403-412 (2015)).

The DNA template insert can also comprise a WPRE element. WPRE elements are generally described in Higashimoto, T., et al. Gene Ther 14, 1298-1304 (2007); and Zufferey, R., et al. J Virol. 1999 April; 73(4):2886-92., both of which are hereby incorporated by reference.

The DNA template insert can also comprise an SV40 or a human growth hormone (GH1) polyA tail.

### Cells

Also provided herein are cells or immune cells comprising at least one DNA template non-virally inserted into a target region of the genome of the cell, wherein DNA template encodes the priming receptor and CAR system as described herein. Also provided herein are immune cells comprising the priming receptor that specifically binds Prostate-Specific Membrane Antigen (PSMA) and the chimeric antigen receptor that specifically binds CA9.

A cell comprising a DNA template insert at a target locus or safe harbor site as described in the present disclosure can be referred to as an engineered cell. In some embodiments, the cell or immune cell is any cell that can give rise to a pluripotent immune cell. In some embodiments, the immune cell is a primary immune cell. In some embodiments, the immune cell can be an induced pluripotent stem cell (iPSC) or a human pluripotent stem cell (HSPC). In some embodiments, the immune cell comprises primary hematopoietic cells or primary hematopoietic stem cells. In some embodiments, that engineered cell is a stem cell, a human cell, a primary cell, an hematopoietic cell, an adaptive immune cell, an innate immune cell, a natural killer (NK) cell, a T cell, a CD8+ cell, a CD4+ cell, or a T cell progenitor. In some embodiments, the immune cells are T cells. In some embodiments, the T cells are regulatory T cells, effector T cells, or naïve T cells. In some embodiments, the T cells are CD8+ T cells. In some embodiments, the T cells are CD4+ T cells. In some embodiments, the T cells are CD4+CD8+ T cells.

In some embodiments, the engineered cell is a stem cell, a human cell, a primary cell, an hematopoietic cell, an hematopoietic stem cell, an adaptive immune cell, an innate immune cell, a T cell or a T cell progenitor. Non-limiting examples of immune cells that are contemplated in the present disclosure include T cell, B cell, natural killer (NK) cell, NKT/iNKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cells is a T cell, NK cells, iPSC, and HSPC. In some embodiments, the engineered cells used in the present disclosure are human cell lines grown in vitro (e.g. deliberately immortalized cell lines, cancer cell lines, etc.).

Also provided herein are populations of cells comprising a plurality of the cells or immune cell. In some embodiments, the genome of at least 20%, 30%, 40%, 50%, 60%, 70%, 80%, 90%, 95%, 99% or greater of the cells comprises the priming receptor and CAR system as described herein.

### Method of Treating Immune-Related Condition of Disease

In another aspect, the disclosure herein provides methods of treating an immune-related condition (e.g., cancer) in an individual comprising administering to the individual an effective amount of a composition comprising a system comprising a priming receptor that specifically binds to PSMA and a chimeric antigen receptor that specifically binds to CA9, such as a cell composition comprising a system comprising a priming receptor that specifically binds to PSMA and a chimeric antigen receptor that specifically binds to CA9. In another aspect, the disclosure herein provides methods of enhancing an immune response in an individual comprising administering to the individual an effective amount of a composition comprising a system comprising a priming receptor that specifically binds to PSMA and a chimeric antigen receptor that specifically binds to CA9, such as a cell composition comprising a system comprising a priming receptor that specifically binds to PSMA and a chimeric antigen receptor that specifically binds to CA9.

In some embodiments, the target cell is a cell that expresses both PSMA and CA9. In some embodiments, the target cell is a cancer cell that expresses both PSMA and CA9. In some embodiments, the cancerous or diseased cell expresses both PSMA and CA9.

In some embodiments, the methods provided herein are useful for the treatment of an immune-related condition in an individual. In one embodiment, the individual is a human.

In some embodiments, the methods provided herein (such as methods of enhancing an immune response) are useful for the treatment of cancer and as such an individual receiving the system described herein has cancer. In some embodiments, the cancer is a solid cancer. In some embodiments, the cancer is immunoevasive. In some embodiments, the cancer is immunoresponsive. In particular embodiments, the cancer is kidney cancer, renal cell carcinoma, clear cell renal cell carcinoma (ccRcc), colorectal cancer, or lung cancer.

In some embodiments, the treatment results in a decrease in the cancer volume or size. In some embodiments, the treatment is effective at reducing a cancer volume as compared to the cancer volume prior to administration of the antibody. In some embodiments, the treatment results in a decrease in the cancer growth rate. In some embodiments, the treatment is effective at reducing a cancer growth rate as compared to the cancer growth rate prior to administration of the antibody. In some embodiments, the treatment is effective at eliminating the cancer.

In some embodiments, CA9 and PSMA are expressed at a higher level in the cancer as compared to a non-cancer cell. Levels of CA9 and PSMA can be assessed by any technique known in the field, including, but not limited to, protein assays or nucleic assays such as FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof.

In some aspects, provided herein are methods of inhibiting a target cell in a subject comprising administering the immune cell or population of immune cells disclosed herein to the subject, wherein the immune cell inhibits the target cell. Inhibition of a target cell includes killing of the cancer cell, or prevention or reduction in cancer cell growth, proliferation, or metastasis.

In some embodiments, the subject is conditioned with 30 mg/m² fludarabine and 300 mg/m² cyclophosphamide prior to administering the immune cell or population of immune cells disclosed herein. Administering the immune cell or population of immune cells disclosed herein to a subject without the conditioning may also be performed. In some embodiments, the subjects are conditioned on one or more of Days-3, -4, and/or -5 prior to prior to administering the immune cell or population of immune cells disclosed herein.

In some embodiments, subjects are administered 100×10⁶, 300×10⁶ or 1000×10⁶ of the immune cell or population of immune cells disclosed herein.

### Method of Immune Modulation

Methods of administration of a cell comprising a system comprising a priming receptor that specifically binds to PSMA and a chimeric antigen receptor that specifically binds to CA9 as described herein can result in modulation of an immune response. Modulation can be an increase or decrease in an immune response. In some embodiments, modulation is an increase in an immune response.

In one aspect, administration of a cell comprising a system comprising a priming receptor that specifically binds to PSMA and a chimeric antigen receptor that specifically binds to CA9 as described herein can result in induction of pro-inflammatory molecules, such as cytokines or chemokines. Generally, induced pro-inflammatory molecules are present at levels greater than that achieved with isotype control. Such pro-inflammatory molecules in turn result in activation of anti-tumor immunity, including, but not limited to, T cell activation, T cell proliferation, T cell differentiation, M1-like macrophage activation, and NK cell activation. Thus, the administration of a system comprising a priming receptor that specifically binds to PSMA and a chimeric antigen receptor that specifically binds to CA9 can induce multiple anti-tumor immune mechanisms that lead to tumor destruction.

In some embodiments, the target cell is a cell that expresses both PSMA and CA9. In some embodiments, the disease cell expresses both PSMA and CA9.

In another aspect, provided herein are methods of increasing an immune response in an individual comprising administering to the individual an effective amount of a cell comprising a system comprising a priming receptor that specifically binds to PSMA and a chimeric antigen receptor that specifically binds to CA9. In some embodiments, the method of increasing an immune response in a subject comprises administering to the subject a cell comprising a system comprising a priming receptor that specifically binds to PSMA and a chimeric antigen receptor that specifically binds to CA9.

In some embodiments, the cell is present in a pharmaceutical composition further comprising a pharmaceutically acceptable excipient.

In any and all aspects of increasing an immune response as described herein, any increase or decrease or alteration of an aspect of characteristic(s) or function(s) is as compared to a cell not comprising a composition comprising a system comprising a priming receptor that specifically binds to PSMA and a chimeric antigen receptor that specifically binds to CA9.

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response. In some embodiments, the immune response is started or initiated by administration of a cell comprising a system comprising a priming receptor that specifically binds to PSMA and a chimeric antigen receptor that specifically binds to CA9. In some embodiments, the immune response is enhanced by administration of cell comprising a system comprising a priming receptor that specifically binds to PSMA and a chimeric antigen receptor that specifically binds to CA9.

In another aspect, the present application provides methods of genetically editing a cell with a system comprising a priming receptor that specifically binds to PSMA and a chimeric antigen receptor that specifically binds to CA9, which results in the modulation of the immune function of the cell. The modulation can be increasing an immune response. In some embodiments, the modulation is an increase in immune function. In some embodiments, the modulation of function leads to the expression of an CA9 CAR. In some embodiments, the modulation of function leads to the activation of a cell comprising the system.

In some embodiments, the cell is a natural killer (NK) cell, a T cell, a CD8+ T cell, a CD4+ T cell, a primary T cell, or a T cell progenitor.

In some embodiments, the modulation of function of the cells comprising the priming receptor and CAR system as described herein leads to an increase in the cells' abilities to stimulate both native and activated T-cells, for example, by increasing cytokine or chemokine secretion by the cells expressing the priming receptor and CAR system. In some embodiments, the modulation of function enhances or increases the cells' ability to produce cytokines, chemokines, CARs, or costimulatory or activating receptors. In some embodiments, the modulation increases the T-cell stimulatory function of the cells expressing the priming receptor and CAR system, including, for example, the cells' abilities to trigger T-cell receptor (TCR) signaling, T-cell proliferation, or T-cell cytokine production.

In some embodiments, the increased immune response is secretion of cytokines and chemokines. In some embodiments, the priming receptor and CAR system induces increased expression of at least one cytokine or chemokine in a cell as compared to an isotype control cell. In some embodiments, the at least one cytokine or chemokine is selected from the group consisting of: IL-2 and IFNγ. In some embodiments, the cytokine or chemokine is IL-2. In some embodiments, the cytokine or chemokine is IFNγ. In some embodiments, the cytokine or chemokine secretion is increased a between bout 1-100-fold 1, 5, 10, 20, 30, 40, 50, 60, 70, 80, 90, 100, 1-10, 10-20, 20-30, 30-40, 40-50, 50-60, 60-70, 70-80, 80-90, or 90-100 fold as compared to an untreated cell or a cell treated with an isotype control antibody. In some embodiments, the chemokine is IL-2 and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the cytokine is IFNγ and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody.

In some embodiments, the enhanced immune response is anti-tumor immune cell recruitment and activation.

In some embodiments, the cell expressing the priming receptor and CAR system induces a memory immune response as compared to an isotype control cell. In general, a memory immune response is a protective immune response upon a subsequent exposure to pathogens or antigens that the immune system encountered previously. Exemplary memory immune responses include the immune response after infection or vaccination with an antigen. In general, memory immune responses are mediated by lymphocytes such as T cells or B cells. In some embodiments, the memory immune response is a protective immune response to cancer, including cancer cell growth, proliferation, or metastasis. In some embodiments, the memory immune response inhibits, prevents, or reduces cancer cell growth, proliferation, or metastasis.

### Methods of Editing Cells

In some aspects, provide herein are methods of editing a cell, comprising: providing a nuclease domain and a guide RNA, wherein the nucleic acid comprises a nucleic acid disclosed herein, and wherein the 5′ and 3′ ends of the nucleic acid comprise nucleotide sequences that are homologous to genomic sequences flanking an insertion site in the genome of the cell; introducing the nuclease domain and nucleic acid into the cell, wherein the guide RNA specifically hybridizes to a target region of the genome of the cell, and wherein the nuclease domain cleaves the target region to create the insertion site in the genome of the cell; and editing the cell via insertion of the nucleic acid into the insertion site in the genome of the cell.

The terms “gene editing” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behavior (e.g. for therapeutic purposes).

Currently available genome editing tools include zinc finger nucleases (ZFN) and transcription activator-like effector nucleases (TALENs) to incorporate genes at safe harbor loci (e.g. the adeno-associated virus integration site 1 (AAVS1) safe harbor locus). The DICE (dual integrase cassette exchange) system utilizing phiC31 integrase and Bxb1 integrase is a tool for target integration. Additionally, clustered regularly interspaced short palindromic repeat/Cas9 (CRISPR/Cas9) techniques can be used for targeted gene insertion.

Site specific gene editing approaches can include homology dependent mechanisms or homology independent mechanisms.

All methods known in the art for targeted insertion of gene sequences are contemplated in the methods described herein to insert constructs at gene targets or safe harbor loci.

Also provided herein are methods of making an immune cell comprising introducing a nucleic acid comprising the priming receptor and CAR system as described herein into a primary immune cell. In some embodiments, the immune cell is an isolated cell. In some embodiments, the immune cell is a mammalian cell (e.g., a human cell).

Provided herein are methods of inserting nucleotide sequences greater than about 5 kilobases in length into the genome of a cell, in the absence of a viral vector. In some embodiments, the nucleotide sequence greater than about 5 kilobase in length can be inserted into the genome of a primary immune cell, in the absence of a viral vector

Integration of large nucleic acids, for example nucleic acids greater than 5 kilobase in size, into cells, can be limited by low efficiency of integration, off-target effects and/or loss of cell viability. Described herein are methods and compositions for achieving integration of a nucleotide sequence, for example, a nucleotide sequence greater than about 5 kilobases in size, into the genome of a cell. In some methods the efficiency of integration is increased, off-target effects are reduced and/or loss of cell viability is reduced.

The plasmid can be introduced into an immune cell with a nuclease, such as a CRISPR-associated system (Cas). The nuclease can be introduced in a ribonucleoprotein format with a guide RNA (gRNA) that targets a specific site on the genome of the immune cell. The nuclease cuts the genomic DNA at this specific site. The specific site may be a portion of the genome that encodes an endogenous immune cell receptor. Thus, cutting the genome at this site will cause the immune cell to no longer express an endogenous immune cell receptor.

The plasmid may include 5′ and 3′ homology-directed repair arms complementary to sequences at a specific site on the genome of the immune cell. The complementary sequences are on either side of the site cut by the nuclease, which allows the plasmid to be incorporated at a specified insertion site on the immune cell's genome. Once the plasmid is incorporated, the cell will express the priming receptor. However, as explained, the design of the transgene cassette ensures that non-virally delivered circuit system receptors do not express CAR until the priming receptor binds to its cognate ligand and releases the cleavable transcription factor.

Initially, a T cell is isolated and optionally activated. The T cell may be obtained from a patient. Thus, the present disclosure provides methods in which immune cells, such as T cells, are harvested from a patient. Then, the plasmid that encodes the CAR and priming receptor are introduced into a T cell. Advantageously, the plasmids of the present disclosure can be introduced using electroporation. When introducing the plasmid via electroporation, the nuclease may also be introduced. By using electroporation, methods of the present disclosure avoid the use of viral vectors for introducing transgenes, which is a known bottleneck in immune cell engineering. The T cells are then expanded and co-cultured to create a sufficient quantity of engineered immune cells to be used as a therapeutic treatment.

Methods for editing the genome of a cell can include a) providing a Cas9 ribonucleoprotein complex (RNP) and a nucleic acid, comprising: (i) the RNP, wherein the RNP comprises a Cas9 nuclease domain and a guide RNA, wherein the guide RNA specifically hybridizes to a target region of the genome of the cell, and wherein the Cas9 nuclease domain cleaves the target region to create an insertion site in the genome of the cell; and (ii) a double-stranded or single-stranded a nucleic acid, such as a DNA template, wherein the size of the nucleic acid (e.g., DNA template) is greater than about 200 nucleotides, wherein the 5′ and 3′ ends of the nucleic acid (e.g., DNA template) comprise nucleotide sequences that are homologous to genomic sequences flanking the insertion site, and wherein the molar ratio of RNP to nucleic acid (e.g., DNA template) in the complex is from about 3:1 to about 100:1; and b) introducing the RNP complex and nucleic acid (e.g., DNA template) into the cell.

In some embodiments, the methods described herein provide an efficiency of delivery of the RNP complex and the nucleic acid (e.g., DNA template) of at least about 10%, 15%, 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the efficiency is determined with respect to cells that are viable after introducing the RNP complex and the nucleic acid (e.g., DNA template) into the cell. In some cases, the efficiency is determined with respect to the total number of cells (viable or non-viable) in which the RNP complex and the nucleic acid (e.g., DNA template) is introduced into the cell.

As another example, the efficiency of delivery can be determined by quantifying the number of genome edited cells in a population of cells (as compared to total cells or total viable cells obtained after the introducing step). Various methods for quantifying genome editing can be utilized. These methods include, but are not limited to, the use of a mismatch-specific nuclease, such as T7 endonuclease I; sequencing of one or more target loci (e.g., by sanger sequencing of cloned target locus amplification fragments); and high-throughput deep sequencing.

In some embodiments, loss of cell viability is reduced as compared to loss of cell viability after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages. In some embodiments, off-target effects of integration are reduced as compared to off-target integration after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages.

In some cases, the methods described herein provide for high cell viability of cells to which the RNP and nucleic acid (e.g., DNA template) has been introduced. In some cases, the viability of the cells to which the RNP and nucleic acid (e.g., DNA template) has been introduced is at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the viability of the cells to which the RNP and nucleic acid (e.g., DNA template) has been introduced is from about 20% to about 99%, from about 30% to about 90%, from about 35% to about 85% or 90% or higher, from about 40% to about 85% or 90% or higher, from about 50% to about 85% or 90% or higher, from about 50% to about 85% or 90% or higher, from about 60% to about 85% or 90% or higher, or from about 70% to about 85% or 90% or higher.

In the methods provided herein, the molar ratio of RNP to nucleic acid (e.g., DNA template) can be from about 3:1 to about 100:1. For example, the molar ratio can be from about 3:1 to 10:1, from about 3:1 to about 15:1, 3:1 to about 20:1; 3:1 to about 25:1; from about 3:1 to 50:1, from about 3:1 to 75:1, from about 3:1 to 100:1; from about 5:1 to 10:1, from about 5:1 to about 15:1, 5:1 to about 20:1; 5:1 to about 25:1; from about 5:1 to 50:1, from about 5:1 to 75:1, from about 5:1 to 100:1; from about 8:1 to about 12:1; from about 8:1 to about 15:1, from about 8:1 to about 20:1, from about 8:1 to about 25:1, from about 8:1 to 50:1, from about 8:1 to 75:1, from about 8:1 to 100:1; from about 10:1 to about 15:1, 10:1 to about 20:1, 10:1 to about 25:1; from about 10:1 to 50:1, from about 10:1 to 75:1, or from about 10:1 to 100:1.

In some embodiments, the nucleic acid (e.g., DNA template) is at a concentration of about 2.5 pM to about 25 pM. For example, the concentration of nucleic acid (e.g., DNA template) can be about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25 pM or any concentration in between these concentrations.

In some embodiments, the size or length of the nucleic acid (e.g., DNA template) is greater than about 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10 kb, 11 kb, 12 kb, 13 kb, 14 kb, 15 kb, or 16 kb or any size of nucleic acid (e.g., DNA template) in between these sizes. For example, the size of the DNA template can be about 4.5 kb to about 15 kb, about 4.5 kb to about 14 kb, about 4.5 kb to about 10 kb, about 5 kb to about 15 kb, about 5 kb to about 14 kb, about 5 kb to about 10 kb, about 5 kb to about 9 kb, about 5 kb to about 8 kb, about 5 kb to about 7 kb, about 5 kb to about 6 kb, about kb 6 to about 15 kb, about kb 6 to about 14 kb, about kb 6 to about 10 kb, about 6 kb to about 9 kb, about 6 kb to about 8 kb, about 6 kb to about 7 kb, about 7 kb to about 15 kb, about 7 kb to about 14 kb, about 7 kb to about 10 kb, about 7 kb to about 9 kb, about 7 kb to about 8 kb, about 8 kb to about 15 kb, about 8 kb to about 14 kb, about 8 kb to about 10 kb, about 8 kb to about 9 kb, about 9 kb to about 15 kb, about 9 kb to about 14 kb, about 9 kb to about 13 kb, about 9 kb to about 12 kb, about 9 kb to about 11 kb, about 9 kb to about 10 kb, about 10 kb to about 15 kb, about 10 kb to about 14 kb, about 10 kb to about 13 kb, about 10 kb to about 12 kb, or about 10 kb to about 11 kb.

In some embodiments, the amount of nucleic acid (e.g., DNA template) is about 1 μg to about 10 μg. For example, the amount of nucleic acid (e.g., DNA template) can be about 1 μg to about 2 μg, about 1 μg to about 3 μg, about 1 μg to about 4 μg, about 1 μg to about 5 μg, about 1 μg to about 6 μg, about 1 μg to about 7 μg, about 1 μg to about 8 μg, about 1 μg to about 9 μg, about 1 μg to about 10 μg. In some embodiments the amount of DNA template is about 2 μg to about 3 μg, about 2 μg to about 4 μg, about 2 μg to about 5 μg, about 2 μg to about 6 μg, about 2 μg to about 7 μg, about 2 μg to about 8 μg, about 2 μg to about 9 μg, or 2 μg to about 10 μg. In some embodiments the amount of nucleic acid (e.g., DNA template) is about 3 μg to about 4 μg, about 3 μg to about 5 μg, about 3 μg to about 6 μg, about 3 μg to about 7 μg, about 3 μg to about 8 μg, about 3 μg to about 9 μg, or about 3 μg to about 10 μg. In some embodiments, the amount of nucleic acid (e.g., DNA template) is about 4 μg to about 5 μg, about 4 μg to about 6 μg, about 4 μg to about 7 μg, about 4 μg to about 8 μg, about 4 μg to about 9 μg, or about 4 μg to about 10 μg. In some embodiments, the amount of DNA template is about 5 μg to about 6 μg, about 5 μg to about 7 μg, about 5 μg to about 8 μg, about 5 μg to about 9 μg, or about 5 μg to about 10 μg. In some embodiments, the amount of DNA template is about 6 μg to about 7 μg, about 6 μg to about 8 μg, about 6 μg to about 9 μg, or about 6 μg to about 10 μg. In some embodiments, the amount of nucleic acid (e.g., DNA template) is about 7 μg to about 8 μg, about 7 μg to about 9 μg, or about 7 μg to about 10 μg. In some embodiments, the amount of DNA template is about 8 μg to about 9 μg, or about 8 μg to about 10 μg. In some embodiments, the amount of DNA template is about 9 μg to about 10 μg.

In some cases, the size of the nucleic acid (e.g., DNA template) is large enough and in sufficient quantity to be lethal as naked DNA. In some embodiments, the DNA template encodes a heterologous protein or a fragment thereof. In some embodiments, the nucleic acid (e.g., DNA template) encodes at least one gene. In some embodiments, the DNA template encodes at least two genes. In some embodiments, the nucleic acid (e.g., DNA template) encodes one, two, three, four, five, six, seven, eight, nine, ten, or more genes.

In some embodiments, the nucleic acid (e.g., DNA template) includes regulatory sequences, for example, a promoter sequence and/or an enhancer sequence to regulate expression of the heterologous protein or fragment thereof after insertion into the genome of a cell. In some embodiments, the promoter is an inducible promoter. In some embodiments, the inducible promoter comprises one or more HNF1α enhancer elements. In some embodiments, the inducible promoter comprises a YB-TATA promoter element. In some embodiments, the inducible promoter comprises a sequence as set forth in SEQ ID NO: 256. In some embodiments, the promoter is an constitutive promoter. In some embodiments, the constitutive promoter is an EF1α promoter. In some embodiments, the constitutive promoter comprises the sequence of SEQ ID NO: 179.

In some cases, the nucleic acid (e.g., DNA template) is a linear DNA template. In some cases, the nucleic acid (e.g., DNA template) is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA.

In some cases, an RNP and nucleic acid (e.g., DNA template) complex is formed by incubating the RNP with the nucleic acid (e.g., DNA template) for less than about one minute to about thirty minutes, at a temperature of about 20° C. to about 25° C. For example, the RNP can be incubated with the DNA template for about 5 seconds, 10 seconds, 15 seconds, 20 seconds, 25 seconds, 30 seconds, 35 seconds, 40 seconds, 45 seconds, 50 seconds, 55 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes or any amount of time in between these times, at a temperature of about 20° C., 21° C., 22° C., 23° C., 24° C., or 25° C. In another example, the RNP can be incubated with the nucleic acid (e.g., DNA template) for less than about one minute to about one minute, for less than about one minute to about 5 minutes, for less than about 1 minute to about 10 minutes, for about 5 minutes to 10 minutes, for about 5 minutes to 15 minutes, for about 10 to about 15 minutes, for about 10 minutes to about 20 minutes, or for about 10 minutes to about 30 minutes, at a temperature of about 20° C. to about 25° C. In some embodiments, the RNP-DNA template complex and the cell are mixed prior to introducing the RNP-DNA template complex into the cell. In some embodiments, the RNP and nucleic acid (e.g., DNA template) and the cell are mixed prior to introducing the RNP and nucleic acid (e.g., DNA template) into the cell.

In some embodiments introducing the RNP complex and nucleic acid (e.g., DNA template) comprises electroporation. Methods, compositions, and devices for electroporating cells to introduce a RNP and nucleic acid (e.g., DNA template) can include those described in the examples herein. Additional or alternative methods, compositions, and devices for electroporating cells to introduce a RNP and nucleic acid (e.g., DNA template) can include those described in WO/2006/001614 or Kim, J. A. et al. Biosens. Bioelectron. 23, 1353-1360 (2008). Additional or alternative methods, compositions, and devices for electroporating cells to introduce a RNP and nucleic acid (e.g., DNA template) can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842, all of which are hereby incorporated by reference. Additional or alternative methods, compositions, and devices for electroporating cells to introduce a RNP and nucleic acid (e.g., DNA template) can include those described in Geng, T. et al. J. Control Release 144, 91-100 (2010); and Wang, J., et al. Lab. Chip 10, 2057-2061 (2010), all of which are hereby incorporated by reference.

In some embodiments, the Cas9 protein can be in an active endonuclease form, such that when bound to target nucleic acid as part of a complex with a guide RNA or part of a complex with a nucleic acid (e.g., DNA template), a double strand break is introduced into the target nucleic acid. The double strand break can be repaired by NHEJ to introduce random mutations, or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to any region of a genome that contains an NGG sequence. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to, CFP1, those described in Nature Methods 10, 1116-1121 (2013), and those described in Zetsche et al., Cell, Volume 163, Issue 3, p759-771, 22 Oct. 2015, both of which are hereby incorporated by reference.

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In some embodiments, the RNP comprises a Cas9 nuclease. In some embodiments, the RNP comprises a Cas9 nickase. In some embodiments, the RNP-DNA template complex comprises at least two structurally different RNP complexes. In some embodiments, the at least two structurally different RNP complexes contain structurally different Cas9 nuclease domains In some embodiments, the at least two structurally different RNP complexes contain structurally different guide RNAs. In some embodiments, wherein the at least two structurally different RNP complexes contain structurally different guide RNAs, each of the structurally different RNP complexes comprises a Cas9 nickase, and the structurally different guide RNAs hybridize to opposite strands of the target region.

In some cases, a plurality of RNP and nucleic acids (e.g., DNA templates) comprising structurally different ribonucleoprotein complexes is introduced into the cell. For example a Cas9 protein can be complexed with a plurality (e.g., 2, 3, 4, 5, or more, e.g., 2-10, 5-100, 20-100) of structurally different guide RNAs to target insertion of a nucleic acid (e.g., DNA template) at a plurality of structurally different target genomic regions.

In the methods and compositions provided herein, cells include, but are not limited to, eukaryotic cells, prokaryotic cells, animal cells, plant cells, fungal cells and the like. Optionally, the cell is a mammalian cell, for example, a human cell. The cell can be in vitro, ex vivo or in vivo. The cell can also be a primary cell, a germ cell, a stem cell or a precursor cell. The precursor cell can be, for example, a pluripotent stem cell, or a hematopoietic stem cell. In some embodiments, the cell is a primary hematopoietic cell or a primary hematopoietic stem cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4⁺ T cell. In some embodiments, the T cell is a CD8⁺ T cell. In some embodiments, the T cell is a CD4⁺CD8⁺ T cell. In some embodiments, the T cell is a CD4⁻CD8⁻ T cell. Populations of any of the cells modified by any of the methods described herein are also provided. In some embodiments, the methods further comprise expanding the population of modified cells.

In some cases, the cells are removed from a subject, modified using any of the methods described herein and administered to the patient. In other cases, any of the constructs described herein is delivered to the patient in vivo. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017), both of which are hereby incorporated by reference.

In some embodiments, the RNP and nucleic acid (e.g., DNA template) is introduced into about 1×10⁵ to about 2×10⁶ cells. For example, the RNP-DNA template complex can be introduced into about 1×10⁵ to about 5×10⁵ cells, about 1×10⁵ to about 1×10⁶, 1×10¹ to about 1.5×10⁶, 1×10¹ to about 2×10⁶, about 1×10⁶ to about 1.5×10⁶ cells or about 1×10⁶ to about 2×10⁶.

In some cases, the methods and compositions described herein can be used for generation, modification, use, or control of recombinant T cells, such as chimeric antigen receptor T cells (CAR T cells). Such CAR T cells can be used to treat or prevent cancer, an infectious disease, or autoimmune disease in a subject. For example, in some embodiments, one or more gene products are inserted or knocked-in to a T cell to express a heterologous protein (e.g., a chimeric antigen receptor (CAR) or a priming receptor).

### Insertion Sites

Methods for editing the genome of a T cell, specifically, include a method of editing the genome of a human T cell comprise inserting a nucleic acid sequence or construct into a target region in exon 1 of the TCR-α subunit (TRAC) gene in the human T cell. In some embodiments, the target region is in exon 1 of the constant domain of TRAC gene. In other embodiments, the target region is in exon 1, exon 2 or exon 3, prior to the start of the sequence encoding the TCR-α transmembrane domain.

Methods for editing the genome of a T cell also include a method of editing the genome of a human T cell comprise inserting a nucleic acid sequence or construct into a target region in exon 1 of a TCR-β subunit (TRBC) gene in the human T cell. In some embodiments, the target region is in exon 1 of the TRBC1 or TRBC2 gene.

Methods for editing the genome of a T cell, specifically, include a method of editing the genome of a human T cell comprise inserting a nucleic acid sequence or construct into a target region of a genomic safe harbor (GSH).

Gene editing therapies include, for example, vector integration and site specific integration. Site-specific integration is a promising alternative to random integration of viral vectors, as it mitigates the risks of insertional mutagenesis or insertional oncogenesis (Kolb et al. Trends Biotechnol. 2005 23:399-406; Porteus et al. Nat Biotechnol. 2005 23:967-973; Paques et al. Curr Gen Ther. 2007 7:49-66). However, site specific integration continues to face challenges such as poor knock-in efficiency, risk of insertional oncogenesis, unstable and/or anomalous expression of adjacent genes or the transgene, low accessibility (e.g. within 20 kB of adjacent genes), etc. These challenges can be addressed, in part, through the identification and use of safe harbor loci or safe harbor sites (SHS), which are sites in which genes or genetic elements can be incorporated without disruption to expression or regulation of adjacent genes.

The most widely used of the putative human safe harbor sites is the AAVS1 site on chromosome 19q, which was initially identified as a site for recurrent adenoassociated virus insertion. Other potential SHS have been identified on the basis of homology, with sites first identified in other species (e.g., the human homolog of the permissive murine Rosa26 locus) or among the growing number of human genes that appear non-essential under some circumstances. One putative SHS of this type is the CCR5 chemokine receptor gene, which, when disrupted, confers resistance to human immunodeficiency virus infection. Additional potential genomic SHS have been identified in human and other cell types on the basis of viral integration site mapping or gene-trap analyses, as was the original murine Rosa26 locus. The three top SHS, AAVS1, CCR5, and Rosa26, are in close proximity to many protein coding genes and regulatory elements. (See Sadelain, M., et al. (2012). Safe harbours for the integration of new DNA in the human genome. Nature reviews Cancer, 12(1), 51-58, the relevant disclosures of which are herein incorporated by reference in their entirety).

The AAVS1 (also known as the PPP1R12C locus) on human chromosome 19 is a known SHS for hosting transgenes (e.g. DNA transgenes) with expected function. It is at position 19q13.42. It has an open chromatin structure and is transcription-competent. The canonical SHS locus for AAVS1 is chr19: 55,625,241-55,629,351. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference. An exemplary AAVS1 target gRNA and target sequence are provided below:

AAVS1-gRNA sequence:
(SEQ ID NO: 268)
ggggccactagggacaggatGTTTTAGAGCTAGAAATAGCAAGTTAAAAT
AAGGCTAGTCCGTTATCAACTTGAAAAAGTGGCACCGAGTCGGTGCTTTT
TTT
AAVS1 target sequence:
(SEQ ID NO: 269)
ggggccactagggacaggat

CCR5, which is located on chromosome 3 at position 3p21.31, encodes the major co-receptor for HIV-1. Disruption at this site in the CCR5 gene has been beneficial in HIV/AIDS therapy and prompted the development of zinc-finger nucleases that target its third exon. The canonical SHS locus for CCR5 is chr3: 46,414,443-46,414,942. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference.

The mouse Rosa26 locus is particularly useful for genetic modification as it can be targeted with high efficiency and is expressed in most cell types tested. Irion et al. 2007 (“Identification and targeting of the ROSA26 locus in human embryonic stem cells.” Nature biotechnology 25.12 (2007): 1477-1482, the relevant disclosure of which are herein incorporated by reference) identified the human homolog, human ROSA26, in chromosome 3 (position 3p25.3). The canonical SHS locus for human Rosa26 (hRosa26) is chr3: 9,415,082-9,414,043. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference.

Additional examples of safe harbor sites are provided in Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference. Examples of additional integration sites are provided in Table D.

In some embodiments, the safe harbor sites allow for high transgene expression (sufficient to allow for transgene functionality or treatment of a disease of interest) and stable expression of the transgene over several days, weeks or months. In some embodiments, knockout of the gene at the safe harbor locus confers benefit to the function of the cell, or the gene at the safe harbor locus has no known function within the cell. In some embodiments the safe harbor locus results in stable transgene expression in vitro with or without CD3/CD28 stimulation, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, and positioned outside of a cancer-related gene.

As used, a “nearby gene” can refer to a gene that is within about 100 kB, about 125 kB, about 150 kB, about 175 kB, about 200 kB, about 225 kB, about 250 kB, about 275 kB, about 300 kB, about 325 kB, about 350 kB, about 375 kB, about 400 kB, about 425 kB, about 450 kB, about 475 kB, about 500 kB, about 525 kB, about 550 kB away from the safe harbor locus (integration site).

In some embodiments, the present disclosure contemplates inserts that comprise one or more transgenes. The transgene can encode a therapeutic protein, an antibody, a peptide, or any other gene of interest. The transgene integration can result in, for example, enhanced therapeutic properties. These enhanced therapeutic properties, as used herein, refer to an enhanced therapeutic property of a cell when compared to a typical immune cell of the same normal cell type. For example, a T cell having “enhanced therapeutic properties” has an enhanced, improved, and/or increased treatment outcome when compared to a typical, unmodified and/or naturally occurring T cell. The therapeutic properties of immune cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the target locus or safe harbor site. In some embodiments, the insert size comprises at least about 4.5 kilobasepairs (kb) to about 10 kilobasepairs (kb). In some embodiments, the insert size comprises about 5000 nucleotides or more basepairs. In some embodiments, the insert size comprises up to 4.5, 4.8, 5, 6, 7, 8, 9, 10, 11, 12, 13, 14, 15, 16, 17, 18, 19, 20 kbp (kilo basepairs) or the sizes in between. In some embodiments, the insert size is greater than 4.5, 4.8, 5, 6, 7, 8, 9, 10, 11, 12, 13, 14, 15, 16, 17, 18, 19, 20 kbp or the sizes in between. In some embodiments, the insert size is within the range of 4.5-15 kbp or is any number in that range. In some embodiments, the insert size is within the range of 4.8-8.3 kbp or is any number in that range. In some embodiments, the insert size is within the range of 5-8.3 kbp or is any number in that range. In some embodiments, the insert size is within the range of 5-15 kbp or is any number in that range. In some embodiments, the insert size is within the range of 4.5-20 kbp or is any number in that range. In some embodiments, the insert size is 5-10 kbp. In some embodiments, the insert size is 4.5-10, 5-10, 6-10, 7-10, 8-10, 9-10 kbp. In some embodiments, the insert size is 4.5-11, 6-11, 7-11, 8-11, 9-11, or 10-11 kbp. In some embodiments, the insert size is 4.5-12, 6-12, 7-12, 8-12, 9-12, 10-12, or 11-12 kbp. In some embodiments, the insert size is 4.5-13, 6-13, 7-13, 8-13, 9-13, 10-13, 11-13, or 12-13 kbp. In some embodiments, the insert size is 4.5-14, 6-14, 7-14, 8-14, 9-14, 10-14, 11-14, 12-14 or 13-14 kbp. In some embodiments, the insert size is 4.5-15, 6-15, 7-15, 8-15, 9-15, 10-15, 11-15, 12-15, 13-15, or 14-15 kbp. In some embodiments, the insert size is 4.5-16, 6-16, 7-16, 8-16, 9-16, 10-16, 11-16, 12-16, 13-16, 14-16 or 15-16 kbp. In some embodiments, the insert size is 4.5-17, 6-17, 7-17, 8-17, 9-17, 10-17, 11-17, 12-17, 13-17, or 14-17, 15-17 or 16-17 kbp. In some embodiments, the insert size is 4.5-18, 6-18, 7-18, 8-18, 9-18, 10-18, 11-18, 12-18, 13-18, 14-18, 15-18, 16-18 or 17-18 kbp. In some embodiments, the insert size is 4.5-19, 6-19, 7-19, 8-19, 9-19, 10-19, 11-19, 12-19, 13-19, 14-19, 15-19, 16-19, 17-19, or 18-19 kbp. In some embodiments, the insert size is 4.5-20, 6-20, 7-20, 8-20, 9-20, 10-20, 11-20, 12-20, 13-20, 14-20, 15-20, 16-20, 17-20, 18-20, or 19-20 kbp.

The inserts of the present disclosure refer to nucleic acid molecules or polynucleotide inserted at a target locus or safe harbor site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. In some embodiments, the insert is an RNA molecule or comprises ribonucleotides. The nucleotides in the insert are contemplated as naturally occurring nucleotides, non-naturally occurring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprises a non-coding sequence (e.g., control elements, e.g., a promoter sequence). In some embodiments, the insert encodes transcription factors. In some embodiments, the insert encodes an antigen binding receptors such as single receptors, T-cell receptors (TCRs), priming receptors, CARs, mAbs, etc. In some embodiments, the the insert is a human sequence. In some embodiments, the insert is chimeric. In some embodiments, the insert is a multi-gene/multi-module therapeutic cassette. A multi-gene/multi-module therapeutic cassette refers to an insert or cassette having one or more than one receptor (e.g., synthetic receptors such as a CAR or a priming receptor), other exogenous protein coding sequences, non-coding RNAs, transcriptional regulatory elements, and/or insulator sequences, etc.

In some embodiments, the nucleic acid sequence is inserted into the genome of the cell such as an immune cell or T cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbor loci include, without limitation, homology-dependent engineering using nucleases and homology independent targeted insertion using Cas9 or other CRISPR endonucleases.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in US patent Nos. U.S. 11033584B2 and U.S. 11814624B2, the relevant disclosures of which are herein incorporated by reference in their entirety.

Examples of integration sites contemplated are provided in Table D.

TABLE D
sgRNA sequences

| | | | | | | Median (% |
|---|---|---|---|---|---|---|
| | | | | | | Modified), |
| | | | | | | summarized |
| | SEQ | | sgRNA start | sgRNA | Integra- | from 2 |
| sgRNA | ID | | coor | Target | tion | donors, 2 |
| ID | NO: | sgRNA Sequence | GRCH38 | Loci | Site | primersets |
| sgRNA | 270 | GCACCTGAATACCAC | chr16:8881181 | APRT | APRT | 79.28 |
| _1 | | GCCTG | 8 | | | |
| sgRNA | 271 | CGCCTGCGATGTAGT | chr16:8881155 | APRT | APRT | 78.60 |
| _2 | | CGATG | 1 | | | |
| sgRNA | 272 | CAGGACGGGCGAGAT | chr16:8881164 | APRT | APRT | 85.25 |
| _3 | | GTCCC | 0 | | | |
| sgRNA | 273 | CTGAATCTTTGGAGT | chr15:4471542 | B2M | B2M | 78.51 |
| _4 | | ACCTG | 5 | | | |
| sgRNA | 274 | GGCCACGGAGCGAGA | chr15:4471155 | B2M | B2M | 94.75 |
| _5 | | CATCT | 0 | | | |
| sgRNA | 275 | AAGTCAACTTCAATG | chr15:4471551 | B2M | B2M | 70.97 |
| _6 | | TCGGA | 5 | | | |
| sgRNA | 276 | GCTTGGAGGCCTGAT | chr19:3614111 | CAPNS1 | CAPNS | 89.34 |
| _7 | | CAGCG | 1 | | 1 | |
| sgRNA | 277 | CTTATCTCTTCGCAGC | chr19:3614230 | CAPNS1 | CAPNS | 91.09 |
| _8 | | GAGG | 1 | | 1 | |
| sgRNA | 278 | CACACATTACTCCAA | chr19:3614267 | CAPNS1 | CAPNS | 71.98 |
| _9 | | CATTG | 6 | | 1 | |
| sgRNA | 279 | TTCCGCAAAATAGAG | chr3:10574601 | CBLB | CBLB | 91.55 |
| _10 | | CCCCA | 9 | | | |
| sgRNA | 280 | TGCACAGAACTATCG | chr3:10575162 | CBLB | CBLB | 91.43 |
| _11 | | TACCA | 2 | | | |
| sgRNA | 281 | GCAATAAGACTCTTT | chr3:10585347 | CBLB | CBLB | 76.18 |
| _12 | | AAAGA | 0 | | | |
| sgRNA | 282 | CAAAGAGATTACGAA | chr1:11675465 | CD2 | CD2 | 89.80 |
| _13 | | TGCCT | 8 | | | |
| sgRNA | 283 | CAAGGCACCCCAGGT | chr1:11675466 | CD2 | CD2 | 92.70 |
| _14 | | TTCCA | 3 | | | |
| sgRNA | 284 | TTACGAATGCCTTGG | chr1:11675466 | CD2 | CD2 | 92.82 |
| _15 | | AAACC | 6 | | | |
| sgRNA | 285 | CAGAGACGCATCTGA | chr11:1183155 | CD3E | CD3E | 90.96 |
| _16 | | CCCTC | 40 | | | |
| sgRNA | 286 | CATGCAGTTCTCACA | chr11:1183137 | CD3E | CD3E | 87.47 |
| _17 | | CACTG | 15 | | | |
| sgRNA | 287 | GTGTGAGAACTGCAT | chr11:1183137 | CD3E | CD3E | 86.65 |
| _18 | | GGAGA | 15 | | | |
| sgRNA | 288 | TCTCATTTCAGGAAA | chr11:1183497 | CD3G | CD3G | 87.24 |
| _19 | | CCACT | 48 | | | |
| sgRNA | 289 | AGTCATACACCTTAA | chr11:1183497 | CD3G | CD3G | 87.99 |
| _20 | | CCAAG | 54 | | | |
| sgRNA | 290 | TTCAAGGAAACCAGT | chr11:1183524 | CD3G | CD3G | 86.55 |
| _21 | | TGAGG | 58 | | | |
| sgRNA | 291 | GAGCCTTGCCTGGAA | chr11:6111817 | CD5 | CD5 | 84.03 |
| _22 | | ATCTG | 7 | | | |
| sgRNA | 292 | AAGCGTCAAAAGTCT | chr11:6111832 | CD5 | CD5 | 89.19 |
| _23 | | GCCAG | 4 | | | |
| sgRNA | 293 | CGTTCCAACTCGAAG | chr11:6111812 | CD5 | CD5 | 83.11 |
| _24 | | TGCCA | 1 | | | |
| sgRNA | 294 | GAGCGACTGGGACAC | chr9:13686624 | EDF1 | EDF1 | 88.84 |
| _25 | | GGTGA | 6 | | | |
| sgRNA | 295 | GCTGCGCAAGAAGGG | chr9:13686621 | EDF1 | EDF1 | 91.04 |
| _26 | | CCCTA | 1 | | | |
| sgRNA | 296 | TTGTTCTGGCCAGCA | chr9:13686343 | EDF1 | EDF1 | 85.98 |
| _27 | | GCCCC | 3 | | | |
| sgRNA | 297 | CTTCCAGAGCCACAT | chr19:4896579 | FTL | FTL | 93.10 |
| _28 | | CATCG | 1 | | | |
| sgRNA | 298 | GGGACTCACCAGAGA | chr19:4896560 | FTL | FTL | 88.86 |
| _29 | | GAGGT | 1 | | | |
| sgRNA | 299 | CGGTCGAAATAGAAG | chr19:4896577 | FTL | FTL | 93.14 |
| _30 | | CCCTA | 0 | | | |
| sgRNA | 300 | AAAAGGATATTGTGC | chr10:8793301 | PTEN | PTEN | 92.37 |
| _31 | | AACTG | 5 | | | |
| sgRNA | 301 | TGTGCATATTTATTAC | chr10:8793318 | PTEN | PTEN | 90.64 |
| _32 | | ATCG | 3 | | | |
| sgRNA | 302 | TTTGTGAAGATCTTG | chr10:8793308 | PTEN | PTEN | 85.36 |
| _33 | | ACCAA | 7 | | | |
| sgRNA | 303 | TGTCATGCTGAACCG | chr18:1283097 | PTPN2 | PTPN2 | 87.94 |
| _34 | | CATTG | 2 | | | |
| sgRNA | 304 | CCACTCTATGAGGAT | chr18:1285921 | PTPN2 | PTPN2 | 92.45 |
| _35 | | AGTCA | 9 | | | |
| sgRNA | 305 | TTGACATAGAAGAGG | chr18:1283682 | PTPN2 | PTPN2 | 93.96 |
| _36 | | CACAA | 8 | | | |
| sgRNA | 306 | GAGTACTACACTCAG | chr12:6952098 | PTPN6 | PTPN6 | 89.61 |
| _37 | | CAGCA | | | | |
| sgRNA | 307 | TCACGCACAAGAAAC | chr12:6954872 | PTPN6 | PTPN6 | 82.74 |
| _38 | | GTCCA | | | | |
| sgRNA | 308 | AGGTCTCGGTGAAAC | chr12:6951610 | PTPN6 | PTPN6 | 91.27 |
| _39 | | CACCT | | | | |
| sgRNA | 309 | AGCATTATCCAAAGA | chr1:19869687 | PTPRC | PTPRC | 88.88 |
| _40 | | GTCCG | 3 | | | |
| sgRNA | 310 | ATATTAATTCTTACCA | chr1:19869237 | PTPRC | PTPRC | 88.95 |
| _41 | | GTGG | 0 | | | |
| sgRNA | 311 | AGCTTTAAATCAAGG | chr1:19875617 | PTPRC | PTPRC | 96.89 |
| _42 | | TTCAT | 6 | | | |
| sgRNA | 312 | ATCCCGAGCCCTAAG | chr11:6743632 | PTPRCAP | PTPRC | 84.08 |
| _43 | | GTGCA | 5 | | AP | |
| sgRNA | 313 | GGCAGCGCGGAGGAC | chr11:6743628 | PTPRCAP | PTPRC | 97.74 |
| _44 | | AGCGT | 5 | | AP | |
| sgRNA | 314 | CTCAGGGGGCTACTA | chr11:6743617 | PTPRCAP | PTPRC | 91.50 |
| _45 | | CCACC | 0 | | AP | |
| sgRNA | 315 | GTCACCGACGAGACC | chr5:82277810 | RPS23 | RPS23 | 79.40 |
| _46 | | AGAAG | | | | |
| sgRNA | 316 | GTCGTGGACTTCGTA | chr5:82277843 | RPS23 | RPS23 | 83.07 |
| _47 | | CTGCT | | | | |
| sgRNA | 317 | TAATTTTTAGGCAAG | chr5:82277860 | RPS23 | RPS23 | 61.94 |
| _48 | | TGTCG | | | | |
| sgRNA | 318 | TTAGCTGTTAGACTT | chr14:5199381 | RTRAF | RTRAF | 85.50 |
| _49 | | GAATA | 0 | | | |
| sgRNA | 319 | CGAGAGCCGTCAACT | chr14:5198965 | RTRAF | RTRAF | 85.64 |
| _50 | | TGCGT | 2 | | | |
| sgRNA | 320 | CGGCTTCAACTGCAA | chr14:5198970 | RTRAF | RTRAF | 88.77 |
| _51 | | AGGTG | 0 | | | |
| sgRNA | 321 | TATGAAAAAGCAGAG | chr15:4379302 | SERF2 | SERF2 | 89.61 |
| _52 | | CGACT | 5 | | | |
| sgRNA | 322 | TCTGGCGGGCGAGCT | chr15:4379298 | SERF2 | SERF2 | 86.73 |
| _53 | | CACGC | 9 | | | |
| sgRNA | 323 | CTCACGCTGGTTACC | chr15:4379297 | SERF2 | SERF2 | 80.57 |
| _54 | | GCCTA | 7 | | | |
| sgRNA | 324 | AAAGATTACGAACTT | chr12:4620755 | SLC38A1 | SLC38 | 92.24 |
| _55 | | CCCTG | 9 | | A1 | |
| sgRNA | 325 | GTTAAAAACAGACAT | chr12:4622923 | SLC38A1 | SLC38 | 91.51 |
| _56 | | GCCTA | 2 | | A1 | |
| sgRNA | 326 | ATGCCTAAGGAGGTT | chr12:4622924 | SLC38A1 | SLC38 | 79.48 |
| _57 | | GTACC | 6 | | A1 | |
| sgRNA | 327 | CTCCAGGTATCCCAT | chr18:4786941 | SMAD2 | SMAD2 | 79.53 |
| _58 | | CGAAA | 8 | | | |
| sgRNA | 328 | CACCAAATACGATAG | chr18:4787053 | SMAD2 | SMAD2 | 86.61 |
| _59 | | ATCAG | 2 | | | |
| sgRNA | 329 | TGGCGGCGTGAATGG | chr18:4789672 | SMAD2 | SMAD2 | 82.91 |
| _60 | | CAAGA | 9 | | | |
| sgRNA | 330 | TAGGATGGTAGCACA | chr16:1125547 | SOCS1 | SOCS1 | 92.25 |
| _61 | | CAACC | 8 | | | |
| sgRNA | 331 | CAGCAGCAGAGCCCC | chr16:1125543 | SOCS1 | SOCS1 | 83.79 |
| _62 | | GACGG | 2 | | | |
| sgRNA | 332 | CGGCGTGCGAACGGA | chr16:1125529 | SOCS1 | SOCS1 | 84.24 |
| _63 | | ATGTG | 6 | | | |
| sgRNA | 333 | TATAGACGCTGCCCG | chr15:4003889 | SRP14 | SRP14 | 95.12 |
| _64 | | ACGTC | 5 | | | |
| sgRNA | 334 | TCCAAAGAAGGGTAC | chr15:4003836 | SRP14 | SRP14 | 92.14 |
| _65 | | TGTGG | 8 | | | |
| sgRNA | 335 | ACAGTACCCTTCTTTG | chr15:4003835 | SRP14 | SRP14 | 65.82 |
| _66 | | GAAT | 8 | | | |
| sgRNA | 336 | GCGACGGGCGCATCT | chr12:1204695 | SRSF9 | SRSF9 | 83.68 |
| _67 | | ACGTG | 72 | | | |
| sgRNA | 337 | CCCGACCTCCATAAG | chr12:1204657 | SRSF9 | SRSF9 | 92.56 |
| _68 | | TCCTG | 00 | | | |
| sgRNA | 338 | GGGGTCCTCGAAGCG | chr12:1204694 | SRSF9 | SRSF9 | 89.94 |
| _69 | | CACGA | 26 | | | |
| sgRNA | 339 | TGCTCTGTTTAGAAG | chr5:32591641 | SUB1 | SUB1 | 79.36 |
| _70 | | ATGAC | | | | |
| sgRNA | 340 | ATATTCTTTTCTAGTT | chr5:32591566 | SUB1 | SUB1 | 70.93 |
| _71 | | AAAG | | | | |
| sgRNA | 341 | CCTGTAAAGAAACAA | chr5:32591614 | SUB1 | SUB1 | 93.66 |
| _72 | | AAGAC | | | | |
| sgRNA | 342 | TGGAGAAAGACGTAA | chr4:10523431 | TET2 | TET2 | 83.53 |
| _73 | | CTTCG | 5 | | | |
| sgRNA | 343 | TCTGCCCTGAGGTAT | chr4:10523474 | TET2 | TET2 | 90.97 |
| _74 | | GCGAT | 7 | | | |
| sgRNA | 344 | ATTCCGCTTGGTGAA | chr4:10523565 | TET2 | TET2 | 89.62 |
| _75 | | AACGA | 6 | | | |
| sgRNA | 345 | CAGGCACAATAGAAA | chr3:11429557 | TIGIT | TIGIT | 92.65 |
| _76 | | CAACG | 1 | | | |
| sgRNA | 346 | CCATTTGTAATGCTG | chr3:11429570 | TIGIT | TIGIT | 60.75 |
| _77 | | ACTTG | 0 | | | |
| sgRNA | 347 | CTGGGTCACTTGTGC | chr3:11429563 | TIGIT | TIGIT | 87.99 |
| _78 | | CGTGG | 4 | | | |
| sgRNA | 348 | GTCAGGGTTCTGGAT | chr14:2254750 | TRAC | TRAC | 98.20 |
| _79 | | ATCTG | 8 | | | |
| sgRNA | 349 | TGGATTTAGAGTCTC | chr14:2254754 | TRAC | TRAC | 88.15 |
| _80 | | TCAGC | 1 | | | |
| sgRNA | 350 | CTGCGGCTGTGGTCC | chr14:2255066 | TRAC | TRAC | 94.77 |
| _81 | | AGCTG | 1 | | | |
| sgRNA | 351 | ACAAAACTGTGCTAG | chr14:2254765 | TRAC | TRAC | 87.86 |
| _82 | | ACATG | 8 | | | |
| sgRNA | 352 | TTCTTCCCCAGCCCA | chr14:2254777 | TRAC | TRAC | 89.85 |
| _83 | | GGTAA | 8 | | | |
| sgRNA | 353 | CGTCATGAGCAGATT | chr14:2255062 | TRAC | TRAC | 95.81 |
| _84 | | AAACC | 5 | | | |
| sgRNA | 354 | GAGAGCGCCTGCGAC | chr19:5854498 | TRIM28 | TRIM2 | 89.44 |
| _85 | | CCGAG | 0 | | 8 | |
| sgRNA | 355 | CCAGCGGGTGAAGTA | chr19:5854486 | TRIM28 | TRIM2 | 94.79 |
| _86 | | CACCA | 9 | | 8 | |
| sgRNA | 356 | GGAGCGCTTTTCGCC | chr19:5854483 | TRIM28 | TRIM2 | 91.81 |
| _87 | | GCCAG | 9 | | 8 | |
| sgRNA | 357 | TGAGGCCTGGACCTT | chr10:3313419 | chr10:3313000 | desert_1 | 69.44 |
| _88 | | ATGCA | 3 | 0-33140000 | (GS88) | |
| sgRNA | 358 | CCTGGTGGAGTGAAC | chr10:3313291 | chr10:3313000 | desert_1 | 95.25 |
| _89 | | CATGA | 7 | 0-33140000 | (GS89) | |
| sgRNA | 359 | CAAGCACTTAGGTTC | chr10:3313463 | chr10:3313000 | desert_1 | 91.13 |
| _90 | | CCCTG | 3 | 0-33140000 | (GS90) | |
| sgRNA | 360 | GGTCTCCCTACAATT | chr10:7229456 | chr10:7229000 | desert_2 | 92.02 |
| _91 | | CAGCG | 8 | 0-72300000 | (GS91) | |
| sgRNA | 361 | CACAGCGCGTGACTG | chr10:7229826 | chr10:7229000 | desert_2 | 90.22 |
| _92 | | CAATG | 8 | 0-72300000 | GS92) | |
| sgRNA | 362 | TCTGGGGCACCAATT | chr10:7229278 | chr10:7229000 | desert_2 | 86.35 |
| _93 | | CTAGG | 6 | 0-72300000 | (GS93) | |
| sgRNA | 363 | GAGCCATGCTTGGCT | chr11:1283425 | chr11:1283400 | desert_3 | 91.24 |
| _94 | | TACGA | 76 | 00-128350000 | (GS94) | |
| sgRNA | 364 | GTACAAGTACTTATC | chr11:1283435 | chr11:1283400 | desert_3 | 89.02 |
| _95 | | TCATG | 92 | 00-128350000 | (GS95) | |
| sgRNA | 365 | GAGATAACAACATAA | chr11:1283471 | chr11:1283400 | desert_3 | 96.47 |
| _96 | | CAACA | 70 | 00-128350000 | (GS96) | |
| sgRNA | 366 | CATATTCCATAGTCTT | chr11:6542500 | chr11:6542500 | desert_4 | 88.54 |
| _97 | | TGGG | 0 | 0-65427000 | (GS97) | |
| | | | | (NEAT1) | | |
| sgRNA | 367 | CTGCCCCTTAGCAAC | chr11:6542550 | chr11:6542500 | desert_4 | 92.76 |
| _98 | | TTAGG | 7 | 0-65427000 | (GS98) | |
| | | | | (NEAT1) | | |
| sgRNA | 368 | TGTTTAAAAATATGT | chr11:6542626 | chr11:6542500 | desert_4 | 90.76 |
| _99 | | TGACA | 4 | 0-65427000 | (GS99) | |
| | | | | (NEAT1) | | |
| sgRNA | 369 | CCAGGAATGGAAACT | chr15:9283031 | chr15:9283000 | desert_5 | 87.84 |
| _100 | | CACGC | 5 | 0-92840000 | (GS100) | |
| sgRNA | 370 | GAGGCCGCTGAATTA | chr15:9283185 | chr15:9283000 | desert_5 | 85.32 |
| _101 | | ACCCG | 0 | 0-92840000 | (GS101) | |
| sgRNA | 371 | ATACACGCACACTTG | chr15:9283113 | chr15:9283000 | desert_5 | 99.92 |
| _102 | | CAGAA | 1 | 0-92840000 | (GS102) | |
| sgRNA | 372 | GAGCAGACAGAAACC | chr16:1122567 | chr16:1122000 | desert_6 | 87.92 |
| _103 | | CAGGG | 0 | 0-11230000 | (GS103) | |
| sgRNA | 373 | TGAGTCTCCAAACAG | chr16:1122628 | chr16:1122000 | desert_6 | 88.53 |
| _104 | | AACAG | 4 | 0-11230000 | (GS104) | |
| sgRNA | 374 | TAATATCACTGACTT | chr16:1122502 | chr16:1122000 | desert_6 | 87.65 |
| _105 | | CACGG | 9 | 0-11230000 | (GS105) | |
| sgRNA | 375 | TACACACAATGTAAG | chr2:87467461 | chr2:87460000 | desert_7 | 71.79 |
| _106 | | CAGCA | | -87470000 | (GS106) | |
| sgRNA | 376 | GGGAGCTCAATTCGA | chr2:87468809 | chr2:87460000 | desert_7 | 65.89 |
| _107 | | AACCA | | -87470000 | (GS107) | |
| sgRNA | 377 | TTGGACAGGTGAGAC | chr2:87467001 | chr2:87460000 | desert_7 | 72.64 |
| _108 | | AGTCG | | -87470000 | (GS108) | |
| sgRNA | 378 | AAGCTCACTCAGATA | chr3:18651131 | chr3:18651000 | desert_8 | 76.89 |
| _109 | | GTGTG | 6 | 0-186520000 | (GS109) | |
| sgRNA | 379 | CAGGAGAACCACCTT | chr3:18651526 | chr3:18651000 | desert_8 | 86.31 |
| _110 | | ACACG | 0 | 0-186520000 | (GS110) | |
| sgRNA | 380 | GGACAGACCCTGATT | chr3:18651965 | chr3:18651000 | desert_8 | 85.47 |
| _111 | | CACAA | 5 | 0-186520000 | (GS111) | |
| sgRNA | 381 | ACATGGCAGTCTATG | chr3:59451154 | chr3:59450000 | desert_9 | 87.77 |
| _112 | | AACAG | | -59460000 | (GS112) | |
| sgRNA | 382 | CCTATAGAGAGTACT | chr3:59456416 | chr3:59450000 | desert_9 | 79.33 |
| _113 | | ACTTG | | -59460000 | (GS113) | |
| sgRNA | 383 | CCAACCGGGTCTTCA | chr3:59457029 | chr3:59450000 | desert_9 | 92.21 |
| _114 | | TTACG | | -59460000 | (GS114) | |
| sgRNA | 384 | TCAAGCGTAGAGTTC | chr8:12799300 | chr8:12798000 | desert_1 | 93.07 |
| _115 | | CGAGT | 6 | 0-128000000 | 0 | |
| | | | | | (GS115) | |
| sgRNA | 385 | TCATGCAATTATGGA | chr8:12799466 | chr8:12798000 | desert_1 | 89.40 |
| _116 | | CCCAG | 3 | 0-128000000 | 0 | |
| | | | | | (GS116) | |
| sgRNA | 386 | CGGGAAAGTGACTGG | chr8:12799676 | chr8:12798000 | desert_1 | 87.45 |
| _117 | | CCATG | 6 | 0-128000000 | 0 | |
| | | | | | (GS117) | |
| sgRNA | 387 | TGAGATTGAAATCAA | chr9:7974159 | chr9:7970000- | desert_1 | 84.84 |
| _118 | | ATCGG | | 7980000 | 1 | |
| | | | | | (GS118) | |
| sgRNA | 388 | TATGCAATATTCATC | chr9:7977914 | chr9:7970000- | desert_1 | 85.44 |
| _119 | | ACGCG | | 7980000 | 1 | |
| | | | | | (GS119) | |
| sgRNA | 389 | AATGTGTTAAATCAA | chr9:7976895 | chr9:7970000- | desert_1 | 83.48 |
| _120 | | ATGCA | | 7980000 | 1 | |
| | | | | | (GS120) | |

### CRISPR-Cas Editing

One effective example of gene editing is the CRISPR-Cas approach (e.g. CRISPR-Cas9). This approach incorporates the use of a guide polynucleotide (e.g. guide ribonucleic acid or gRNA) and a cas endonuclease (e.g. Cas9 endonuclease).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a nuclease such as a cas endonuclease (e.g. Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g. Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540(7631), 144-149, the relevant disclosures of which are herein incorporated in their entirety).

Methods for conducing gene editing using CRISPR-Cas approaches are known to those of ordinary skill in the art. (See, for example, US application Nos. U.S. Ser. No. 16/312,676, U.S. Ser. No. 15/303,722, and U.S. Ser. No. 15/628,533, the disclosures of which are herein incorporated by reference in their entirety). Additionally, uses of endonucleases for inserting transgenes into safe harbor loci are described, for example, in U.S. application Ser. No. 13/036,343, the disclosures of which are herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example US Patent Publication No. 20180127786, the disclosure of which is herein incorporated by reference in its entirety.

### Therapeutic Applications

For therapeutic applications, the engineered cells, populations thereof, or compositions thereof are administered to a subject, generally a mammal, generally a human, in an effective amount. The engineered cells may be administered to a subject by infusion (e.g., continuous infusion over a period of time) or other modes of administration known to those of ordinary skill in the art.

The engineered cells provided herein not only find use in gene therapy but also in non-pharmaceutical uses such as, e.g., production of animal models and production of recombinant cell lines expressing a protein of interest.

The engineered cells of the present disclosure can be any cell, generally a mammalian cell, generally a human cell that has been modified by integrating a transgene at a safe harbor locus described herein. Exemplary cells are provided in the Recombinant Cells section.

The engineered cells, compositions and methods of the present disclosure are useful for therapeutic applications such as CAR T cell therapy and TCR T cell therapy. In some embodiments, the insertion of a sequence encoding a transgene within a safe harbor locus maintains the TCR expression relative to instances when there is no insertion and enables transgene expression while maintaining TCR function.

In some embodiments, the present disclosure provides methods of treating a subject in need of treatment by administering to the subject a composition comprising any of the engineered cells described herein. In some embodiments, administration of the engineered cell composition results in a desired pharmacological and/or physiological effect. That effect can be partial or complete cure of the disease and/or adverse effects resulting from the disease. In some embodiments, treatment encompasses any treatment of a disease in a subject (e.g., mammal, e.g., human). Further, treatment may stabilize or reduce undesirable clinical symptoms in subjects (e.g., patients). The cells provided herein populations thereof, or compositions thereof may be administered during or after the occurrence of the disease.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition.

### Method of Administration

An effective amount of the immune cell comprising the system may be administered for the treatment of cancer. The appropriate dosage of the immune cell comprising the system may be determined based on the type of cancer to be treated, the type of the immune cell comprising the system, the severity and course of the cancer, the clinical condition of the individual, the individual's clinical history and response to the treatment, and the discretion of the attending physician.

Determining Expression of PSMA and/or CA9

Also provided herein are methods of treating a cancer in a subject in need thereof comprising: determining or having determined the expression of PSMA in the subject; determining or having determined the expression of CA9 in the subject; and administering or having administered to the subject a system comprising a priming receptor comprising a first extracellular antigen-binding domain that specifically binds to PSMA, a chimeric antigen receptor comprising a second extracellular antigen-binding domain that specifically binds to CA9.

Also provided herein are methods of treating a cancer in a subject in need thereof comprising: determining or having determined the expression of PSMA in the subject; determining or having determined the expression of CA9 in the subject; and administering or having administered to the subject a cell comprising a priming receptor comprising a first extracellular antigen-binding domain that specifically binds to PSMA, a chimeric antigen receptor comprising a second extracellular antigen-binding domain that specifically binds to CA9, and optionally a synthetic pathway activator inserted into a target region of the genome of the cell. In some embodiments, the cell is an immune cell. In some embodiments, the immune cell is a primary immune cell.

In some embodiments, the method further comprises determining or having determined the expression level of PSMA and/or CA9 in a biological sample from the individual. In some embodiments the biological sample includes, but is not limited to a body fluid, a tissue sample, an organ sample, urine, feces, blood, saliva, CSF and any combination thereof. In some embodiments the biological sample is derived from a tumor tissue. In some embodiments, the expression level of PSMA and/or CA9 comprises the mRNA expression level of PSMA and/or CA9. In some embodiments, the expression level of PSMA and/or CA9 comprises the protein expression level of PSMA and/or CA9. In some embodiments the expression level of PSMA and/or CA9 is detected in the sample using a method selected from the group consisting of FACS, Western blot, ELISA, immunoprecipitation, immunohistochemistry, monoplex immunohistochemistry, multiplex immunohistochemistry, immunofluorescence, radioimmunoassay, dot blotting, immunodetection methods, HPLC, surface plasmon resonance, optical spectroscopy, mass spectrometry, qPCR, RT-qPCR, multiplex qPCR or RT-qPCR, RNA-seq, microarray analysis, SAGE, MassARRAY technique, Luminex, MSD, and FISH, and combinations thereof.

In some aspects, provided herein are methods of determining an expression level of PSMA and/or CA9 protein in a sample from a subject comprising contacting the sample with an anti-PSMA and/or CA9 antibody and performing an immunohistochemistry assay. In some embodiments, the anti-PSMA antibody comprises the PSMA 1 antibody as set forth in SEQ ID NOs: 117-125. In some embodiments, the anti-PSMA antibody comprises the PSMA 2 antibody as set forth in SEQ ID NOs: 129-137. In some embodiments, the anti-PSMA antibody comprises the Clone 3E6 antibody. In some embodiments, the anti-CA9 antibody comprises the CA9 1 antibody as set forth in SEQ ID NOs: 99-106. In some embodiments, the anti-CA9 antibody comprises the CA9 2 antibody as set forth in SEQ ID NOs: 110-113. In some embodiments, the anti-CA9 antibody comprises the Clone M75 antibody.

In another aspect, the present invention provides methods for identifying an individual who may respond to immunotherapy (e.g. with the PSMA and/or CA9 protein system or a cell comprising a PSMA and/or CA9 protein system described herein) for the treatment of an immune-related condition (e.g. cancer) comprising: detecting the expression level of PSMA in a biological sample from the individual; detecting the expression level of CA9 in a biological sample from the individual; and determining based on the expression level of PSMA and/or CA9, whether the individual may respond immunotherapy, wherein co-expression of PSMA and CA9 in a tumor sample from the individual indicates that the individual may respond to immunotherapy. In some embodiments, the PSMA and/or CA9 expression in the individual has already been determined. In some embodiments, the expression level of PSMA and/or CA9 comprises the mRNA expression level of PSMA and/or CA9. In other embodiments, the expression level of PSMA and/or CA9 comprises the protein expression level of PSMA and/or CA9.

In some embodiments the expression level of PSMA and/or CA9 is detected in the sample using a nucleic acid or protein assay. Exemplary a nucleic acid or protein assays include, but are not limited to, FACS, Western blot, ELISA, immunoprecipitation, immunohistochemistry, monoplex immunohistochemistry, multiplex immunohistochemistry, immunofluorescence, radioimmunoassay, dot blotting, immunodetection methods, HPLC, surface plasmon resonance, optical spectroscopy, mass spectrometry, qPCR, RT-qPCR, multiplex qPCR or RT-qPCR, RNA-seq, microarray analysis, SAGE, MassARRAY technique, Luminex, MSD, and FISH, and combinations thereof. In these embodiments, the anti-PSMA and/or CA9 antibody binds to the PSMA and/or CA9 protein, but does not necessarily have to effect a biological response, such as ADCC. In some embodiments the biological sample is derived from a tumor tissue. In some embodiments the biological sample includes, but is not limited to a body fluid, a tissue sample, an organ sample, urine, feces, blood, saliva, CSF and any combination thereof.

In some embodiments, the assay is an immunohistochemistry assay and the anti-PSMA antibody comprises the PSMA 1 antibody as set forth in SEQ ID NOs: 117-125; the anti-PSMA antibody comprises the PSMA 2 antibody as set forth in SEQ ID NOs: 129-137; or anti-PSMA antibody comprises the Clone 3E6 antibody. In some embodiments, the assay is an immunohistochemistry assay and the anti-CA9 antibody comprises the CA9 1 antibody as set forth in SEQ ID NOs: 99-106; the CA9 2 antibody as set forth in SEQ ID NOs: 110-113; or the anti-CA9 antibody comprises the Clone M75 antibody.

### Pharmaceutical Compositions

The engineered recombinant cells provided herein can be administered as part of a pharmaceutical compositions. These compositions can comprise, in addition to one or more of the recombinant cells, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration.

A composition can be administered alone or in combination with other treatments, either simultaneously or sequentially dependent upon the condition to be treated.

### Kits and Articles of Manufacture

The present application provides kits comprising any one or more of the system or cell compositions described herein along with instructions for use. The instructions for use can be present in the kits as a package insert, in the labeling of the container of the kit or components thereof, or can be in digital form (e.g. on a CD-ROM, via a link on the internet). A kit can include one or more of a genome-targeting nucleic acid, a polynucleotide encoding a genome-targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the antibody compositions described herein, with one or more pharmaceutically acceptable excipients.

The present application also provides articles of manufacture comprising any one of the antibody compositions or kits described herein. Examples of an article of manufacture include vials (including sealed vials).

### Additional Embodiments

Embodiment 1 At least one polypeptide comprising at least one of:
a first antigen-binding domain that specifically binds Prostate-Specific Membrane Antigen (PSMA) (SEQ ID NO: 2) wherein the first antigen-binding domain comprises a first variable heavy (VH) chain sequence comprising three heavy chain CDR sequences, CDR-H1, CDR-H2, and CDR-H3, of the VH sequence set forth in SEQ ID NO: 118 or 130, and a first variable light (VL) chain sequence comprising three light chain CDR sequences, CDR-L1, CDR-L2, and CDR-L3, of the VL sequence set forth in SEQ ID NOs: 119 or 131; and/or
a second antigen-binding domain that specifically binds to Carbonic Anhydrase IX (CA9) (SEQ ID NO: 1) wherein the second antigen-binding domain comprises a second variable heavy (VH) chain sequence comprising three heavy chain CDR sequences, CDR-H1, CDR-H2, and CDR-H3, of the VH sequence set forth in SEQ ID NOs: 99 or 110, and, optionally, a second variable light (VL) chain sequence comprising three light chain CDR sequences, CDR-L1, CDR-L2, and CDR-L3, of the VL sequence set forth in SEQ ID NO: 100).

Embodiment 2 The at least one polypeptide of embodiment 1, wherein:
a. the first variable heavy (VH) chain sequence comprises:
   i. a CDR-H1 comprising the sequence set forth in SEQ ID NO: 120, a CDR-H2 comprising the sequence set forth in SEQ ID NO: 121, and a CDR-H3 comprising the sequence set forth in SEQ ID NO: 122, and the first variable light (VL) chain sequence comprises a CDR-L1 comprising the sequence set forth in SEQ ID NO: 123, a CDR-L2 comprises the sequence set forth in SEQ ID NO: 124, and a CDR-L3 comprising the sequence set forth in SEQ ID NO: 125; or
   ii. a CDR-H1 comprising the sequence set forth in SEQ ID NO: 132, a CDR-H2 comprising the sequence set forth in SEQ ID NO: 133, and a CDR-H3 comprising the sequence set forth in SEQ ID NO: 134, and the first variable light (VL) chain sequence comprises a CDR-L1 comprising the sequence set forth in SEQ ID NO: 135, a CDR-L2 comprises the sequence set forth in SEQ ID NO: 136, and a CDR-L3 comprising the sequence set forth in SEQ ID NO: 137 and/or
b. the second variable heavy (VH) chain sequence comprises:
   i. a CDR-H1 comprising the sequence set forth in SEQ ID NO: 101, a CDR-H2 comprising the sequence set forth in SEQ ID NO: 102, and a CDR-H3 comprising the sequence set forth in SEQ ID NO: 103, and the second variable light (VL) chain sequence comprises a CDR-L1 comprising the sequence set forth in SEQ ID NO: 104, a CDR-L2 comprises the sequence set forth in SEQ ID NO: 105, and a CDR-L3 comprising the sequence set forth in SEQ ID NO: 106; or
   ii. a CDR-H1 comprising the sequence set forth in SEQ ID NO: 111, a CDR-H2 comprising the sequence set forth in SEQ ID NO: 112, and a CDR-H3 comprising the sequence set forth in SEQ ID NO: 113.

Embodiment 3 The at least one polypeptide of embodiment 1, wherein the first VH chain sequence comprises the sequence set forth in SEQ ID NOs: 118 or 130, and the first VL chain sequence comprises the sequence set forth in SEQ ID NOs: 119 or 131.

Embodiment 4 The at least one polypeptide of embodiments 1-3, wherein the second VH comprises the sequence as set forth in SEQ ID NOs: 99 or 110, and, optionally wherein the second VL comprises the sequence set forth in SEQ ID NO: 100.

Embodiment 5 A system comprising:
a first chimeric polypeptide comprising a priming receptor comprising the first antigen-binding domain that specifically binds PSMA (SEQ ID NO: 2); and
a second chimeric polypeptide comprising a chimeric antigen receptor (CAR) comprising the second antigen-binding domain that specifically binds to CA9 (SEQ ID NO: 1).

Embodiment 6 The system of embodiment 5, wherein:
the first antigen-binding domain comprises a first variable heavy (VH) chain sequence comprising three heavy chain CDR sequences, CDR-H1, CDR-H2, and CDR-H3, of the VH sequence set forth in SEQ ID NOs: 118 or 130, and a first variable light (VL) chain sequence comprising three light chain CDR sequences, CDR-L1, CDR-L2, and CDR-L3, of the VL sequence set forth in SEQ ID NOs: 119 or 131; and/or
the second antigen-binding domain comprises a second variable heavy (VH) chain sequence comprising three heavy chain CDR sequences, CDR-H1, CDR-H2, and CDR-H3, of the VH sequence set forth in SEQ ID NOs: 99 or 110, and, optionally, a second variable light (VL) chain sequence comprising three light chain CDR sequences, CDR-L1, CDR-L2, and CDR-L3, of the VL sequence set forth in SEQ ID NO: 100.

Embodiment 7 The system of embodiment 6, wherein:
a. the first variable heavy (VH) chain sequence comprises:
   i. a CDR-H1 comprising the sequence set forth in SEQ ID NO: 120, a CDR-H2 comprising the sequence set forth in SEQ ID NO: 121, and a CDR-H3 comprising the sequence set forth in SEQ ID NO: 122, and the first variable light (VL) chain sequence comprises a CDR-L1 comprising the sequence set forth in SEQ ID NO: 123, a CDR-L2 comprises the sequence set forth in SEQ ID NO: 124, and a CDR-L3 comprising the sequence set forth in SEQ ID NO: 125; or
   ii. a CDR-H1 comprising the sequence set forth in SEQ ID NO: 132, a CDR-H2 comprising the sequence set forth in SEQ ID NO: 133, and a CDR-H3 comprising the sequence set forth in SEQ ID NO: 134, and the first variable light (VL) chain sequence comprises a CDR-L1 comprising the sequence set forth in SEQ ID NO: 135, a CDR-L2 comprises the sequence set forth in SEQ ID NO: 136, and a CDR-L3 comprising the sequence set forth in SEQ ID NO: 137 and/or
b. the second variable heavy (VH) chain sequence comprises:
   i. a CDR-H1 comprising the sequence set forth in SEQ ID NO: 101, a CDR-H2 comprising the sequence set forth in SEQ ID NO: 102, and a CDR-H3 comprising the sequence set forth in SEQ ID NO: 103, and the second variable light (VL) chain sequence comprises a CDR-L1 comprising the sequence set forth in SEQ ID NO: 104, a CDR-L2 comprises the sequence set forth in SEQ ID NO: 105, and a CDR-L3 comprising the sequence set forth in SEQ ID NO: 106 or
   ii. a CDR-H1 comprising the sequence set forth in SEQ ID NO: 111, a CDR-H2 comprising the sequence set forth in SEQ ID NO: 112, and a CDR-H3 comprising the sequence set forth in SEQ ID NO: 113.

Embodiment 8 The system of embodiments 6 or 7, wherein the first VH chain sequence comprises the sequence set forth in SEQ ID NO: 118 or 130 and the first VL chain sequence comprises the sequence set forth in SEQ ID NO: 119 or 131.

Embodiment 9 The system of embodiments 6-8, wherein the second VH comprises the sequence as set forth in SEQ ID NO: 99 or 110 and wherein the second VL comprises the sequence set forth in SEQ ID NO: 100.

Embodiment 10 The system of embodiments 5-9, wherein the priming receptor comprises from N-terminus to C-terminus:
the first antigen-binding domain;
a transmembrane domain comprising one or more ligand-inducible proteolytic cleavage sites; and
an intracellular domain comprising a transcriptional effector, wherein binding of PSMA by the first antigen-binding domain results in cleavage at the one or more ligand-inducible proteolytic cleavage sites.

Embodiment 11 The system of embodiments 5-10, wherein the priming receptor comprises a sequence as set forth in SEQ ID NO: 127, 252, 138, or 253.

Embodiment 12 The system of embodiments 5-11, wherein the CAR comprises, from N-terminus to C-terminus:
the antigen-binding domain;
a transmembrane domain;
an optional intracellular co-stimulatory domain; and
an intracellular activation domain.

Embodiment 13 The system of embodiments 5-12, wherein the CAR comprises a sequence as set forth in SEQ ID NOs: 108, 250, 115, or 251.

Embodiment 14 The system of embodiments 5-13, further comprising a third polypeptide comprising a SPA comprising the sequence as set forth in SEQ ID NOs: 108, 250, 115, or 251.

Embodiment 15 The system of embodiments 5-14, wherein the system is encoded by a nucleic acid comprising a sequence with at least 80%, 85%, 90%, 91%, 92%, 93%, 94%, 95%, 96%, 97%, 98%, 99%, or 100% identity to a sequence as set forth in SEQ ID NOs: 143, 144, 145, or 146.

Embodiment 16 A system comprising the at least one polypeptide of embodiments 1-4 and further comprising at least one or more nucleic acids comprising a nucleic acid sequence at least 15 nucleotides in length complementary to at least one of:
a nucleic acid sequence encoding human Fas Cell Surface Death Receptor (FAS) comprising the sequence set forth in SEQ ID NO: 3; and
a nucleic acid sequence encoding human transforming growth factor (TGF)-β Receptor 2 (TGFBR2) comprising the sequence set forth in SEQ ID NO: 4.

Embodiment 17 The system of embodiment 16, wherein the nucleic acid sequence complementary to human FAS comprises a sequence selected from the group consisting of the sequences set forth in SEQ ID NOs: 6-20.

Embodiment 18 The system of embodiments 16-17, wherein the nucleic acid sequence complementary to human TGFBR2 comprises a sequence selected from the group consisting of the sequences set forth in SEQ ID NOs: 34-82.

Embodiment 19 One or more nucleic acid(s) comprising a nucleotide sequence encoding the at least one polypeptide of embodiments 1-4.

Embodiment 20 The one or more nucleic acid(s) of embodiment 19, further comprising at least one or more nucleic acids comprising a nucleic acid sequence at least 15 nucleotides in length complementary to at least one of:
a. a nucleic acid sequence encoding human Fas Cell Surface Death Receptor (FAS) comprising the sequence set forth in SEQ ID NO: 3; and
b. a nucleic acid sequence encoding human transforming growth factor (TGF)-β Receptor 2 (TGFBR2) comprising the sequence set forth in SEQ ID NO: 4.

Embodiment 21 One or more nucleic acid(s) comprising a nucleotide sequence encoding the system of embodiments 5-18.

Embodiment 22 The one or more nucleic acid(s) of embodiment 21, further comprising at least one or more nucleic acids comprising a nucleic acid sequence at least 15 nucleotides in length complementary to at least one of:
a. a nucleic acid sequence encoding human Fas Cell Surface Death Receptor (FAS) comprising the sequence set forth in SEQ ID NO: 3; and
b. a nucleic acid sequence encoding human transforming growth factor (TGF)-β Receptor 2 (TGFBR2) comprising the sequence set forth in SEQ ID NO: 4.

Embodiment 23 One or more vector(s) comprising the one or more nucleic acid(s) of embodiments 19-22.

Embodiment 24 A cell comprising the at least one polypeptide of embodiments 1-6.

Embodiment 25 The cell of embodiment 24, wherein the cell is a primary human immune cell.

Embodiment 26 A pharmaceutical composition comprising the cell of embodiment 24 or 25, and a pharmaceutically acceptable excipient.

Embodiment 27 A cell comprising the system of embodiments 7-21, optionally wherein the cell is a primary human immune cell.

Embodiment 28 A method of editing an immune cell, comprising:
a. providing a ribonucleoprotein complex (RNP) and a nucleic acid, wherein the RNP comprises a nuclease domain and a guide RNA, wherein the nucleic acid comprises the nucleic acid of embodiment 19, and wherein the 5′ and 3′ ends of the nucleic acid comprise nucleotide sequences that are homologous to genomic sequences flanking an insertion site in the genome of the immune cell;
b. non-virally introducing the RNP complex and nucleic acid into the immune cell, wherein the guide RNA specifically hybridizes to a target region of the genome of the primary immune cell, and wherein the nuclease domain cleaves the target region to create the insertion site in the genome of the immune cell; and
c. editing the immune cell via insertion of the nucleic acid into the insertion site in the genome of the immune cell.

Embodiment 29 A method of treating a disease or increasing an immune response in a subject comprising administering to the subject a cell comprising at least one polypeptide comprising at least one of:
a first antigen-binding domain that specifically binds Prostate-Specific Membrane Antigen (PSMA) (SEQ ID NO: 2); and/or
a second antigen-binding domain that specifically binds to Carbonic Anhydrase IX (CA9) (SEQ ID NO: 1,
   optionally wherein the disease is cancer.

Embodiment 30 The method of embodiment 29, wherein the target cell is a cancer cell, optionally wherein the cancer cell is a cancer cell of kidney cancer, clear cell renal cell carcinoma (ccRcc), colorectal cancer, or lung cancer.

### EXAMPLES

Below are examples of specific embodiments for carrying out the present disclosure.

The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd Ed}. (Plenum Press) Vols A and B (1992).

### Example 1: CA9 Binder Generation and Synthesis

The CA9 1 binder is a fully human antibody, generated following an immunization and screening campaign with humanized transgenic mice. A total of 12 mice were immunized with recombinant human CA9 protein and serum titers evaluated on day 49 for anti-CA9 binding. The draining lymph nodes were harvested from the top 6 responding mice, with selection based on sera-reactivity to recombinant human CA9 by ELISA and human CA9-engineered HEK293 cells by flow cytometry. Single Cell Technology, Inc., performed all single cell screening work utilizing their AbTheneum™ platform. The draining lymph nodes were harvested from the top 6 responding mice, with selection based on sera-reactivity to recombinant human CA9 by ELISA and human CA9-engineered HEK293 cells by flow cytometry. CD138 positive plasma cells were isolated from lymph nodes and subsequently single cells were deposited into microwells of a slide. Secreted IgG from the single cells was screened for binding to recombinant human CA9 antigen. The plasma cell-secreted antibody was separated from cells, captured and screened for binding to fluorescently-labeled target antigen, with binding events captured through imaging. Single cell mRNA was recovered and cDNA was sequenced by NGS to retrieve antibody VH and VL sequences. Antibody binding and sequence data were paired for further analysis.

Fully-human VH and VL sequences were recovered from monoclonal plasma cells via next-generation sequencing (NGS) and paired with single cell screening data to identify binder sequences of interest. The VH sequence of CA9 1 is provided in SEQ ID NO: 99. The VL sequence of CA9 1 is provided in SEQ ID NO: 100. An scFv of CA9 1 was also generated, provided in SEQ ID NO: 98.

The CA9 2 binder is a human, single-domain, anti-CA9 VH-only antibody fragment isolated from a proprietary phage display library. Three iterative rounds of phage selection were performed against HEK293 cells engineered to overexpress human CA9. Monoclonal phage were prepared, screened for binding to recombinant human CA9 by ELISA and ELISA-positive hits sequenced by NGS. The VH sequence of CA9 2 is provided in SEQ ID NO: 110.

To further characterize the sequences, VH and VL pairs or VH single domains were cloned into mammalian expression vectors containing mouse IgG2a constant domains. The resulting chimeric CA9 1-mIgG2a and CA9 2-mIgG2a antibodies were transiently expressed from CHO cultures and purified via affinity chromatography.

Binding of recombinant CA9 1-mIgG2a and CA9 2-mIgG2a to CA9 was assessed by cell binding dose curves on parental HEK293T cells and HEK293T-CA9 expressing cell lines. Briefly, HEK293T parental and HEK293T-CA9 engineered cells were first blocked with human Fc blocker for 10 min at room temperature, stained for 30 min on ice with recombinant antibodies in an 8-point serial dilution, and washed 3 times with BD Stain Buffer. Subsequently, cells were stained for 30 min on ice with anti-mouse IgG2a-PE secondary antibody. Cell surface binding was measured on an Attune flow cytometer, with geometric mean fluorescence intensity (gMFI) data analyzed in FlowJo and plotted using GraphPad Prism. The dose-response curves and EC50 value were analyzed and obtained using the nonlinear fit model of (agonist) versus response (three parameters).

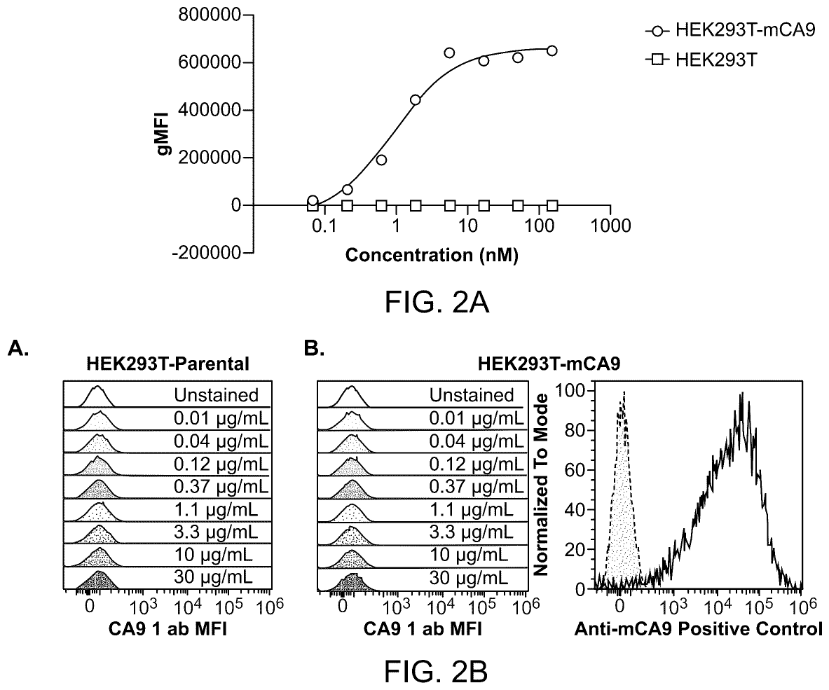

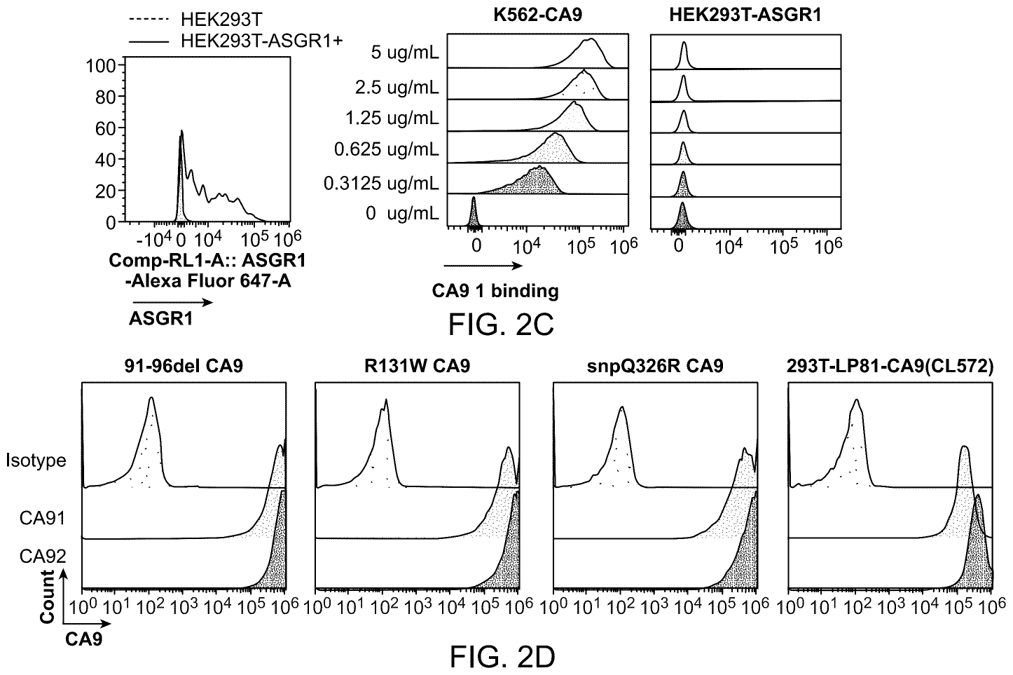

As shown in FIG. 2A, CA9 1-mIgG2a bound in a dose-dependent manner to HEK293T-CA9, with an EC50 value of 0.92 nM. There was no binding to parental HEK293T cells.

The binding affinity of recombinant CA9 antibodies to human CA9 target antigen and cross-reactivity to mouse homologs was measured on the Octet R8. Briefly, recombinant antibody was loaded onto anti-mouse capture (AMC) Octet biosensors. Biosensors were then dipped into different concentrations of recombinant target antigen (analyte) solution, and the rate of protein-protein complex formation (association) measured. Protein-protein dissociation rates were measured when the biosensors were dipped into kinetic buffer alone. The Octet Analysis Studio 13.0 software calculated the response in equilibrium (Req) using the association and dissociation signal. The KD value and other kinetics parameters were determined by the values of Req for each analyte concentration (Table 1).

CA9 1-mIgG2a bound recombinant human CA9 with high affinity, resulting in a K_{D} value of 0.15 nM. There was no detectable binding to recombinant mouse CA9.

TABLE 1
Binding kinetics of CA9 1

| Ligand | Analyte | K_{D} | k_{association} | k_{dissociation} |
|---|---|---|---|---|
| CA9 | Human | 0.15 nM | 3.246 × 10⁵ M⁻¹s⁻¹ | 4.90 × 10⁻⁵ s⁻¹ |
| 1_mIgG2a | CA9 | | | |

To assess cross-reactivity of CA9 1 to mouse CA9 and further confirm the binding kinetics to human CA9, HEK293T-mCA9 cells were stained with increasing concentrations of CA9 1-mIgG2a then analyzed by flow cytometry. Recombinant CA9 1-mIgG2a protein was titrated against parental and engineered HEK293T cell lines expressing mouse CA9. Target cells were also stained with positive control antibodies to confirm expression of mouse CA9 on the engineered HEK293T cells. Following primary staining, the cells were washed, stained with secondary antibody, and subsequently analyzed by flow cytometry. Data were analyzed using FlowJo. Target cells were also stained using an anti-mouse CA9 positive control antibody to confirm expression of mouse CA9 on the HEK293T-mCA9 cells.

No cross-reactivity of CA9 1 mouse CA9 was observed as evidenced by a lack of increase in MFI relative to the HEK293T parental cell line and unstained controls (FIG. 2B). Staining of HEK293T-mCA9 with positive control antibodies confirmed target CA9 antigen expression on the cell line. Thus, CA9 1 was not cross react with mouse CA9 (FIG. 2B).

CA9 1 and CA9 2 were specific to the CA9-expressing cell line and did not bind to the CA9-negative parental cells (FIG. 2B and data not shown). CA9 1 and CA9 2 also exhibited potent anti-CA9 activity in CAR T cells that contain the CA9 1 or CA9 2 binding domains as well as the compatibility of the CA9 1 or CA9 2 CAR T cells with a logic gate with a PSMA priming receptor (primeR) (data not shown).

Cell microarray technology was used to screen for specific off-target binding interactions for the antibodies CA9 1 and CA9 2. CA9 1 and CA9 2 were screened for binding against fixed human HEK293 cells, individually expressing 5861 full-length human plasma membrane proteins and cell surface-tethered human secreted proteins plus a further 371 human heterodimers. CA9 1 showed specific interactions with CA9, the primary target, on both fixed and live microarrays (data not shown). Although classed as non-specific due to the binding of controls, CA9 1 also showed an interaction with ASGR1 but only on fixed cells. CA9 2 showed specific interactions with CA9, the primary target, on both fixed and live microarrays. CA9 2 showed no non-specific binding above controls. (data not shown)

To further validate the interactions with ASGR1 observed with the above results, flow cytometry was used to determine whether CA9 1 shows any binding with HEK293T cell lines overexpressing ASGR1, with CA9-engineered K562 cells acting as a positive control. FIG. 2C shows that CA9 1 has no interaction with HEK293T-ASGR1+ cells across a range of concentration up to 5 ug/mL, while bindings were detected on engineered K562 expressing CA9. Taken together, CA9 1 demonstrated specific interactions with CA9 but not ASGR1 expressing cell lines.

HEK293T cell lines were generated to express CA9 isoforms with an R131 W single nucleotide polymorphism (SNP), a Q326R SNP or a 91-96 deletion (91-96del). CA9 1 and CA9 2 scFvs were used to stain the CA9-91-96del, CA9-R131 W, and CA9-Q326R cell lines followed by a secondary mouse anti human IgG2 stain. As shown in FIG. 2D, all three cell lines expressing the CA9 isoforms showed very strong positive staining with both CA9 1 and CA9 2. Thus, both CA9 1 and CA9 2 could bind to various isoforms of CA9.

In sum, immunization and screening of transgenic mice were successful in isolating anti-CA9 binding domains that are specific to human CA9, with an EC50 value of 0.92 nM binding to HEK293T cells expressing human CA9, and with no cross-reactivity detected against the murine homologs.

### Example 2: PSMA Binder Generation and Synthesis

Two binders for PSMA, PSMA 1 and PSMA 2, were generated following an immunization and screening campaign with humanized transgenic mice. A total of 12 mice were immunized with recombinant human PSMA protein and serum titers evaluated on day 35 for anti-PSMA binding. Single Cell Technology, Inc., performed all single cell screening work utilizing their AbTheneum™ platform. The draining lymph nodes were harvested from the top 6 responding mice, with selection based on sera-reactivity to recombinant human PSMA by ELISA and human PSMA-engineered HEK293 cells by flow cytometry. CD138 positive plasma cells were isolated from lymph nodes and subsequently single cells were deposited into microwells of a slide. Secreted IgG from the single cells was screened for binding to recombinant human PSMA antigen. The plasma cell-secreted antibody was separated from cells, captured and screened for binding to fluorescently-labeled target antigen, with binding events captured through imaging. Single cell mRNA was recovered and cDNA was sequenced by NGS to retrieve antibody VH and VL sequences. Antibody binding and sequence data were paired for further analysis.

Fully-human VH and VL sequences were recovered from monoclonal plasma cells via next-generation sequencing (NGS) and paired with single cell screening data to identify binder sequences of interest. The VH sequence of PSMA 1 is provided in SEQ ID NO: 118. The VL sequence of PSMA 1 is provided in SEQ ID NO: 119. The VH sequence of PSMA 2 is provided in SEQ ID NO: 130. The VL sequence of PSMA 2 is provided in SEQ ID NO: 131.

To further characterize the sequences, identified VH and VL pairs were cloned into mammalian expression vectors containing mouse IgG2a constant domains. The resulting chimeric PSMA 1-mIgG2a and PSMA 2-mIgG2a constructs were transiently expressed from CHO cultures and purified via affinity chromatography.

PSMA expressing HEK293T cell lines were used to assess the binding of recombinant antibodies to cell surface expressed PSMA. Briefly, HEK293T parental and HEK293T-PSMA engineered cells were first blocked with human Fc blocker for 10 min at room temperature, stained for 30 min on ice with recombinant antibodies in an 8-point serial dilution, and washed 3 times with BD Stain Buffer. Subsequently, cells were stained for 30 min on ice with anti-mouse IgG2a-PE secondary antibody. Cell surface binding was measured on an Attune flow cytometer, with geometric mean fluorescence intensity (gMFI) data analyzed in FlowJo and plotted using GraphPad Prism. The dose-response curves and EC50 value were analyzed and obtained using the nonlinear fit model of (agonist) versus response (three parameters).

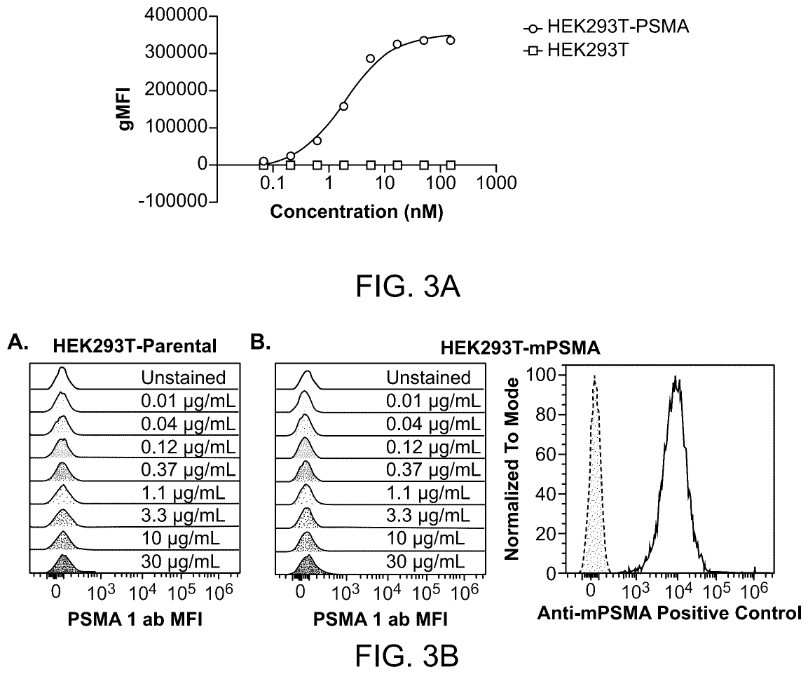

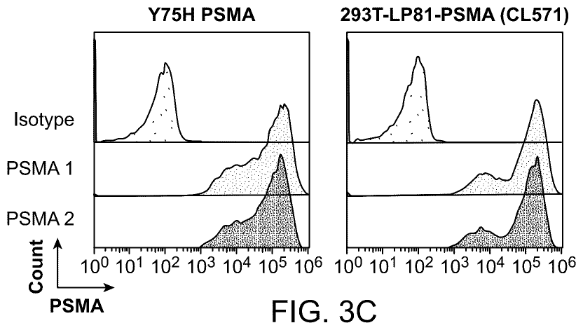

As shown in FIG. 3A, PSMA 1-mIgG2a bound in a dose-dependent manner to HEK293T-PSMA, with an EC50 value of 1.86 nM (Table 2). There was no binding to parental HEK293T cells.

The binding affinity of recombinant antibodies to human PSMA target antigen and cross-reactivity to mouse homologs was measured on the Octet R8 instrument. Briefly, recombinant antibody was loaded onto anti-mouse capture (AMC) Octet biosensors. Biosensors were then dipped into different concentrations of recombinant target antigen (analyte) solution, and the rate of protein-protein complex formation (association) measured. Protein-protein dissociation rates were measured when the biosensors were dipped into kinetic buffer alone. The Octet Analysis Studio 13.0 software calculated the response in equilibrium (Req) using the association and dissociation signal. The K_{D} value and other kinetics parameters were determined by the values of Req for each analyte concentration (Table 2).

PSMA 1-mIgG2a bound recombinant human PSMA with high affinity, resulting in a K_{D} value of 0.30 nM. There was no detectable binding to recombinant mouse PSMA.

TABLE 2
Binding kinetics of PSMA 1

| Ligand | Analyte | K_{D} | k_{association} | k_{dissociation} |
|---|---|---|---|---|
| PSMA 1- | Human | 0.30 nM | 6.326 × 10⁴ M⁻¹s⁻¹ | 1.887 × 10⁻⁵ s⁻¹ |
| mIgG2a | PSMA | | | |

Cross-reactivity of PSMA 1 to mouse PSMA was evaluated using flow cytometry. In brief, HEK293T cells expressing mouse PSMA (HEK293T-mPSMA) were stained for 30 min on ice with increasing concentrations of PSMA 1-mIgG2a. Target cells were also stained with positive control antibodies to confirm expression of mouse PSMA on the engineered HEK293T cells. Following primary staining, the cells were washed, stained with secondary antibody, and subsequently analyzed by flow cytometry. Data were analyzed using FlowJo.

No cross-reactivity of PSMA 1 to mouse PSMA (FIG. 3B) was observed as evidenced by a lack of increase in MFI relative to the HEK293T parental cell line. Staining of HEK293T-mPSMA with positive control antibodies confirmed target PSMA antigen expression on the cell line. Thus, PSMA 1 was not cross react with mouse PSMA (FIG. 3B).

Binding of PSMA 1-mIgG2a or PSMA 2-mIgG2a to PSMA was assessed by cell binding dose curves on parental HEK293T cells and HEK293T-PSMA expressing cell lines. PSMA 1 and PSMA 2 were both specific to the PSMA-expressing cell line and did not bind to the PSMA-negative parental cells. (data not shown) PSMA 1 and PSMA 2 also exhibited specific and strong anti-PSMA induction of the CA9 CAR when incorporated into a logic gate. (data not shown)

Specific off-target binding interactions for the recombinant binding proteins, PSMA 1 and PSMA 2, was assessed via cell microarray technology. PSMA 1 was screened for binding against fixed human HEK293 cells, individually expressing 5861 full-length human plasma membrane proteins and cell surface-tethered human secreted proteins plus a further 371 human heterodimers. PSMA 1 showed specific interactions with PSMA, the primary target, on both fixed and live microarrays. No other target was detected from the screen. (data not shown)

PSMA 2 was screened for binding against fixed human HEK293 cells, individually expressing 5861 full-length human plasma membrane proteins and cell surface-tethered human secreted proteins plus a further 371 human heterodimers. PSMA 2 showed specific interactions with PSMA, the primary target, on both fixed and live microarrays. CD70 was identified as an off target but only on fixed cells; when fresh CD70+ HEKs were stained with AVD2157, no binding was detected. (data not shown)

HEK293T cell lines were generated to express PSMA with a Y75H single nucleotide polymorphism (SNP). PSMA 1 and PSMA 2 scFvs were used to stain the PSMA-Y75H cell line followed by a secondary mouse anti human IgG2 stain. As shown in FIG. 3C, both PSMA 1 and PSMA 2 scFvs bound to cells expressing the protein with the PSMA SNP Y75H. Thus, both PSMA 1 and PSMA 2 could bind to various isoforms of PSMA.

In sum, immunization and screening of transgenic mice were successful in isolating anti-PSMA binding domains that are specific to human PSMA, with an EC50 value of 1.86 nM binding to HEK293T cells expressing human PSMA, and with no cross-reactivity detected against the murine homologs.

### Example 3: Logic Gate Synthesis and In Vitro Characterization

### Materials and Methods

### Logic Gate Screening

Hundreds of novel scFv and VH/VHH binders targeting PSMA (as a priming target) and CA9 (as a cytolytic target) were generated via two parallel de novo binder discovery efforts: 1) transgenic mice immunizations and 2) phage display panning campaigns. Two independent arrayed screens with 500 PSMA prime receptors (PrimeR) and 750 CA9 CARs were conducted to find PrimeRs with high inducibility and CARs with strong on-target potency. Characterization of the top PSMA and CA9 binding proteins are provided in Examples 1 and 2.

From these screens, the top 25 PSMA PrimeRs and 20 CA9 CARs were combined with an exemplary shRNA cassette for targeted knockdowns along with two variations of a synthetic pathway activator (SPA). A fully-automated workcell was used to perform end-to-end arrayed screening of the resulting 1,000 member library in T cells engineered from four human donors. Non-viral editing techniques were used to electroporate primary CD4/CD8 cells and robotic handlers were used to set up co-cultures. Circuit specificity and potency were assessed by flow cytometry and cytokine secretion and resistance to exhaustion was assessed in a seven day killing assay. Although the library was built from components that functioned well independently, it was found that when combined, many of the circuits displayed suboptimal function. (data not shown) Integrated screening identified 20 variants that each far exceeded the performance of a small set of initial prototypes built from “best-guess” selections of individual components.

The final logic gate candidates were significantly superior to constitutive CAR-T cells in a long term killing assay, show potent cytotoxicity of low expressing antigen lines, and display background levels of cytotoxicity against single antigen targets. (data not shown) Engineering multiple features into T cell products can be limited by unpredictable negative interactions between components. This high-throughput screening generated development-ready candidates for ccRCC with finely tuned desirability criteria in less than 18 months.

### ICT Construct Expression in T Cells

Integrated circuit T (ICT) cells were generated through site directed CRISPR mediated knock in (KI). T cells were activated for two days using CD3-CD28 beads. At day 2, beads were removed followed by the delivery of the ICT transgene to the GS94 site in the genome of the T cells. Transgene integration was performed using a CRISPR-based process and electroporation step that combined activated T cells, CRISPR/Cas9 RNP targeting the GS94 non-coding autosomal integration site, and plasmid DNA constituting a repair template to effect insertion of the transgene cassette via cellular DNA repair machinery.

The GS94 CRISPR/Cas9 RNP used was generated by complexing single guide RNA (sgRNA) with recombinant Streptococcus pyogenes Cas9 (SpCas9). The sgRNA contained a protospacer sequence directing the CRISPR/Cas9 RNP to the GS94-transgene integration site. The plasmid DNA repair template contained the ICT transgene cassette, flanked by 450 base pair (bp) sequences homologous to the regions flanking the integration site to effect repair-mediated insertion.

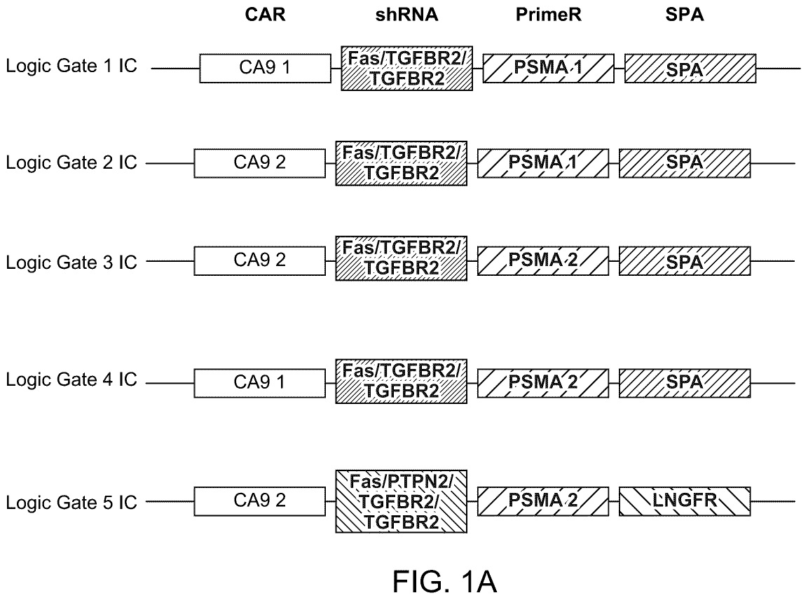

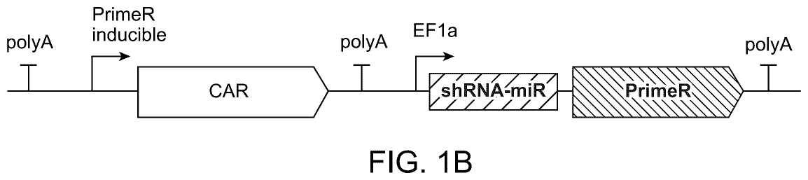

A diagram of the various ICT transgene cassettes generated is provided in FIG. 1A FIG. 1B provides a schematic including the inducible and constitutive promoters and polyA sequences. The ICT constructs 1, 2, 3, and 4 comprised a constitutively expressed PSMA priming receptor, an inducible CA9 CAR (forming a Logic Gate or “LG”), constitutively expressed shRNAs targeting FAS and TGFBR2, and a synthetic pathway activator (SPA). One ICT also included an shRNA targeting PTPN2 and LNGFR in place of the SPA (LG 5 IC).

The sequence of the PSMA priming receptor 1 (primeR 1) is provided in SEQ ID NO: 127 and 252. The PSMA 1 primeR sequence provided in SEQ ID NO: 127 includes the leader sequence, while the PSMA 1 primeR sequence provided in SEQ ID NO: 252 excludes the leader sequence. The sequence of the PSMA priming receptor 2 (primeR 2) is provided in SEQ ID NO: 138 and 253. The PSMA 2 primeR sequence provided in SEQ ID NO: 138 includes the leader sequence, while the PSMA 2 primeR sequence provided in SEQ ID NO: 253 excludes the leader sequence.

The sequence of the CA9 1 CAR (CAR 1) is provided in SEQ ID NO: 108 and 250. The CA9 1 CAR sequence provided in SEQ ID NO: 108 includes the leader sequence, while the CA9 1 CAR sequence provided in SEQ ID NO: 250 excludes the leader sequence. The sequence of the CA9 2 CAR (CAR 2) is provided in SEQ ID NO: 115 and 251. The CA9 2 CAR sequence provided in SEQ ID NO: 115 includes the leader sequence, while the CA9 2 CAR sequence provided in SEQ ID NO: 251 excludes the leader sequence. The sequence of the SPA is provided in SEQ ID NO: 141 and 254. The SPA sequence provided in SEQ ID NO: 141 includes the leader sequence, while the SPA sequence provided in SEQ ID NO: 254 excludes the leader sequence. The sequence of LNGFR is provided in SEQ ID NO: 142 and 255. The LNGFR sequence provided in SEQ ID NO: 142 includes the leader sequence, while the LNGFR sequence provided in SEQ ID NO: 255 excludes the leader sequence.

The sequence of the FAS and dual TGFBR2 shRNA cassette is provided in SEQ ID NO: 83, while the sequence of the FAS/PTPN2/dual TGFBR2 shRNA cassette is provided in SEQ ID NO: 84. The full transgene cassettes comprising a logic gate with the shRNA and optional SPA are termed Logic Gate 1 integrated circuit (“IC” or LG 1 IC) (SEQ ID NO: 143), Logic Gate 2 IC (LG 2 IC) (SEQ ID NO: 144), Logic Gate 3 IC (LG 3 IC) (SEQ ID NO: 145), Logic Gate 4 IC (LG 4 IC) (SEQ ID NO: 146), or Logic Gate 5 IC (LG 5 IC) (SEQ ID NO: 147).

Following electroporation, cells were recovered and expanded in T cell media for 7 days. When indicated, negative control T cells were generated using a mock electroporation process that edited T cells with ribonucleoprotein (RNP) in the absence of donor plasmid and are referred to as “RNP control”.

ICT cells were assessed for transgene KI and the expression of the PrimeR and CAR using flow based staining. Constructs contained tags myc and flag on the distal extracellular portion of the PrimeR and CAR respectively following the signal peptide. ICT cells at day 7 post activation were stained with myc, flag and CD3 antibodies for 30 min at 4c. Following activation, cells were washed in FACs buffer and run by flow cytometry. ICTs were analyzed for PrimeR and CAR expression following gating each sample for live CD3+ cells.

### ICT Induction of CARs

ICTs were generated as described above from the T cells of 2 donors. On day 11 post activation, ICTs were measured for CAR and PrimeR expression by Flag and Myc staining. % KI was quantify by summing the % of T cells in a sample that were PrimeR+ or CAR+. Before co-culture setup, ICTs were normalized to the same % KI using the addition of donor matched RNP only cell. 1×10⁷ ICTs were co-cultured with 1×10⁷ target cells or media for 72 hours and stained to calculate the % of CAR+ cells using flag staining. Basal CAR expression was measured during assay set up.

shRNA Knockdown

ICT cells contain a constitutive shRNA module targeting knockdown of FAS and TGFBR2, whereas cells without a transgene KI (PrimeR negative cells) have normal expression of FAS and TGFBR2 and can be used as an internal control. Multicolor flow cytometry was performed on four productions of ICT cells to characterize transgene expression and assess shRNA-miR knockdown of FAS and TGFBR2. Antibodies against CD4, CD8, CD95 (FAS) and TGFBR2 were used in the flow cytometry. The panel also included rhPSMA for PrimeR detection and rhCA9 for CAR detection as well as Zombi NIR for live vs dead cell staining.

Surface protein knockdown of FAS and TGFBR2 in ICT cells was determined using flow cytometry. Cells were stained with anti-FAS and anti-TGFBR2 antibodies, and geometric mean fluorescence intensity (gMFI) was measured for both PrimeR-positive and PrimeR-negative subsets of ICT cells. Data are representative of 4 donors. The formula used to calculate % KD (percent knockdown)=100%(1−(MFI PrimeR+)/(MFI PrimeR−)).

### Synthetic Pathway Activators

Synthetic Pathway Activators (SPAs) constitutively drive STAT signaling without the need for external cytokine input. SPAs can be designed to engage activity of multiple STAT family transcription factors at variable levels through rational design. Exemplary Class I SPAs primarily increase pSTAT3 activity and exemplary Class II SPAs primarily increase pSTAT5 activity.

A synthetic pathway activator (SPA) based on gp130 was constructed. The SPA comprises the transmembrane region and intracellular domain of gp130 linked to an ectodomain derived from the cell adhesion protein CD34. An unpaired cysteine residue was introduced into the receptor ectodomain to permit formation of a covalent bond and subsequent dimerization of individual synthetic gp130 monomers. The SPA drives constitutive recruitment and phosphorylation of STAT1 and STAT3 transcription factors. (data not shown)

To demonstrate the ability of the SPA module to drive constitutive STAT3 phosphorylation, ICTs expressing the SPA module under non-stimulated conditions were fixed, permeabilized, and stained for pSTAT3 and the PrimeR protein using rhPSMA protein to distinguish between edited and non-edited cells.

### Cytotoxicity, Engineered K562 Cells

ICT cells expressing the integrated circuits comprising Logic Gate 1 IC (SEQ ID NO: 143), Logic Gate 2 IC (SEQ ID NO:144), Logic Gate 3 IC (SEQ ID NO:145), Logic Gate 4 IC (SEQ ID NO:146), or Logic Gate 5 IC (SEQ ID NO: 147) with shRNA and optionally a SPA were co-cultured with K562_EFG, K562_EFG_CA9, K562_EFG_PSMA, or K562_EFG_CA9_PSMA at varying E:T ratios for 72 hours at 37° C. Following incubation, cytotoxicity was measured using a luciferase reporter assay. Data are presented as the mean±standard deviation of 4 donors.

### Cytokine Secretion

To further assess the specificity and function of ICT cells expressing Logic Gate 1-5 ICs, supernatants were collected from K562 target cytotoxicity co-cultures (Effector:Target ratio of 1:1, 72 hour co-culture). Following incubation, supernatants were collected at endpoint and cytokine release levels were measured using a Luminex assay. Data from 4 donors are shown.

### Cytotoxicity in A498 Cells

ICT cells expressing LG 1-5 ICs were co-cultured with A498_PSMA cells (A498 cells endogenously expresses CA9 and engineered to express PSMA) at varying E:T ratios for 72 hours at 37° C. Following incubation, cytotoxicity was measured using a luciferase reporter assay. Data are presented as the mean±standard deviation of 4 donors. Prior to luciferase readout described above, supernatants were collected at endpoint and cytokines (B) IFN-γ, (C) TNFα, (D) GM-CSF and (E) IL-2 were measured using a Luminex assay. Data from 4 donors are shown.

### Mixed Co-Culture Cytotoxicity

ICT cells expressing Logic Gates 1-5 were co-cultured with PSMA+/CA9− HUVEC and luciferase expressing PSMA−/CA9+ cells (K562-EFG-CA9) at varying E:T ratios for 72 hours at 37° C. Following incubation, cytotoxicity was measured using a luciferase reporter assay. Data are from one normal donor. ICT-mediated CA9+ target cell killing was evaluated relative to an RNP-electroporated negative control using a luciferase reporter assay.

### Soluble CA9 and PSMA

ICT cells expressing Logic Gates 1-5 were co-cultured with luciferase expressing 786-O-PSMA-CA9 cells at a 1:1 effector: target ratio with titrating levels of (A) soluble rhCA9 or (B) soluble rhPSMA for 72 hours at 37° C. Following incubation, cytotoxicity was measured using a luciferase reporter assay. Data are presented as one representative donor of 2 tested.

### Transwell Assay

On Day −3 HUVEC+-PSMA endothelial cells were plated to form a monolayer on the upper layer of the transwell plate insert and K562-CA9 tumor cells were plated on the lower layer of the transwell insert below the cell permeable membrane. Monolayers were confirmed to be impermeable to 70 KDa FITC-dextran on day 0. On Day 0, ICT cells expressing Logic Gate 1, the shRNA, and SPA were added to the transwell plate. Total cells at the bottom of the well were counted 3 days after addition of the ICT cells. LG 1 ICT cells were co-cultured were co-cultured with 5K PSMA+ HUVEC cells and 15K K562-CA9 tumor cells for 72 hours at 37° C. Following incubation, cytotoxicity of K562-EFG-CA9 was measured by luciferase reporter assay. Data presented in triplicate from one healthy donor.

### Results

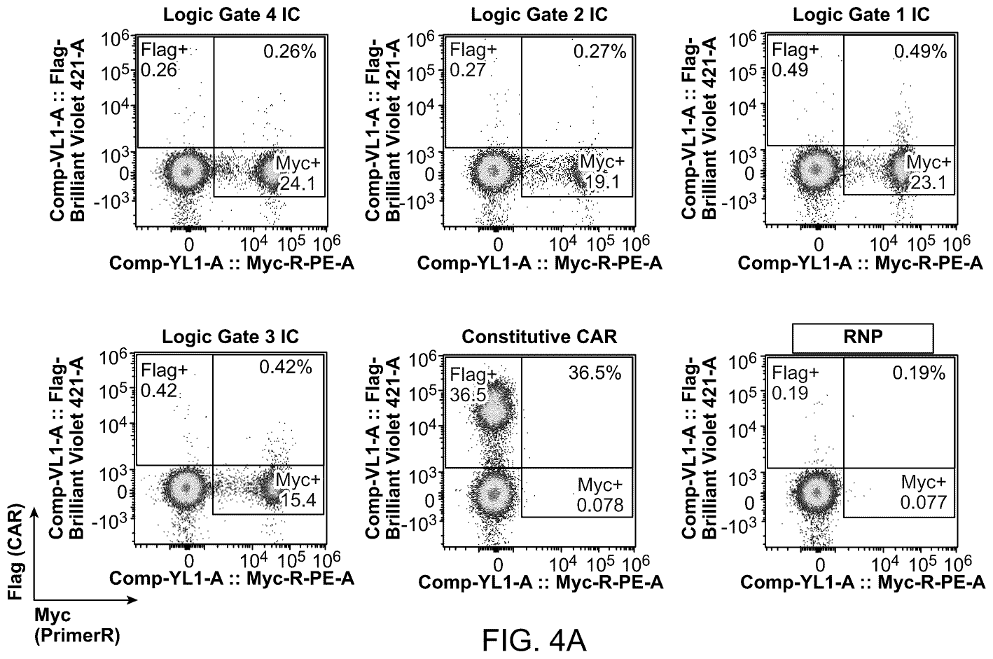

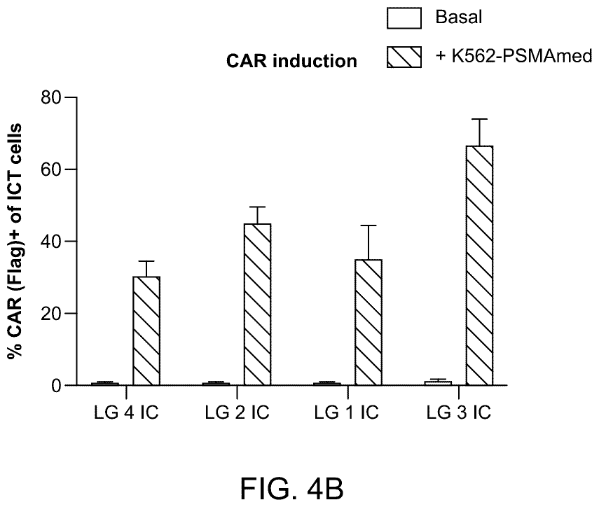

All ICT cells constitutively expressed the PrimeR construct as shown by myc expression (FIG. 4A). The inducible CAR was not expressed at basal state in the ICT cells, as indicated by the lack of FLAG expression (FIG. 4A), indicating that the priming receptor had not induced expression of the CAR.

As shown in FIG. 4B, the ICT cells induced CA9 CAR expression when co-cultured with PSMA expressing cell lines. Numbers shown in FIG. 4B are the (% CAR)/(% KI normalized to at the start of the assay)*100. Thus, the logic gate circuit functioned correctly by not expressing the CA9 CAR in the absence of binding of the primeR to PSMA (FIG. 4A) and induction of expression of the CA9 CAR upon binding of the PSMA primeR to its cognate ligand on a target cell (FIG. 4B).

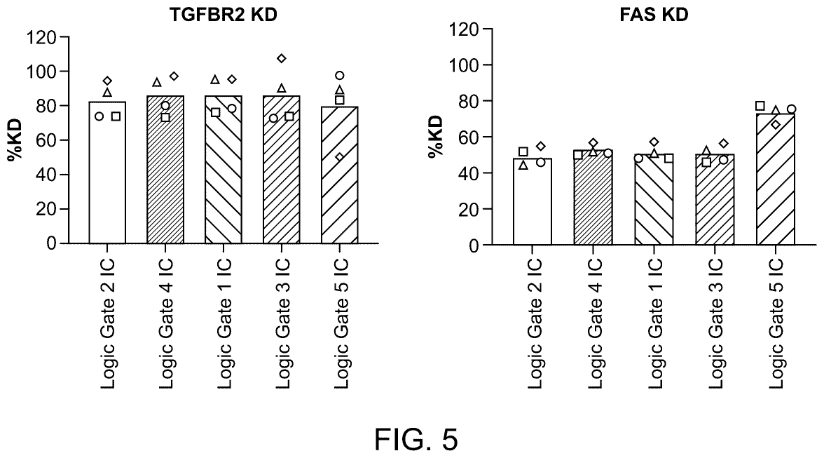

Inclusion of the shRNA module in ICT cells showed lower MFI for both FAS and TGFBR2 in ICT cells expressing the priming receptor-CAR logic gate (PrimeR+) normalized to non-edited cells (PrimeR−), indicating knockdown of both FAS and TGFBR2 in ICT PrimeR+ cells (FIG. 5).

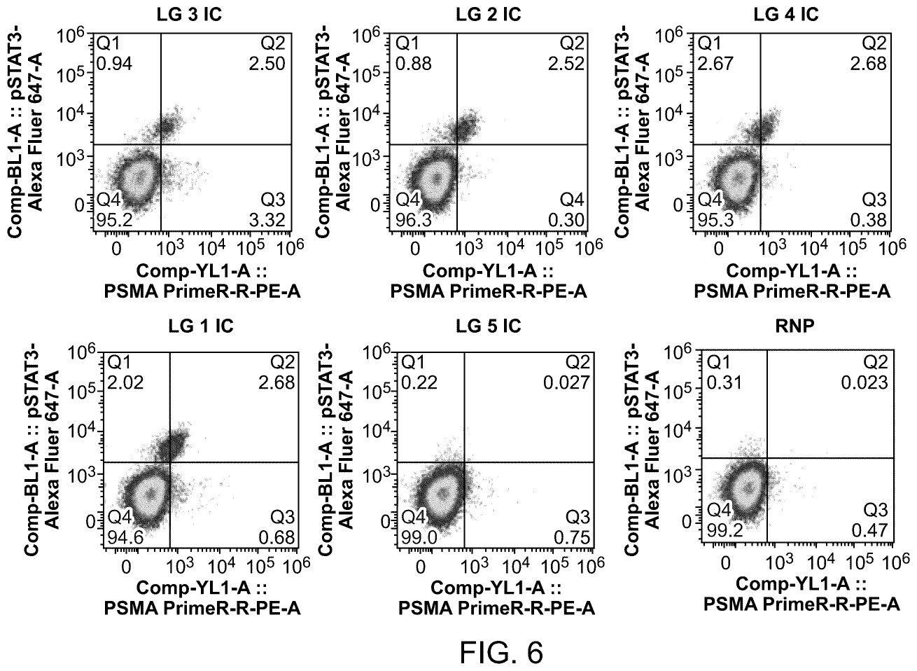

As shown in FIG. 6, flow cytometry analysis revealed that ICT cells expressing the SPA (LG 1-4 ICs) exhibit approximately two logs higher pSTAT3 expression when compared to the PrimeR− cells (RNP). In contrast, edited T cells with a EGFRt (non-signaling) module in the place of a SPA (LG 5 IC) does not exhibit increased pSTAT3 staining when compared to PrimeR− cells. Overall, the results indicate that ICTs expressing the SPA module exhibit increased STAT3 phosphorylation.

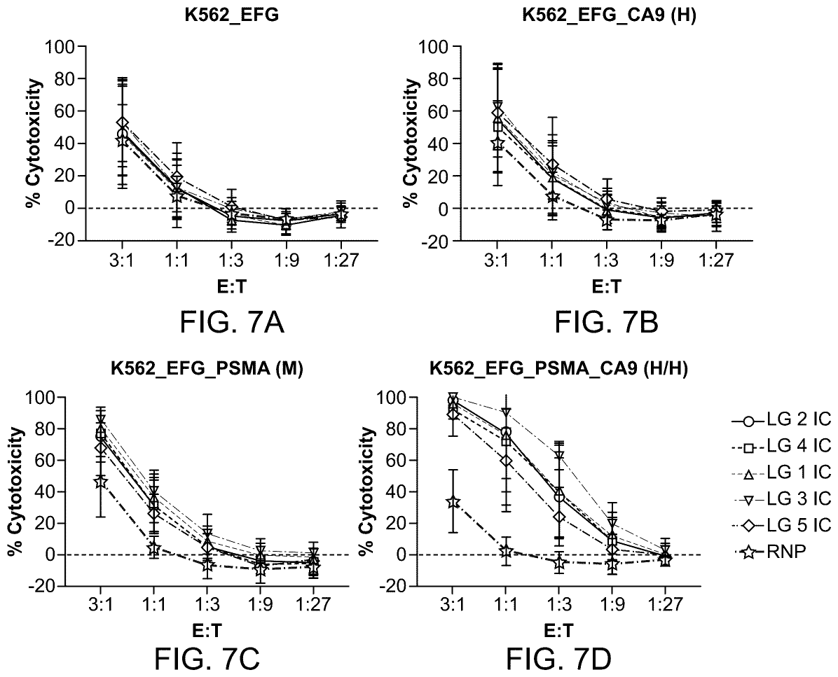

ICTs expressing LG 1-5 ICs demonstrated cytotoxicity against only dual CA9 and PSMA expressing cells as compared to unedited control cells (RNP). FIG. 7A shows cytotoxicity against parental K562 cells expressing neither CA9 or PSMA, FIG. 7B shows cytotoxicity against K562 cells expressing only CA9, FIG. 7C shows cytotoxicity against K562 cells expressing only PSMA, and FIG. 7D shows cytotoxicity against K562 cells expressing both PSMA and CA9. As shown in FIG. 7D, the ICTs exhibited cytotoxicity against only cells expressing both PSMA and CA9 as compared to unedited cells (RNP).

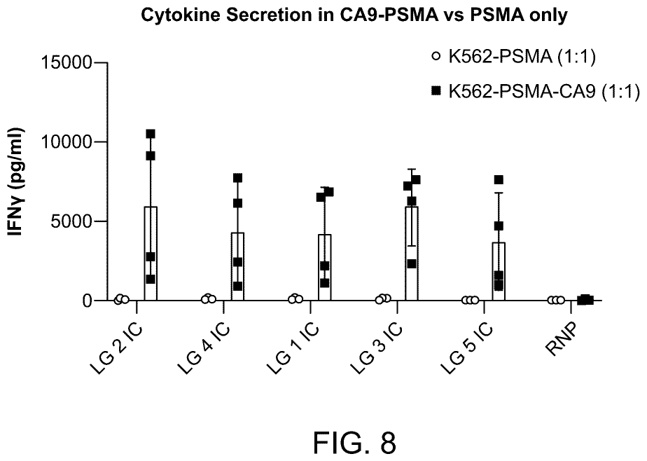

IFN-γ production from ICTs expressing LG 1-5 ICs was observed only in supernatants taken from co-cultures where the target cells expressed both PSMA and CA9 (FIG. 8). Results from the cytokine analysis were consistent with the cytotoxicity data. Together, these data further demonstrate that ICT activity is driven by co-expression of PSMA and CA9.

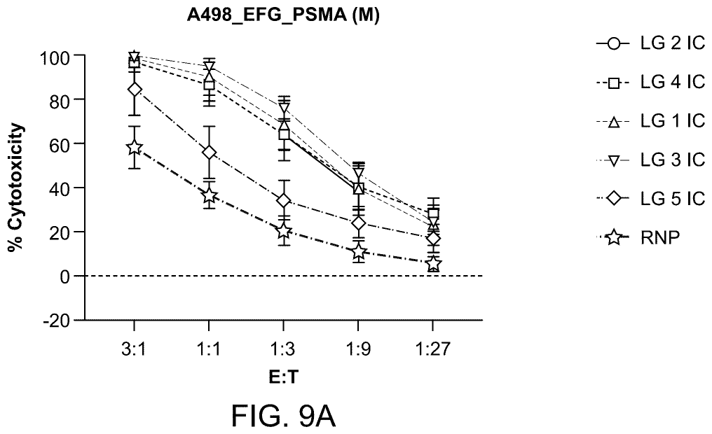

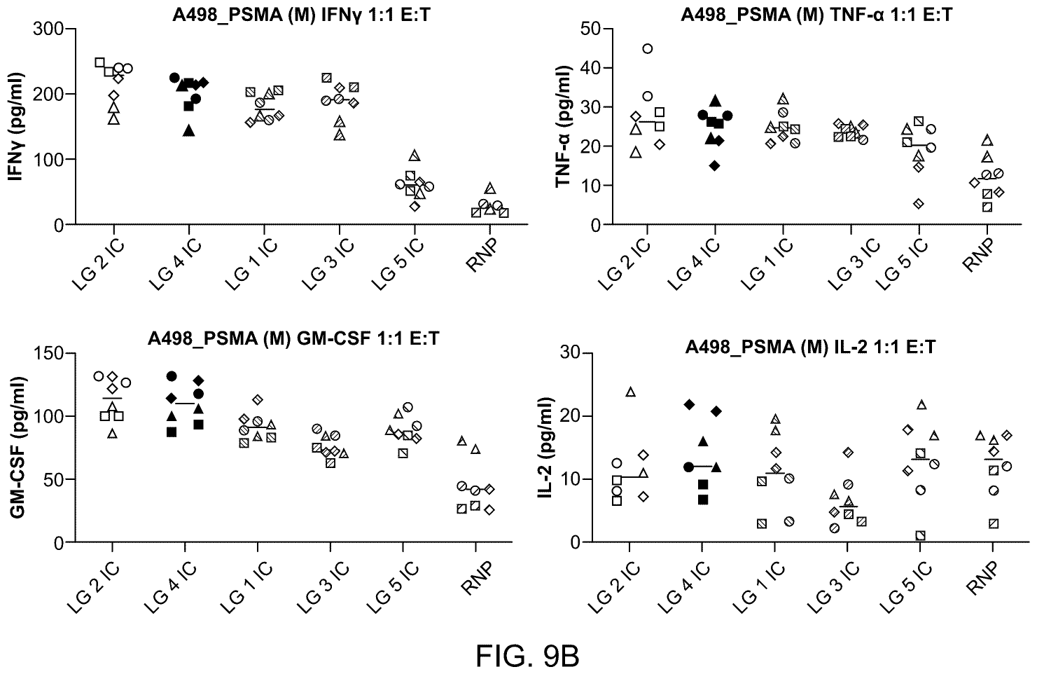

ICTs expressing LG 1-5 ICs demonstrated in vitro cytotoxicity against the A498-PSMAmed cell line expressing endogenous CA9 antigen (FIG. 9A). ICTs also secrete cytokines after A498-PSMA co-culture. FIG. 9B shows IFNγ, TNFα, GM-CSF, and IL-2 secretion by ICT cells after co-culture with A498-PSMA cells. Thus, ICTs expressing LG 1-5 ICs secreted cytokines and killed ccRCC cell lines that express endogenous CA9 antigen in the presence of PSMA.

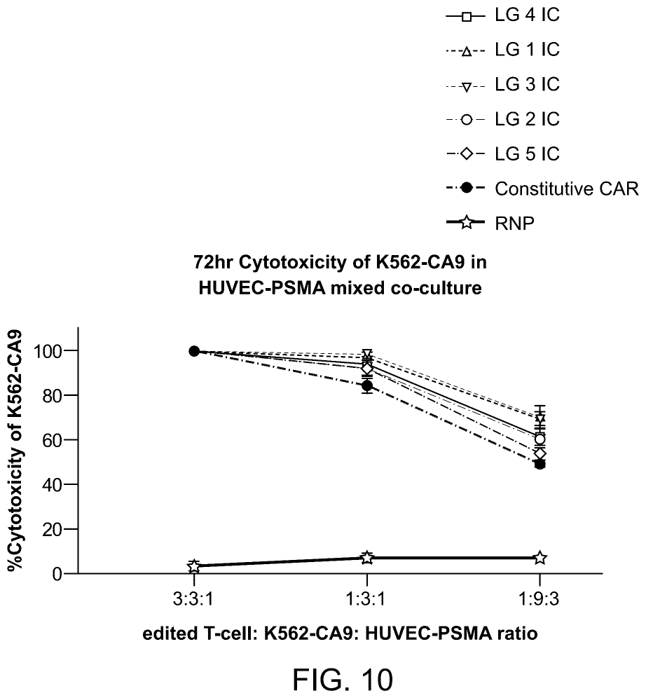

Co-culture with HUVEC-PSMA induced expression of the CAR protein on ICT cells and specific killing of CA9+ cells was confirmed (FIG. 10). Thus, ICTs expressing LG 1-5 ICs were capable of inducing CAR expression through interaction with PSMA+ endothelial cells and subsequently specifically engaging and killing CA9+ tumor cells. Therefore, without wishing to be bound by theory, ICTs can be primed by binding to endothelial cells expressing PSMA in order to express the CAR and then kill CA9+ target tumor cells.

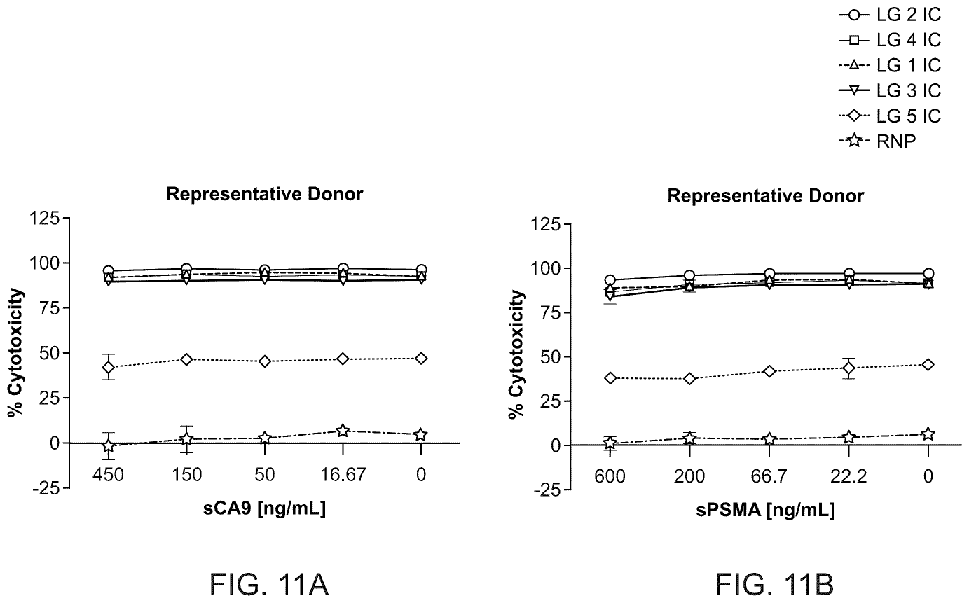

ICT cytotoxicity was not affected by soluble PSMA (FIG. 11B) or soluble CA9 (FIG. 11A).

Thus, logic gated ICT cells that utilize the presence of two antigens to trigger tumor cell killing to improve the therapeutic index of CA9 CAR T cells were developed, thereby enhancing tumor specificity. Induction of the CA9 CAR was gated on the expression of PSMA found on the tumor neovasculature of ccRCC. PSMA and CA9 are not known to be co-expressed in normal tissues. When the anti-PSMA priming receptor (PrimeR) binds PSMA, PrimeR engagement triggers proteolytic release of a transcription factor that induces expression of a CA9 CAR.

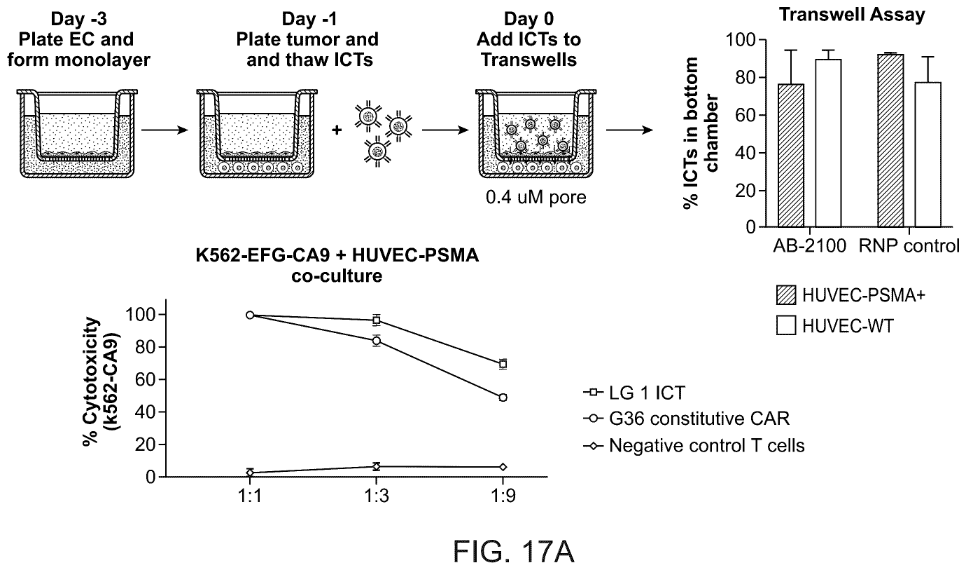

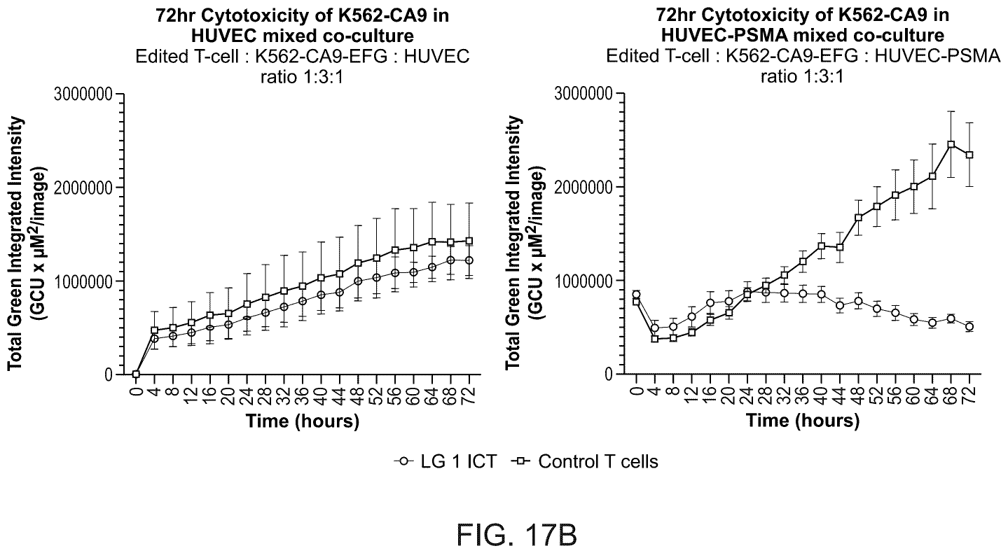

The feasibility of vascular priming was confirmed using a transwell assay where ICTs expressing LG 1 IC were primed by a PSMA expressing endothelial cell line and then migrated across a transwell membrane to kill CA9 expressing RCC cells (FIGS. 17A and 17B). As shown in FIG. 17A, T cells expressing a PSMA/CA9 logic gate can be primed during transmigration and can kill CA9+ tumor cells. As shown in FIG. 17B, the LG 1 ICT cells co-cultured with the HUVEC cells did not result in cytotoxicity of the K562-CA9 cells as the K562-CA9 cells continued to grow during the assay time course. In contrast, co-culturing the LG 1 ICT cells with HUVEC-PSMA cells did result in cytotoxicity of the K562-CA9 cells during the assay.

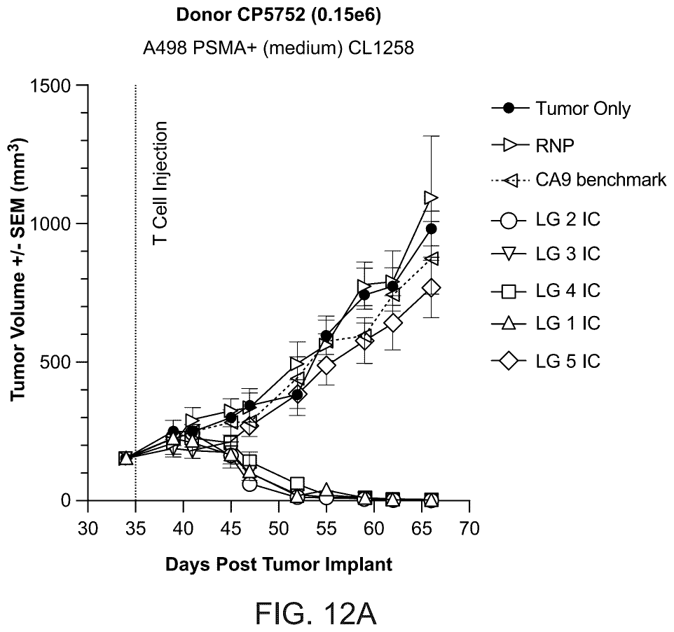

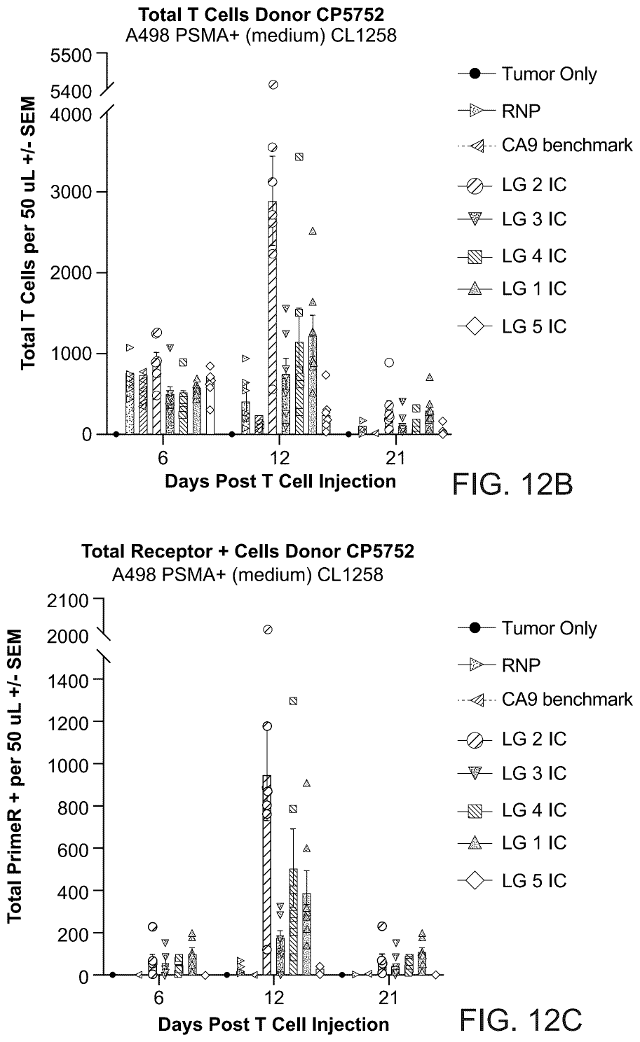

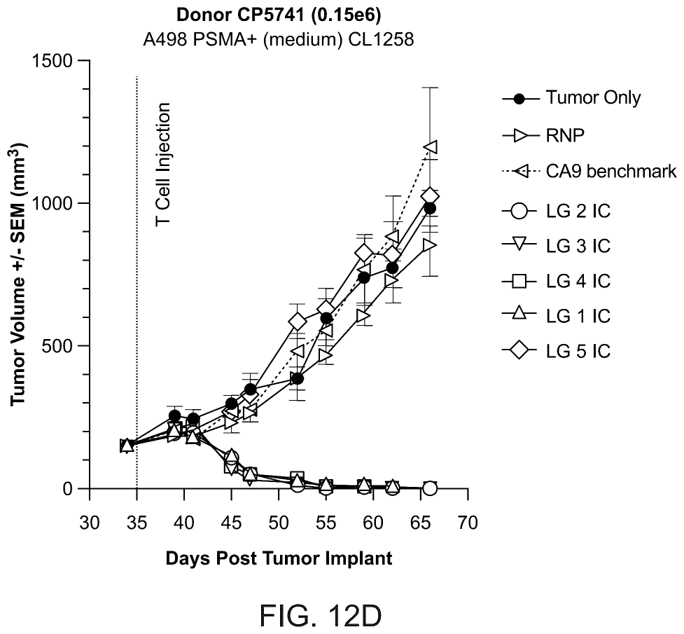

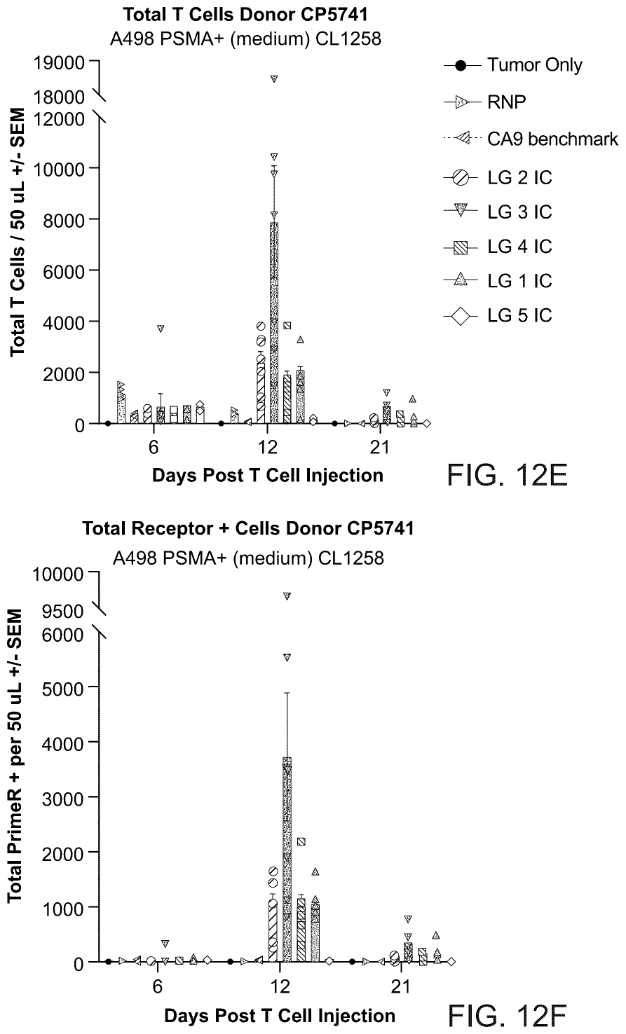

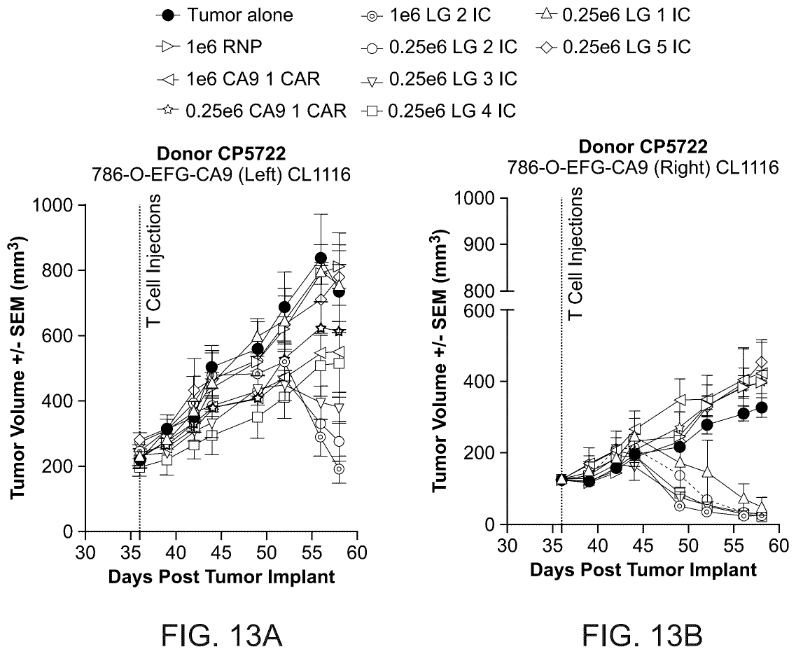

When constitutively expressed in the ICT cells, the SPA resulted in significant enhancements in T-cell potency and expansion. Repetitive stimulation assays, wherein T cells were challenged with tumor cells every 2 days, show that Class I SPAs result in 6-log or higher improved tumor cell clearance over a 2-week assay period. (data not shown) Across various mouse xenograft models (FIGS. 12A, 12D, 13B, and data not shown), SPA-expressing ICTs reach at least 6-fold improved tumor growth inhibition. RNAseq and ATACseq analysis indicate changes to gene expression profiles in T cells expressing Class I SPAs, with maintenance of T cell stem-like phenotypes, and restricted accessibility of various exhaustion marker genes (data not shown). Importantly, despite significantly increased levels of expansion, ICTs equipped with SPAs are not immortalized, showing no signs of cytokine-independent outgrowth. (data not shown) In addition, SPA-expressing ICT cells rapidly contract following tumor clearance in-vivo (FIG. 12B, 12C, 12E, 12F).

To further increase the potency and persistence of the ICT cells, an shRNA cassette targeting both FAS and TGFBR2, a receptor used for TGFB signaling in T cells, was inserted into the ICTs. Addition of FAS/TGFBR2 shRNAs enhanced antitumor activity of PSMAxCA9 logic gate expressing T cells during in vitro chronic stimulation assays conducted in the presence of exogenous TGFb (data not shown). Furthermore, FAS/TGFBR shRNA containing ICTs demonstrated enhanced antitumor activity in multiple xenograft RCC models (Example 4). Collectively, these results demonstrate that PSMAxCA9 ICT cells can (i) selectively target antigens that cannot generally be safely targeted by conventional CARs; and (ii) overcome multiple suppressive mechanisms in the tumor microenvironment.

### Example 4: In Vivo Efficacy of PSMA primeR and CA9 CAR Logic Gate T Cells

### Materials and Methods

### A498 RCC Efficacy Model

Human ccRCC A498 cells express endogenous levels of CA-9 and were engineered to express physiological levels of PSMA antigen. 2×10⁶ A498 PSMA cells were inoculated into the right dorsal flank of five-six weeks old, female NSG MHC I/II DKO mice. Day 35 post tumor inoculation, mean tumor volume of 150 mm³ was reached and tumor-bearing animals were randomized into various treatment groups such that mean tumor volume per group was within 10% of the overall mean. Seven mice/group were injected intravenously with a single dose of 0.15×10⁶ of PrimeR+ ICT cells expressing one of the five LG ICTs described in Example 3 (LG 1 IC, LG 2 IC, LG 3 IC, LG 4 IC, or LG 5 IC), RNP or PBS. The study was repeated with ICTs generated from two different normal donors. Tumor volumes and body weight were recorded bi-weekly. Tumor volume was calculated as per formula ½*L*W², where L is tumor length and W is tumor width.

Blood pharmacokinetics demonstrated the expansion of ICTs on day 14 followed by complete contraction by day 42 post T cell injection. PrimeR+ ICTs in mouse blood were quantified to track expansion of ICTs using flow cytometry with count bright beads for T cell quantification/volume. Mean and SEM plotted.

### Dual Flank Model

Human ccRCC 786-O cells were engineered to express either CA9 and PSMA or CA9 only. 2×10⁶786-O-CA9+ and 786-O-CA9+-PSMA+ cells were inoculated into the left and right dorsal flank respectively of five-six weeks old, female NSG MHC I/II DKO mice. Day 35 post tumor inoculation, mean tumor volume of 150-200 mm³ was reached on each flank and tumor-bearing animals were randomized into various treatment groups such that mean tumor volume per group on the right flank was within 10% of the overall mean. Seven mice/group were injected intravenously with a single dose of 0.25×10⁶ or 1×10⁶ of PrimeR+ ICT cells, constitutive CAR T cells, RNP or PBS control. Tumor volumes and body weight were recorded bi-weekly. Tumor volume was calculated as per formula ½*L*W², where L is tumor length and W is tumor width. (B) tumor volumes on the 786-O CA9 only flank (left), and (C) tumor volumes on the 786-O-CA9+PSMA+ flank (right). Data represents a single donor study with 7 mice per group, mean and SEM plotted.

### Results

### A498 RCC Efficacy Model

ICTs expressing LG 1-5 ICs showed tumor elimination in a ccRCC model. FIGS. 12A and 12D show the tumor volume post tumor implant in mice treated with ICTs expressing Logic Gates 1-5, RNP or PBS generated from T cells from either donor 1 (FIGS. 12A-C) or donor 2 (FIGS. 12D-F). FIGS. 12B and 12E show the total T cells and expansion of the ICTs on day 12 post inoculation followed by contraction by day 21. FIGS. 12C and 12F show total T cells expressing the priming receptor on days 12 and 21. In both replicates, the ICT cells demonstrated significant tumor-growth inhibition in mice (P<0.05).

### Dual Flank Model

The ICTs expressing LG 1-5 ICs showed specificity in a dual flank model (FIG. 13A-B). Greater tumor growth inhibition (TGI) was observed in the dual positive PSMA-CA9 flank (FIG. 13B) than the single positive CA9-only flank (FIG. 13A). Thus, the dual flank xenograft model shows that logic gated circuits (ICTs) more selectively killed tumors that express both CA9 and PSMA, and not tumors that express CA9 alone.

### Example 5: Synthesis and Characterization of PSMA primeR and CA9 CAR Logic Gate T Cells with TGFBR Knockdown or a Synthetic Pathway Activator

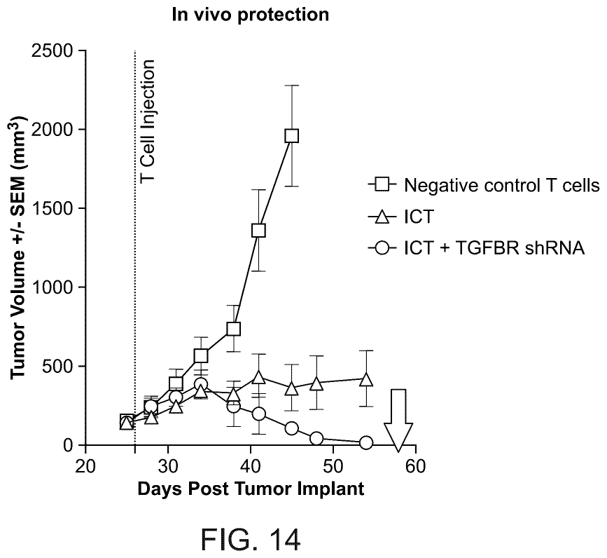

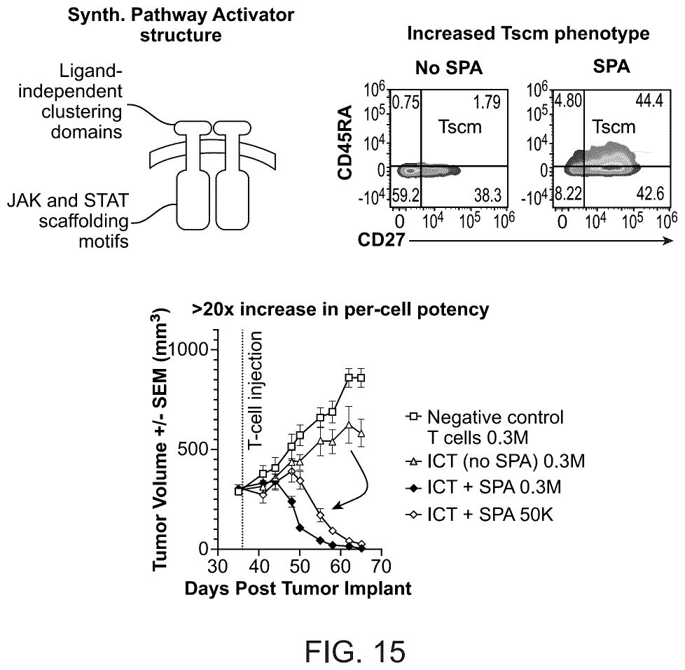

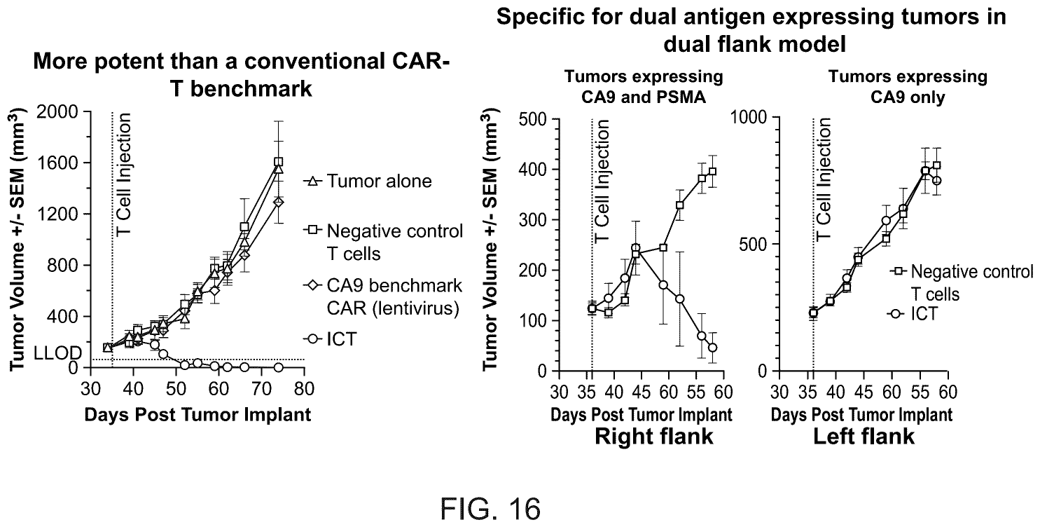

T cells expressing the PSMA and CA9 logic gate (also called integrated circuit T cells (ICTs)) as well as a synthetic pathway activator (SPA) and/or FAS and/or TGFBR shRNA were constructed and characterized. The results are provided in FIGS. 14, 15, and 16. FIG. 14 shows that TGFBR knockdown protected ICT cells against TGFβ-mediated inhibition in vitro. FIG. 15 shows the structure of an exemplary synthetic pathway activator and that synthetic pathway activators can increase cell anti-tumor potency and T stem cell memory phenotype. FIG. 16 shows that ICT cells are a potent and specific cell therapy in a Renal Cell Carcinoma model in vivo. ICT cells expressing a CA9 and PSMA logic gate demonstrated significant and specific tumor reduction against tumor cells expressing both CA9 and PSMA in a dual flank model as described in Example 4. The ICT cells were also more potent than a conventional CA9 CAR T cell used as a benchmark.

### Example 6: PSMA and CA9 Expression in Renal Carcinoma (ccRCC)

### Materials and Methods

### PSMA IHC Staining

Fixed human tumor tissue microarray (TMA) slides KD20811a, KD20812a, KD603, and KD951a ccRCC tumor samples were commercially purchased from Tissue Array LLC (Tissue Array.com LLC, Derwood, MD). Briefly, TMA and control slides were baked at 60° C. for 1 hour in an oven. The slides were deparaffinized and rehydrated using standard methods. Antigen retrieval was performed using the Diva Decloaker buffer in a pressure cooker at 110° C. for 15 minutes. The slides were then placed inside an Biocare IntelliPATH FLX autostainer for staining. Briefly, the slides were subject to IntelliPATH Peroxidase Blocking Reagent, IntelliPATH Background Punisher, anti-PSMA antibody (Clone 3E6 or negative control), DAB chromogen, and lastly hematoxylin. The slides were then dehydrated, and the coverslips were applied onto the slides.

### CA9 IHC Staining

The ccRCC tumor samples as described above were deparaffinized and rehydrated using standard methods. Antigen retrieval was performed using the Borg Decloaker buffer in a pressure cooker at 110° C. for 15 minutes. The slides were then placed inside an Biocare IntelliPATH FLX autostainer for staining. Briefly, the slides were subject to IntelliPATH Peroxidase Blocking Reagent, IntelliPATH Background Punisher, anti-CA9 antibody (Clone M75) or negative control), DAB chromogen, and lastly hematoxylin. The slides were then dehydrated, and the coverslips were applied onto the slides.

### IHC Scoring

IHC scoring was manually conducted using brightfield microscopy. Positive CA9 staining was defined by either ≥50 or ≥80% of the evaluable tumor cells exhibiting 1+ or greater staining intensity on plasma membrane. Positive PSMA staining was defined by 1+ or greater staining of PSMA in ≥1% of endothelial cell staining in tumor cores. H-scores on tumors were calculated by standard methods.

### Results

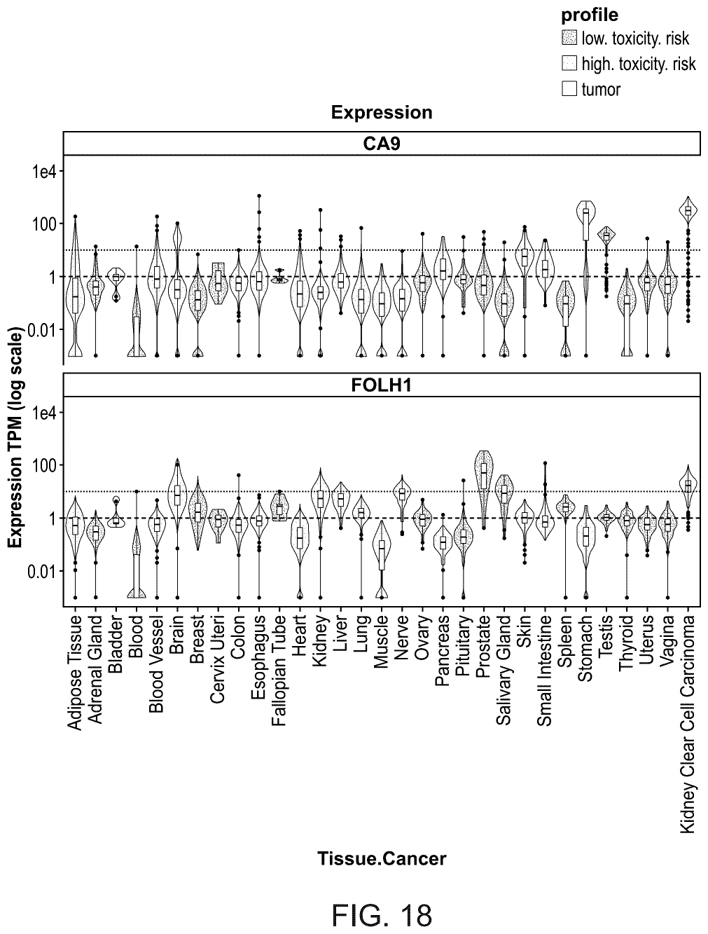

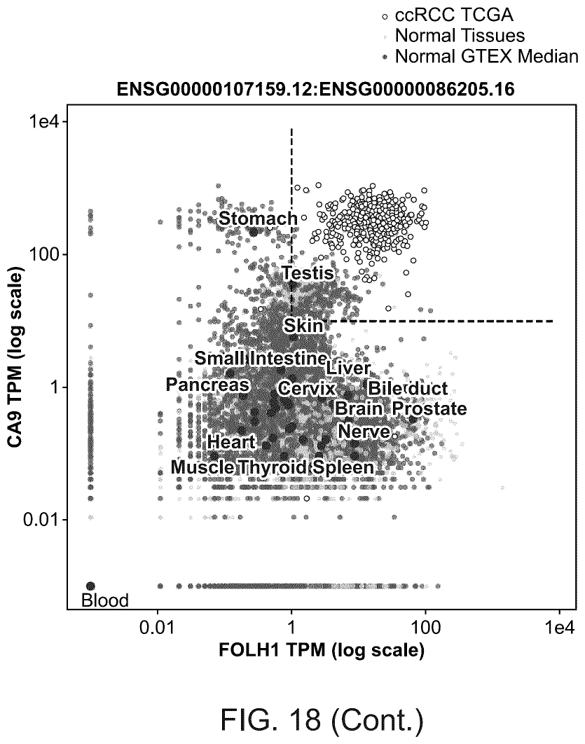

More than 2000 cytoplasmic membrane proteins and more than 4,00,000 potential antigens combinations were identified and compared to a tumor and primary sample bulk RNAseq database with more than 20,000 samples. Cytolytic targets had high expression in tumors with limited expression in normal tissues. Priming targets were expressed in tumors and not expressed in normal tissues with cytolytic targets. Exemplary target pairs had cytolytic and priming targets exclusively co-expressed in tumors. Out of this identification strategy, PSMA and CA9 were identified due to the co-expression of PSMA and CA9 mRNA in ccRCC with limited overlapping expression in normal tissues (FIG. 18).

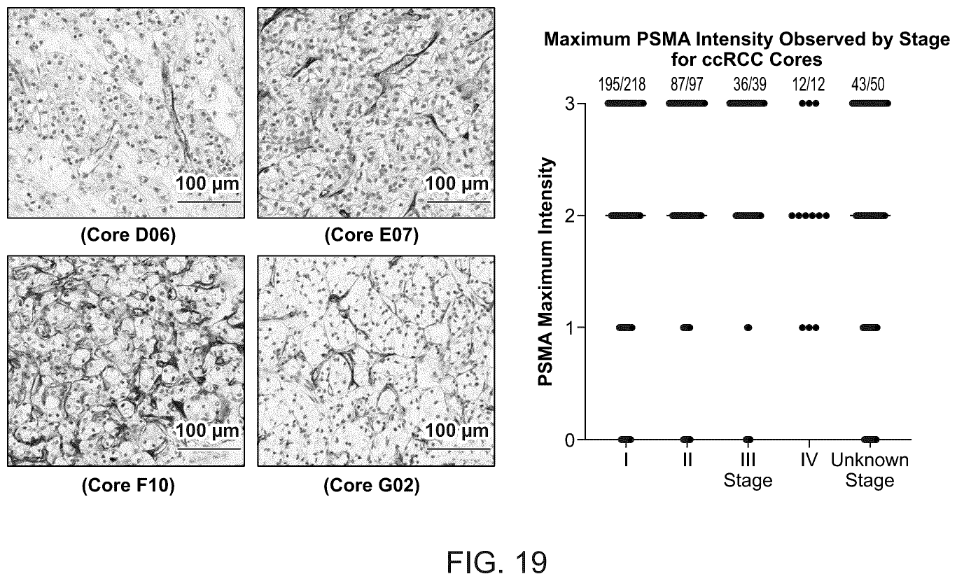

IHC staining of endothelial cells in ccRCC tumor samples with anti-PSMA clone 3E6 (Agilent Technologies, Inc., Santa Clara, CA) showed that ccRCC vasculature is PSMA-positive (PSMA+). Representative images at different staining intensities with 40× magnification are provided in FIG. 19. PSMA IHC intensity was scored on ccRCC tumor microarrays KD20811a, KD20812a, KD603, and KD951a and binned by AJCC disease stage (FIG. 19). As shown in FIG. 19, PSMA expression was observed on cells in all stages of ccRCC.

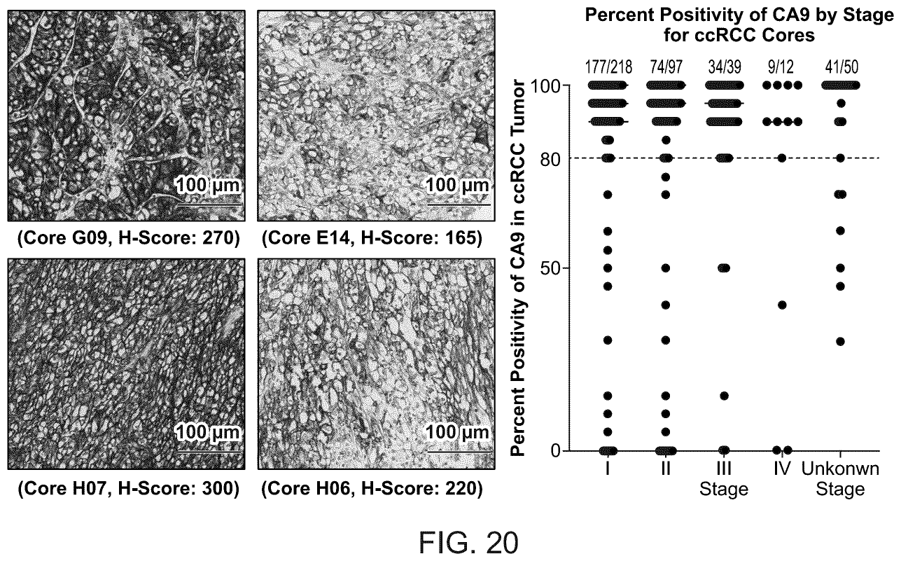

IHC staining of endothelial cells in ccRCC tumor samples with anti-CA9 clone M75 (Creative Biolabs, Shirley, NY) showed that ccRCC tumors are CA9-positive (CA9+). Representative images at different staining intensities with 40× magnification are provided in FIG. 20. CA9 IHC intensity was scored on ccRCC tumor microarrays KD20811a, KD20812a, KD603, and KD951a and binned by AJCC disease stage (FIG. 20). As shown in FIG. 20, CA9 expression was observed on ccRCC cells in all stages of ccRCC.

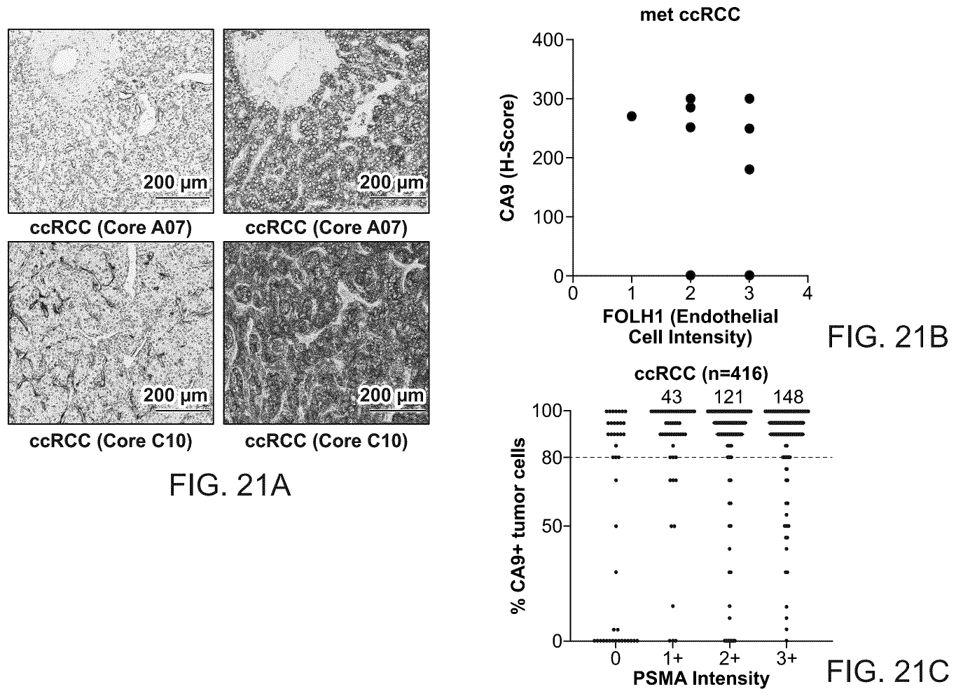

PSMA and CA9 were commonly co-positive in ccRCC. Representative images of ccRCC tumor samples co-stained for PSMA and CA9 with 20× magnification are provided in FIG. 21A. Co-expression of PSMA and CA9 in ccRCC via IHC (n=416) is provided in FIG. 21B. Positivity of PSMA was defined at 1+ or greater intensity of IHC signal observed in ccRCC endothelial compartment. Positivity of CA9 was set as 80% of the tumor expressing 1+ or greater intensity of IHC signal. Using these relative cutoff criteria for PSMA and CA9 positivity, co-positivity of PSMA and CA9 was 75% (312/416) in ccRCC by IHC. FIG. 21C provides co-positivity measurement of PSMA and CA9 in metastatic specimens according to the previous cutoff criteria. In these samples, co-positivity was 73% (8/11).

Normal adjacent kidney tissue was negative for CA9 staining, indicating that CA9 positivity is associated with ccRCC tumors. FIG. 22 provides images of CA9 and PSMA IHC in ccRCC samples versus normal adjacent kidney samples. Representative images of patient-matched ccRCC and normal adjacent kidney specimens were stained with PSMA and CA-9 antibodies as described above. Stained sections may not be sequential. PSMA staining was observed in both normal adjacent kidney samples and tumor samples, while CA9 expression was limited to tumor samples only.

PSMA and CA9 were identified as priming and cytolytic antigen targets, respectively, in clear cell renal cell carcinoma (ccRCC). Using a bioinformatic discovery path, PSMA and CA9 mRNA are co-expressed in 94% of ccRCC tumors (n=530, TCGA). Immunohistochemical (IHC) assessments further reveal robust membranous PSMA and CA9 protein co-expression in 80% of ccRCC (n=416). Expression of CA9 and PSMA was evaluated across disease stage and co-expression was confirmed throughout disease progression. Distant metastases similarly showed evidence of common expression. In contrast, IHC assessment of normal tissues revealed limited membranous co-expression of PSMA and CA9 in normal high-risk toxicity tissues. Without wishing to be bound by theory, these findings collectively support the utility of PSMA and CA9 as target antigens for AND-logic-gated therapeutics for the treatment of ccRCC.

### Example 7: PSMA and CA9 Expression in Colorectal Cancer (CRC)

### Materials and Methods

### PSMA IHC Staining

Fixed human tumor tissue microarray (TMA) slides (CO1922) were commercially purchased from US Biomax. Briefly, TMA and control slides were baked at 60° C. for 1 hour in an oven. The slides were deparaffinized and rehydrated using standard methods. Antigen retrieval was performed using the Diva Decloaker buffer in a pressure cooker at 110° C. for 15 minutes. The slides were then placed inside an Biocare IntelliPATH FLX autostainer for staining. Briefly, the slides were subject to IntelliPATH Peroxidase Blocking Reagent, IntelliPATH Background Punisher, anti-PSMA antibody (Clone 3E6 (Agilent Technologies, Inc., Santa Clara, CA) or negative control), DAB chromogen, and lastly hematoxylin. The slides were then dehydrated, and the coverslips were applied onto the slides.

### CA9 IHC Staining

Fixed human tumor tissue microarray (TMA) slides (described above) were deparaffinized and rehydrated using standard methods. Antigen retrieval was performed using the Borg Decloaker buffer in a pressure cooker at 110° C. for 15 minutes. The slides were then placed inside an Biocare IntelliPATH FLX autostainer for staining. Briefly, the slides were subject to IntelliPATH Peroxidase Blocking Reagent, IntelliPATH Background Punisher, anti-CA9 antibody (Clone M75 (Creative Biolabs, Shirley, NY)) or negative control), DAB chromogen, and lastly hematoxylin. The slides were then dehydrated, and the coverslips were applied onto the slides.

### IHC Scoring

IHC scoring was manually conducted using brightfield microscopy. Positive CA9 staining was defined by either ≥50 or ≥80% of the evaluable tumor cells exhibiting 1+ or greater staining intensity on plasma membrane. Positive PSMA staining was defined by 1+ or greater staining of PSMA in ≥1% of endothelial cell staining in tumor cores. H-scores on tumors were calculated by standard methods.

### Results

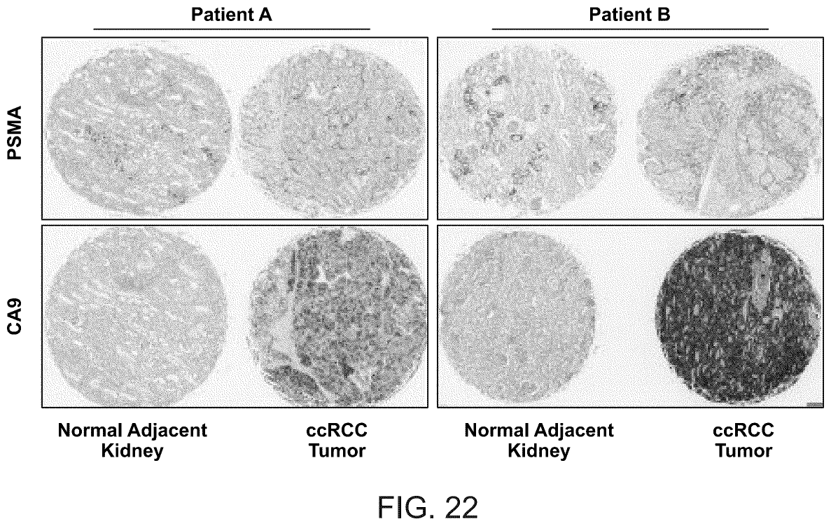

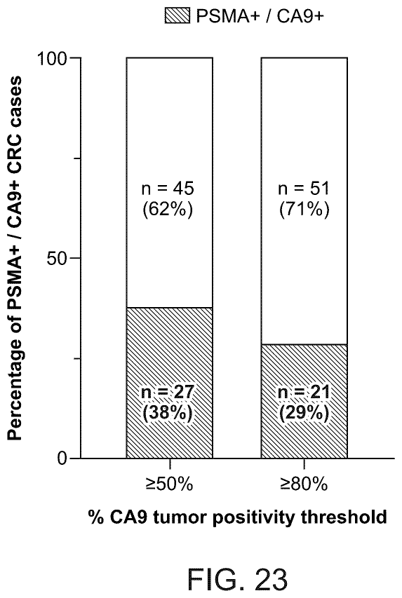

38% of colorectal cancer (CRC) tumor samples exhibited co-positivity of both PSMA and CA9 after applying a >50% tumor positivity IHC threshold. Increasing the IHC positivity threshold to 80% resulted in 29% of the CRC tumor samples exhibiting co-positivity of both PSMA and CA9 (FIG. 23). FIG. 23 shows the percentage of colorectal cancer patient samples, based on the CO1922 tissue microarray (TMA), that exhibited expression of both PSMA and CA9. The number of patient specimens exhibiting positivity for both PSMA and CA9 is listed as n, with the percentage (%) underneath.

### Example 8: PSMA and CA9 Expression in Lung Cancer

### Materials and Methods

### PSMA IHC Staining

Fixed human tumor tissue microarray (TMA) slides for lung adenocarcinoma (LU-AD) and lung squamous cell carcinoma (LU-SSC) (tissue array LC819a) were commercially purchased from Tissue Array LLC (Tissue Array.com LLC, Derwood, MD). PSMA staining was performed as described in Examples 6 and 7. Lung adenocarcinoma (LU-AD) and lung squamous cell carcinoma (LU-SSC) samples were purchased from.

### CA9 IHC Staining

Fixed human tumor tissue microarray (TMA) slides for lung adenocarcinoma (LU-AD) and lung squamous cell carcinoma (LU-SSC) (tissue array LC819a) were commercially purchased from Tissue Array LLC (Tissue Array.com LLC, Derwood, MD). CA9 staining was performed as described in Examples 6 and 7.

### IHC Scoring

IHC scoring was manually conducted using brightfield microscopy. Positive CA9 staining was defined by either ≥50 or ≥80% of the evaluable tumor cells exhibiting 1+ or greater staining intensity on plasma membrane. Positive PSMA staining was defined by 1+ or greater staining of PSMA in ≥1% of endothelial cell staining in tumor cores. H-scores on tumors were calculated by standard methods.

### Results

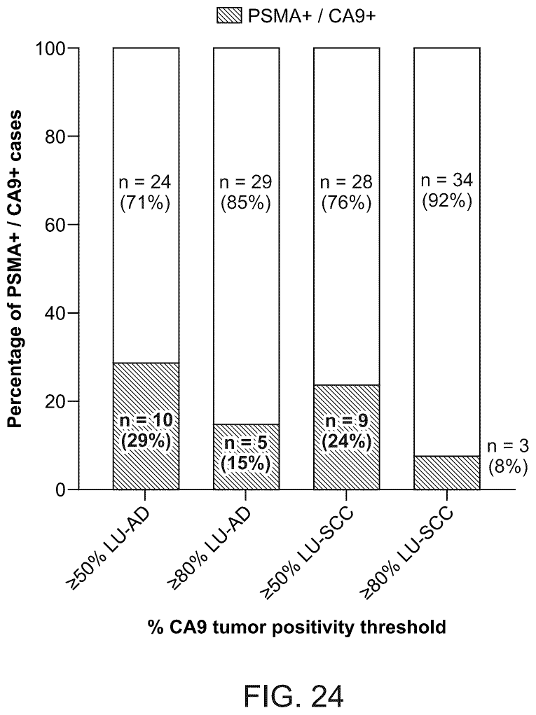

The prevalence of PSMA and CA9 expression in lung adenocarcinoma (LU-AD) and lung squamous cell carcinoma (LU-SSC), subtypes that account for approximately 70% of all lung cancer, was analyzed via immunohistochemistry (IHC) (FIG. 24). FIG. 24 shows the percentage of lung cancer patients, based on the LC819a TMA, that exhibited expression of both PSMA and CA9. The number of patient specimens exhibiting positivity for both PSMA and CA9 is listed as n, with the percentage (%) underneath.

29% of LU-AD patient samples exhibited co-positivity of both PSMA and CA9 after applying a >50% tumor positivity IHC threshold. Increasing the IHC positivity threshold to 80% resulted in 15% of the LU-AD patient samples exhibiting co-positivity of both PSMA and CA9 (FIG. 24).

24% of LU-SCC patient samples exhibited co-positivity of both PSMA and CA9 after applying a >50% tumor positivity IHC threshold. Increasing the IHC positivity threshold to 80% resulted in 8% of the LU-SCC patient samples exhibiting co-positivity of both PSMA and CA9 (FIG. 24).

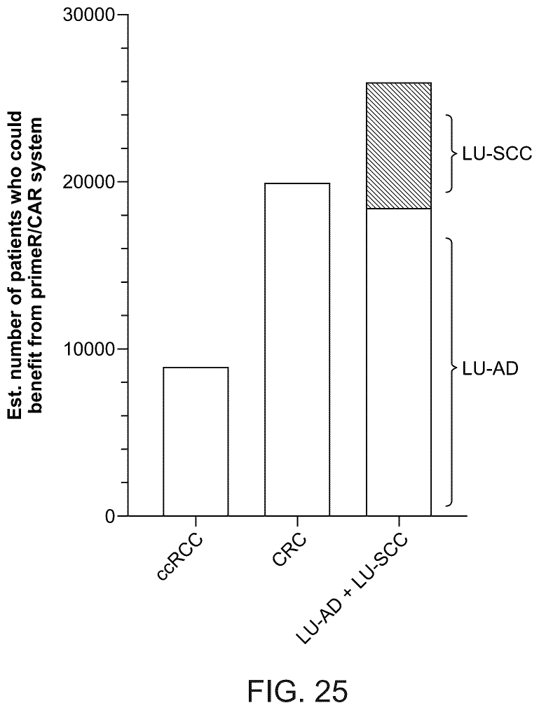

To assess the number of ccRcc, colorectal and lung cancer patients who may benefit from PSMA primeR and CA9 CAR Logic Gate T cells described in Examples 1 to 5, an analysis based on the estimated new deaths for each indication in 2023 and percentage of patients that express PSMA and CA9 was conducted. Based on this calculation, approximately 9,000, 20,000, 64,000 and 32,000 ccRcc, CRC, LU-AD and LU-SCC patients, respectively, may benefit from the PSMA primeR and CA9 CAR Logic Gate T cells disclosed herein (FIG. 25).

### Example 9: Human Clinical Trial Study Design

### Materials and Methods

Patients will be enrolled into a phase ½ open label, multicenter study for safety and efficacy of the LG T cells (PrimeR⁺ cells) described in Examples 1 to 5 to treat advanced/metastatic clear cell renal cell carcinoma (ccRCC). The targeted patient population will be patients with advanced/metastatic clear cell renal cell carcinoma (ccRCC) after immune checkpoint inhibitor and VEGF-targeted therapy. No initial selection for PSMA and CA9 expression will be performed.

The study will consist of phase 1 for safety/escalation dosing and phase 2 for efficacy. The Phase 1 study design is a First-in-Human (FIH) escalation with patient backfill in a 3+3 design. There will be approximately 60 patients in the phase 1 cohort. The phase 1 backfill cohorts can be up to 12 patients/cohort. There will be up to 70 patients in the phase 2 cohort.

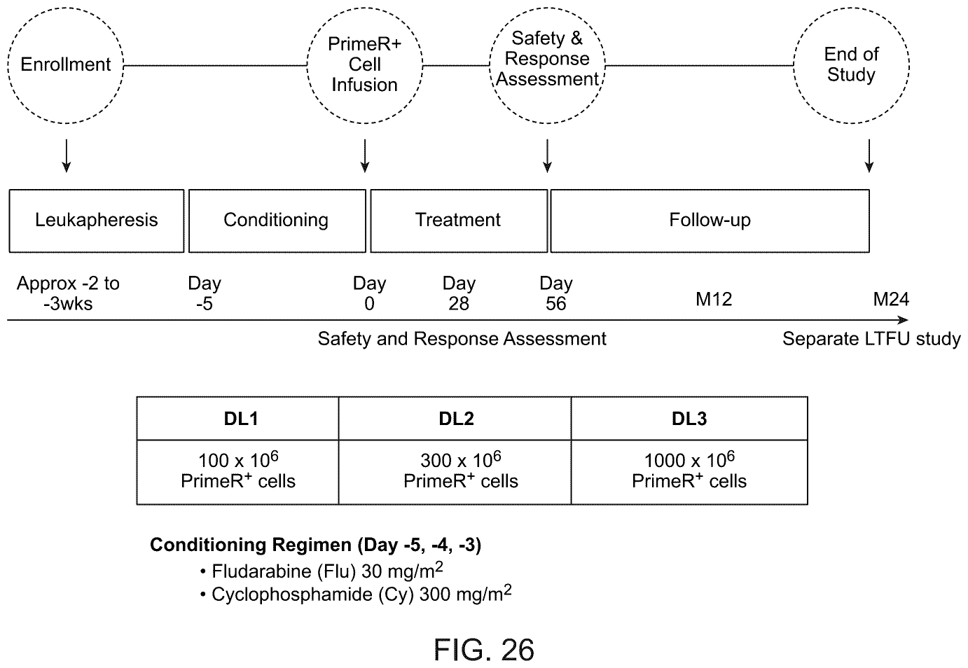

The phase 1 study design is provided in FIG. 26. Patients will be enrolled and undergo leukapheresis for T cell collection. This stage will take about 2-3 weeks.

In the phase 1 study, patients will be conditioned prior to LG T cell infusion. On Days −5, −4, and −3 patients will be conditioned with 30 mg/m² fludarabine and 300 mg/m² cyclophosphamide. Dosing of the LG T cells in the phase 1 study without the conditioning may also be performed.

LG T cells (PrimeR⁺ cells) will be infused on Day 0 and treatment will run until Day 56. LG T cells (PrimeR⁺ cells) will be dosed at 100×10⁶, 300×10⁶ or 1000×10⁶ cells. Safety and response assessments will be taken on Day 56 as well as through the study. Post-treatment follow up appointments will be done at month 12 and month 24 post Day 0. A separate Long term follow up (LTFU) study will also be performed.

While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

1. At least one polypeptide comprising at least one of:
a. a first antigen-binding domain that specifically binds Prostate-Specific Membrane Antigen (PSMA) (SEQ ID NO: 2) wherein the first antigen-binding domain comprises a first variable heavy (VH) chain sequence comprising three heavy chain CDR sequences, CDR-H1, CDR-H2, and CDR-H3, of the VH sequence set forth in SEQ ID NO: 118 or 130, and a first variable light (VL) chain sequence comprising three light chain CDR sequences, CDR-L1, CDR-L2, and CDR-L3, of the VL sequence set forth in SEQ ID NOs: 119 or 131; and/or
b. a second antigen-binding domain that specifically binds to Carbonic Anhydrase IX (CA9) (SEQ ID NO: 1) wherein the second antigen-binding domain comprises a second variable heavy (VH) chain sequence comprising three heavy chain CDR sequences, CDR-H1, CDR-H2, and CDR-H3, of the VH sequence set forth in SEQ ID NOs: 99 or 110, and, optionally, a second variable light (VL) chain sequence comprising three light chain CDR sequences, CDR-L1, CDR-L2, and CDR-L3, of the VL sequence set forth in SEQ ID NO: 100.

2. The at least one polypeptide of claim 1, wherein:
a. the first variable heavy (VH) chain sequence comprises:
i. a CDR-H1 comprising the sequence set forth in SEQ ID NO: 120, a CDR-H2 comprising the sequence set forth in SEQ ID NO: 121, and a CDR-H3 comprising the sequence set forth in SEQ ID NO: 122, and the first variable light (VL) chain sequence comprises a CDR-L1 comprising the sequence set forth in SEQ ID NO: 123, a CDR-L2 comprises the sequence set forth in SEQ ID NO: 124, and a CDR-L3 comprising the sequence set forth in SEQ ID NO: 125; or
ii. a CDR-H1 comprising the sequence set forth in SEQ ID NO: 132, a CDR-H2 comprising the sequence set forth in SEQ ID NO: 133, and a CDR-H3 comprising the sequence set forth in SEQ ID NO: 134, and the first variable light (VL) chain sequence comprises a CDR-L1 comprising the sequence set forth in SEQ ID NO: 135, a CDR-L2 comprises the sequence set forth in SEQ ID NO: 136, and a CDR-L3 comprising the sequence set forth in SEQ ID NO: 137 and/or
b. the second variable heavy (VH) chain sequence comprises:
i. a CDR-H1 comprising the sequence set forth in SEQ ID NO: 101, a CDR-H2 comprising the sequence set forth in SEQ ID NO: 102, and a CDR-H3 comprising the sequence set forth in SEQ ID NO: 103, and the second variable light (VL) chain sequence comprises a CDR-L1 comprising the sequence set forth in SEQ ID NO: 104, a CDR-L2 comprises the sequence set forth in SEQ ID NO: 105, and a CDR-L3 comprising the sequence set forth in SEQ ID NO: 106; or
ii. a CDR-H1 comprising the sequence set forth in SEQ ID NO: 111, a CDR-H2 comprising the sequence set forth in SEQ ID NO: 112, and a CDR-H3 comprising the sequence set forth in SEQ ID NO: 113.

3. The at least one polypeptide of claim 1, wherein the first VH chain sequence comprises the sequence set forth in SEQ ID NOs: 118 or 130, and the first VL chain sequence comprises the sequence set forth in SEQ ID NOs: 119 or 131.

4. The at least one polypeptide of claim 1, wherein the second VH comprises the sequence as set forth in SEQ ID NOs: 99 or 110, and, optionally wherein the second VL comprises the sequence set forth in SEQ ID NO: 100.

5. A system comprising:
a. a first chimeric polypeptide comprising a priming receptor comprising the first antigen-binding domain that specifically binds PSMA (SEQ ID NO: 2); and
b. a second chimeric polypeptide comprising a chimeric antigen receptor (CAR) comprising the second antigen-binding domain that specifically binds to CA9 (SEQ ID NO: 1).

6. The system of claim 5, wherein:
a. the first antigen-binding domain comprises a first variable heavy (VH) chain sequence comprising three heavy chain CDR sequences, CDR-H1, CDR-H2, and CDR-H3, of the VH sequence set forth in SEQ ID NOs: 118 or 130, and a first variable light (VL) chain sequence comprising three light chain CDR sequences, CDR-L1, CDR-L2, and CDR-L3, of the VL sequence set forth in SEQ ID NOs: 119 or 131; and/or
b. the second antigen-binding domain comprises a second variable heavy (VH) chain sequence comprising three heavy chain CDR sequences, CDR-H1, CDR-H2, and CDR-H3, of the VH sequence set forth in SEQ ID NOs: 99 or 110, and, optionally, a second variable light (VL) chain sequence comprising three light chain CDR sequences, CDR-L1, CDR-L2, and CDR-L3, of the VL sequence set forth in SEQ ID NO: 100.

7. The system of claim 6, wherein:
a. the first variable heavy (VH) chain sequence comprises:
i. a CDR-H1 comprising the sequence set forth in SEQ ID NO: 120, a CDR-H2 comprising the sequence set forth in SEQ ID NO: 121, and a CDR-H3 comprising the sequence set forth in SEQ ID NO: 122, and the first variable light (VL) chain sequence comprises a CDR-L1 comprising the sequence set forth in SEQ ID NO: 123, a CDR-L2 comprises the sequence set forth in SEQ ID NO: 124, and a CDR-L3 comprising the sequence set forth in SEQ ID NO: 125; or
ii. a CDR-H1 comprising the sequence set forth in SEQ ID NO: 132, a CDR-H2 comprising the sequence set forth in SEQ ID NO: 133, and a CDR-H3 comprising the sequence set forth in SEQ ID NO: 134, and the first variable light (VL) chain sequence comprises a CDR-L1 comprising the sequence set forth in SEQ ID NO: 135, a CDR-L2 comprises the sequence set forth in SEQ ID NO: 136, and a CDR-L3 comprising the sequence set forth in SEQ ID NO: 137 and/or
b. the second variable heavy (VH) chain sequence comprises:
i. a CDR-H1 comprising the sequence set forth in SEQ ID NO: 101, a CDR-H2 comprising the sequence set forth in SEQ ID NO: 102, and a CDR-H3 comprising the sequence set forth in SEQ ID NO: 103, and the second variable light (VL) chain sequence comprises a CDR-L1 comprising the sequence set forth in SEQ ID NO: 104, a CDR-L2 comprises the sequence set forth in SEQ ID NO: 105, and a CDR-L3 comprising the sequence set forth in SEQ ID NO: 106 or
ii. a CDR-H1 comprising the sequence set forth in SEQ ID NO: 111, a CDR-H2 comprising the sequence set forth in SEQ ID NO: 112, and a CDR-H3 comprising the sequence set forth in SEQ ID NO: 113.

8. The system of claim 6, wherein the first VH chain sequence comprises the sequence set forth in SEQ ID NOs: 118 or 130, and the first VL chain sequence comprises the sequence set forth in SEQ ID NOs: 119 or 131.

9. The system of claim 6, wherein the second VH comprises the sequence as set forth in SEQ ID NOs: 99 or 110, and, optionally, wherein the second VL comprises the sequence set forth in SEQ ID NO: 100.

10. The system of claim 5, wherein the priming receptor comprises from N-terminus to C-terminus:
a. the first antigen-binding domain;
b. a transmembrane domain comprising one or more ligand-inducible proteolytic cleavage sites; and
c. an intracellular domain comprising a transcriptional effector, wherein binding of PSMA by the first antigen-binding domain results in cleavage at the one or more ligand-inducible proteolytic cleavage sites.

11. The system of claim 5, wherein the priming receptor comprises a sequence as set forth in SEQ ID NOs: 127, 252, 138, or 253.

12. The system of claim 5, wherein the CAR comprises, from N-terminus to C-terminus:
a. the antigen-binding domain;
b. a transmembrane domain;
c. an optional intracellular co-stimulatory domain; and
d. an intracellular activation domain.

13. The system of claim 5, wherein the CAR comprises a sequence as set forth in SEQ ID NOs: 108, 250, 115, or 251.

14. The system of claim 5, further comprising a third polypeptide comprising a synthetic pathway activator (SPA) comprising the sequence as set forth in SEQ ID NOs: 141 or 254.

15. The system of claim 5, wherein the system is encoded by a nucleic acid comprising a sequence with at least 80%, 85%, 90%, 91%, 92%, 93%, 94%, 95%, 96%, 97%, 98%, 99%, or 100% identity to a sequence as set forth in SEQ ID NOs: 143, 144, 145, or 146.

16. A system comprising the at least one polypeptide of claim 1 and further comprising at least one or more nucleic acids comprising a nucleic acid sequence at least 15 nucleotides in length complementary to at least one of:
a. a nucleic acid sequence encoding human Fas Cell Surface Death Receptor (FAS) comprising the sequence set forth in SEQ ID NO: 3; and
b. a nucleic acid sequence encoding human transforming growth factor (TGF)-β Receptor 2 (TGFBR2) comprising the sequence set forth in SEQ ID NO: 4.

17. The system of claim 16, wherein the nucleic acid sequence complementary to human FAS comprises a sequence selected from the group consisting of the sequences set forth in SEQ ID NOs: 6-20.

18. The system of claim 16, wherein the nucleic acid sequence complementary to human TGFBR2 comprises a sequence selected from the group consisting of the sequences set forth in SEQ ID NOs: 34-82.

19. One or more nucleic acid(s) comprising a nucleotide sequence encoding the at least one polypeptide of claim 1.

20. The one or more nucleic acid(s) of claim 19, further comprising at least one or more nucleic acids comprising a nucleic acid sequence at least 15 nucleotides in length complementary to at least one of:
a. a nucleic acid sequence encoding human Fas Cell Surface Death Receptor (FAS) comprising the sequence set forth in SEQ ID NO: 3; and
b. a nucleic acid sequence encoding human transforming growth factor (TGF)-β Receptor 2 (TGFBR2) comprising the sequence set forth in SEQ ID NO: 4.

21. One or more nucleic acid(s) comprising a nucleotide sequence encoding the system of claim 5.

22. The one or more nucleic acid(s) of claim 21, further comprising at least one or more nucleic acids comprising a nucleic acid sequence at least 15 nucleotides in length complementary to at least one of:
a. a nucleic acid sequence encoding human Fas Cell Surface Death Receptor (FAS) comprising the sequence set forth in SEQ ID NO: 3; and
b. a nucleic acid sequence encoding human transforming growth factor (TGF)-β Receptor 2 (TGFBR2) comprising the sequence set forth in SEQ ID NO: 4.

23. One or more vector(s) comprising the one or more nucleic acid(s) of claim 19.

24. A cell comprising the at least one polypeptide of claim 1.

25. The cell of claim 24, wherein the cell is a primary human immune cell.

26. A pharmaceutical composition comprising the cell of claim 24, and a pharmaceutically acceptable excipient.

27. A cell comprising the system of claim 5, optionally wherein the cell is a primary human immune cell.

28. A method of editing a cell, comprising:
a. providing a nuclease domain and a guide RNA, wherein the nucleic acid comprises the nucleic acid of claim 19, and wherein the 5′ and 3′ ends of the nucleic acid comprise nucleotide sequences that are homologous to genomic sequences flanking an insertion site in the genome of the cell;
b. introducing the nuclease domain and nucleic acid into the cell, wherein the guide RNA specifically hybridizes to a target region of the genome of the cell, and wherein the nuclease domain cleaves the target region to create the insertion site in the genome of the cell; and
c. editing the cell via insertion of the nucleic acid into the insertion site in the genome of the cell.

29. A method of treating a disease or increasing an immune response in a subject comprising administering to the subject a cell comprising at least one polypeptide comprising at least one of:
a. a first antigen-binding domain that specifically binds Prostate-Specific Membrane Antigen (PSMA) (SEQ ID NO: 2); and/or
b. a second antigen-binding domain that specifically binds to Carbonic Anhydrase IX (CA9) (SEQ ID NO: 1),
optionally wherein the disease is cancer.

30. The method of claim 29, wherein the cancer is kidney cancer, clear cell renal cell carcinoma (ccRcc), colorectal cancer, or lung cancer.


SEQUENCE LISTING (389 sequences)

SEQ ID NO: 1 - protein, 459 aa, Homo sapiens
MAPLCPSPWLPLLIPAPAPGLTVQLLLSLLLLVPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 2 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 3 - RNA, 3696 nt, Homo sapiens
  source 1..3696: mol_type = mRNA
ctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataacttgtctttaatgcttcttggatcccttagaaggtacttcctttttaaccttaacccttttagtagttaaataattatttccataggttgctattgccaagaagacctcttccaaacagcacatgattattcgtcaaacagtttcgtattccagatactggaatgtggataagaaagtatacatttcaaggggtaggttttattattaagaaagccaaatgaggattttgaaatattctttcctgcatattatccattctagctacatgctggccagtgggccacctttcttttctgcaatttaatgctagtaatatattctatttaacccatgagtcccaaagtattagcatttcaacatgtaagcatgtcggtaagatagttgtgctttgcttagggttccctcctgtgttatggtctggaaagtgtctttaggcagaaagtctgagtgatcacagggttcactcattaatttctcttttctgagccatcatagtctgtgctgtctgctctccagttttctatttctagacagaagtagggcaagttaggtactagttattcttcatggccagaagtgcaagttctactttgcaagacaagattaagttagagaacaccctattccactttggtgaactcagagcaagaactttgagttcctttgggaggaagacagtggagaagtctttgtacttggtgatgtggtttttttcctcatggcttcacctagtggccccaagcatgacttctcccatgtcaatgagcacagccacattcccgagttgaggtgaccccacggtccagaatcatcctcattctggtgaacctggttctctttgtggtgggcatactgggtaggagaatcacccaaaggtcacccatgagctgcagaaaaaaaggctatttgcagaaggagctcacagatcacattgaaagcattgcatattcaaacatcttggtcttctttattggcatgcccacagggtcttctgacctctgattagatcagacactttttagatattgaatcatcagtttctgtacaactatctgaataaggtatataatcaatgaaatttagaatttttttctatgcttactcctgattggtaatttgtttgggtttagaattctatacaaggccatttgtaattttcctcagcactttaaaaatattaaaccatgttttcttaa

SEQ ID NO: 4 - DNA, 4530 bp, Homo sapiens
  source 1..4530: mol_type = genomic DNA
actcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 5 - RNA, 3470 nt, Homo sapiens
  source 1..3470: mol_type = mRNA
gcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggtggttatattggcaacctattctcactaagatggggtttatgtcagtcattttggttggcgcttttgttggctggacactgttttttcagcaaaatgccctataaacaattaattttgcccagcaagcttctgcactagtaactgacagtgctacattaatcataggggtttgtctgcagcaaacgcctcatatcccaaaaacggtgcagtagaatagacatcaaccagataagtgatatttacagtcacaagcccaacatctcaggactcttgactgcaggttcctctgaaccccaaactgtaaatggctgtctaaaataaagacattcatgtttgttaaaaactggtaaattttgcaactgtattcatacatgtcaaacacagtatttcacctgaccaacattgagatatcctttatcacaggatttgtttttggaggctatctggattttaacctgcacttgatataagcaataaatattgtggttttatctacgttattggaaagaaaatgacatttaaataatgtgtgtaatgtataatgtactattgacatgggcatcaacacttttattcttaagcatttcagggtaaatatattttataagtatctatttaatcttttgtagttaactgtactttttaagagctcaatttgaaaaatctgttactaaaaaaataaattgtatgtcgattgaattgtactggatacattttccatttttctaaagagaagtttgatatgagcagttagaagttggaataagcaatttctactatatattgcatttcttttatgttttacagttttccccattttaaaaagaaaagcaaacaaagaaacaaaagtttttcctaaaaatatctttgaaggaaaattctccttactgggatagtcaggtaaacagttggtcaagactttgtaaagaaattggtttctgtaaatcccattattgatatgtttatttttcatgaaaatttcaatgtagttggggtagattatgatttaggaagcaaaagtaagaagcagcattttatgattcataatttcagtttactagactgaagttttgaagtaaacacttttcagtttctttctacttcaataaatagtatgattatatgcaaaccttacattgtcattttaacttaatgaatattttttaaagcaaactgtttaatgaatttaactgctcatttgaatgctagctttcctcagatttcaacattccattcagtgtttaatttgtcttacttaaacttgaaattgttgttacaaatttaattgctaggaggcatggatagcatacattattatggatagcataccttatttcagtggttttcaaactatgctcattggatgtccaggtgggtcaagaggttactttcaaccacagcatctctgccttgtctctttatatgccacataagatttctgcataaggcttaagtattttaaagggggcagttatcatttaaaaacagtttggtcgggcgcggtggctcatgcctgtaatcccagcactttgggaggctgaagtgggcagatcacctgaggtcaggagttcaagaccagcctggccaacgtggtgaaacaccatctctactaaaaatgcaaaaattagctgggcatggtggagggcacctgtaatctcagctactcaggaggctgaggtaggagaattgcttgaacccaggagatggaggttgcagtgagctgagatcacgtcactgcactccagccagggcgacagagcgagactccatctcaaaagaaacaaacaaaaaaaacagtttgggccgggtgtggtggctcacgcttgtaatcccagcacttcggaaggccaaggcgggcggatcacgaggtcaagagatggagactgtcctggccaacatggtgaaatcccttctttactaaaaatacaaaaattatctgggcgtggtggtgcatgcctgtagtcccagctccttgggaggctaaggcaggagaatcacttgaacccgggaggcagaggttgcagtgagccgagattgcaccactgcactccagcctggcaacagagcaagacttcgtctcaaaaaaaaaaaaaaaaaaagtttgaaaaccattggtatagatagatattttgaattgatttgcatagtctccttgaatgtgttaaattatgttgaaagtatgaaagcaggatgtaggtggtactacatattaaataagatttatataaca

SEQ ID NO: 6 - RNA, 22 nt, synthetic construct
  source 1..22: mol_type = other RNA
tagattttaaacatccttggag

SEQ ID NO: 7 - RNA, 22 nt, synthetic construct
  source 1..22: mol_type = other RNA
tagattttaaacatccttggag

SEQ ID NO: 8 - RNA, 22 nt, synthetic construct
  source 1..22: mol_type = other RNA
ttactcttgatgaagtttgttt

SEQ ID NO: 9 - RNA, 22 nt, synthetic construct
  source 1..22: mol_type = other RNA
ttgaactttctgttctgctgtg

SEQ ID NO: 10 - RNA, 22 nt, synthetic construct
  source 1..22: mol_type = other RNA
ttgtctgtgtactccttccctt

SEQ ID NO: 11 - RNA, 22 nt, synthetic construct
  source 1..22: mol_type = other RNA
tctttgattgcaaacatgggtt

SEQ ID NO: 12 - RNA, 22 nt, synthetic construct
  source 1..22: mol_type = other RNA
ttgatctcatctattttggctt

SEQ ID NO: 13 - RNA, 22 nt, synthetic construct
  source 1..22: mol_type = other RNA
ttaagaatcttttcaaacacta

SEQ ID NO: 14 - RNA, 22 nt, synthetic construct
  source 1..22: mol_type = other RNA
ttctattgattcatagagtggg

SEQ ID NO: 15 - RNA, 22 nt, synthetic construct
  source 1..22: mol_type = other RNA
taatcttaatctttcatcctct

SEQ ID NO: 16 - RNA, 22 nt, synthetic construct
  source 1..22: mol_type = other RNA
ttaacttgacttagtgtcatga

SEQ ID NO: 17 - RNA, 22 nt, synthetic construct
  source 1..22: mol_type = other RNA
ttacataaatatgatcttctca

SEQ ID NO: 18 - RNA, 22 nt, synthetic construct
  source 1..22: mol_type = other RNA
tacataaatatgatcttctcag

SEQ ID NO: 19 - RNA, 22 nt, synthetic construct
  source 1..22: mol_type = other RNA
taaaaatctacaaatatgttgg

SEQ ID NO: 20 - RNA, 22 nt, synthetic construct
  source 1..22: mol_type = other RNA
tttggtttacatctgcacttgg

SEQ ID NO: 21 - RNA, 22 nt, synthetic construct
  source 1..22: mol_type = other RNA
tataatacgacttcacatcttc

SEQ ID NO: 22 - RNA, 22 nt, synthetic construct
  source 1..22: mol_type = other RNA
tagaaagttctttccatcgttt

SEQ ID NO: 23 - RNA, 22 nt, synthetic construct
  source 1..22: mol_type = other RNA
ttctatgtcaactaaactggca

SEQ ID NO: 24 - RNA, 22 nt, synthetic construct
  source 1..22: mol_type = other RNA
ttaaacagcatctcttggtcat

SEQ ID NO: 25 - RNA, 22 nt, synthetic construct
  source 1..22: mol_type = other RNA
tcgaatttccaagtacagcggc

SEQ ID NO: 26 - RNA, 22 nt, synthetic construct
  source 1..22: mol_type = other RNA
ttagaaagttctttccatcgtt

SEQ ID NO: 27 - RNA, 22 nt, synthetic construct
  source 1..22: mol_type = other RNA
tagatgtactgtataatacgac

SEQ ID NO: 28 - RNA, 22 nt, synthetic construct
  source 1..22: mol_type = other RNA
tctgtatactagaatctccctt

SEQ ID NO: 29 - RNA, 22 nt, synthetic construct
  source 1..22: mol_type = other RNA
ttttatgttaatatcatctcct

SEQ ID NO: 30 - RNA, 22 nt, synthetic construct
  source 1..22: mol_type = other RNA
tgagaatctcagttgatctggg

SEQ ID NO: 31 - RNA, 22 nt, synthetic construct
  source 1..22: mol_type = other RNA
tctgacaagagcttcacactga

SEQ ID NO: 32 - RNA, 22 nt, synthetic construct
  source 1..22: mol_type = other RNA
ttctattatagccatgtatgag

SEQ ID NO: 33 - RNA, 22 nt, synthetic construct
  source 1..22: mol_type = other RNA
tgatattttctaattgtagtag

SEQ ID NO: 34 - RNA, 22 nt, synthetic construct
  source 1..22: mol_type = other RNA
tttcttacaagggaccttggtt

SEQ ID NO: 35 - RNA, 22 nt, synthetic construct
  source 1..22: mol_type = other RNA
tataagcaaaagaaacttgggc

SEQ ID NO: 36 - RNA, 22 nt, synthetic construct
  source 1..22: mol_type = other RNA
tttctggttgtcacaggtggaa

SEQ ID NO: 37 - RNA, 22 nt, synthetic construct
  source 1..22: mol_type = other RNA
ttgttgattaacaagtttccta

SEQ ID NO: 38 - RNA, 22 nt, synthetic construct
  source 1..22: mol_type = other RNA
tagagtgagaatttgctccttt

SEQ ID NO: 39 - RNA, 22 nt, synthetic construct
  source 1..22: mol_type = other RNA
ttcaggaatcttctcctccgag

SEQ ID NO: 40 - RNA, 22 nt, synthetic construct
  source 1..22: mol_type = other RNA
tttaatacatgagtacagctga

SEQ ID NO: 41 - RNA, 22 nt, synthetic construct
  source 1..22: mol_type = other RNA
tttctaaactaggttgagggag

SEQ ID NO: 42 - RNA, 22 nt, synthetic construct
  source 1..22: mol_type = other RNA
ttgtcagtgactatcatgtcgt

SEQ ID NO: 43 - RNA, 22 nt, synthetic construct
  source 1..22: mol_type = other RNA
tattttaatacatgagtacagc

SEQ ID NO: 44 - RNA, 22 nt, synthetic construct
  source 1..22: mol_type = other RNA
taagcaaaagaaacttgggcta

SEQ ID NO: 45 - RNA, 22 nt, synthetic construct
  source 1..22: mol_type = other RNA
ttttaatacatgagtacagctg

SEQ ID NO: 46 - RNA, 22 nt, synthetic construct
  source 1..22: mol_type = other RNA
ttaaaaaagagattacttgcag

SEQ ID NO: 47 - RNA, 22 nt, synthetic construct
  source 1..22: mol_type = other RNA
tattcatatttatatacaggca

SEQ ID NO: 48 - RNA, 22 nt, synthetic construct
  source 1..22: mol_type = other RNA
ttttaaaaaagagattacttgc

SEQ ID NO: 49 - RNA, 22 nt, synthetic construct
  source 1..22: mol_type = other RNA
ttatgtaaaagacaaacaatgc

SEQ ID NO: 50 - RNA, 22 nt, synthetic construct
  source 1..22: mol_type = other RNA
ttaaatgaaaagtccaggagaa

SEQ ID NO: 51 - RNA, 22 nt, synthetic construct
  source 1..22: mol_type = other RNA
ttgattaacaagtttcctatgg

SEQ ID NO: 52 - RNA, 22 nt, synthetic construct
  source 1..22: mol_type = other RNA
tttaattcctagactggggtcc

SEQ ID NO: 53 - RNA, 22 nt, synthetic construct
  source 1..22: mol_type = other RNA
tcaaaatcatattatataccca

SEQ ID NO: 54 - RNA, 22 nt, synthetic construct
  source 1..22: mol_type = other RNA
tatatatagagctatagacata

SEQ ID NO: 55 - RNA, 22 nt, synthetic construct
  source 1..22: mol_type = other RNA
tgtaatgaaaaagtaaacacga

SEQ ID NO: 56 - RNA, 22 nt, synthetic construct
  source 1..22: mol_type = other RNA
ttctaaactaggttgagggaga

SEQ ID NO: 57 - RNA, 22 nt, synthetic construct
  source 1..22: mol_type = other RNA
tattctagaaactcaccactag

SEQ ID NO: 58 - RNA, 22 nt, synthetic construct
  source 1..22: mol_type = other RNA
ttgaaatcctcttcatcaggcc

SEQ ID NO: 59 - RNA, 22 nt, synthetic construct
  source 1..22: mol_type = other RNA
tgagaatactcttgaatcttga

SEQ ID NO: 60 - RNA, 22 nt, synthetic construct
  source 1..22: mol_type = other RNA
ttgaatatctcatgaatggacc

SEQ ID NO: 61 - RNA, 22 nt, synthetic construct
  source 1..22: mol_type = other RNA
tttaaaaaagagattacttgca

SEQ ID NO: 62 - RNA, 22 nt, synthetic construct
  source 1..22: mol_type = other RNA
ttcactttttgaaacgctgtgc

SEQ ID NO: 63 - RNA, 22 nt, synthetic construct
  source 1..22: mol_type = other RNA
taatcttttacttctcccactg

SEQ ID NO: 64 - RNA, 22 nt, synthetic construct
  source 1..22: mol_type = other RNA
tatatataaaacatagctattc

SEQ ID NO: 65 - RNA, 22 nt, synthetic construct
  source 1..22: mol_type = other RNA
tatatatataaaacatagctat

SEQ ID NO: 66 - RNA, 22 nt, synthetic construct
  source 1..22: mol_type = other RNA
ttgtggttgatgttgttggcac

SEQ ID NO: 67 - RNA, 22 nt, synthetic construct
  source 1..22: mol_type = other RNA
tagaataaaaataagtgcttga

SEQ ID NO: 68 - RNA, 22 nt, synthetic construct
  source 1..22: mol_type = other RNA
taatgtttacagtgagaagtga

SEQ ID NO: 69 - RNA, 22 nt, synthetic construct
  source 1..22: mol_type = other RNA
tagggaaagatcttgactgcca

SEQ ID NO: 70 - RNA, 22 nt, synthetic construct
  source 1..22: mol_type = other RNA
tagagtttctaaactaggttga

SEQ ID NO: 71 - RNA, 22 nt, synthetic construct
  source 1..22: mol_type = other RNA
ttttagaaaaggatgaaggctg

SEQ ID NO: 72 - RNA, 22 nt, synthetic construct
  source 1..22: mol_type = other RNA
tttagaaaaggatgaaggctgg

SEQ ID NO: 73 - RNA, 22 nt, synthetic construct
  source 1..22: mol_type = other RNA
atagaataaaaataagtgcttg

SEQ ID NO: 74 - RNA, 22 nt, synthetic construct
  source 1..22: mol_type = other RNA
tcttgaatcttgaatatctcat

SEQ ID NO: 75 - RNA, 22 nt, synthetic construct
  source 1..22: mol_type = other RNA
tttcattagtagatggtggggc

SEQ ID NO: 76 - RNA, 22 nt, synthetic construct
  source 1..22: mol_type = other RNA
ttagaaaaggatgaaggctggg

SEQ ID NO: 77 - RNA, 22 nt, synthetic construct
  source 1..22: mol_type = other RNA
atatatatataaaacatagcta

SEQ ID NO: 78 - RNA, 22 nt, synthetic construct
  source 1..22: mol_type = other RNA
ttatattcttctgagaagatga

SEQ ID NO: 79 - RNA, 22 nt, synthetic construct
  source 1..22: mol_type = other RNA
taaaaatcaatctcatttcctg

SEQ ID NO: 80 - RNA, 22 nt, synthetic construct
  source 1..22: mol_type = other RNA
taataaacattttatttatgta

SEQ ID NO: 81 - RNA, 22 nt, synthetic construct
  source 1..22: mol_type = other RNA
tgattaacaagtttcctatgga

SEQ ID NO: 82 - RNA, 22 nt, synthetic construct
  source 1..22: mol_type = other RNA
ttaaaaatctcaataaaatgag

SEQ ID NO: 83 - RNA, 638 nt, synthetic construct
  source 1..638: mol_type = other RNA
gtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacaga

SEQ ID NO: 84 - RNA, 995 nt, synthetic construct
  source 1..995: mol_type = other RNA
gtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacag

SEQ ID NO: 85 - protein, 27 aa, synthetic construct
TTTPAPRPPTPAPTIASQPLSLRPEAC

SEQ ID NO: 86 - protein, 22 aa, synthetic construct
FMYVAAAAFVLLFFVGCGVLLS

SEQ ID NO: 87 - protein, 43 aa, synthetic construct
RKRRRQHGQLWFPEGFKVSEASKKKRREPLGEDSVGLKPLKNA

SEQ ID NO: 88 - protein, 283 aa, synthetic construct
MVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAM

SEQ ID NO: 89 - protein, 191 aa, synthetic construct
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 90 - protein, 477 aa, synthetic construct
MVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 91 - protein, 21 aa, synthetic construct
MALPVTALLLPLALLLHAARP

SEQ ID NO: 92 - protein, 45 aa, synthetic construct
TTTPAPRPPTPAPTIASQPLSLRPEACRPAAGGAVHTRGLDFACD

SEQ ID NO: 93 - protein, 24 aa, synthetic construct
IYIWAPLAGTCGVLLLSLVITLYC

SEQ ID NO: 94 - protein, 39 aa, synthetic construct
IEVMYPPPYLDNEKSNGTIIHVKGKHLCPSPLFPGPSKP

SEQ ID NO: 95 - protein, 27 aa, synthetic construct
FWVLVVVGGVLACYSLLVTVAFIIFWV

SEQ ID NO: 96 - protein, 42 aa, synthetic construct
KRGRKKLLYIFKQPFMRPVQTTQEEDGCSCRFPEEEEGGCEL

SEQ ID NO: 97 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 240 aa, synthetic construct
QVQLVQSGAEVKKPGASVKVSCKASGYTFTSYYMHWVRQAPGQGLEWMGIINPSRGSASYAQKFQGRVTMARDTSTSTVYMELSSLRSEDTAVYYCARDRNYYYYMDVWGKGTTVTVSSGGGGSGGGGSGGGGSEIVMTQSPATLSVSPGERATLSCRASQNISSNLAWYQQKPGQAPRLLIYGASTRATDIPARFSGSGSGTEFTLTISSLQSEDFAVYYCQQYITWYTFGQGTKLEIK

SEQ ID NO: 99 - protein, 119 aa, synthetic construct
QVQLVQSGAEVKKPGASVKVSCKASGYTFTSYYMHWVRQAPGQGLEWMGIINPSRGSASYAQKFQGRVTMARDTSTSTVYMELSSLRSEDTAVYYCARDRNYYYYMDVWGKGTTVTVSS

SEQ ID NO: 100 - protein, 106 aa, synthetic construct
EIVMTQSPATLSVSPGERATLSCRASQNISSNLAWYQQKPGQAPRLLIYGASTRATDIPARFSGSGSGTEFTLTISSLQSEDFAVYYCQQYITWYTFGQGTKLEIK

SEQ ID NO: 101 - protein, 7 aa, synthetic construct
GYTFTSY

SEQ ID NO: 102 - protein, 6 aa, synthetic construct
NPSRGS

SEQ ID NO: 103 - protein, 10 aa, synthetic construct
DRNYYYYMDV

SEQ ID NO: 104 - protein, 11 aa, synthetic construct
RASQNISSNLA

SEQ ID NO: 105 - protein, 7 aa, synthetic construct
GASTRAT

SEQ ID NO: 106 - protein, 8 aa, synthetic construct
QQYITWYT

SEQ ID NO: 107 - DNA, 720 bp, synthetic construct
  source 1..720: mol_type = other DNA
caggtccagcttgtccagtccggagcggaggtgaaaaaaccgggcgcgtccgtgaaagtatcctgcaaggcctccggttatactttcacatcctactacatgcattgggtccggcaggcacctgggcagggcctggaatggatggggattattaatccctctcgcggttctgcatcatatgctcagaagtttcaagggagagtgacaatggccagagacactagcacctcaaccgtttacatggaactcagctcgctaaggagcgaggacacagccgtctactattgtgctcgtgatcgcaactactattactacatggatgtgtggggcaaagggactaccgttacagtttcatctggcggcggcggaagcggtggaggcggttctggcggaggaggaagtgaaattgtgatgacccagtctcccgctacactcagtgtgtcgcctggagagcgagctaccctgagttgcagggcctctcaaaacatctcaagtaatctggcctggtatcagcaaaagcccgggcaggcaccaaggctgctgatatacggagccagcactagagctaccgatataccagcacggttttccggcagcggttcagggaccgagtttacgttgacgatcagtagcttacaatccgaggacttcgccgtgtactattgtcaacagtatatcacctggtacactttcggccaagggacaaagttggaaatcaag

SEQ ID NO: 108 - protein, 484 aa, synthetic construct
MALPVTALLLPLALLLHAARPQVQLVQSGAEVKKPGASVKVSCKASGYTFTSYYMHWVRQAPGQGLEWMGIINPSRGSASYAQKFQGRVTMARDTSTSTVYMELSSLRSEDTAVYYCARDRNYYYYMDVWGKGTTVTVSSGGGGSGGGGSGGGGSEIVMTQSPATLSVSPGERATLSCRASQNISSNLAWYQQKPGQAPRLLIYGASTRATDIPARFSGSGSGTEFTLTISSLQSEDFAVYYCQQYITWYTFGQGTKLEIKAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - DNA, 1455 bp, synthetic construct
  source 1..1455: mol_type = other DNA
atggccctgccagtaacggctctgctgctgccacttgctctgctcctccatgcagccaggcctcaggtccagcttgtccagtccggagcggaggtgaaaaaaccgggcgcgtccgtgaaagtatcctgcaaggcctccggttatactttcacatcctactacatgcattgggtccggcaggcacctgggcagggcctggaatggatggggattattaatccctctcgcggttctgcatcatatgctcagaagtttcaagggagagtgacaatggccagagacactagcacctcaaccgtttacatggaactcagctcgctaaggagcgaggacacagccgtctactattgtgctcgtgatcgcaactactattactacatggatgtgtggggcaaagggactaccgttacagtttcatctggcggcggcggaagcggtggaggcggttctggcggaggaggaagtgaaattgtgatgacccagtctcccgctacactcagtgtgtcgcctggagagcgagctaccctgagttgcagggcctctcaaaacatctcaagtaatctggcctggtatcagcaaaagcccgggcaggcaccaaggctgctgatatacggagccagcactagagctaccgatataccagcacggttttccggcagcggttcagggaccgagtttacgttgacgatcagtagcttacaatccgaggacttcgccgtgtactattgtcaacagtatatcacctggtacactttcggccaagggacaaagttggaaatcaaggcggcagcaattgaagttatgtaccctccaccttacctcgacaatgagaaaagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggttaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgctaa

SEQ ID NO: 110 - protein, 127 aa, synthetic construct
EVQLVESGGGLVQPGGSLRLSCAASGFSFSDYSGMSWVRQAPGKGEELVSAISPGGGDTYYADSVKGRFTISRDNSKNTLYLQMNSLRAEDTAVYYCARRWWYYSNHSGDYDYFDYRGQGTLVTVSS

SEQ ID NO: 111 - protein, 8 aa, synthetic construct
GFSFSDYS

SEQ ID NO: 112 - protein, 6 aa, synthetic construct
SPGGGD

SEQ ID NO: 113 - protein, 17 aa, synthetic construct
RWWYYSNHSGDYDYFDY

SEQ ID NO: 114 - DNA, 381 bp, synthetic construct
  source 1..381: mol_type = other DNA
gaggtccagctcgtcgagagcggcggtgggctggttcagcctggaggaagtctccggctgagctgtgctgcaagtggattctcattttcagattactccgggatgtcctgggtgcgtcaggcgccagggaaaggcgaagaattagtgtctgccatttcgcccggtggtggggatacttattatgcagacagcgtaaagggccgattcacaatctcaagggacaattccaagaatacgctgtacttgcagatgaacagcctaagggctgaggataccgccgtgtactattgcgccagaagatggtggtattactctaaccattcaggcgactacgactattttgattaccgcggccaaggaacacttgttaccgtgtctagt

SEQ ID NO: 115 - protein, 374 aa, synthetic construct
MALPVTALLLPLALLLHAARPEVQLVESGGGLVQPGGSLRLSCAASGFSFSDYSGMSWVRQAPGKGEELVSAISPGGGDTYYADSVKGRFTISRDNSKNTLYLQMNSLRAEDTAVYYCARRWWYYSNHSGDYDYFDYRGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1122 bp, synthetic construct
  source 1..1122: mol_type = other DNA
atggccctgccagtaacggctctgctgctgccacttgctctgctcctccatgcagccaggcctgaggtccagctcgtcgagagcggcggtgggctggttcagcctggaggaagtctccggctgagctgtgctgcaagtggattctcattttcagattactccgggatgtcctgggtgcgtcaggcgccagggaaaggcgaagaattagtgtctgccatttcgcccggtggtggggatacttattatgcagacagcgtaaagggccgattcacaatctcaagggacaattccaagaatacgctgtacttgcagatgaacagcctaagggctgaggataccgccgtgtactattgcgccagaagatggtggtattactctaaccattcaggcgactacgactattttgattaccgcggccaaggaacacttgttaccgtgtctagtgcggcagcaaccacgacgccagcgccgcgaccaccaacaccggcgcctaccatcgcgtcgcagccactgtcactgcgcccagaagcgtgccggccagcggcgggtggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgc

SEQ ID NO: 117 - protein, 246 aa, synthetic construct
EIVLTQSPGTLSLSPGERATVSCRASQSVSSSYLAWYQQKPGQAPRLLIYGASSRATGIPDRFSGSGSGTDFTLTISRLEPEDFAVYYCQQYGSSPYTFGQGTKLEIKGGGGSGGGGSGGGGSQVQLVQSGPEVKKPGASVKVSCKASGYTFTSYGITWVRQAPGQGLEWMGWISEYNGNTNYAQKFQGRVTMTIDTSTTTAYMELRSLRSDDTAVYFCARGGILPYYFFYYMDVWGKGTTVTVSS

SEQ ID NO: 118 - protein, 123 aa, synthetic construct
QVQLVQSGPEVKKPGASVKVSCKASGYTFTSYGITWVRQAPGQGLEWMGWISEYNGNTNYAQKFQGRVTMTIDTSTTTAYMELRSLRSDDTAVYFCARGGILPYYFFYYMDVWGKGTTVTVSS

SEQ ID NO: 119 - protein, 108 aa, synthetic construct
EIVLTQSPGTLSLSPGERATVSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 120 - protein, 7 aa, synthetic construct
GYTFTSY

SEQ ID NO: 121 - protein, 6 aa, synthetic construct
SEYNGN

SEQ ID NO: 122 - protein, 14 aa, synthetic construct
GGILPYYFFYYMDV

SEQ ID NO: 123 - protein, 12 aa, synthetic construct
RASQSVSSSYLA

SEQ ID NO: 124 - protein, 7 aa, synthetic construct
GASSRAT

SEQ ID NO: 125 - protein, 9 aa, synthetic construct
QQYGSSPYT

SEQ ID NO: 126 - DNA, 738 bp, synthetic construct
  source 1..738: mol_type = other DNA
gagatcgtgctgacccagtctcccggcaccctgtccctgtctcccggcgaaagggctaccgtgagctgtagggcctcccagtccgtgagcagcagctacctggcctggtatcagcaaaagcccggccaggctcccaggctgctgatctacggagccagcagcagggctacaggaataccagaccggttcagcggcagcggcagcggcaccgacttcacactgacaataagccggctggaacccgaggacttcgccgtgtactattgtcaacagtacggcagcagcccttacaccttcggccagggaaccaagctcgagatcaagggcggcggaggatctggcggaggaggatctggaggaggaggcagccaagtgcaactggtgcagagcggccctgaagtgaagaagcccggcgccagcgtgaaagtgtcctgcaaagccagcggctacaccttcaccagctacggcatcacctgggtgcgacaggctcccggccagggactggagtggatgggctggatcagcgagtacaatggcaacaccaactacgcccagaagttccagggaagggtgacaatgaccatcgacaccagcaccaccaccgcctacatggagctgaggagcctcaggagcgacgacaccgccgtgtacttctgcgccagaggcggcatcctgccctactacttcttctactacatggacgtgtggggcaagggcaccaccgtgaccgtgtccagc

SEQ ID NO: 127 - protein, 837 aa, synthetic construct
MALPVTALLLPLALLLHAARPEIVLTQSPGTLSLSPGERATVSCRASQSVSSSYLAWYQQKPGQAPRLLIYGASSRATGIPDRFSGSGSGTDFTLTISRLEPEDFAVYYCQQYGSSPYTFGQGTKLEIKGGGGSGGGGSGGGGSQVQLVQSGPEVKKPGASVKVSCKASGYTFTSYGITWVRQAPGQGLEWMGWISEYNGNTNYAQKFQGRVTMTIDTSTTTAYMELRSLRSDDTAVYFCARGGILPYYFFYYMDVWGKGTTVTVSSATTTPAPRPPTPAPTIASQPLSLRPEACFMYVAAAAFVLLFFVGCGVLLSRKRRRQHGQLWFPEGFKVSEASKKKRREPLGEDSVGLKPLKNAMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 128 - DNA, 738 bp, synthetic construct; identical to SEQ ID NO: 126
  source 1..738: mol_type = other DNA

SEQ ID NO: 129 - protein, 247 aa, synthetic construct
EIVLTQSPATLSLSPGERATLSCGASQSVSSSYLAWYQQKPGLAPRLLIYDASSRATGIPDRFSGSGSGTDFTLTISRLEPEDFAVYYCQQYGSSPYTFGQGTKLEIKGGGGSGGGGSGGGGSQVQLVQSGAEVKKPGASVKVSCKASGYTFSSYGVSWVRQAPGQGLEWMGWISKYNGNTNYAQKFQGRVTMTTDTSTSTAYMELRSLRSDDTAVYFCARGGIHGDSYYFYYLDVWGKGTTVTVSS

SEQ ID NO: 130 - protein, 124 aa, synthetic construct
QVQLVQSGAEVKKPGASVKVSCKASGYTFSSYGVSWVRQAPGQGLEWMGWISKYNGNTNYAQKFQGRVTMTTDTSTSTAYMELRSLRSDDTAVYFCARGGIHGDSYYFYYLDVWGKGTTVTVSS

SEQ ID NO: 131 - protein, 108 aa, synthetic construct
EIVLTQSPATLSLSPGERATLSCGASQSVSSSYLAWYQQKPGLAPRLLIYDASSRATGIPDRFSGSGSGTDFTLTISRLEPEDFAVYYCQQYGSSPYTFGQGTKLEIK

SEQ ID NO: 132 - protein, 7 aa, synthetic construct
GYTFSSY

SEQ ID NO: 133 - protein, 6 aa, synthetic construct
SKYNGN

SEQ ID NO: 134 - protein, 15 aa, synthetic construct
GGIHGDSYYFYYLDV

SEQ ID NO: 135 - protein, 12 aa, synthetic construct
GASQSVSSSYLA

SEQ ID NO: 136 - protein, 7 aa, synthetic construct
DASSRAT

SEQ ID NO: 137 - protein, 9 aa, synthetic construct
QQYGSSPYT

SEQ ID NO: 138 - protein, 837 aa, synthetic construct
MALPVTALLLPLALLLHAARPEIVLTQSPATLSLSPGERATLSCGASQSVSSSYLAWYQQKPGLAPRLLIYDASSRATGIPDRFSGSGSGTDFTLTISRLEPEDFAVYYCQQYGSSPYTFGQGTKLEIKGGGGSGGGGSGGGGSQVQLVQSGAEVKKPGASVKVSCKASGYTFSSYGVSWVRQAPGQGLEWMGWISKYNGNTNYAQKFQGRVTMTTDTSTSTAYMELRSLRSDDTAVYFCARGGIHGDSYYFYYLDVWGKGTTVTVSSATTTPAPRPPTPAPTIASQPLSLRPEACFMYVAAAAFVLLFFVGCGVLLSRKRRRQHGQLWFPEGFKVSEASKKKRREPLGEDSVGLKPLKNAMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQIS

SEQ ID NO: 139 - DNA, 2512 bp, synthetic construct
  source 1..2512: mol_type = other DNA
atggccttaccagtgaccgccttgctcctgccgctggccttgctgctccacgccgccaggcctgagatcgtgctgacccagtctcccgccaccctgtccctgtctcccggcgaaagggctaccctgtcttgcggcgcctcccagtccgtgagcagcagctacctggcctggtatcagcaaaagcccggcctggctcccaggctgctgatctacgacgccagcagcagggctacaggaataccagaccggttcagcggcagcggcagcggcaccgacttcacactgacaataagccggctggaacccgaggacttcgccgtgtactattgtcaacagtacggcagcagcccttacaccttcggccagggaaccaagctcgagatcaagggcggcggaggatctggcggaggaggatctggaggaggaggcagccaagtgcaactggtgcagagcggcgccgaagtgaagaagcccggcgccagcgtgaaagtgtcctgcaaagccagcggatacacattcagctcctacggcgtgagctgggtgcgacaggctcccggacaaggcctggagtggatgggctggatcagcaagtataacggcaacaccaactacgcccagaagttccagggaagggtgacaatgaccaccgacaccagcaccagcaccgcctacatggagctgaggagcctcaggagcgacgacaccgcagtgtacttctgcgcacgcggcggcatccacggcgacagctactacttctattacctggacgtgtggggcaagggcaccaccgtgaccgtgtccagcgcaaccacaacacccgctcccagacctcccacaccagcaccaacaatagcaagtcaacccttgagcttgcgccctgaggcctgcttcatgtacgtggccgccgccgccttcgtgttgctgttcttcgtgggctgtggcgtgctgttgtccagaaagcgccgccgccagcacggacagctctggttccctgagggcttcaaagtgtcagaggccagcaagaagaagcggcgcgaacctctgggcgaggacagcgtgggcctgaagcctctgaaaaacgctatggtgtcaaagctcagccagctccagacagagctgctggccgccctgctggaaagcggcctgagcaaggaggccctgttgcaggctctgggagagccaggcccttacctgctggctggcgagggacctttggataagggcgagagctgcggaggcggacgcggagagctggccgaactgcctaatggcttgggcgagacacgcggcagcgaggacgagacagacgacgacggcgaggatttcacacctcccatcctcaaggagttggagaatctgtctcccgaggaggccgcccaccagaaggccgtggtggagacactgctgcaagaggacccttggcgcgtggccaagatggtgaagagctacctgcaacagcacaacatccctcagcgcgaggtggtggataccaccggcctgaaccagagccacctgtcccagcacctgaacaagggcacacccatgaagacccagaagcgcgccgccctctacacctggtacgtgcgcaagcagcgcgaggttgctcaacagttcacccacgccggccagggcggcttgattgaggaacccacaggcgacgagctgcccaccaagaagggccgccgcaaccgcttcaaatggggacccgcctcgcagcagatactgttccaagcctacgagcgccagaagaatcccagcaaggaggagcgcgagacactcgtggaggagtgcaaccgcgctgagtgcatccagaggggcgtgagccctagccaagcccagggcttgggcagcaacctggtgacagaagtgcgcgtgtataactggttcgccaaccgccgcaaggaggaggccttccgccacaagctggccatgacttgccgcgacgagttccctacaatggtctttcccagcggccagatcagccaggcctctgccctggcaccagcaccaccacaagtgttacctcaagctcccgctcccgctcccgctcctgctatggtgtcggcattggctcaagctcctgctcccgtgccggtgttggcacctggaccgcctcaagctgttgctcctcccgctcccaagcccacacaagccggcgaaggcaccctgtctgaggccctgctgcaactgcaattcgacgacgaggatctgggcgccctgctgggcaacagcaccgatcccgctgtctttaccgatctggccagcgtggataatagcgagttccagcagctcctgaaccagggtatccctgtggcgccgcacacaacggagcccatgctgatggagtaccctgaggccatcaccagactcgtgactggcgcacaacgccctcctgatccagcaccagcgccgttaggcgcacctggcctgcctaacggcttgctgagcggcgacgaggatttctccagcatcgccgatatggatttcagcgcactgctgagtcaaatctcca

SEQ ID NO: 140 - DNA, 741 bp, synthetic construct
  source 1..741: mol_type = other DNA
gagatcgtgctgacccagtctcccgccaccctgtccctgtctcccggcgaaagggctaccctgtcttgcggcgcctcccagtccgtgagcagcagctacctggcctggtatcagcaaaagcccggcctggctcccaggctgctgatctacgacgccagcagcagggctacaggaataccagaccggttcagcggcagcggcagcggcaccgacttcacactgacaataagccggctggaacccgaggacttcgccgtgtactattgtcaacagtacggcagcagcccttacaccttcggccagggaaccaagctcgagatcaagggcggcggaggatctggcggaggaggatctggaggaggaggcagccaagtgcaactggtgcagagcggcgccgaagtgaagaagcccggcgccagcgtgaaagtgtcctgcaaagccagcggatacacattcagctcctacggcgtgagctgggtgcgacaggctcccggacaaggcctggagtggatgggctggatcagcaagtataacggcaacaccaactacgcccagaagttccagggaagggtgacaatgaccaccgacaccagcaccagcaccgcctacatggagctgaggagcctcaggagcgacgacaccgcagtgtacttctgcgcacgcggcggcatccacggcgacagctactacttctattacctggacgtgtggggcaagggcaccaccgtgaccgtgtccagc

SEQ ID NO: 141 - protein, 341 aa, synthetic construct
MALPVTALLLPLALLLHAARPELPTQGTFSNVSTNVSELCGGAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 142 - protein, 277 aa, synthetic construct [sequence omitted]

SEQ ID NO: 143 - RNA, 8819 nt, synthetic construct
  source 1..8819: mol_type = other RNA
gagccatgcttggcttacgagggcgaccaacccatcaaactccccgcccccagcacttttatttctcctctttaggaagtacacttcagtatctttggcacagtgcatgagcacgactaaagtaaaacatcgcagaaaacatagctttagtctacccttcgtgtcctaaaaggaaaaccagtagcttcccaggccaccggaagggcaacacatgtcctctgcagtttctgcacacgggaaggtaaagacagagagaggacctactcctcaacacagaaacatttcaaaatctttcctcgcctgcaacccaagctgaagtcattctccccagaaataacaaaagttggaagagaagccggagacaggataggtgcaggaagcccacactttgagggcagcactcagacaccctctcctgtgtgcaggacgtgccgaatgttcaggtgcaatgagaatgagccatgcttggcttataaggtacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggataagcttgatatcgaattcatcgatgttaataattaacatatatgttaatcattaacatatagttaattattaaccgctatgttaatgattaacaacggttaataattaacatatatgttaatcattaacatataactagtctagagggtatataatgggggccactagtctactaccagagttcatcgctagcgctaccggatccgccaccatggccctgccagtaacggctctgctgctgccacttgctctgctcctccatgcagccaggcctcaggtccagcttgtccagtccggagcggaggtgaaaaaaccgggcgcgtccgtgaaagtatcctgcaaggcctccggttatactttcacatcctactacatgcattgggtccggcaggcacctgggcagggcctggaatggatggggattattaatccctctcgcggttctgcatcatatgctcagaagtttcaagggagagtgacaatggccagagacactagcacctcaaccgtttacatggaactcagctcgctaaggagcgaggacacagccgtctactattgtgctcgtgatcgcaactactattactacatggatgtgtggggcaaagggactaccgttacagtttcatctggcggcggcggaagcggtggaggcggttctggcggaggaggaagtgaaattgtgatgacccagtctcccgctacactcagtgtgtcgcctggagagcgagctaccctgagttgcagggcctctcaaaacatctcaagtaatctggcctggtatcagcaaaagcccgggcaggcaccaaggctgctgatatacggagccagcactagagctaccgatataccagcacggttttccggcagcggttcagggaccgagtttacgttgacgatcagtagcttacaatccgaggacttcgccgtgtactattgtcaacagtatatcacctggtacactttcggccaagggacaaagttggaaatcaaggcggcagcaattgaagttatgtaccctccaccttacctcgacaatgagaaaagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggttaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgctaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggatccttgacttgcggccaacttgtttattgcagcttataatggttacaaataaagcaatagcatcacaaatttcacaaataaagcatttttttcactgcattctagttgtggtttgtccaaactcatcaatgtatcttatcatgtctgggatccttgacttgcggccgcaactcccacctgcaacatgcgtgactgactgaggccgcgactctagagtcgaccggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccatggccttaccagtgaccgccttgctcctgccgctggccttgctgctccacgccgccaggcctgagatcgtgctgacccagtctcccggcaccctgtccctgtctcccggcgaaagggctaccgtgagctgtagggcctcccagtccgtgagcagcagctacctggcctggtatcagcaaaagcccggccaggctcccaggctgctgatctacggagccagcagcagggctacaggaataccagaccggttcagcggcagcggcagcggcaccgacttcacactgacaataagccggctggaacccgaggacttcgccgtgtactattgtcaacagtacggcagcagcccttacaccttcggccagggaaccaagctcgagatcaagggcggcggaggatctggcggaggaggatctggaggaggaggcagccaagtgcaactggtgcagagcggccctgaagtgaagaagcccggcgccagcgtgaaagtgtcctgcaaagccagcggctacaccttcaccagctacggcatcacctgggtgcgacaggctcccggccagggactggagtggatgggctggatcagcgagtacaatggcaacaccaactacgcccagaagttccagggaagggtgacaatgaccatcgacaccagcaccaccaccgcctacatggagctgaggagcctcaggagcgacgacaccgccgtgtacttctgcgccagaggcggcatcctgccctactacttcttctactacatggacgtgtggggcaagggcaccaccgtgaccgtgtccagcgcaaccacaacacccgctcccagacctcccacaccagcaccaacaatagcaagtcaacccttgagcttgcgccctgaggcctgcttcatgtacgtggccgccgccgccttcgtgttgctgttcttcgtgggctgtggcgtgctgttgtccagaaagcgccgccgccagcacggacagctctggttccctgagggcttcaaagtgtcagaggccagcaagaagaagcggcgcgaacctctgggcgaggacagcgtgggcctgaagcctctgaaaaacgctatggtgtcaaagctcagccagctccagacagagctgctggccgccctgctggaaagcggcctgagcaaggaggccctgttgcaggctctgggagagccaggcccttacctgctggctggcgagggacctttggataagggcgagagctgcggaggcggacgcggagagctggccgaactgcctaatggcttgggcgagacacgcggcagcgaggacgagacagacgacgacggcgaggatttcacacctcccatcctcaaggagttggagaatctgtctcccgaggaggccgcccaccagaaggccgtggtggagacactgctgcaagaggacccttggcgcgtggccaagatggtgaagagctacctgcaacagcacaacatccctcagcgcgaggtggtggataccaccggcctgaaccagagccacctgtcccagcacctgaacaagggcacacccatgaagacccagaagcgcgccgccctctacacctggtacgtgcgcaagcagcgcgaggttgctcaacagttcacccacgccggccagggcggcttgattgaggaacccacaggcgacgagctgcccaccaagaagggccgccgcaaccgcttcaaatggggacccgcctcgcagcagatactgttccaagcctacgagcgccagaagaatcccagcaaggaggagcgcgagacactcgtggaggagtgcaaccgcgctgagtgcatccagaggggcgtgagccctagccaagcccagggcttgggcagcaacctggtgacagaagtgcgcgtgtataactggttcgccaaccgccgcaaggaggaggccttccgccacaagctggccatgacttgccgcgacgagttccctacaatggtctttcccagcggccagatcagccaggcctctgccctggcaccagcaccaccacaagtgttacctcaagctcccgctcccgctcccgctcctgctatggtgtcggcattggctcaagctcctgctcccgtgccggtgttggcacctggaccgcctcaagctgttgctcctcccgctcccaagcccacacaagccggcgaaggcaccctgtctgaggccctgctgcaactgcaattcgacgacgaggatctgggcgccctgctgggcaacagcaccgatcccgctgtctttaccgatctggccagcgtggataatagcgagttccagcagctcctgaaccagggtatccctgtggcgccgcacacaacggagcccatgctgatggagtaccctgaggccatcaccagactcgtgactggcgcacaacgccctcctgatccagcaccagcgccgttaggcgcacctggcctgcctaacggcttgctgagcggcgacgaggatttctccagcatcgccgatatggatttcagcgcactgctgagtcaaatctccagccgggccaagcggtccggatccggagagggcaggggatctctccttacttgtggggacgtcgaggaaaaccctggaccaatggccctgcccgtgacagccctgctcctgcctctggccctgttgctgcatgcagcacggcctgagctgcccacacaaggcacattcagcaacgtgtccaccaacgtgtccgagctgtgcggcggcgctatcgtggtgcccgtgtgcttggccttcctgctgaccaccctgctgggcgtgctgttctgcttcaacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcagtaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttccgagggcaatctggcccatcaagtggccttcgcctctgggagtaacaaaaatgcacttcaaaatagcttctgtaatcaagctgcatgggtggagtactccccagctgactccaggaagttctctatccaaagctattcattaggccagagctgtgcaaataattagtcacccacttgctccataaccctccatgacagcccaggcattgagtccaggtgggaccatcaagccatgctctggtggctcatgcattatcatagaaatgggaggctttatttattttactaaaaagaacaaaaacaacagactgctgtcctttagacaataggatcacgtcatctgagccctctgtgccccaggtgacaagcccagccccaagttctctttcctcagcctccccacacatgttctggaggagatgggcccagcaggctgctctgaggcctggcccctcgtaagccaagcatggctc

SEQ ID NO: 144 - RNA, 8492 nt, synthetic construct
  source 1..8492: mol_type = other RNA
gagccatgcttggcttacgagggcgaccaacccatcaaactccccgcccccagcacttttatttctcctctttaggaagtacacttcagtatctttggcacagtgcatgagcacgactaaagtaaaacatcgcagaaaacatagctttagtctacccttcgtgtcctaaaaggaaaaccagtagcttcccaggccaccggaagggcaacacatgtcctctgcagtttctgcacacgggaaggtaaagacagagagaggacctactcctcaacacagaaacatttcaaaatctttcctcgcctgcaacccaagctgaagtcattctccccagaaataacaaaagttggaagagaagccggagacaggataggtgcaggaagcccacactttgagggcagcactcagacaccctctcctgtgtgcaggacgtgccgaatgttcaggtgcaatgagaatgagccatgcttggcttataaggtacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggataagcttgatatcgaattcatcgatgttaataattaacatatatgttaatcattaacatatagttaattattaaccgctatgttaatgattaacaacggttaataattaacatatatgttaatcattaacatataactagtctagagggtatataatgggggccactagtctactaccagagttcatcgctagcgctaccggatccgccaccatggccctgccagtaacggctctgctgctgccacttgctctgctcctccatgcagccaggcctgaggtccagctcgtcgagagcggcggtgggctggttcagcctggaggaagtctccggctgagctgtgctgcaagtggattctcattttcagattactccgggatgtcctgggtgcgtcaggcgccagggaaaggcgaagaattagtgtctgccatttcgcccggtggtggggatacttattatgcagacagcgtaaagggccgattcacaatctcaagggacaattccaagaatacgctgtacttgcagatgaacagcctaagggctgaggataccgccgtgtactattgcgccagaagatggtggtattactctaaccattcaggcgactacgactattttgattaccgcggccaaggaacacttgttaccgtgtctagtgcggcagcaaccacgacgccagcgccgcgaccaccaacaccggcgcctaccatcgcgtcgcagccactgtcactgcgcccagaagcgtgccggccagcggcgggtggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgctaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggatccttgacttgcggccaacttgtttattgcagcttataatggttacaaataaagcaatagcatcacaaatttcacaaataaagcatttttttcactgcattctagttgtggtttgtccaaactcatcaatgtatcttatcatgtctgggatccttgacttgcggccgcaactcccacctgcaacatgcgtgactgactgaggccgcgactctagagtcgaccggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccatggccttaccagtgaccgccttgctcctgccgctggccttgctgctccacgccgccaggcctgagatcgtgctgacccagtctcccgccaccctgtccctgtctcccggcgaaagggctaccctgtcttgcggcgcctcccagtccgtgagcagcagctacctggcctggtatcagcaaaagcccggcctggctcccaggctgctgatctacgacgccagcagcagggctacaggaataccagaccggttcagcggcagcggcagcggcaccgacttcacactgacaataagccggctggaacccgaggacttcgccgtgtactattgtcaacagtacggcagcagcccttacaccttcggccagggaaccaagctcgagatcaagggcggcggaggatctggcggaggaggatctggaggaggaggcagccaagtgcaactggtgcagagcggcgccgaagtgaagaagcccggcgccagcgtgaaagtgtcctgcaaagccagcggatacacattcagctcctacggcgtgagctgggtgcgacaggctcccggacaaggcctggagtggatgggctggatcagcaagtataacggcaacaccaactacgcccagaagttccagggaagggtgacaatgaccaccgacaccagcaccagcaccgcctacatggagctgaggagcctcaggagcgacgacaccgcagtgtacttctgcgcacgcggcggcatccacggcgacagctactacttctattacctggacgtgtggggcaagggcaccaccgtgaccgtgtccagcgcaaccacaacacccgctcccagacctcccacaccagcaccaacaatagcaagtcaacccttgagcttgcgccctgaggcctgcttcatgtacgtggccgccgccgccttcgtgttgctgttcttcgtgggctgtggcgtgctgttgtccagaaagcgccgccgccagcacggacagctctggttccctgagggcttcaaagtgtcagaggccagcaagaagaagcggcgcgaacctctgggcgaggacagcgtgggcctgaagcctctgaaaaacgctatggtgtcaaagctcagccagctccagacagagctgctggccgccctgctggaaagcggcctgagcaaggaggccctgttgcaggctctgggagagccaggcccttacctgctggctggcgagggacctttggataagggcgagagctgcggaggcggacgcggagagctggccgaactgcctaatggcttgggcgagacacgcggcagcgaggacgagacagacgacgacggcgaggatttcacacctcccatcctcaaggagttggagaatctgtctcccgaggaggccgcccaccagaaggccgtggtggagacactgctgcaagaggacccttggcgcgtggccaagatggtgaagagctacctgcaacagcacaacatccctcagcgcgaggtggtggataccaccggcctgaaccagagccacctgtcccagcacctgaacaagggcacacccatgaagacccagaagcgcgccgccctctacacctggtacgtgcgcaagcagcgcgaggttgctcaacagttcacccacgccggccagggcggcttgattgaggaacccacaggcgacgagctgcccaccaagaagggccgccgcaaccgcttcaaatggggacccgcctcgcagcagatactgttccaagcctacgagcgccagaagaatcccagcaaggaggagcgcgagacactcgtggaggagtgcaaccgcgctgagtgcatccagaggggcgtgagccctagccaagcccagggcttgggcagcaacctggtgacagaagtgcgcgtgtataactggttcgccaaccgccgcaaggaggaggccttccgccacaagctggccatgacttgccgcgacgagttccctacaatggtctttcccagcggccagatcagccaggcctctgccctggcaccagcaccaccacaagtgttacctcaagctcccgctcccgctcccgctcctgctatggtgtcggcattggctcaagctcctgctcccgtgccggtgttggcacctggaccgcctcaagctgttgctcctcccgctcccaagcccacacaagccggcgaaggcaccctgtctgaggccctgctgcaactgcaattcgacgacgaggatctgggcgccctgctgggcaacagcaccgatcccgctgtctttaccgatctggccagcgtggataatagcgagttccagcagctcctgaaccagggtatccctgtggcgccgcacacaacggagcccatgctgatggagtaccctgaggccatcaccagactcgtgactggcgcacaacgccctcctgatccagcaccagcgccgttaggcgcacctggcctgcctaacggcttgctgagcggcgacgaggatttctccagcatcgccgatatggatttcagcgcactgctgagtcaaatctccagccgggccaagcggtccggatccggagagggcaggggatctctccttacttgtggggacgtcgaggaaaaccctggaccaatggccctgcccgtgacagccctgctcctgcctctggccctgttgctgcatgcagcacggcctgagctgcccacacaaggcacattcagcaacgtgtccaccaacgtgtccgagctgtgcggcggcgctatcgtggtgcccgtgtgcttggccttcctgctgaccaccctgctgggcgtgctgttctgcttcaacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcagtaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttccgagggcaatctggcccatcaagtggccttcgcctctgggagtaacaaaaatgcacttcaaaatagcttctgtaatcaagctgcatgggtggagtactccccagctgactccaggaagttctctatccaaagctattcattaggccagagctgtgcaaataattagtcacccacttgctccataaccctccatgacagcccaggcattgagtccaggtgggaccatcaagccatgctctggtggctcatgcattatcatagaaatgggaggctttatttattttactaaaaagaacaaaaacaacagactgctgtcctttagacaataggatcacgtcatctgagccctctgtgccccaggtgacaagcccagccccaagttctctttcctcagcctccccacacatgttctggaggagatgggcccagcaggctgctctgaggcctggcccctcgtaagccaagcatggctc

SEQ ID NO: 145 - RNA, 8822 nt, synthetic construct
  source 1..8822: mol_type = other RNA
gagccatgcttggcttacgagggcgaccaacccatcaaactccccgcccccagcacttttatttctcctctttaggaagtacacttcagtatctttggcacagtgcatgagcacgactaaagtaaaacatcgcagaaaacatagctttagtctacccttcgtgtcctaaaaggaaaaccagtagcttcccaggccaccggaagggcaacacatgtcctctgcagtttctgcacacgggaaggtaaagacagagagaggacctactcctcaacacagaaacatttcaaaatctttcctcgcctgcaacccaagctgaagtcattctccccagaaataacaaaagttggaagagaagccggagacaggataggtgcaggaagcccacactttgagggcagcactcagacaccctctcctgtgtgcaggacgtgccgaatgttcaggtgcaatgagaatgagccatgcttggcttataaggtacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggataagcttgatatcgaattcatcgatgttaataattaacatatatgttaatcattaacatatagttaattattaaccgctatgttaatgattaacaacggttaataattaacatatatgttaatcattaacatataactagtctagagggtatataatgggggccactagtctactaccagagttcatcgctagcgctaccggatccgccaccatggccctgccagtaacggctctgctgctgccacttgctctgctcctccatgcagccaggcctcaggtccagcttgtccagtccggagcggaggtgaaaaaaccgggcgcgtccgtgaaagtatcctgcaaggcctccggttatactttcacatcctactacatgcattgggtccggcaggcacctgggcagggcctggaatggatggggattattaatccctctcgcggttctgcatcatatgctcagaagtttcaagggagagtgacaatggccagagacactagcacctcaaccgtttacatggaactcagctcgctaaggagcgaggacacagccgtctactattgtgctcgtgatcgcaactactattactacatggatgtgtggggcaaagggactaccgttacagtttcatctggcggcggcggaagcggtggaggcggttctggcggaggaggaagtgaaattgtgatgacccagtctcccgctacactcagtgtgtcgcctggagagcgagctaccctgagttgcagggcctctcaaaacatctcaagtaatctggcctggtatcagcaaaagcccgggcaggcaccaaggctgctgatatacggagccagcactagagctaccgatataccagcacggttttccggcagcggttcagggaccgagtttacgttgacgatcagtagcttacaatccgaggacttcgccgtgtactattgtcaacagtatatcacctggtacactttcggccaagggacaaagttggaaatcaaggcggcagcaattgaagttatgtaccctccaccttacctcgacaatgagaaaagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggttaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgctaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggatccttgacttgcggccaacttgtttattgcagcttataatggttacaaataaagcaatagcatcacaaatttcacaaataaagcatttttttcactgcattctagttgtggtttgtccaaactcatcaatgtatcttatcatgtctgggatccttgacttgcggccgcaactcccacctgcaacatgcgtgactgactgaggccgcgactctagagtcgaccggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccatggccttaccagtgaccgccttgctcctgccgctggccttgctgctccacgccgccaggcctgagatcgtgctgacccagtctcccgccaccctgtccctgtctcccggcgaaagggctaccctgtcttgcggcgcctcccagtccgtgagcagcagctacctggcctggtatcagcaaaagcccggcctggctcccaggctgctgatctacgacgccagcagcagggctacaggaataccagaccggttcagcggcagcggcagcggcaccgacttcacactgacaataagccggctggaacccgaggacttcgccgtgtactattgtcaacagtacggcagcagcccttacaccttcggccagggaaccaagctcgagatcaagggcggcggaggatctggcggaggaggatctggaggaggaggcagccaagtgcaactggtgcagagcggcgccgaagtgaagaagcccggcgccagcgtgaaagtgtcctgcaaagccagcggatacacattcagctcctacggcgtgagctgggtgcgacaggctcccggacaaggcctggagtggatgggctggatcagcaagtataacggcaacaccaactacgcccagaagttccagggaagggtgacaatgaccaccgacaccagcaccagcaccgcctacatggagctgaggagcctcaggagcgacgacaccgcagtgtacttctgcgcacgcggcggcatccacggcgacagctactacttctattacctggacgtgtggggcaagggcaccaccgtgaccgtgtccagcgcaaccacaacacccgctcccagacctcccacaccagcaccaacaatagcaagtcaacccttgagcttgcgccctgaggcctgcttcatgtacgtggccgccgccgccttcgtgttgctgttcttcgtgggctgtggcgtgctgttgtccagaaagcgccgccgccagcacggacagctctggttccctgagggcttcaaagtgtcagaggccagcaagaagaagcggcgcgaacctctgggcgaggacagcgtgggcctgaagcctctgaaaaacgctatggtgtcaaagctcagccagctccagacagagctgctggccgccctgctggaaagcggcctgagcaaggaggccctgttgcaggctctgggagagccaggcccttacctgctggctggcgagggacctttggataagggcgagagctgcggaggcggacgcggagagctggccgaactgcctaatggcttgggcgagacacgcggcagcgaggacgagacagacgacgacggcgaggatttcacacctcccatcctcaaggagttggagaatctgtctcccgaggaggccgcccaccagaaggccgtggtggagacactgctgcaagaggacccttggcgcgtggccaagatggtgaagagctacctgcaacagcacaacatccctcagcgcgaggtggtggataccaccggcctgaaccagagccacctgtcccagcacctgaacaagggcacacccatgaagacccagaagcgcgccgccctctacacctggtacgtgcgcaagcagcgcgaggttgctcaacagttcacccacgccggccagggcggcttgattgaggaacccacaggcgacgagctgcccaccaagaagggccgccgcaaccgcttcaaatggggacccgcctcgcagcagatactgttccaagcctacgagcgccagaagaatcccagcaaggaggagcgcgagacactcgtggaggagtgcaaccgcgctgagtgcatccagaggggcgtgagccctagccaagcccagggcttgggcagcaacctggtgacagaagtgcgcgtgtataactggttcgccaaccgccgcaaggaggaggccttccgccacaagctggccatgacttgccgcgacgagttccctacaatggtctttcccagcggccagatcagccaggcctctgccctggcaccagcaccaccacaagtgttacctcaagctcccgctcccgctcccgctcctgctatggtgtcggcattggctcaagctcctgctcccgtgccggtgttggcacctggaccgcctcaagctgttgctcctcccgctcccaagcccacacaagccggcgaaggcaccctgtctgaggccctgctgcaactgcaattcgacgacgaggatctgggcgccctgctgggcaacagcaccgatcccgctgtctttaccgatctggccagcgtggataatagcgagttccagcagctcctgaaccagggtatccctgtggcgccgcacacaacggagcccatgctgatggagtaccctgaggccatcaccagactcgtgactggcgcacaacgccctcctgatccagcaccagcgccgttaggcgcacctggcctgcctaacggcttgctgagcggcgacgaggatttctccagcatcgccgatatggatttcagcgcactgctgagtcaaatctccagccgggccaagcggtccggatccggagagggcaggggatctctccttacttgtggggacgtcgaggaaaaccctggaccaatggccctgcccgtgacagccctgctcctgcctctggccctgttgctgcatgcagcacggcctgagctgcccacacaaggcacattcagcaacgtgtccaccaacgtgtccgagctgtgcggcggcgctatcgtggtgcccgtgtgcttggccttcctgctgaccaccctgctgggcgtgctgttctgcttcaacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcagtaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttccgagggcaatctggcccatcaagtggccttcgcctctgggagtaacaaaaatgcacttcaaaatagcttctgtaatcaagctgcatgggtggagtactccccagctgactccaggaagttctctatccaaagctattcattaggccagagctgtgcaaataattagtcacccacttgctccataaccctccatgacagcccaggcattgagtccaggtgggaccatcaagccatgctctggtggctcatgcattatcatagaaatgggaggctttatttattttactaaaaagaacaaaaacaacagactgctgtcctttagacaataggatcacgtcatctgagccctctgtgccccaggtgacaagcccagccccaagttctctttcctcagcctccccacacatgttctggaggagatgggcccagcaggctgctctgaggcctggcccctcgtaagccaagcatggctc

SEQ ID NO: 146 - RNA, 8490 nt, synthetic construct
  source 1..8490: mol_type = other RNA
gagccatgcttggcttacgagggcgaccaacccatcaaactccccgcccccagcacttttatttctcctctttaggaagtacacttcagtatctttggcacagtgcatgagcacgactaaagtaaaacatcgcagaaaacatagctttagtctacccttcgtgtcctaaaaggaaaaccagtagcttcccaggccaccggaagggcaacacatgtcctctgcagtttctgcacacgggaaggtaaagacagagagaggacctactcctcaacacagaaacatttcaaaatctttcctcgcctgcaacccaagctgaagtcattctccccagaaataacaaaagttggaagagaagccggagacaggataggtgcaggaagcccacactttgagggcagcactcagacaccctctcctgtgtgcaggacgtgccgaatgttcaggtgcaatgagaatgagccatgcttggcttataaggtacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggataagcttgatatcgaattcatcgatgttaataattaacatatatgttaatcattaacatatagttaattattaaccgctatgttaatgattaacaacggttaataattaacatatatgttaatcattaacatataactagtctagagggtatataatgggggccactagtctactaccagagttcatcgctagcgctaccggatccgccaccatggccctgccagtaacggctctgctgctgccacttgctctgctcctccatgcagccaggcctgaggtccagctcgtcgagagcggcggtgggctggttcagcctggaggaagtctccggctgagctgtgctgcaagtggattctcattttcagattactccgggatgtcctgggtgcgtcaggcgccagggaaaggcgaagaattagtgtctgccatttcgcccggtggtggggatacttattatgcagacagcgtaaagggccgattcacaatctcaagggacaattccaagaatacgctgtacttgcagatgaacagcctaagggctgaggataccgccgtgtactattgcgccagaagatggtggtattactctaaccattcaggcgactacgactattttgattaccgcggccaaggaacacttgttaccgtgtctagtgcggcagcaaccacgacgccagcgccgcgaccaccaacaccggcgcctaccatcgcgtcgcagccactgtcactgcgcccagaagcgtgccggccagcggcgggtggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgctaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggatccttgacttgcggccaacttgtttattgcagcttataatggttacaaataaagcaatagcatcacaaatttcacaaataaagcatttttttcactgcattctagttgtggtttgtccaaactcatcaatgtatcttatcatgtctgggatccttgacttgcggccgcaactcccacctgcaacatgcgtgactgactgaggccgcgactctagagtcgaccggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccatggccttaccagtgaccgccttgctcctgccgctggccttgctgctccacgccgccaggcctgagatcgtgctgacccagtctcccggcaccctgtccctgtctcccggcgaaagggctaccgtgagctgtagggcctcccagtccgtgagcagcagctacctggcctggtatcagcaaaagcccggccaggctcccaggctgctgatctacggagccagcagcagggctacaggaataccagaccggttcagcggcagcggcagcggcaccgacttcacactgacaataagccggctggaacccgaggacttcgccgtgtactattgtcaacagtacggcagcagcccttacaccttcggccagggaaccaagctcgagatcaagggcggcggaggatctggcggaggaggatctggaggaggaggcagccaagtgcaactggtgcagagcggccctgaagtgaagaagcccggcgccagcgtgaaagtgtcctgcaaagccagcggctacaccttcaccagctacggcatcacctgggtgcgacaggctcccggccagggactggagtggatgggctggatcagcgagtacaatggcaacaccaactacgcccagaagttccagggaagggtgacaatgaccatcgacaccagcaccaccaccgcctacatggagctgaggagcctcaggagcgacgacaccgccgtgtacttctgcgccagaggcggcatcctgccctactacttcttctactacatggacgtgtggggcaagggcaccaccgtgaccgtgtccagcgcaaccacaacacccgctcccagacctcccacaccagcaccaacaatagcaagtcaacccttgagcttgcgccctgaggcctgcttcatgtacgtggccgccgccgccttcgtgttgctgttcttcgtgggctgtggcgtgctgttgtccagaaagcgccgccgccagcacggacagctctggttccctgagggcttcaaagtgtcagaggccagcaagaagaagcggcgcgaacctctgggcgaggacagcgtgggcctgaagcctctgaaaaacgctatggtgtcaaagctcagccagctccagacagagctgctggccgccctgctggaaagcggcctgagcaaggaggccctgttgcaggctctgggagagccaggcccttacctgctggctggcgagggacctttggataagggcgagagctgcggaggcggacgcggagagctggccgaactgcctaatggcttgggcgagacacgcggcagcgaggacgagacagacgacgacggcgaggatttcacacctcccatcctcaaggagttggagaatctgtctcccgaggaggccgcccaccagaaggccgtggtggagacactgctgcaagaggacccttggcgcgtggccaagatggtgaagagctacctgcaacagcacaacatccctcagcgcgaggtggtggataccaccggcctgaaccagagccacctgtcccagcacctgaacaagggcacacccatgaagacccagaagcgcgccgccctctacacctggtacgtgcgcaagcagcgcgaggttgctcaacagttcacccacgccggccagggcggcttgattgaggaacccacaggcgacgagctgcccaccaagaagggccgccgcaaccgcttcaaatggggacccgcctcgcagcagatactgttccaagcctacgagcgccagaagaatcccagcaaggaggagcgcgagacactcgtggaggagtgcaaccgcgctgagtgcatccagaggggcgtgagccctagccaagcccagggcttgggcagcaacctggtgacagaagtgcgcgtgtataactggttcgccaaccgccgcaaggaggaggccttccgccacaagctggccatgacttgccgcgacgagttccctacaatggtctttcccagcggccagatcagccaggcctctgccctggcaccagcaccaccacaagtgttacctcaagctcccgctcccgctcccgctcctgctatggtgtcggcattggctcaagctcctgctcccgtgccggtgttggcacctggaccgcctcaagctgttgctcctcccgctcccaagcccacacaagccggcgaaggcaccctgtctgaggccctgctgcaactgcaattcgacgacgaggatctgggcgccctgctgggcaacagcaccgatcccgctgtctttaccgatctggccagcgtggataatagcgagttccagcagctcctgaaccagggtatccctgtggcgccgcacacaacggagcccatgctgatggagtaccctgaggccatcaccagactcgtgactggcgcacaacgccctcctgatccagcaccagcgccgttaggcgcacctggcctgcctaacggcttgctgagcggcgacgaggatttctccagcatcgccgatatggatttcagcgcactgctgagtcaaatctccagccgggccaagcggtccggatccggagagggcaggggatctctccttacttgtggggacgtcgaggaaaaccctggaccaatggccctgcccgtgacagccctgctcctgcctctggccctgttgctgcatgcagcacggcctgagctgcccacacaaggcacattcagcaacgtgtccaccaacgtgtccgagctgtgcggcggcgctatcgtggtgcccgtgtgcttggccttcctgctgaccaccctgctgggcgtgctgttctgcttcaacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcagtaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttccgagggcaatctggcccatcaagtggccttcgcctctgggagtaacaaaaatgcacttcaaaatagcttctgtaatcaagctgcatgggtggagtactccccagctgactccaggaagttctctatccaaagctattcattaggccagagctgtgcaaataattagtcacccacttgctccataaccctccatgacagcccaggcattgagtccaggtgggaccatcaagccatgctctggtggctcatgcattatcatagaaatgggaggctttatttattttactaaaaagaacaaaaacaacagactgctgtcctttagacaataggatcacgtcatctgagccctctgtgccccaggtgacaagcccagccccaagttctctttcctcagcctccccacacatgttctggaggagatgggcccagcaggctgctctgaggcctggcccctcgtaagccaagcatggctca

SEQ ID NO: 147 - RNA, 8659 nt, synthetic construct
  source 1..8659: mol_type = other RNA
gagccatgcttggcttacgagggcgaccaacccatcaaactccccgcccccagcacttttatttctcctctttaggaagtacacttcagtatctttggcacagtgcatgagcacgactaaagtaaaacatcgcagaaaacatagctttagtctacccttcgtgtcctaaaaggaaaaccagtagcttcccaggccaccggaagggcaacacatgtcctctgcagtttctgcacacgggaaggtaaagacagagagaggacctactcctcaacacagaaacatttcaaaatctttcctcgcctgcaacccaagctgaagtcattctccccagaaataacaaaagttggaagagaagccggagacaggataggtgcaggaagcccacactttgagggcagcactcagacaccctctcctgtgtgcaggacgtgccgaatgttcaggtgcaatgagaatgagccatgcttggcttataaggtacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggataagcttgatatcgaattcatcgatgttaataattaacatatatgttaatcattaacatatagttaattattaaccgctatgttaatgattaacaacggttaataattaacatatatgttaatcattaacatataactagtctagagggtatataatgggggccactagtctactaccagagttcatcgctagcgctaccggatccgccaccatggccctgccagtaacggctctgctgctgccacttgctctgctcctccatgcagccaggcctgaggtccagctcgtcgagagcggcggtgggctggttcagcctggaggaagtctccggctgagctgtgctgcaagtggattctcattttcagattactccgggatgtcctgggtgcgtcaggcgccagggaaaggcgaagaattagtgtctgccatttcgcccggtggtggggatacttattatgcagacagcgtaaagggccgattcacaatctcaagggacaattccaagaatacgctgtacttgcagatgaacagcctaagggctgaggataccgccgtgtactattgcgccagaagatggtggtattactctaaccattcaggcgactacgactattttgattaccgcggccaaggaacacttgttaccgtgtctagtgcggcagcaaccacgacgccagcgccgcgaccaccaacaccggcgcctaccatcgcgtcgcagccactgtcactgcgcccagaagcgtgccggccagcggcgggtggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgctaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggatccttgacttgcggccaacttgtttattgcagcttataatggttacaaataaagcaatagcatcacaaatttcacaaataaagcatttttttcactgcattctagttgtggtttgtccaaactcatcaatgtatcttatcatgtctgggatccttgacttgcggccgcaactcccacctgcaacatgcgtgactgactgaggccgcgactctagagtcgaccggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccatggccttaccagtgaccgccttgctcctgccgctggccttgctgctccacgccgccaggcctgagatcgtgctgacccagtctcccgccaccctgtccctgtctcccggcgaaagggctaccctgtcttgcggcgcctcccagtccgtgagcagcagctacctggcctggtatcagcaaaagcccggcctggctcccaggctgctgatctacgacgccagcagcagggctacaggaataccagaccggttcagcggcagcggcagcggcaccgacttcacactgacaataagccggctggaacccgaggacttcgccgtgtactattgtcaacagtacggcagcagcccttacaccttcggccagggaaccaagctcgagatcaagggcggcggaggatctggcggaggaggatctggaggaggaggcagccaagtgcaactggtgcagagcggcgccgaagtgaagaagcccggcgccagcgtgaaagtgtcctgcaaagccagcggatacacattcagctcctacggcgtgagctgggtgcgacaggctcccggacaaggcctggagtggatgggctggatcagcaagtataacggcaacaccaactacgcccagaagttccagggaagggtgacaatgaccaccgacaccagcaccagcaccgcctacatggagctgaggagcctcaggagcgacgacaccgcagtgtacttctgcgcacgcggcggcatccacggcgacagctactacttctattacctggacgtgtggggcaagggcaccaccgtgaccgtgtccagcgcaaccacaacacccgctcccagacctcccacaccagcaccaacaatagcaagtcaacccttgagcttgcgccctgaggcctgcttcatgtacgtggccgccgccgccttcgtgttgctgttcttcgtgggctgtggcgtgctgttgtccagaaagcgccgccgccagcacggacagctctggttccctgagggcttcaaagtgtcagaggccagcaagaagaagcggcgcgaacctctgggcgaggacagcgtgggcctgaagcctctgaaaaacgctatggtgtcaaagctcagccagctccagacagagctgctggccgccctgctggaaagcggcctgagcaaggaggccctgttgcaggctctgggagagccaggcccttacctgctggctggcgagggacctttggataagggcgagagctgcggaggcggacgcggagagctggccgaactgcctaatggcttgggcgagacacgcggcagcgaggacgagacagacgacgacggcgaggatttcacacctcccatcctcaaggagttggagaatctgtctcccgaggaggccgcccaccagaaggccgtggtggagacactgctgcaagaggacccttggcgcgtggccaagatggtgaagagctacctgcaacagcacaacatccctcagcgcgaggtggtggataccaccggcctgaaccagagccacctgtcccagcacctgaacaagggcacacccatgaagacccagaagcgcgccgccctctacacctggtacgtgcgcaagcagcgcgaggttgctcaacagttcacccacgccggccagggcggcttgattgaggaacccacaggcgacgagctgcccaccaagaagggccgccgcaaccgcttcaaatggggacccgcctcgcagcagatactgttccaagcctacgagcgccagaagaatcccagcaaggaggagcgcgagacactcgtggaggagtgcaaccgcgctgagtgcatccagaggggcgtgagccctagccaagcccagggcttgggcagcaacctggtgacagaagtgcgcgtgtataactggttcgccaaccgccgcaaggaggaggccttccgccacaagctggccatgacttgccgcgacgagttccctacaatggtctttcccagcggccagatcagccaggcctctgccctggcaccagcaccaccacaagtgttacctcaagctcccgctcccgctcccgctcctgctatggtgtcggcattggctcaagctcctgctcccgtgccggtgttggcacctggaccgcctcaagctgttgctcctcccgctcccaagcccacacaagccggcgaaggcaccctgtctgaggccctgctgcaactgcaattcgacgacgaggatctgggcgccctgctgggcaacagcaccgatcccgctgtctttaccgatctggccagcgtggataatagcgagttccagcagctcctgaaccagggtatccctgtggcgccgcacacaacggagcccatgctgatggagtaccctgaggccatcaccagactcgtgactggcgcacaacgccctcctgatccagcaccagcgccgttaggcgcacctggcctgcctaacggcttgctgagcggcgacgaggatttctccagcatcgccgatatggatttcagcgcactgctgagtcaaatctccagccgggccaagcggtccggatccggagagggcaggggatctctccttacttgtggggacgtcgaggaaaaccctggaccaatgggagccggcgccactggccgcgctatggacggaccaagacttctcctgcttctgctgttgggtgtctctctaggtggggcgaaagaagcctgtcccactggtctatatactcattctggggagtgctgtaaggcctgtaaccttggcgagggagtggcacagccctgtggggcgaatcagaccgtgtgcgagccttgtctggacagtgtgaccttctcggacgtagtgtctgccactgaaccttgtaaaccgtgcacagagtgcgtagggctccaatcaatgtccgctccctgcgtggaagcagacgacgccgtgtgccgatgcgcatacggctattaccaggatgaaaccacaggccgctgcgaggcctgtagggtttgcgaggccggaagcggattagtgttttcatgccaagacaagcagaatacggtgtgcgaggagtgccctgacggaacttactctgatgaggccaaccacgtcgatccctgtttgccgtgtacggtttgtgaggataccgaacgacagctcagagagtgcacccggtgggctgatgcagaatgcgaggagatcccagggcgttggatcaccaggtcaacaccacccgaagggtccgacagtaccgctcctagtacccaggaaccagaagctccaccggagcaggatctgattgcgagtacagtagccggcgtggtcacaacagttatgggaagcagccaacctgtcgtgacacgcggaactacagataatctcatacccgtgtattgttcaatcctggccgccgtagtggtcggtctggtggcttacattgcgttcaagcggtggaactcctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttccgagggcaatctggcccatcaagtggccttcgcctctgggagtaacaaaaatgcacttcaaaatagcttctgtaatcaagctgcatgggtggagtactccccagctgactccaggaagttctctatccaaagctattcattaggccagagctgtgcaaataattagtcacccacttgctccataaccctccatgacagcccaggcattgagtccaggtgggaccatcaagccatgctctggtggctcatgcattatcatagaaatgggaggctttatttattttactaaaaagaacaaaaacaacagactgctgtcctttagacaataggatcacgtcatctgagccctctgtgccccaggtgacaagcccagccccaagttctctttcctcagcctccccacacatgttctggaggagatgggcccagcaggctgctctgaggcctggcccctcgtaagccaagcatggctca

SEQ ID NO: 148 - DNA, 6492 bp, synthetic construct
  source 1..6492: mol_type = other DNA
ggcggctagggaggtggggcgaggcgaggtttgctggggtgaggcagcggcgcggccgggccgggccgggccacaggcggtggcggcgggaccatggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaaccgcactgtcattcaccatcgagtgccaaatgaagaggacccttcattagatcgcccttttatttcagagggtactacgttgaaagacttaatttatgatatgacaacgtcaggttctggctcaggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatgtaattctacagctttgcctgaactctccttttttcttcagatctgctcctgggttttaatttgggaggtcaattgttctacctcactgagagggaacagaaggatattgcttccttttgcagcagtgtaataaagtcaattaaaaacttcccaggatttctttggacccaggaaacagccatgtgggtcctttctgtgcactatgaacgcttctttcccaggacagaaaatgtgtagtctacctttattttttattaacaaaacttgttttttaaaaagatgattgctggtcttaactttaggtaactctgctgtgctggagatcatctttaagggcaaaggagttggattgctgaattacaatgaaacatgtcttattactaaagaaagtgatttactcctggttagtacattctcagaggattctgaaccactagagtttccttgattcagactttgaatgtactgttctatagtttttcaggatcttaaaactaacacttataaaactcttatcttgagtctaaaaatgacctcatatagtagtgaggaacataattcatgcaattgtattttgtatactattattgttctttcacttattcagaacattacatgccttcaaaatgggattgtactataccagtaagtgccacttctgtgtctttctaatggaaatgagtagaattgctgaaagtctctatgttaaaacctatagtgtttgaattcaaaaagcttatttatctgggtaacccaaactttttctgttttgtttttggaagggtttttgtggtatgtcatttggtattctattctgaaaatgcctttctcctaccaaaatgtgcttaagccactaaagaaatgaagtggcattaattagtaaattattagcatggtcatgtttgaatattctcacatcaagcttttgcattttaattgtgttgtctaagtatacttttaaaaaatcaagtggcactctagatgcttatagtactttaatatttgtagcatacagactaatttttctaaaagggaaagtctgtctagctgcttgtgaaaagttatgtggtattctgtaagccatttttttctttatctgttcaaagacttattttttaagacatgaattacatttaaaattagaatatggttaatattaaataataggcctttttctaggaaggcgaaggtagttaataatttgaatagataacagatgtgcaagaaagtcacatttgttatgtatgtaggagtaaacgttcggtggatcctctgtctttgtaactgaggttagagctagtgtggttttgaggtctcactacactttgaggaaggcagcttttaattcagtgtttccttatgtgtgcgtacattgcaactgcttacatgtaatttatgtaatgcattcagtgcacccttgttacttgggagaggtggtagctaaagaacattctgagtataggtttttctccatttacagatgtctttggtcaaatattgaaagcaaacttgtcatggtcttcttacattaagttgaaactagcttataataactggtttttacttccaatgctatgaagtctctgcagggcttttacagttttcgaagtccttttatcactgtgatcttattctgaggggagaaaaaactatcatagctctgaggcaagacttcgactttatagtgctatcagttccccgatacagggtcagagtaacccatacagtattttggtcaggaagagaaagtggccatttacactgaatgagttgcattctgataatgtcttatctcttatacgtagaataaatttgaaagactatttgatcttaaaaccaaagtaattttagaatgagtgacatattacataggaatttagtgtcaatttcatgtgtttaaaaacatcatgggaaaaatgcttagaggttactattttgactacaaagttgagtttttttctgtagttaccataatttcattgaagcaaatgaatgagtttgagaggtttgtttttatagttgtgttgtattacttgtttaataataatctctaattctgtgatcaggtactttttttgtgggggttttttttttgtttttttttttttgttgttgtttttgggccatttctaagcctaccagatctgctttatgaaatccaggggaccaatgcattttatcactaaaactatttttatataattttaagaatataccaaaagttgtctgatttaaagttgtaatacatgatttctcactttcatgtaaggttatccacttttgctgaagatattttttattgaatcaaagattgagttacaattatacttttcttacctaagtggataaaatgtacttttgatgaatcagggaatttttttaaagttggagtttagttctaaattgactttacgtattactgcagttaattccttttttggctagggatggtttgataaaccacaattggctgatattgaaaatgaaagaaacttaaaaggtgggatggatcatgattactgtcgataactgcagataaatttgattagagtaataattttgtcatttaaaaacacagttgtttatactgcccatcctaggatgctcaccttccaagattcaacgtggctaaaacatcttctggtaaattgtgcgtccatattcattttgtcagtagccaggagaaatggggatgggggaaatacgacttagtgaggcatagacatccctggtccatcctttctgtctccagctgtttcttggaacctgctctcctgcttgctggtccctgacgcagagaccgttgcctcccccacagccgtttgactgaaggctgctctggagacctagagtaaaacggctgatggaagttgtgggacccacttccatttccttcagtcattagaggtggaagggaggggtctccaagtttggagattgagcagatgaggcttgggatgcccctgctttgacttcagccatggatgaggagtgggatggcagcaaggtggctcctgtggcagtggagttgtgccagaaacagtggccagttgtatcgcctataagacagggtaaggtctgaagagctgagcctgtaattctgctgtaataatgatagtgctcaagaagtgccttgagttggtgtacagtgccatggccatcaagaatcccagatttcaggttttattacaaaatgtaagtggtcacttggcgattttgtagtacatgcatgagttaccttttttctctatgtctgagaactgtcagattaaaacaagatggcaaagagatcgttagagtgcacaacaaaatcactatcccattagacacatcatcaaaagcttatttttattcttgcactggaagaatcgtaagtcaactgtttcttgaccatggcagtgttctggctccaaatggtagtgattccaaataatggttctgttaacactttggcagaaaatgccagctcagatattttgagatactaaggattatctttggacatgtactgcagcttcttgtctctgttttggattactggaatacccatgggccctctcaagagtgctggacttctaggacattaagatgattgtcagtacattaaacttttcaatcccattatgcaatcttgtttgtaaatgtaaacttctaaaaatatggttaataacattcaacctgtttattacaacttaaaaggaacttcagtgaatttgtttttattttttaacaagatttgtgaactgaatatcatgaaccatgttttgatacccctttttcacgttgtgccaacggaatagggtgtttgatatttcttcatatgttaaggagatgcttcaaaatgtcaattgctttaaacttaaattacctctcaagagaccaaggtacatttacctcattgtgtatataatgtttaatatttgtcagagcattctccaggtttgcagttttatttctataaagtatgggtattatgttgctcagttactcaaatggtactgtattgtttatatttgtaccccaaataacatcgtctgtactttctgttttctgtattgtatttgtgcaggattctttaggctttatcagtgtaatctctgccttttaagatatgtacagaaaatgtccatataaatttccattgaagtcgaatgatactgagaagcctgtaaagaggagaaaaaaacataagctgtgtttccccataagtttttttaaattgtatattgtatttgtagtaatattccaaaagaatgtaaataggaaatagaagagtgatgcttatgttaagtcctaacactacagtagaagaatggaagcagtgcaaataaattacatttttcccaagtgccagtggcatattttaaaataaagtgtatacgttggaatgagtcatgccatatgtagttgctgtagatggcaactagaacctttgagttacaagagtctttagaagttttctaaccctgcctagtgcaagttacaatattatagcgtgttcggggagtgccctcctgtctgcaggtgtgtctctgtgcctgggggcttttctccacatgcttaggggtgtgggtcttccattggggcatgatggacctgtctacaggtgatctctgttgcctttgggtcagcacatttgttagtctcctgggggtgaaaacttggcttacaagagaactggaaaaatgatgagatgtggtccccaaacccttgattgactctggggaggggctttgtgaataggattgctctcacattaaagatagttacttcaatttgaaggctggatttagggatttttttttttccttataacaaagacatcaccaggatatgaagcttttgttgaaagttggaaaaaaagtgaaattaaagacattcccagacaaa

SEQ ID NO: 149 - DNA, 22 bp, synthetic construct
  source 1..22: mol_type = other DNA
ttaaatgtaattcatgtcttaa

SEQ ID NO: 150 - DNA, 22 bp, synthetic construct
  source 1..22: mol_type = other DNA
ttcaatgaaattatggtaacta

SEQ ID NO: 151 - DNA, 22 bp, synthetic construct
  source 1..22: mol_type = other DNA
tataattgtaactcaatctttg

SEQ ID NO: 152 - DNA, 22 bp, synthetic construct
  source 1..22: mol_type = other DNA
tttgatttcaaatctctatgag

SEQ ID NO: 153 - DNA, 22 bp, synthetic construct
  source 1..22: mol_type = other DNA
tttaaatgtaattcatgtctta

SEQ ID NO: 154 - DNA, 22 bp, synthetic construct
  source 1..22: mol_type = other DNA
ttaagttgtaataaacaggttg

SEQ ID NO: 155 - DNA, 22 bp, synthetic construct
  source 1..22: mol_type = other DNA
tttaattgactttattacactg

SEQ ID NO: 156 - DNA, 22 bp, synthetic construct
  source 1..22: mol_type = other DNA
ttagtatctcaaaatatctgag

SEQ ID NO: 157 - DNA, 22 bp, synthetic construct
  source 1..22: mol_type = other DNA
ttaattgactttattacactgc

SEQ ID NO: 158 - DNA, 22 bp, synthetic construct
  source 1..22: mol_type = other DNA
ttaaatgacaaaattattactc

SEQ ID NO: 159 - DNA, 22 bp, synthetic construct
  source 1..22: mol_type = other DNA
ttagtaataagacatgtttcat

SEQ ID NO: 160 - DNA, 22 bp, synthetic construct
  source 1..22: mol_type = other DNA
tattcaaattattaactacctt

SEQ ID NO: 161 - DNA, 22 bp, synthetic construct
  source 1..22: mol_type = other DNA
taagtaatcaaaaagggatcca

SEQ ID NO: 162 - DNA, 22 bp, synthetic construct
  source 1..22: mol_type = other DNA
ttgtgtataactttgtctgtgg

SEQ ID NO: 163 - DNA, 22 bp, synthetic construct
  source 1..22: mol_type = other DNA
ttttaaatgtaattcatgtctt

SEQ ID NO: 164 - DNA, 22 bp, synthetic construct
  source 1..22: mol_type = other DNA
ttaacataagcatcactcttct

SEQ ID NO: 165 - DNA, 22 bp, synthetic construct
  source 1..22: mol_type = other DNA
ttaaagtactataagcatctag

SEQ ID NO: 166 - DNA, 22 bp, synthetic construct
  source 1..22: mol_type = other DNA
tatctattcaaattattaacta

SEQ ID NO: 167 - DNA, 22 bp, synthetic construct
  source 1..22: mol_type = other DNA
tcttgtaacacaatagttctcg

SEQ ID NO: 168 - DNA, 22 bp, synthetic construct
  source 1..22: mol_type = other DNA
ttaaacaagtaatacaacacaa

SEQ ID NO: 169 - DNA, 22 bp, synthetic construct
  source 1..22: mol_type = other DNA
taataaaacctgaaatctggga

SEQ ID NO: 170 - DNA, 22 bp, synthetic construct
  source 1..22: mol_type = other DNA
tacattttgtaataaaacctga

SEQ ID NO: 171 - DNA, 22 bp, synthetic construct
  source 1..22: mol_type = other DNA
tagaattacattttgatgcctt

SEQ ID NO: 172 - DNA, 22 bp, synthetic construct
  source 1..22: mol_type = other DNA
tttaagatcaaatagtctttca

SEQ ID NO: 173 - DNA, 22 bp, synthetic construct
  source 1..22: mol_type = other DNA
tacaatttaaaaaaacttatgg

SEQ ID NO: 174 - DNA, 22 bp, synthetic construct
  source 1..22: mol_type = other DNA
taaaattgtcttttgtacagag

SEQ ID NO: 175 - DNA, 22 bp, synthetic construct
  source 1..22: mol_type = other DNA
taagttgtaataaacaggttga

SEQ ID NO: 176 - DNA, 22 bp, synthetic construct
  source 1..22: mol_type = other DNA
taatgtactgacaatcatctta

SEQ ID NO: 177 - DNA, 22 bp, synthetic construct
  source 1..22: mol_type = other DNA
tttcattttcaatatcagccaa

SEQ ID NO: 178 - DNA, 22 bp, synthetic construct
  source 1..22: mol_type = other DNA
ttttaagatcaaatagtctttc

SEQ ID NO: 179 - DNA, 374 bp, synthetic construct
  source 1..374: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctag

SEQ ID NO: 180 - DNA, 479 bp, synthetic construct
  source 1..479: mol_type = other DNA
cgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 181 - DNA, 613 bp, synthetic construct
  source 1..613: mol_type = other DNA
gtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttttcatctgaccagtagtggactagtgtgacgctgctgacccctttctttcccttctacag

SEQ ID NO: 182 - DNA, 354 bp, synthetic construct
  source 1..354: mol_type = other DNA
ccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgac

SEQ ID NO: 183 - DNA, 906 bp, synthetic construct
  source 1..906: mol_type = other DNA
ccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgac

SEQ ID NO: 184 - RNA, 1541 nt, synthetic construct
  source 1..1541: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 185 - RNA, 1055 nt, synthetic construct
  source 1..1055: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacc

SEQ ID NO: 186 - RNA, 486 nt, synthetic construct
  source 1..486: mol_type = other RNA
taaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 187 - RNA, 1578 nt, synthetic construct
  source 1..1578: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacacttcaaggggcttgcggccgcaaccatctccatggcgacggtgacttaggagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgacgactgtgacagcagagtatgccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 188 - RNA, 1092 nt, synthetic construct
  source 1..1092: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacacttcaaggggcttgcggccgcaaccatctccatggcgacggtgacttaggagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgacgactgtgacagcagagtatgccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacc

SEQ ID NO: 189 - RNA, 486 nt, synthetic construct; identical to SEQ ID NO: 186
  source 1..486: mol_type = other RNA

SEQ ID NO: 190 - RNA, 1732 nt, synthetic construct
  source 1..1732: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggcgacggtgacttaggagtatgtgtttgaatgaggcttcagtactttacagaatcgttgcctgcacatcttggaaacacttgctgggattacttcgacttcttaacccaacagaaggctcgagaaggtatattgctgttgacagtgagcgcaggaaatgagattgatttttttagtgaagccacagatgtataaaaatcaatctcatttcctgtgcctactgcctcggacttcaaggggctagaattcgagcaattatcttgtttactaaaactgaataccttgctatctctttgatacatttttacaaagctgaattaaaatggtataaattaaatcactttgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 191 - RNA, 1246 nt, synthetic construct
  source 1..1246: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggcgacggtgacttaggagtatgtgtttgaatgaggcttcagtactttacagaatcgttgcctgcacatcttggaaacacttgctgggattacttcgacttcttaacccaacagaaggctcgagaaggtatattgctgttgacagtgagcgcaggaaatgagattgatttttttagtgaagccacagatgtataaaaatcaatctcatttcctgtgcctactgcctcggacttcaaggggctagaattcgagcaattatcttgtttactaaaactgaataccttgctatctctttgatacatttttacaaagctgaattaaaatggtataaattaaatcactttgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacc

SEQ ID NO: 192 - RNA, 486 nt, synthetic construct; identical to SEQ ID NO: 186
  source 1..486: mol_type = other RNA

SEQ ID NO: 193 - RNA, 731 nt, synthetic construct
  source 1..731: mol_type = other RNA
cggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggcgacggtgacttaggagtatgtgtttgaatgaggcttcagtactttacagaatcgttgcctgcacatcttggaaacacttgctgggattacttcgacttcttaacccaacagaaggctcgagaaggtatattgctgttgacagtgagcgcaggaaatgagattgatttttttagtgaagccacagatgtataaaaatcaatctcatttcctgtgcctactgcctcggacttcaaggggctagaattcgagcaattatcttgtttactaaaactgaataccttgctatctctttgatacatttttacaaagctgaattaaaatggtataaattaaatcactttgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgac

SEQ ID NO: 194 - DNA, 508 bp, synthetic construct
  source 1..508: mol_type = other DNA
tgtttgaatgaggcttcagtactttacagaatcgttgcctgcacatcttggaaacacttgctgggattacttcgacttcttaacccaacagaaggctcgagaaggtatattgctgttgacagtgagcgcaggaaatgagattgatttttttagtgaagccacagatgtataaaaatcaatctcatttcctgtgcctactgcctcggacttcaaggggctagaattcgagcaattatcttgtttactaaaactgaataccttgctatctctttgatacatttttacaaagctgaattaaaatggtataaattaaatcactttgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgac

SEQ ID NO: 195 - RNA, 578 nt, synthetic construct
  source 1..578: mol_type = other RNA
ccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacacttcaaggggcttgcggccgcaaccatctccatggcgacggtgacttaggagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgacgactgtgacagcagagtatgccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgac

SEQ ID NO: 196 - RNA, 1899 nt, synthetic construct
  source 1..1899: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 197 - RNA, 1413 nt, synthetic construct
  source 1..1413: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacc

SEQ ID NO: 198 - RNA, 486 nt, synthetic construct; identical to SEQ ID NO: 186
  source 1..486: mol_type = other RNA

SEQ ID NO: 199 - protein, 10 aa, synthetic construct
GYTFTSYGIT

SEQ ID NO: 200 - protein, 5 aa, synthetic construct
SYGIT

SEQ ID NO: 201 - protein, 6 aa, synthetic construct
TSYGIT

SEQ ID NO: 202 - protein, 8 aa, synthetic construct
GYTFTSYG

SEQ ID NO: 203 - protein, 10 aa, synthetic construct
WISEYNGNTN

SEQ ID NO: 204 - protein, 17 aa, synthetic construct
WISEYNGNTNYAQKFQG

SEQ ID NO: 205 - protein, 13 aa, synthetic construct
WMGWISEYNGNTN

SEQ ID NO: 206 - protein, 8 aa, synthetic construct
ISEYNGNT

SEQ ID NO: 207 - protein, 15 aa, synthetic construct
ARGGILPYYFFYYMD

SEQ ID NO: 208 - protein, 16 aa, synthetic construct
ARGGILPYYFFYYMDV

SEQ ID NO: 209 - protein, 8 aa, synthetic construct
SSSYLAWY

SEQ ID NO: 210 - protein, 7 aa, synthetic construct
QSVSSSY

SEQ ID NO: 211 - protein, 10 aa, synthetic construct
LLIYGASSRA

SEQ ID NO: 212 - protein, 8 aa, synthetic construct
QQYGSSPY

SEQ ID NO: 213 - protein, 5 aa, synthetic construct
SYGVS

SEQ ID NO: 214 - protein, 6 aa, synthetic construct
SSYGVS

SEQ ID NO: 215 - protein, 8 aa, synthetic construct
GYTFSSYG

SEQ ID NO: 216 - protein, 10 aa, synthetic construct
WISKYNGNTN

SEQ ID NO: 217 - protein, 17 aa, synthetic construct
WISKYNGNTNYAQKFQG

SEQ ID NO: 218 - protein, 13 aa, synthetic construct
WMGWISKYNGNTN

SEQ ID NO: 219 - protein, 8 aa, synthetic construct
ISKYNGNT

SEQ ID NO: 220 - protein, 16 aa, synthetic construct
ARGGIHGDSYYFYYLD

SEQ ID NO: 221 - protein, 17 aa, synthetic construct
ARGGIHGDSYYFYYLDV

SEQ ID NO: 222 - protein, 8 aa, synthetic construct
SSSYLAWY

SEQ ID NO: 223 - protein, 7 aa, synthetic construct
QSVSSSY

SEQ ID NO: 224 - protein, 10 aa, synthetic construct
LLIYDASSRA

SEQ ID NO: 225 - protein, 8 aa, synthetic construct
QQYGSSPY

SEQ ID NO: 226 - protein, 10 aa, synthetic construct
GYTFTSYYMH

SEQ ID NO: 227 - protein, 5 aa, synthetic construct
SYYMH

SEQ ID NO: 228 - protein, 6 aa, synthetic construct
TSYYMH

SEQ ID NO: 229 - protein, 8 aa, synthetic construct
GYTFTSYY

SEQ ID NO: 230 - protein, 10 aa, synthetic construct
IINPSRGSAS

SEQ ID NO: 231 - protein, 17 aa, synthetic construct
IINPSRGSASYAQKFQG

SEQ ID NO: 232 - protein, 13 aa, synthetic construct
WMGIINPSRGSAS

SEQ ID NO: 233 - protein, 8 aa, synthetic construct
INPSRGSA

SEQ ID NO: 234 - protein, 11 aa, synthetic construct
ARDRNYYYYMD

SEQ ID NO: 235 - protein, 12 aa, synthetic construct
ARDRNYYYYMDV

SEQ ID NO: 236 - protein, 7 aa, synthetic construct
SSNLAWY

SEQ ID NO: 237 - protein, 6 aa, synthetic construct
QNISSN

SEQ ID NO: 238 - protein, 10 aa, synthetic construct
LLIYGASTRA

SEQ ID NO: 239 - protein, 7 aa, synthetic construct
QQYITWY

SEQ ID NO: 240 - protein, 11 aa, synthetic construct
GFSFSDYSGMS

SEQ ID NO: 241 - protein, 6 aa, synthetic construct
DYSGMS

SEQ ID NO: 242 - protein, 7 aa, synthetic construct
SDYSGMS

SEQ ID NO: 243 - protein, 9 aa, synthetic construct
GFSFSDYSG

SEQ ID NO: 244 - protein, 10 aa, synthetic construct
AISPGGGDTY

SEQ ID NO: 245 - protein, 17 aa, synthetic construct
AISPGGGDTYYADSVKG

SEQ ID NO: 246 - protein, 13 aa, synthetic construct
LVSAISPGGGDTY

SEQ ID NO: 247 - protein, 8 aa, synthetic construct
ISPGGGDT

SEQ ID NO: 248 - protein, 18 aa, synthetic construct
ARRWWYYSNHSGDYDYFD

SEQ ID NO: 249 - protein, 19 aa, synthetic construct
ARRWWYYSNHSGDYDYFDY

SEQ ID NO: 250 - protein, 463 aa, synthetic construct
QVQLVQSGAEVKKPGASVKVSCKASGYTFTSYYMHWVRQAPGQGLEWMGIINPSRGSASYAQKFQGRVTMARDTSTSTVYMELSSLRSEDTAVYYCARDRNYYYYMDVWGKGTTVTVSSGGGGSGGGGSGGGGSEIVMTQSPATLSVSPGERATLSCRASQNISSNLAWYQQKPGQAPRLLIYGASTRATDIPARFSGSGSGTEFTLTISSLQSEDFAVYYCQQYITWYTFGQGTKLEIKAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 251 - protein, 353 aa, synthetic construct
EVQLVESGGGLVQPGGSLRLSCAASGFSFSDYSGMSWVRQAPGKGEELVSAISPGGGDTYYADSVKGRFTISRDNSKNTLYLQMNSLRAEDTAVYYCARRWWYYSNHSGDYDYFDYRGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 252 - protein, 816 aa, synthetic construct
EIVLTQSPGTLSLSPGERATVSCRASQSVSSSYLAWYQQKPGQAPRLLIYGASSRATGIPDRFSGSGSGTDFTLTISRLEPEDFAVYYCQQYGSSPYTFGQGTKLEIKGGGGSGGGGSGGGGSQVQLVQSGPEVKKPGASVKVSCKASGYTFTSYGITWVRQAPGQGLEWMGWISEYNGNTNYAQKFQGRVTMTIDTSTTTAYMELRSLRSDDTAVYFCARGGILPYYFFYYMDVWGKGTTVTVSSATTTPAPRPPTPAPTIASQPLSLRPEACFMYVAAAAFVLLFFVGCGVLLSRKRRRQHGQLWFPEGFKVSEASKKKRREPLGEDSVGLKPLKNAMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 253 - protein, 816 aa, synthetic construct
EIVLTQSPATLSLSPGERATLSCGASQSVSSSYLAWYQQKPGLAPRLLIYDASSRATGIPDRFSGSGSGTDFTLTISRLEPEDFAVYYCQQYGSSPYTFGQGTKLEIKGGGGSGGGGSGGGGSQVQLVQSGAEVKKPGASVKVSCKASGYTFSSYGVSWVRQAPGQGLEWMGWISKYNGNTNYAQKFQGRVTMTTDTSTSTAYMELRSLRSDDTAVYFCARGGIHGDSYYFYYLDVWGKGTTVTVSSATTTPAPRPPTPAPTIASQPLSLRPEACFMYVAAAAFVLLFFVGCGVLLSRKRRRQHGQLWFPEGFKVSEASKKKRREPLGEDSVGLKPLKNAMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQIS

SEQ ID NO: 254 - protein, 320 aa, synthetic construct
ELPTQGTFSNVSTNVSELCGGAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 255 - protein, 249 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 256 - DNA, 140 bp, synthetic construct
  source 1..140: mol_type = other DNA
gttaataattaacatatatgttaatcattaacatatagttaattattaaccgctatgttaatgattaacaacggttaataattaacatatatgttaatcattaacatataactagtctagagggtatataatgggggcca

SEQ ID NO: 257 - protein, 277 aa, synthetic construct
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 258 - protein, 22 aa, synthetic construct
AIVVPVCLAFLLTTLLGVLFCF

SEQ ID NO: 259 - protein, 378 aa, synthetic construct
MALPVTALLLPLALLLHAARPDYKDDDDKELCGGRIARLEEKVKTLKAQNSELASTANMLREQVAQLKQKVMNAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 260 - protein, 16 aa, synthetic construct
ELPTQGTFSNVSTNVS

SEQ ID NO: 261 - DNA, 960 bp, synthetic construct
  source 1..960: mol_type = other DNA
gagctgcccacacaaggcacattcagcaacgtgtccaccaacgtgtccgagctgtgcggcggcgctatcgtggtgcccgtgtgcttggccttcctgctgaccaccctgctgggcgtgctgttctgcttcaacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcag

SEQ ID NO: 262 - DNA, 1023 bp, synthetic construct
  source 1..1023: mol_type = other DNA
atggccctgcccgtgacagccctgctcctgcctctggccctgttgctgcatgcagcacggcctgagctgcccacacaaggcacattcagcaacgtgtccaccaacgtgtccgagctgtgcggcggcgctatcgtggtgcccgtgtgcttggccttcctgctgaccaccctgctgggcgtgctgttctgcttcaacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcag

SEQ ID NO: 263 - protein, 10 aa, synthetic construct
GYTFSSYGVS

SEQ ID NO: 264 - protein, 7 aa, synthetic construct
ENLYTQS

SEQ ID NO: 265 - protein, 5 aa, synthetic construct
DDDDK

SEQ ID NO: 266 - protein, 4 aa, synthetic construct
LVPR

SEQ ID NO: 267 - protein, 4 aa, synthetic construct
CPPC

SEQ ID NO: 268 - RNA, 103 nt, synthetic construct
  source 1..103: mol_type = other RNA
ggggccactagggacaggatgttttagagctagaaatagcaagttaaaataaggctagtccgttatcaacttgaaaaagtggcaccgagtcggtgcttttttt

SEQ ID NO: 269 - DNA, 20 bp, Adeno-associated virus
  source 1..20: mol_type = genomic DNA
ggggccactagggacaggat

SEQ ID NO: 270 - RNA, 20 nt, synthetic construct
  source 1..20: mol_type = other RNA
gcacctgaataccacgcctg

SEQ ID NO: 271 - RNA, 20 nt, synthetic construct
  source 1..20: mol_type = other RNA
cgcctgcgatgtagtcgatg

SEQ ID NO: 272 - RNA, 20 nt, synthetic construct
  source 1..20: mol_type = other RNA
caggacgggcgagatgtccc

SEQ ID NO: 273 - RNA, 20 nt, synthetic construct
  source 1..20: mol_type = other RNA
ctgaatctttggagtacctg

SEQ ID NO: 274 - RNA, 20 nt, synthetic construct
  source 1..20: mol_type = other RNA
ggccacggagcgagacatct

SEQ ID NO: 275 - RNA, 20 nt, synthetic construct
  source 1..20: mol_type = other RNA
aagtcaacttcaatgtcgga

SEQ ID NO: 276 - RNA, 20 nt, synthetic construct
  source 1..20: mol_type = other RNA
gcttggaggcctgatcagcg

SEQ ID NO: 277 - RNA, 20 nt, synthetic construct
  source 1..20: mol_type = other RNA
cttatctcttcgcagcgagg

SEQ ID NO: 278 - RNA, 20 nt, synthetic construct
  source 1..20: mol_type = other RNA
cacacattactccaacattg

SEQ ID NO: 279 - RNA, 20 nt, synthetic construct
  source 1..20: mol_type = other RNA
ttccgcaaaatagagcccca

SEQ ID NO: 280 - RNA, 20 nt, synthetic construct
  source 1..20: mol_type = other RNA
tgcacagaactatcgtacca

SEQ ID NO: 281 - RNA, 20 nt, synthetic construct
  source 1..20: mol_type = other RNA
gcaataagactctttaaaga

SEQ ID NO: 282 - RNA, 20 nt, synthetic construct
  source 1..20: mol_type = other RNA
caaagagattacgaatgcct

SEQ ID NO: 283 - RNA, 20 nt, synthetic construct
  source 1..20: mol_type = other RNA
caaggcaccccaggtttcca

SEQ ID NO: 284 - RNA, 20 nt, synthetic construct
  source 1..20: mol_type = other RNA
ttacgaatgccttggaaacc

SEQ ID NO: 285 - RNA, 20 nt, synthetic construct
  source 1..20: mol_type = other RNA
cagagacgcatctgaccctc

SEQ ID NO: 286 - RNA, 20 nt, synthetic construct
  source 1..20: mol_type = other RNA
catgcagttctcacacactg

SEQ ID NO: 287 - RNA, 20 nt, synthetic construct
  source 1..20: mol_type = other RNA
gtgtgagaactgcatggaga

SEQ ID NO: 288 - RNA, 20 nt, synthetic construct
  source 1..20: mol_type = other RNA
tctcatttcaggaaaccact

SEQ ID NO: 289 - RNA, 20 nt, synthetic construct
  source 1..20: mol_type = other RNA
agtcatacaccttaaccaag

SEQ ID NO: 290 - RNA, 20 nt, synthetic construct
  source 1..20: mol_type = other RNA
ttcaaggaaaccagttgagg

SEQ ID NO: 291 - RNA, 20 nt, synthetic construct
  source 1..20: mol_type = other RNA
gagccttgcctggaaatctg

SEQ ID NO: 292 - RNA, 20 nt, synthetic construct
  source 1..20: mol_type = other RNA
aagcgtcaaaagtctgccag

SEQ ID NO: 293 - RNA, 20 nt, synthetic construct
  source 1..20: mol_type = other RNA
cgttccaactcgaagtgcca

SEQ ID NO: 294 - RNA, 20 nt, synthetic construct
  source 1..20: mol_type = other RNA
gagcgactgggacacggtga

SEQ ID NO: 295 - RNA, 20 nt, synthetic construct
  source 1..20: mol_type = other RNA
gctgcgcaagaagggcccta

SEQ ID NO: 296 - RNA, 20 nt, synthetic construct
  source 1..20: mol_type = other RNA
ttgttctggccagcagcccc

SEQ ID NO: 297 - RNA, 20 nt, synthetic construct
  source 1..20: mol_type = other RNA
cttccagagccacatcatcg

SEQ ID NO: 298 - RNA, 20 nt, synthetic construct
  source 1..20: mol_type = other RNA
gggactcaccagagagaggt

SEQ ID NO: 299 - RNA, 20 nt, synthetic construct
  source 1..20: mol_type = other RNA
cggtcgaaatagaagcccta

SEQ ID NO: 300 - RNA, 20 nt, synthetic construct
  source 1..20: mol_type = other RNA
aaaaggatattgtgcaactg

SEQ ID NO: 301 - RNA, 20 nt, synthetic construct
  source 1..20: mol_type = other RNA
tgtgcatatttattacatcg

SEQ ID NO: 302 - RNA, 20 nt, synthetic construct
  source 1..20: mol_type = other RNA
tttgtgaagatcttgaccaa

SEQ ID NO: 303 - RNA, 20 nt, synthetic construct
  source 1..20: mol_type = other RNA
tgtcatgctgaaccgcattg

SEQ ID NO: 304 - RNA, 20 nt, synthetic construct
  source 1..20: mol_type = other RNA
ccactctatgaggatagtca

SEQ ID NO: 305 - RNA, 20 nt, synthetic construct
  source 1..20: mol_type = other RNA
ttgacatagaagaggcacaa

SEQ ID NO: 306 - RNA, 20 nt, synthetic construct
  source 1..20: mol_type = other RNA
gagtactacactcagcagca

SEQ ID NO: 307 - RNA, 20 nt, synthetic construct
  source 1..20: mol_type = other RNA
tcacgcacaagaaacgtcca

SEQ ID NO: 308 - RNA, 20 nt, synthetic construct
  source 1..20: mol_type = other RNA
aggtctcggtgaaaccacct

SEQ ID NO: 309 - RNA, 20 nt, synthetic construct
  source 1..20: mol_type = other RNA
agcattatccaaagagtccg

SEQ ID NO: 310 - RNA, 20 nt, synthetic construct
  source 1..20: mol_type = other RNA
atattaattcttaccagtgg

SEQ ID NO: 311 - RNA, 20 nt, synthetic construct
  source 1..20: mol_type = other RNA
agctttaaatcaaggttcat

SEQ ID NO: 312 - RNA, 20 nt, synthetic construct
  source 1..20: mol_type = other RNA
atcccgagccctaaggtgca

SEQ ID NO: 313 - RNA, 20 nt, synthetic construct
  source 1..20: mol_type = other RNA
ggcagcgcggaggacagcgt

SEQ ID NO: 314 - RNA, 20 nt, synthetic construct
  source 1..20: mol_type = other RNA
ctcagggggctactaccacc

SEQ ID NO: 315 - RNA, 20 nt, synthetic construct
  source 1..20: mol_type = other RNA
gtcaccgacgagaccagaag

SEQ ID NO: 316 - RNA, 20 nt, synthetic construct
  source 1..20: mol_type = other RNA
gtcgtggacttcgtactgct

SEQ ID NO: 317 - RNA, 20 nt, synthetic construct
  source 1..20: mol_type = other RNA
taatttttaggcaagtgtcg

SEQ ID NO: 318 - RNA, 20 nt, synthetic construct
  source 1..20: mol_type = other RNA
ttagctgttagacttgaata

SEQ ID NO: 319 - RNA, 20 nt, synthetic construct
  source 1..20: mol_type = other RNA
cgagagccgtcaacttgcgt

SEQ ID NO: 320 - RNA, 20 nt, synthetic construct
  source 1..20: mol_type = other RNA
cggcttcaactgcaaaggtg

SEQ ID NO: 321 - RNA, 20 nt, synthetic construct
  source 1..20: mol_type = other RNA
tatgaaaaagcagagcgact

SEQ ID NO: 322 - RNA, 20 nt, synthetic construct
  source 1..20: mol_type = other RNA
tctggcgggcgagctcacgc

SEQ ID NO: 323 - RNA, 20 nt, synthetic construct
  source 1..20: mol_type = other RNA
ctcacgctggttaccgccta

SEQ ID NO: 324 - RNA, 20 nt, synthetic construct
  source 1..20: mol_type = other RNA
aaagattacgaacttccctg

SEQ ID NO: 325 - RNA, 20 nt, synthetic construct
  source 1..20: mol_type = other RNA
gttaaaaacagacatgccta

SEQ ID NO: 326 - RNA, 20 nt, synthetic construct
  source 1..20: mol_type = other RNA
atgcctaaggaggttgtacc

SEQ ID NO: 327 - RNA, 20 nt, synthetic construct
  source 1..20: mol_type = other RNA
ctccaggtatcccatcgaaa

SEQ ID NO: 328 - RNA, 20 nt, synthetic construct
  source 1..20: mol_type = other RNA
caccaaatacgatagatcag

SEQ ID NO: 329 - RNA, 20 nt, synthetic construct
  source 1..20: mol_type = other RNA
tggcggcgtgaatggcaaga

SEQ ID NO: 330 - RNA, 20 nt, synthetic construct
  source 1..20: mol_type = other RNA
taggatggtagcacacaacc

SEQ ID NO: 331 - RNA, 20 nt, synthetic construct
  source 1..20: mol_type = other RNA
cagcagcagagccccgacgg

SEQ ID NO: 332 - RNA, 20 nt, synthetic construct
  source 1..20: mol_type = other RNA
cggcgtgcgaacggaatgtg

SEQ ID NO: 333 - RNA, 20 nt, synthetic construct
  source 1..20: mol_type = other RNA
tatagacgctgcccgacgtc

SEQ ID NO: 334 - RNA, 20 nt, synthetic construct
  source 1..20: mol_type = other RNA
tccaaagaagggtactgtgg

SEQ ID NO: 335 - RNA, 20 nt, synthetic construct
  source 1..20: mol_type = other RNA
acagtacccttctttggaat

SEQ ID NO: 336 - RNA, 20 nt, synthetic construct
  source 1..20: mol_type = other RNA
gcgacgggcgcatctacgtg

SEQ ID NO: 337 - RNA, 20 nt, synthetic construct
  source 1..20: mol_type = other RNA
cccgacctccataagtcctg

SEQ ID NO: 338 - RNA, 20 nt, synthetic construct
  source 1..20: mol_type = other RNA
ggggtcctcgaagcgcacga

SEQ ID NO: 339 - RNA, 20 nt, synthetic construct
  source 1..20: mol_type = other RNA
tgctctgtttagaagatgac

SEQ ID NO: 340 - RNA, 20 nt, synthetic construct
  source 1..20: mol_type = other RNA
atattcttttctagttaaag

SEQ ID NO: 341 - RNA, 20 nt, synthetic construct
  source 1..20: mol_type = other RNA
cctgtaaagaaacaaaagac

SEQ ID NO: 342 - RNA, 20 nt, synthetic construct
  source 1..20: mol_type = other RNA
tggagaaagacgtaacttcg

SEQ ID NO: 343 - RNA, 20 nt, synthetic construct
  source 1..20: mol_type = other RNA
tctgccctgaggtatgcgat

SEQ ID NO: 344 - RNA, 20 nt, synthetic construct
  source 1..20: mol_type = other RNA
attccgcttggtgaaaacga

SEQ ID NO: 345 - RNA, 20 nt, synthetic construct
  source 1..20: mol_type = other RNA
caggcacaatagaaacaacg

SEQ ID NO: 346 - RNA, 20 nt, synthetic construct
  source 1..20: mol_type = other RNA
ccatttgtaatgctgacttg

SEQ ID NO: 347 - RNA, 20 nt, synthetic construct
  source 1..20: mol_type = other RNA
ctgggtcacttgtgccgtgg

SEQ ID NO: 348 - RNA, 20 nt, synthetic construct
  source 1..20: mol_type = other RNA
gtcagggttctggatatctg

SEQ ID NO: 349 - RNA, 20 nt, synthetic construct
  source 1..20: mol_type = other RNA
tggatttagagtctctcagc

SEQ ID NO: 350 - RNA, 20 nt, synthetic construct
  source 1..20: mol_type = other RNA
ctgcggctgtggtccagctg

SEQ ID NO: 351 - RNA, 20 nt, synthetic construct
  source 1..20: mol_type = other RNA
acaaaactgtgctagacatg

SEQ ID NO: 352 - RNA, 20 nt, synthetic construct
  source 1..20: mol_type = other RNA
ttcttccccagcccaggtaa

SEQ ID NO: 353 - RNA, 20 nt, synthetic construct
  source 1..20: mol_type = other RNA
cgtcatgagcagattaaacc

SEQ ID NO: 354 - RNA, 20 nt, synthetic construct
  source 1..20: mol_type = other RNA
gagagcgcctgcgacccgag

SEQ ID NO: 355 - RNA, 20 nt, synthetic construct
  source 1..20: mol_type = other RNA
ccagcgggtgaagtacacca

SEQ ID NO: 356 - RNA, 20 nt, synthetic construct
  source 1..20: mol_type = other RNA
ggagcgcttttcgccgccag

SEQ ID NO: 357 - RNA, 20 nt, synthetic construct
  source 1..20: mol_type = other RNA
tgaggcctggaccttatgca

SEQ ID NO: 358 - RNA, 20 nt, synthetic construct
  source 1..20: mol_type = other RNA
cctggtggagtgaaccatga

SEQ ID NO: 359 - RNA, 20 nt, synthetic construct
  source 1..20: mol_type = other RNA
caagcacttaggttcccctg

SEQ ID NO: 360 - RNA, 20 nt, synthetic construct
  source 1..20: mol_type = other RNA
ggtctccctacaattcagcg

SEQ ID NO: 361 - RNA, 20 nt, synthetic construct
  source 1..20: mol_type = other RNA
cacagcgcgtgactgcaatg

SEQ ID NO: 362 - RNA, 20 nt, synthetic construct
  source 1..20: mol_type = other RNA
tctggggcaccaattctagg

SEQ ID NO: 363 - RNA, 20 nt, synthetic construct
  source 1..20: mol_type = other RNA
gagccatgcttggcttacga

SEQ ID NO: 364 - RNA, 20 nt, synthetic construct
  source 1..20: mol_type = other RNA
gtacaagtacttatctcatg

SEQ ID NO: 365 - RNA, 20 nt, synthetic construct
  source 1..20: mol_type = other RNA
gagataacaacataacaaca

SEQ ID NO: 366 - RNA, 20 nt, synthetic construct
  source 1..20: mol_type = other RNA
catattccatagtctttggg

SEQ ID NO: 367 - RNA, 20 nt, synthetic construct
  source 1..20: mol_type = other RNA
ctgccccttagcaacttagg

SEQ ID NO: 368 - RNA, 20 nt, synthetic construct
  source 1..20: mol_type = other RNA
tgtttaaaaatatgttgaca

SEQ ID NO: 369 - RNA, 20 nt, synthetic construct
  source 1..20: mol_type = other RNA
ccaggaatggaaactcacgc

SEQ ID NO: 370 - RNA, 20 nt, synthetic construct
  source 1..20: mol_type = other RNA
gaggccgctgaattaacccg

SEQ ID NO: 371 - RNA, 20 nt, synthetic construct
  source 1..20: mol_type = other RNA
atacacgcacacttgcagaa

SEQ ID NO: 372 - RNA, 20 nt, synthetic construct
  source 1..20: mol_type = other RNA
gagcagacagaaacccaggg

SEQ ID NO: 373 - RNA, 20 nt, synthetic construct
  source 1..20: mol_type = other RNA
tgagtctccaaacagaacag

SEQ ID NO: 374 - RNA, 20 nt, synthetic construct
  source 1..20: mol_type = other RNA
taatatcactgacttcacgg

SEQ ID NO: 375 - RNA, 20 nt, synthetic construct
  source 1..20: mol_type = other RNA
tacacacaatgtaagcagca

SEQ ID NO: 376 - RNA, 20 nt, synthetic construct
  source 1..20: mol_type = other RNA
gggagctcaattcgaaacca

SEQ ID NO: 377 - RNA, 20 nt, synthetic construct
  source 1..20: mol_type = other RNA
ttggacaggtgagacagtcg

SEQ ID NO: 378 - RNA, 20 nt, synthetic construct
  source 1..20: mol_type = other RNA
aagctcactcagatagtgtg

SEQ ID NO: 379 - RNA, 20 nt, synthetic construct
  source 1..20: mol_type = other RNA
caggagaaccaccttacacg

SEQ ID NO: 380 - RNA, 20 nt, synthetic construct
  source 1..20: mol_type = other RNA
ggacagaccctgattcacaa

SEQ ID NO: 381 - RNA, 20 nt, synthetic construct
  source 1..20: mol_type = other RNA
acatggcagtctatgaacag

SEQ ID NO: 382 - RNA, 20 nt, synthetic construct
  source 1..20: mol_type = other RNA
cctatagagagtactacttg

SEQ ID NO: 383 - RNA, 20 nt, synthetic construct
  source 1..20: mol_type = other RNA
ccaaccgggtcttcattacg

SEQ ID NO: 384 - RNA, 20 nt, synthetic construct
  source 1..20: mol_type = other RNA
tcaagcgtagagttccgagt

SEQ ID NO: 385 - RNA, 20 nt, synthetic construct
  source 1..20: mol_type = other RNA
tcatgcaattatggacccag

SEQ ID NO: 386 - RNA, 20 nt, synthetic construct
  source 1..20: mol_type = other RNA
cgggaaagtgactggccatg

SEQ ID NO: 387 - RNA, 20 nt, synthetic construct
  source 1..20: mol_type = other RNA
tgagattgaaatcaaatcgg

SEQ ID NO: 388 - RNA, 20 nt, synthetic construct
  source 1..20: mol_type = other RNA
tatgcaatattcatcacgcg

SEQ ID NO: 389 - RNA, 20 nt, synthetic construct
  source 1..20: mol_type = other RNA
aatgtgttaaatcaaatgca